(19) United States
(12) Patent Application Publication
(10) Pub. No.: US 2026/0265766 A1
(43) Pub. Date: Sep. 10, 2026

(54) 3-HYDROXYPROPIONIC ACID PRODUCTION USING YEAST AT AN INDUSTRIALLY RELEVANT SCALE

(71) Applicant: The Board of Trustees of the University of Illinois, Urbana, IL (US)

(72) Inventors: Huimin Zhao, Champaign, IL (US); Teresa Martin, Urbana, IL (US); Shi-I Tan, Champaign, IL (US); Chandan Mahata, Urbana, IL (US); Somesh Mishra, Urbana, IL (US); Vijay Singh, Champaign, IL (US)

(73) Assignee: The Board of Trustees of the University of Illinois, Urbana, IL (US)

(21) Appl. No.: 19/556,670

(22) Filed: Mar. 4, 2026

Related U.S. Application Data
(60) Provisional application No. 63/766,638, filed on Mar. 4, 2025.

(51) Int. Cl. C12N 15/81; C12N 1/16; C12N 9/00; C12N 9/04; C12N 9/10; C12N 9/88; C12P 7/52; C12R 1/84

(52) U.S. Cl. CPC C12N 15/815; C12N 1/16; C12N 9/0006; C12N 9/1096; C12N 9/88; C12N 9/93; C12P 7/52; C12R 2001/84; C12Y 101/01008; C12Y 101/01059; C12Y 206/01001; C12Y 206/01018; C12Y 401/01001; C12Y 401/01011; C12Y 604/01001

(57) ABSTRACT

The present disclosure provides a microbial fermentation process to cost-effectively produce an industrially important commodity chemical 3-hydroxypropionic acid at industrial pilot scale. Also provided are yeast strains capable of converting glucose and carbon dioxide to 3-hydroxypropionic acid with high titer and yield at low pH. A method for a simple downstream processing strategy to recover 3-hydroxypropionic acid as sodium salt is further provided.

### PRIORITY

This application claims the benefit of U.S. Ser. No. 63/766,638, filed on Mar. 4, 2025, which is incorporated herein by reference in its entirety.

### GOVERNMENT SUPPORT

This invention was made with government support under DE-SC0018420 awarded by the Department of Energy. The government has certain rights in the invention.

### SEQUENCE LISTING

The specification incorporates by reference a Sequence Listing submitted via Patent Center. The Sequence Listing .xml file, identified as 775595_UIUC-059.xml, is 264,905 bytes in size and was created on Apr. 20, 2026.

### BACKGROUND

Biomanufacturing has been increasingly used to produce chemicals and materials from renewable plant biomass¹⁻³. 3-Hydroxypropionic acid (3HP) is one of the top 12 sugar-based platform chemicals that were identified by the United States Department of Energy to have great potential for commercial applications.⁴⁻⁸ For example, it can be converted to acrylic acid, 1,3-propanediol, methyl acrylate, acrylamide, ethyl 3HP, malonic acid, propiolactone, and acrylonitrile.^{6,9,10} In particular, acrylic acid represents a large market opportunity (approximately US$20 billion), with a global annual demand of approximately 6.6 million tons in 2019 and is expected to grow by more than 5% every year.¹¹ Currently, both 3HP and acrylic acid are produced almost exclusively via chemical synthesis routes, which rely on fossil-derived feedstocks and involve energy-intensive processes that contribute to greenhouse gas emissions and environmental pollution.^{9,12,13}

Both bacteria and yeasts have been extensively explored for biological production of 3HP in the past few decades¹⁴. Bacteria such as Escherichia coli have been engineered to produce 3HP with high yields and titers (0.6 g/g yield with a titer of 72 g/L¹⁵). However, the inhibitory effects of low pH on bacterial growth necessitates the use of neutralization agents such as calcium carbonate to maintain fermentations at neutral pH, i.e. above the pKa of 3HP (4.5). As a result, an acidification step is necessary to recover 3HP, which leads to the formation of gypsum, rendering downstream processing complex and costly (FIG. 6). In contrast, due to their tolerance to low pH, yeasts can be engineered to produce 3HP at low pH, which greatly simplifies the whole 3HP production process and eliminates the formation of gypsum. A previous techno-economic analysis of the theoretical fermentation space suggested a yield of >0.78 g/g combined with a titer of >80 g/L or a yield of >0.62 g/g combined with a titer of >60 g/L would be needed to achieve financial viability under neutral and low-pH regimes, respectively¹⁶. However, to the best of our knowledge, the highest titer and yield achieved using a yeast are 100.4 g/L (with a yield of 0.2 g/g)¹⁷ and 0.56 g/g (with a titer of 11.25 g/L)¹⁸, respectively. Methods are needed in the art to increase the yield and titer of 3HP production.

### SUMMARY

Provided herein are recombinant yeast comprising one or more heterologous polynucleotides encoding 2, 3, 4, 5, 6, 7, or 8 copies of an aspartate 1-decarboxylase (PAND) polypeptide. The one or more heterologous polynucleotides encoding one or more copies of an PAND polypeptide can be derived from Apolygus lucorum. The one or more heterologous polynucleotides can encode a PAND polypeptide comprising 90% or greater sequence identity to the sequence set forth in SEQ ID NO:213. A pyruvate decarboxylase (PDC) gene of the recombinant yeast can be inactivated. A glycerol-3-phosphate dehydrogenase (GPD) gene of the recombinant yeast can be inactivated. The recombinant yeast can further comprise one or more heterologous polynucleotides encoding one or more copies of a pyruvate carboxylase (PYC) polypeptide, or a PYC polypeptide or gene that is overexpressed. The recombinant yeast can further comprise one or more heterologous polynucleotides encoding two or more copies of a pyruvate carboxylase (PYC) polypeptide, or a PYC polypeptide or gene that is overexpressed. The recombinant yeast can further comprise one or more heterologous polynucleotides encoding one or more copies of an aspartate aminotransferase (AAT) polypeptide or an ATT polypeptide or gene that is overexpressed. The recombinant yeast can further comprise one or more heterologous polynucleotides encoding two or more copies of an aspartate aminotransferase (AAT) polypeptide or an ATT polypeptide or gene that is overexpressed. The recombinant yeast can further comprise one or more heterologous polynucleotides encoding one or more copies of a β-alanine-pyruvate aminotransferase (BAPAT) polypeptide. The one or more heterologous polynucleotides encoding a BAPAT polypeptide can comprise 90% or greater sequence identity to the sequence set forth in SEQ ID NO:228-234. The one or more heterologous polynucleotides encoding one or more copies of BAPAT can be derived from Pseudomonas sp., Pichia sp., Burkholderia sp., Mesorhizobium sp., Falkowbacteria sp., and/or Klebsiella sp. The recombinant yeast can further comprise one or more heterologous polynucleotides encoding one or more copies of a 3HP dehydrogenase (YDFG) polypeptide. The one or more heterologous polynucleotides encoding a YDFG polypeptide can comprise 90% or greater sequence identity to the sequence set forth in SEQ ID NO:218-227. The one or more heterologous polynucleotides encoding one or more copies of a YDFG polypeptide can be derived from Beauveria sp., Plautia sp., Erwinia sp., Helicobacter sp., Pseudomonas sp., Acetobacter sp., Gluconobacter sp., Oleiagrimonas sp., Geobacter sp., and/or Campylobacter sp. The one or more heterologous polynucleotides encoding one or more copies of a BAPAT polypeptide or a YDFG polypeptide can be integrated into the recombinant yeast's chromosome. The recombinant yeast can be Issatchenkia sp. The recombinant yeast can be Issatchenkia orientalis.

Another aspect provides a recombinant yeast comprising: one or more heterologous polynucleotides encoding 2, 3, 4, 5, 6, 7, or 8 copies of an aspartate 1-decarboxylase (PAND) polypeptide; an inactivated pyruvate decarboxylase (PDC) gene; an inactivated glycerol-3-phosphate dehydrogenase (GPD) gene; one or more heterologous polynucleotides encoding one or more copies of a pyruvate carboxylase (PYC) polypeptide or a PYC polypeptide or gene is overexpressed; one or more heterologous polynucleotides encoding one or more copies of an aspartate aminotransferase (AAT) polypeptide or an AAT polypeptide or gene that is overexpressed; one or more heterologous polynucleotides encoding one or more copies of a β-alanine-pyruvate aminotransferase (BAPAT) polypeptide; and one or more heterologous polynucleotides encoding one or more copies of a 3HP dehydrogenase (YDFG) polypeptide.

Yet another aspect provides a method of producing 3-hydroxypropionic acid (3HP) comprising adding any of the recombinant yeast described herein to cell culture media, cultivating the recombinant yeast to a desired cell density, and harvesting the 3HP. The 3HP can be produced at 75 g/L or more, or at about 90 g/L or more. A yield of the 3HP can be 0.5 g/g glucose or more, or 0.7 g/g glucose or more. The pH of the cell culture media can be 3.75 to 4.25. Dissolved oxygen of the cell culture media can be 4-6%. The cell culture media can be corn steep liquor. Less than 0.7 g/L of pyruvate can be produced. The method can further comprise providing CO₂ to the cell culture media during a 3HP production phase.

Even another aspect provides a method of isolating sodium 3-hydroxypropionic acid from a fermentation broth comprising: (a) removing cells from the fermentation broth to form a cell-free fermentation broth; (b) decolorizing the cell-free fermentation broth to form a cell-free, decolorized fermentation broth; (c) removing cations and anions from the cell-free, decolorized fermentation broth; and (d) adding NaOH to the product of step (c) to form sodium 3-hydroxypropionic acid. The cell-free fermentation broth can be concentrated prior to step (b). The method can further comprise evaporating and drying the 3-hydroxypropionic acid to form a solid. The method can further comprise isolating a 20-40% (w/v) 3-hydroxypropionic acid solution through evaporation or concentration. The cations can be removed using a cation exchange resin and the anions are removed using an anion exchange resin. The method can be solvent-free.

The sodium 3-hydroxypropionic acid can be 80% w/w or more pure. The sodium 3-hydroxypropionic acid can be recovered at 70% w/w or more. The fermentation broth can be produced by fed-batch fermentation, continuous fermentation, batch fermentation, or combinations thereof.

Another aspect provides polynucleotide molecule encoding a polypeptide as set forth in SEQ ID NO:1-10 or 193-208, wherein the polynucleotide molecule is operably linked to a heterologous yeast promoter. The polynucleotide can further comprise a heterologous yeast terminator.

Another aspect provides a polynucleotide molecule encoding a polypeptide as set forth in SEQ ID NO:1-10 or 193-208, wherein the polynucleotide molecule is operably linked to a marker polynucleotide.

### BRIEF DESCRIPTION OF FIGURES

FIG. 1 Shows simulated production envelopes for 3HP pathways. Pathway numbers, indicated by roman numerals, follow the numbering in Table 2 in a previous study⁷, i.e., I: pyruvate→lactate→lactoyl-CoA→acryloyl-CoA→3HP-CoA→3HP; II: pyruvate→acetyl-CoA-malonyl-CoA→3-oxopropanoate→3HP; III: pyruvate→oxaloacetate-→aspartate→β-alanine→3-oxopropanoate-→3HP; IV: pyruvate→oxaloacetate-→aspartate→-alanine→β-alanyl-CoA→acryloyl-CoA→3HP-CoA→3HP; V: pyruvate→succinate→propionyl-CoA→acryloyl-CoA→3HP-CoA→3HP; VI: pyruvate→α-alanine→β-alanine→3-oxopropanoate→3HP; VII: pyruvate→α-alanine→-alanine→β-alanyl-CoA→acryloyl-CoA→3HP-CoA→3HP. Pathway III corresponds to the β-alanine pathway in the current work, with superscript p indicating NADPH utilization. Biomass yield (left) and oxygen uptake (right) envelopes were generated using the genome-scale metabolic model ilsor850²³ with reactions added as needed to enable 3HP production by the indicated pathway, and with the indicated gene deletions. Shading in the biomass yield denotes the corresponding minimal oxygen uptake rate.

FIG. 2 panels A-C show discovery and characterization of new pathway genes in the β-alanine pathway for 3HP production. (A) β-alanine accumulation in the strain overexpressing the PAND candidate genes. (B) in vitro assay for the BAPAT and YDFG genes. (C) OD₅₄₀ measurement for in vitro BAPAT assay. (D) 3HP measurement for in vitro YDFG assay. Data present mean±S.D. of three biologically independent samples (n=3). Created in BioRender. Zhao, H. (2025) BioRender.com/vkz7w01.

FIG. 3 panels A-B show metabolic engineering strategies used to increase 3HP production in I. orientalis SD108. (A) 3HP production using the β-alanine pathway. GPD, glycerol-3-phosphate dehydrogenase; PDC, pyruvate decarboxylase; PYC, pyruvate carboxylase; PAND, aspartate decarboxylase; BAPATA, β-alanine aminotransferase; YDFG, 3HP dehydrogenase. OAA, oxaloacetate; ASP, aspartate; β-ala, β-alanine; MSA, malonic semialdehyde, 3HP, 3-hydroxypropionic acid. (B) 3HP production using various engineered strains. Episomal plasmids PBY, P, B and Y means the strains harbored an episomal plasmid encoding PBY, P, B and Y, respectively. Horizontal hatch marks are for comparison of the original gene set (column 1) and new gene set from SSN (column 2). Right diagonal hatch marks are for discovering the copy number effect and rate-limiting step. Left diagonal hatch marks are for PAND copy effect. The empty bars reflect further metabolic engineering strategies, including knocking out the PDC and GPD genes, integrating all pathway genes, and overexpressing the PYC and AAT genes. Data present mean±S.D. of at least three biologically independent samples (n=3) unless specified. For column 3 and 4, data present mean±S.D. of five biologically independent samples (n=5). For column 5, data present mean±S.D. of seven biologically independent samples (n=7). Created in BioRender. Zhao, H. (2025) BioRender.com/vkz7w01.

FIG. 4 panels A-D show optimization for fed-batch fermentation using 2AP strain. The profile of the 2AP strain for glucose consumption (A), pyruvate accumulation (B), and 3HP production (C) in both the CSM medium and the CSL medium in shake flask. Data present mean±S.D. of three biologically independent samples (n=3). (D) fed-batch fermentation a 300 mL. Glucose feeding began at 24 hours post inoculation. The 2AP strain was used for fermentation. During fermentation, pyruvate, and glucose were monitored. No pyruvate accumulation or other metabolites greater than 1 g/L were observed. The cultivation was set at DO ranging from 5% to 15% and pH at 4 with CO₂ gassing. For fed-batch fermentation, two independent biological replicates were performed (n=2) and mean value from two replicates is presented. Source data for this Created in BioRender. Zhao, H. (2025) BioRender.com/vkz7w01

FIG. 5 panels A-G show uncertainty and sensitivity of system cost and carbon intensity for the production of acrylic acid via 3-hydroxypropionic acid from several alternative feedstocks. Uncertainties (box-and-whisker plots) for (A) minimum product selling price (MPSP) and (B) cradle-to-grave carbon intensity (CI) quantified as 100-year global warming potential per kg of acrylic acid produced by catalytic upgrading of 3HP produced via microbial conversion of sugars by I. orientalis. Box-and-whisker plots show results for four scenarios (markers report results for baseline values): (i) fermentation performance achieved in the DASbox using dextrose (dextrose, diamond markers); (ii) corn-based 3HP biomanufacturing under the same assumptions for fermentation performance as that achieved in the DASbox (corn, square markers); (iii) sugarcane based 3HP biomanufacturing under the same assumptions for fermentation performance as that achieved in the DASbox (sugarcane, triangle markers); and (iv) corn stover based 3HP biomanufacturing under the same assumptions for fermentation performance as that achieved in the DASbox (corn stover, pentagon markers; baseline values and distributions for all parameters included in the uncertainty analysis for each scenario are detailed in Table 19). On box-and-whisker plots, whiskers, boxes, and the middle line represent 5^{th}/95^{th} 25^{th}/75^{th}, and 50^{th} percentiles, respectively, from 6000 Monte Carlo simulations for each scenario. (C,E) MPSP and (D,F) Cl for dextrose-accepting biomanufacturing across 2500 fermentation yield-titer combinations at the baseline productivity (0.548 g/L/h) for neutral (middle panels; C,D) and low-pH (right panels; E,F) fermentation. Yield is shown as % theoretical maximum (% theoretical) scaled to the theoretical maximum yield of 1.00 g/g-glucose-equivalent (based on carbon balance). Diamond markers report results for baseline values for the dextrose scenario. Shaded gray and hatched regions show (A,C,E) market price range for acrylic acid ($1.40-1.65/kg)³⁵ and (B,D,F) a range bounded by Cl values reported for fossil-based acrylic acid^{36, 37} (end-of-life impact of 1.83 kg CO₂-eq./kg was added where the reported value was indicated as cradle-to-gate, assuming degradation of produced acrylic acid entirely by passive oxidation to CO₂). For a given point on the contour plots (C,E,D,F), the x-axis value represents yield, the y-axis value represents titer, and the color and contour lines represent the value of MPSP (C,E) or Cl (D,F). Tabulated data breaking down capital and material costs, utilities, and Cl for each scenario are available online⁵⁰.

FIG. 6 shows the engineered stain can survive readily at low pH and simultaneously produce 3HP, avoiding the need to neutralize the medium for cell growth and to re-acidulate 3HP downstream, facilitating low-cost and environmentally beneficial 3HP production. Created in BioRender. Zhao, H. (2025) BioRender.com/vkz7w01.

FIG. 7A-7B show 3HP tolerance tests. (7A) a starting OD of 0.1 and (7B) a starting OD of 1. Data present mean±S.D. of three biologically independent samples (n=3). The initial pH was set at 5.6. Each curve was normalized by the highest OD obtained from individual replicate. As shown in FIG. 6A, the cells began to grow slowly as the 3HP concentration increased to 50 g/L, and a concentration of 150 g/L delayed exponential growth by one day. However, higher levels of 3HP typically occurred only at high ODs during the fermentation process. Therefore, we tested an initial OD of 1 (FIG. 6B), which showed no significant growth retardation when cells were grown in medium with up to 125 g/L of 3HP. Although there was a 12-hour delay in reaching the exponential growth phase using a medium supplemented with 150 g/L 3HP, starting with a higher OD effectively mitigated the toxicity from high levels of 3HP (i.e., up to 125 g/L).

FIG. 8 shows in vivo test for PAND gene screening. BcBAPAT (upper left arrow) and EcYDFG (upper right arrow) were integrated to SD108 first to create the PAND screening strain and PAND gene candidate plasmids were transformed into PAND screening strain. Each strain with different PAND candidate plasmid was cultured in SC-URA with 50 g/L glucose for 5 days and 3HP was measured. Data present mean±S.D. of three biologically independent samples (n=3). Created in BioRender. Zhao, H. (2025) BioRender.com/vkz7w01.

FIG. 9 shows in vivo test for BAPAT gene screening. TcPAND (upper left arrow) and EcYDFG (upper right arrow) were integrated to SD108 first to create the BAPAT screening strain and BAPAT gene candidate plasmids were transformed into BAPAT screening strain. Each strain with different PAND candidate plasmid was cultured in SC-URA with 50 g/L glucose for 5 days and 3HP was measured. Data present mean±S.D. of six biologically independent samples (n=6). Created in BioRender. Zhao, H. (2025) BioRender.com/vkz7w01.

FIG. 10 shows in vivo test for YDFG gene screening. TcPAND (upper left arrow) and BsBAPAT (upper right arrow) were integrated to SD108 first to create the YDFG screening strain and YDFG gene candidate plasmids were transformed into YDFG screening strain. Each strain with different YDFG candidate plasmid was cultured in SC-URA with 50 g/L glucose for 5 days and 3HP was measured. Data present mean±S.D. of five biologically independent samples (n=5). Created in BioRender. Zhao, H. (2025) BioRender.com/vkz7w01.

FIG. 11 shows measurement of aldehyde product using PurpaId as a purple-color-forming agent. Formaldehyde was used as positive control and for the negative control, cell lysate without expression of BAPAT was used. There was no significant purple product formed in the negative control.

FIG. 12 shows qPCR analysis of PAND and PVC gene expression. Experiment was done with one biological replicate (n=1).

FIG. 13 shows ethanol and glycerol accumulation of three strains, including pPBY/8P/ura3Δ, pPBY/8P/ura3Δ/pdcA, pPBY/8P/ura3Δ/pdcA/gpdA. Data present mean±S.D. of three biologically independent samples (n=3).

FIG. 14 shows 3HP production using pPBY/8P-AP/ura3Δ/pdcΔ/gpdΔ, pHT/1AP/Δura3 and pPBY/1AP/ura3Δ. Data present mean±S.D. of three biologically independent samples (n=3).

FIG. 15 panels A-C show fermentation Profiles of 1AP/ura3Δ and 2AP/ura3Δ Strains in CSM Medium. (A) Glucose consumption, (B) pyruvate accumulation and (C) 3HP production profile of 1AP/ura3Δ and 2AP/ura3Δ in CSM medium. Data present mean±S.D. of three biologically independent samples (n=3).

FIG. 16 shows 3HP production profile of 2AP/ura3Δ in CSL with or without uracil. Data present mean±S.D. of three biologically independent samples (n=3).

FIG. 17 shows cell growth of 2AP strain in fed-batch fermentation at 300 mL scales using CSL medium. Two independent biological replicates were performed (n=2) and mean value from two replicate is presented. Created in BioRender. Zhao, H. (2025) BioRender.com/vkz7w01.

FIG. 18 shows fermentation profile of 2AP in CSL in 3 L bioreactor. Experiment was done with one biological replicate (n=1).

FIG. 19A-19C show simplified block flow diagrams of the designed biomanufacturing facilities. (19A) fermentation, (19B) separation, and (19C) catalytic upgrading processes for all scenarios. Acronyms denote wastewater treatment (WWT) and solid-liquid separation (S/L separation). While a limited amount of lime is needed under low-pH regimes to maintain a pH of 4.0 (Table 19), no sulfuric acid is required and no gypsum is formed (as re-acidulation is not performed) under low-pH regimes.

FIG. 20 shows sensitivity analysis results for the biomanufacturing facility in the dextrose scenario. Spearman's rank order correlation coefficients (Spearman's p; x-axis, left; from −1.00 to 1.00) and corresponding p-values (x-axis, right; from 0.00 to 1.00) for minimum product selling price (MPSP), carbon intensity (CI) and fossil energy consumption (FEC) of acrylic acid (produced via catalytic upgrading of 3HP) with respect to each of 33 parameters included in the uncertainty analysis (y-axes). The vertical dashed line represents a p-value of 0.05.

FIG. 21 panels A-C show yield-titer landscape of economic and environmental metrics for corn-accepting biomanufacturing. (A) Minimum product selling price (MPSP), (B) carbon intensity (CI, as 100-year global warming potential), and (C) fossil energy consumption (FEC) for corn-accepting biomanufacturing facilities across 3,600 fermentation yield-titer combinations at the baseline productivity (0.548 g/L/h) for low-pH fermentation. Yield is shown as % of the theoretical maximum (% theoretical) scaled to the theoretical maximum yield of 1.00 g/g-glucose-equivalent (based on carbon balance). The hatched region shows the market price range for acrylic acid ($1.40-1.65/kg)²¹. For a given point on the figure, the x-axis value represents the yield, the y-axis value represents the titer, and the color and contour lines represent the value of MPSP, Cl, or FEC.

FIG. 22 panels A-C show yield-titer landscape of economic and environmental metrics for sugarcane-accepting biomanufacturing. (A) Minimum product selling price (MPSP), (B) carbon intensity (CI, as 100-year global warming potential), and (C) fossil energy consumption (FEC) for sugarcane-accepting biomanufacturing facilities across 3,600 fermentation yield-titer combinations at the baseline productivity (0.548 g/L/h) for low-pH fermentation. Yield is shown as % of the theoretical maximum (% theoretical) scaled to the theoretical maximum yield of 1.00 g/g-glucose-equivalent (based on carbon balance). The hatched region shows the market price range for acrylic acid ($1.40-1.65/kg)²¹. For a given point on the figure, the x-axis value represents the yield, the y-axis value represents the titer, and the color and contour lines represent the value of MPSP, Cl, or FEC.

FIG. 23 panels A-C show yield-titer landscape of economic and environmental metrics for corn stover-accepting biomanufacturing. (A) Minimum product selling price (MPSP), (B) carbon intensity (CI, as 100-year global warming potential), and (C) fossil energy consumption (FEC) for corn stover-accepting biomanufacturing facilities across 3,600 fermentation yield-titer combinations at the baseline productivity (0.548 g/L/h) for low-pH fermentation. Yield is shown as % of the theoretical maximum (% theoretical) scaled to the theoretical maximum yield of 1.00 g/g-glucose-equivalent (based on carbon balance). The hatched region shows the market price range for acrylic acid ($1.40-1.65/kg)²¹. For a given point on the figure, the x-axis value represents the yield, the y-axis value represents the titer, and the color and contour lines represent the value of MPSP, Cl, or FEC.

FIG. 24 shows effect of 3HP productivity on economic and environmental metrics in the dextrose scenario. Minimum product selling price (MPSP), total capital investment (TCI), annual operating cost (AOC), carbon intensity (CI; y-axes), and fossil energy consumption (FEC) for biorefineries producing acrylic acid (produced via catalytic upgrading of 3HP) in the dextrose scenario at alternative values for fermentation 3HP productivity (x-axis). Vertical dashed lines indicate productivity equivalent to 20%, 100%, and 500% of the baseline productivity (0.548 g/L/h).

FIG. 25 panels A-B show fossil energy consumption (FEC) for dextrose-accepting biomanufacturing facilities across 3,600 fermentation yield-titer combinations at the baseline productivity of dextrose scenario (i.e., at a productivity of 0.548 g/L/h) for (A) neutral and (B) low-pH fermentation. Yield is shown as % of the theoretical maximum (% theoretical) scaled to the theoretical maximum yield of 1.00 g/g-glucose-equivalent (based on carbon balance). The hatched region shows a range bounded by FEC values reported for fossil-based acrylic acid^{22,23}. For a given point on the figure, the x-axis value represents the yield, the y-axis value represents the titer, and the color and contour lines represent the value of FEC.

FIG. 26 shows uncertainty analysis of fossil energy consumption for 3HP-derived acrylic acid across four feedstock scenarios. Uncertainties (box-and-whisker plots) for fossil energy consumption (FEC) per kg of acrylic acid produced by catalytic upgrading of 3HP produced via microbial conversion of glucose and sucrose by I. orientalis. Box and whisker plots show results for four scenarios (markers report results for baseline values): (i) fermentation performance achieved in the DASbox using dextrose (dextrose, diamond markers); (ii) corn-based 3HP biomanufacturing under the same assumptions for fermentation performance as that achieved in the DASbox (corn, square markers); (v) sugarcane based 3HP biomanufacturing under the same assumptions for fermentation performance as that achieved in the DASbox (sugarcane, triangle markers); and (vi) corn stover based 3HP biomanufacturing under the same assumptions for fermentation performance as that achieved in the DASbox (corn stover, pentagon markers; baseline values and distributions for all parameters included in the uncertainty analysis for each scenario are detailed in Table 19). On box-and-whisker plots, whiskers, boxes, and the middle line represent 5^{th}/95^{th} 25^{th}/75^{th}, and 50^{th} percentiles, respectively, from 6000 Monte Carlo simulations for each scenario. respectively, from 5000 Monte Carlo simulations for each scenario. The shaded gray region shows a range bounded by FEC values reported for fossil-based acrylic acid^{22,23}.

FIG. 27A-27C show economic and environmental breakdowns for acrylic acid production via dextrose-to-3HP fermentation. Breakdowns for (27A) system costs and utility demands, (27B) carbon intensity (CI) quantified as 100-year global warming potential (GWP100), and (27C) fossil energy consumption (FEC) of acrylic acid produced via fermentation of dextrose to 3HP by engineered I. orientalis in the dextrose scenario. For the biomanufacturing facility's operating cost (MM$·y⁻¹), contributions from fixed operating costs, sales revenue from excess electricity, purchase of natural gas for product drying, and material costs by process area are shown. Values above stacked bars are totals including offsets. For heating duty, cooling duty, and electricity consumption, values indicate totals during operation. The biomanufacturing facility is assumed to operate 330 days annually at the baseline condition (baseline values and distributions with literature references for all parameters are detailed in Table 19).

FIG. 28 shows effect of fermentation working volume fraction on economic and environmental metrics in the dextrose scenario. Minimum product selling price (MPSP), total capital investment (TCI), annual operating cost (AOC), carbon intensity (CI; y-axes), and fossil energy consumption (FEC) for biorefineries producing acrylic acid (produced via catalytic upgrading of 3HP) in the dextrose scenario at alternative values for assumed fermentation working volume fraction (x-axis). Vertical dashed lines indicate the fermentation working volume fraction assumed by this study.

FIG. 29 panels A-D preliminary studies of 3HP isolation. (A) Selection of activated charcoal for color removal, (B) Effect of pH on anion exchange resin adsorption, (C) Effect of operating temperature on anion exchange resin adsorption, (D) Kinetics of 3-HP adsorption in anion exchange resin.

FIG. 30 shows a process flow diagram of 3-HP recovery illustrating unit operations, operating conditions, and the stepwise mass balance of 3-HP.

### DETAILED DESCRIPTION

To develop a cost-effective biological process for 3HP production, we sought to engineer a low-pH tolerant yeast capable of producing 3HP at high titer and yield and establish an end-to-end pipeline consisting of biomanufacturing facility design, simulation, techno-economic analysis (TEA), life cycle assessment (LCA), strain development, downstream processing, and scale-up. In this work, we identify the rate limiting enzyme in the 3HP biosynthetic pathway, PAND enzymes that catalyze the transformation of aspartate to β-alanine. We then introduce multiple copies of the PAND gene, block the ethanol and glycerol production pathways and overexpress pyruvate carboxylase (PYC) and aspartate amino transferase (AAT) enzymes. The final strain produced 29 g/L of 3HP with a yield of 0.58 g/g in shake flask fermentation. Next, we perform fed-batch fermentation at the lab scale, which resulted in a titer of 92 g/L and a 0.7 g/g glucose yield.

Despite decades of effort, financially viable biomanufacturing of 3HP from renewable plant biomass remains a significant challenge. In this study, we engineered yeast to overproduce 3HP to overcome this challenge. 3HP can be synthesized from at least four different metabolic intermediates: glycerol, lactate, malonyl-CoA, and β-alanine. The glycerol route is challenging in yeast because glycerol dehydratase is dependent on the co-factor vitamin-B₁₂ which is not natively synthesized. The lactate route is likely to result in a mixture of lactate and 3HP. Previous examinations identified both thermodynamically favorable (i.e., β-alanine and malonyl-CoA) and unfavorable (i.e., acryloyl-CoA) pathway alternatives, but were performed at neutral pH.^{24,29,34} Our thermodynamic analysis suggest that increased PAND activity could offset the small reductions in favorability of the β-alanine pathway occurring through acidification of the intracellular space during 3HP production at low external pH. Moreover, the analysis of the GSM model highlights the high theoretical yields alongside low and nearly monotonic oxygen uptake unique to the β-alanine pathway, further suggesting that industrial processes employing micro-aerobic conditions can potentially reach high yields.

To achieve high-level 3HP production, we designed an optimal β-alanine pathway. Although we successfully discovered a highly efficient PAND enzyme, PAND was still a significant rate-limiting step. We were able to integrate multiple copies PAND gene in two rounds of transformation and the resulting strain (i.e., 8P strain) reached a significantly higher titer (i.e., from 3.2 g/L to 8.4 g/L). We also increased 3HP production by knocking out the PDC/GPD gene, integrating all pathway genes, and overexpressing the PYC-AAT genes. Knocking out the PDC/GPD gene reduces the byproduct formation, thus enhancing the yield. Integration of BAPAT and YDFG circumvented the use of an unstable episomal plasmid thus an enhancement of production is observed. To further pull the flux toward β-alanine, overexpression of PYC-AAT significantly increased 3HP production. Notably, we still have not encountered any cofactor limitations as only one NADPH is required to produce one molecule of 3HP through the β-alanine pathway, whereas the malonyl-CoA pathway requires two molecules of NADPH per molecule of 3HP. Our final strain achieved the highest titer, yield and productivity among reported engineered yeast strains. In terms of titer, yield, and productivity, if using bacteria as hosts, a higher titer and productivity could be reached (Table 3). The best heterologous bacterial host of 3HP production, E. coli, reached 72.2 g/L of 3HP with the high productivity of 1.64 g/L/hr. Although the yield was acceptable (0.6 g/g)¹⁵, the fermentation process is still not financially viable because 0.85 g/g yield was required to be economically viable in neutral pH fermentation¹⁶. In general, bacteria as a host can reach higher productivity due to faster bacterial growth, but fermentation requires the maintenance of pH at 7, which increases the expense of DSP. A better way to reach industrial scale production is to perform low pH fermentation. Using yeast as a host circumvented the low pH fermentation issue. Many efforts have been made in yeast hosts previously, but have failed to generate 3HP in an economically viable manner For yeast as a host, two main pathways were chosen in the past, including the β-alanine pathway and the malonyl-CoA pathway. The malonyl-CoA pathway when used in yeast as a host, the best titer was 100.4 g/L using a non-model yeast, Y. lipolytica, but the yield was low (0.2 g/g)¹⁷. A recent study used an engineered S. cerevisiae that produced 3HP at an industrially relevant yield of 0.6 g/g, however, no fed-batch fermentation was included in the study¹⁸. As for the β-alanine pathway in yeast, the pioneering work was conducted in S. cerevisiae²⁹, however, the performance was lower than published results using the malonyl-CoA pathway. Although these studies performed fermentation near acidic conditions (i.e., pH 5.0, pH 5.6 or no control), none of the existing studies performed their fermentations below the pKa value of 3HP (i.e., 4.5), which resulted in costly DSP due to reacidification post fermentation. Here, we performed the fermentation at pH 4 and showed the robustness of our final strain to produce 3HP at high titer and yield in a low pH condition. Our study represents the highest reported 3HP yield among all engineered yeast hosts.

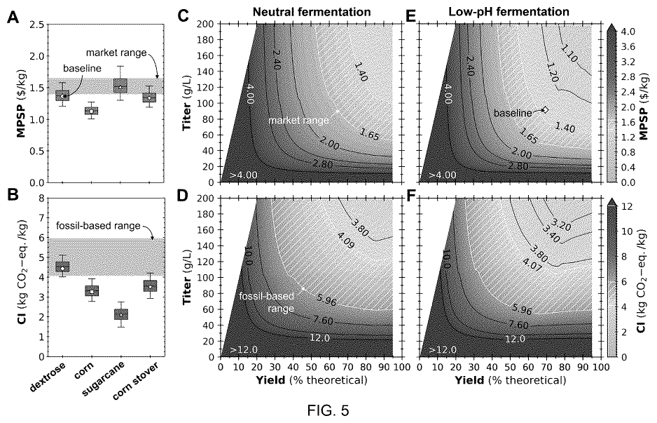

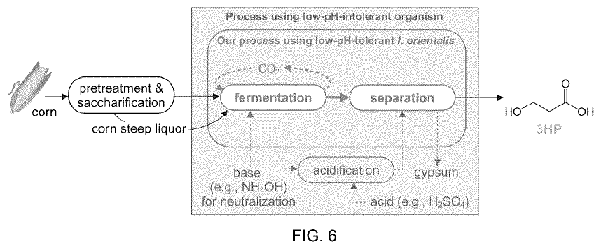

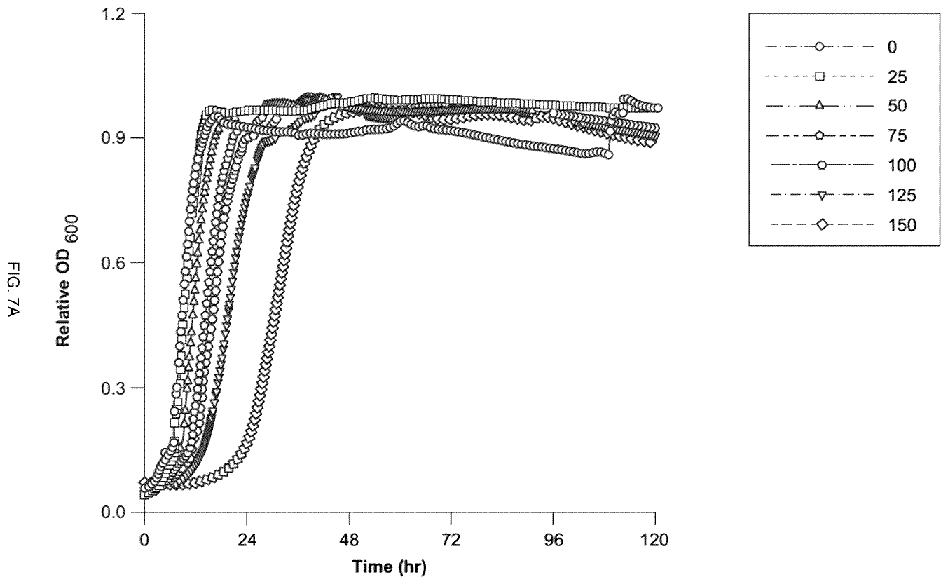

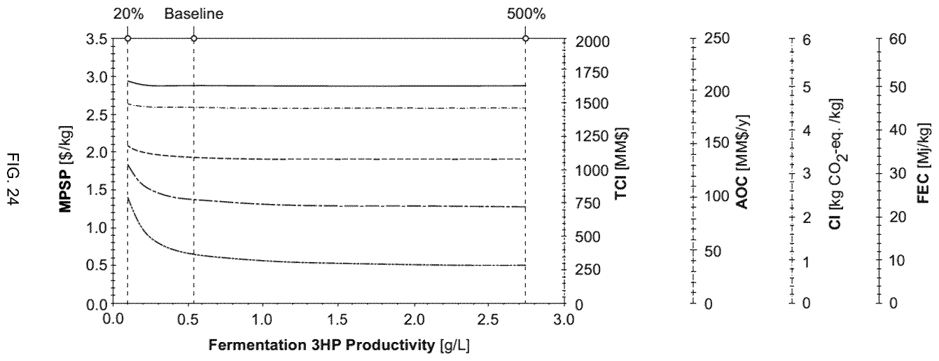

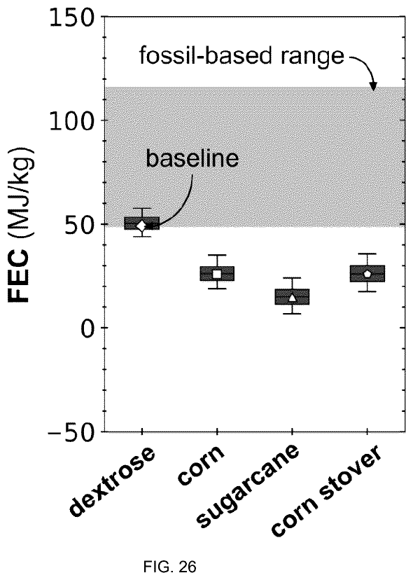

Further, we performed TEA and LCA to determine the financial viability and environmental benefits of 3HP biomanufacturing facilities that leverage our developed fermentation process, purify the produced 3HP, and catalytically upgrade it to acrylic acid. Using dextrose as a substrate, the MPSP remained below the high end of the reported market price range ($1.65/kg)^{16,35} in 99.0% of simulations, while the facility's CI (4.01-5.10 kg CO₂-eq./kg) was comparable to reported values for fossil-based acrylic acid (4.07-5.96 kg CO₂-eq./kg^{36,37}) and the FEC was on the low end of the fossil-based range (FIG. 26). Low-pH fermentation had consistently lower MPSP, Cl, and FEC values than neutral fermentation at the same yield, titer, and productivity combinations (FIG. 5, FIG. 24-26) due to reduced base and acid requirements (Table 19), and continued improvements to 3HP yield and titer combined with on-site conversion of biomass feedstocks to sugars represent the greatest opportunities to reduce system costs and environmental impacts. Sugarcane can unlock further, substantial environmental benefits over dextrose, corn stover, and corn, enabling the sustainable production of bio-based 3HP and acrylic acid.

We engineered a low-pH tolerant non-model yeast strain enabling cost-effective production of 3HP from CSL, glucose and CO₂ at low pH condition. We used TEA and LCA to assess this 3HP biomanufacturing process and found our process was financially viable at bench scale with the potential for reduced life cycle carbon intensity and fossil energy consumption relative to fossil-derived acrylic acid. Overall, this work establishes yeast as a next-generation host and provides a clear roadmap for scaling and integrating DSP toward industrial 3HP and acrylic-acid production.

### Polynucleotides

Polynucleotides contain less than an entire microbial genome and can be single- or double-stranded nucleic acids. A polynucleotide can be RNA, DNA, cDNA, genomic DNA, chemically synthesized RNA or DNA or combinations thereof. A polynucleotide can comprise, for example, a gene, open reading frame, non-coding region, or regulatory element.

A gene is any polynucleotide molecule that encodes a polypeptide, protein, or fragments thereof, optionally including one or more regulatory elements preceding (5′ non-coding sequences) and following (3′ non-coding sequences) the coding sequence. In one aspect, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein (e.g., SEQ ID NO:1-10 and 193-208).

Polynucleotides can comprise additional heterologous nucleotides that do not naturally occur contiguously with the polynucleotides. As used herein the term “heterologous” refers to a combination of elements that are not naturally occurring or that are obtained from different sources.

Polynucleotides can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides.

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can be obtained from nucleic acid sequences present in, for example, a yeast, bacteria, or other organism. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can comprise non-coding sequences or coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

A polynucleotide can be a cDNA sequence or a genomic sequence. A “genomic sequence” is a sequence that is present or that can be found in the genome of an organism or a sequence that has been isolated from the genome of an organism. A cDNA polynucleotide can include one or more of the introns of a genomic sequence from which the cDNA sequence is derived. As another example, a cDNA sequence can include all of the introns of the genomic sequence from which the cDNA sequence is derived. Complete or partial intron sequences can be included in a cDNA sequence.

Polynucleotides can encode a polypeptide disclosed herein (e.g., SEQ ID NO:1-10; 193-208, a functional fragment thereof; or having at least 95% identity to SEQ ID NO:1-10, 193-208), are provided herein.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some aspects the length of a reference sequence aligned for comparison purposes is at least 80% of the length of the comparison sequence, and in some aspects is at least 90% or 100%. In an aspect, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence.

Polypeptides and polynucleotides that are sufficiently similar to polypeptides and polynucleotides described herein can be used herein. Polypeptides and polynucleotides that are about 90, 91, 92, 93, 94 95, 96, 97, 98, 99, 99.5% or more identical to polypeptides and polynucleotides described herein can also be used herein. For example, a polypeptide can have 80% 85%, 90%, 91%, 92%, 93%, 94%, 95%, 96%, 97%, 98%, 99% or more identity to any of the SEQ ID NOs described herein.

In an aspect, a polynucleotide molecule is provided. The polynucleotide can encode a polypeptide as set forth in SEQ ID NO:1-10 or 193-208, wherein the polynucleotide molecule can be operably linked to a heterologous yeast promoter and/or a heterologous yeast terminator.

In an aspect, a polynucleotide molecule is provided. The polynucleotide can encode a polypeptide a polypeptide as set forth in SEQ ID NO:1-10 or 193-208, wherein the polynucleotide molecule is operably linked to a marker polynucleotide.

### Polypeptides

A polypeptide is a polymer of two or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

As used herein, the term “polypeptide of interest” or “polypeptides of interest”, “protein of interest”, “proteins of interest” includes any or a plurality of any of the polypeptides or other polypeptides (including variant polypeptides) described herein.

A mutated protein or polypeptide comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar Variants will be sufficiently similar to the amino acid sequence of the polypeptides described herein. Such variants generally retain the functional activity of the polypeptides described herein. Variants include peptides that differ in amino acid sequence from the native and wild-type peptide, respectively, by way of one or more amino acid deletion(s), addition(s), and/or substitution(s). These may be naturally occurring variants as well as artificially designed ones.

In an aspect, a variant polypeptide Variant polypeptides can have one or more conservative amino acid variations or other minor modifications and retain 90%-110% or more biological activity. A conservative amino acid substitution refers to the replacement of one amino acid residue in a polypeptide with another amino acid that possesses similar physicochemical properties, such as charge, hydrophobicity, polarity, or steric bulk. Such substitutions are understood to preserve the overall structure, folding, and biological activity of the polypeptide because the substituted residue performs a similar functional role within the molecule. Conservative substitutions include, but are not limited to, exchanges within groups of amino acids that share similar side-chain characteristics.

Conservative amino acid substitutions include substitutions within groups of amino acids having similar side-chain properties, such as: (i) hydrophobic residues: A, V, L, I, M, F, W, P; (ii) polar uncharged residues: S, T, N, Q, C, Y, G; positively charged residues: K, R, H; (iv) negatively charged residues: D, E; and (v) aromatic residues: F, Y, W. Substitutions within a group are expected to maintain the functional and structural integrity of the polypeptide.

In an aspect, a polypeptide comprises 80, 85, 90, 91, 92, 93, 94, 95, 96, 97, 98, 99% or greater sequence identity to SEQ ID NO:209-236. In an aspect, a polypeptide comprises 80, 85, 90, 91, 92, 93, 94, 95, 96, 97, 98, 99% or greater sequence identity to SEQ ID NO:209-236, wherein any variation in sequence identity comprises one or more conservative amino acid substitutions.

### Constructs and Cassettes

A recombinant construct is a polynucleotide having two or more heterologous polynucleotide elements. Heterologous polynucleotide elements are polynucleotides that do not occur together in nature. Any sequence of any origin can be a heterologous polynucleotide element in the polynucleotides provided herein. Exemplary heterologous polynucleotide elements include, for example, expression cassettes, cDNA sequences, genomic sequences, open reading frames (ORFs), regulatory elements, and others. Recombinant constructs include expression cassettes or expression constructs, which refer to an assembly that is capable of directing the expression of a polynucleotide or gene of interest. An expression cassette generally includes regulatory elements such as a promoter that is operably linked to (so as to direct transcription of) a polynucleotide and often includes a polyadenylation sequence or other regulatory elements as well.

In an aspect, an expression cassette can comprise a fragment of DNA comprising a coding sequence of a selected gene or gene fragment or other polynucleotide (e.g. a polynucleotide encoding a polypeptide) and optionally, regulatory elements preceding (5′ non-coding sequences) and following (3′ non-coding sequences) the coding sequence that are required for expression of the selected gene product, fragment thereof, or other polynucleotide. In an aspect, an expression cassette can be included within a vector to facilitate cloning and transformation. Different expression cassettes can be transformed into different organisms including bacteria, yeast, plants and mammalian cells, as long as the correct regulatory elements are used for each host. A vector comprising one or more expression cassettes can include, for example, one or more selectable markers, a signal that allows the vector to exist as single-stranded DNA (e.g., a M13 origin of replication), at least one multiple cloning site, and/or an origin of replication (e.g., a SV40 or adenovirus origin of replication).

Generally, a polynucleotide or gene that is introduced into an organism is part of a recombinant construct. A polynucleotide can comprise a gene of interest, e.g., a coding sequence for a protein (e.g., SEQ ID NO:209-236), or can be a sequence that is capable of regulating expression of a gene, such as a regulatory element. A recombinant construct can include, for example, regulatory elements operably linked 5′ or 3′ to a polynucleotide encoding one or more polypeptides of interest (SEQ ID NO:209-236). For example, a promoter can be operably linked with a polynucleotide encoding one or more polypeptides of interest or a polynucleotide of interest (e.g., RNA) when it is capable of affecting the expression of the polynucleotide (i.e., the polynucleotide is under the transcriptional control of the promoter). Polynucleotides (e.g., SEQ ID NO: 1-10, 193-217) can be operably linked to regulatory elements in sense or antisense orientation. The expression cassettes or recombinant constructs can additionally contain a 5′ leader polynucleotide. A leader polynucleotide can contain a promoter as well as an upstream region of a gene. The regulatory elements (i.e., promoters, enhancers, transcriptional regulatory regions, translational regulatory regions, translational termination regions, etc.) and/or the polynucleotide encoding a signal anchor can be native/endogenous to the host cell or to each other. Alternatively, the regulatory elements can be heterologous to the host cell or to each other. The expression cassette or recombinant construct can additionally contain one or more selectable marker genes.

A polynucleotide can be operably linked when it is positioned adjacent to or close to one or more regulatory elements, which direct transcription and/or translation of the polynucleotide. A polynucleotide as described herein can be operably linked to one or more polynucleotides encoding a marker.

A vector or expression cassette can be a circular or linear nucleic acid molecule. In some cases, an expression cassette is delivered to cells (e.g., a plurality of different cells or cell types including target cells or cell types and/or non-target cell types) in a vector (e.g., an expression vector).

A vector or expression cassette can comprise one or more polynucleotides of interest, encoding for one or more products of interest, or any combination thereof.

A polynucleotide can transcribed from a nucleic acid template into product of interest, such as a sgRNA, tRNA or mRNA for example; and a transcribed mRNA can subsequently be translated into peptides, polypeptides, or proteins of interest. Transcripts and encoded polypeptides can be collectively referred to as “gene product.” A polypeptide is a linear polymer of amino acids that are linked by peptide bonds.

An expression cassette can comprise a fragment of DNA comprising a coding sequence of a selected gene (e.g. PAND, PYC, ATT, BAPAT, YDFG, PDC, GPD, or combinations thereof) and regulatory elements preceding (5′ non-coding sequences) and following (3′ non-coding sequences) the coding sequence that are required for expression of the selected gene product. Thus, an expression cassette is can be composed of: 1) a promoter sequence; 2) one or more coding sequences [“ORF” ](e.g., SEQ ID NO:209-236); and 3) a 3′ untranslated region (i.e., a terminator) that, in eukaryotes, usually contains a polyadenylation site.

### Promoters and Terminators

A promoter is a nucleotide sequence that is capable of controlling the expression of a coding sequence or gene. Promoters are generally located 5′ of the sequence that they regulate. Promoters can be derived in their entirety from a native gene, or be composed of different elements derived from promoters found in nature, and/or comprise synthetic nucleotide segments. Different promoters can regulate expression of a coding sequence or gene in response to a particular stimulus, e.g., in a cell- or tissue-specific manner, in response to different environmental or physiological conditions, or in response to specific compounds. Promoters are typically classified into two classes: inducible and constitutive. A constitutive promoter refers to a promoter that allows for continual transcription of the coding sequence or gene under its control.

An inducible promoter can initiate increased levels of transcription of the coding sequence or gene under its control in response to a stimulus or an exogenous environmental condition. If inducible, there are inducer polynucleotides present therein that mediate regulation of expression so that the associated polynucleotide is transcribed only when an inducer molecule is present. A directly inducible promoter refers to a regulatory region, wherein the regulatory region is operably linked to a gene encoding a protein or polypeptide, where, in the presence of an inducer of the regulatory region, the protein or polypeptide is expressed. An indirectly inducible promoter refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by inducible promoter.

A promoter can be any polynucleotide that shows transcriptional activity in the chosen host microorganism. A promoter can be naturally-occurring, can be composed of portions of various naturally-occurring promoters, or may be partially or totally synthetic. Guidance for the design of promoters is derived from studies of promoter structure, such as that of Harley and Reynolds, Nucleic Acids Res., 15, 2343-61 (1987). In addition, the location of the promoter relative to the transcription start can be optimized. Many suitable promoters for use in microorganisms and yeast are well known in the art, as are polynucleotides that enhance expression of an associated expressible polynucleotide.

Yeast specific promoters include, for example, P_{TPI1}, P_{PGK1}, P_{TDH3}, P_{HXT7}, P_{TEF1}, P_{PYK1}, and P_{ADH1}, P_{ADH2}, GAL1, GAL7, GAL10, P_{CUP1} A yeast promoter can also be, for example, g247, g5025, g853, g917, g3376, g2204, g3504, g3824, g43, g3767, g172, g973, g4288 or other promoter listed in, e.g., US Pat. Publ. 2021/0348175, incorporated herein by reference. Additionally 859 endogenous yeast promoters from a wide variety of cellular functions, processes, and compartments are described in Keren et al., Mol Syst Biol 9:701.2013 and Lubliner et al., Nucleic Acids Res; 41:5569-81.2013. A polynucleotide encoding one or more polypeptides described herein can operably linked to an inducible or constitutive promoter. A polynucleotide encoding one or more polypeptides described herein can operably linked to a yeast-specific promoter.

A transcription termination region of a recombinant construct or expression cassette is a downstream regulatory region including a stop codon and a transcription terminator sequence. Transcription termination regions that can be used can be homologous to the transcriptional initiation region, can be homologous to the polynucleotide encoding a polypeptide of interest, or can be heterologous (i.e., derived from another source). A transcription termination region or can be naturally occurring, or wholly or partially synthetic. 3′ non-coding sequences encoding transcription termination regions may be provided in a recombinant construct or expression construct and may be from the 3′ region of the gene from which the initiation region was obtained or from a different gene. A large number of termination regions are known and function satisfactorily in a variety of hosts when utilized in both the same and different genera and species from which they were derived. Termination regions may also be derived from various genes native to the preferred hosts. In an aspect, a polynucleotide encoding a polypeptide as described herein can be operably linked to a terminator sequence, e.g., a yeast terminator sequence. A yeast-specific terminator sequence can be, e.g., g4288t, g697t, g1414t, g4282t, g2204t, g3767t, g5025t, g3824t, g527t, g4194t, g853t, g5125t, g3376t, or g3540t or other yeast-specific terminator listed in, e.g., US Pat. Publ. 2021/0348175, incorporated herein by reference. Other terminator regions include RPL41B, RPL15A, DIT1, RPL3, and IDP1, PGK1.

### Markers

A selectable marker can provide a means to identify microorganisms that express a desired product. Marker proteins include, but are not limited to, proteins that confer resistance to antibiotics, herbicides, or other compounds, which would be lethal to cells, organelles or tissues not expressing the resistance gene or allele. Selection of transformants is accomplished by growing the cells or tissues under selective pressure, i.e., on media containing the antibiotic, herbicide or other compound. If the marker protein is a “lethal” marker, cells which express the marker protein will live, while cells lacking the marker protein will die. If the marker protein is “non-lethal,” transformants (i.e., cells expressing the selectable marker) will be identifiable by some means from non-transformants, but both transformants and non-transformants will live in the presence of the selection pressure.

Selective pressure refers to the influence exerted by some factor (such as an antibiotic, heat, light, pressure, or a marker protein) on natural selection to promote one group of organisms or cells over another. In the case of antibiotic resistance, applying antibiotics cause a selective pressure by killing susceptible cells, allowing antibiotic-resistant cells to survive and multiply.

Selective pressure can be applied by contacting the cells with an antibiotic and selecting the cells that survive. The antibiotic can be, for example, kanamycin, puromycin, spectinomycin, streptomycin, ampicillin, carbenicillin, bleomycin, erythromycin, polymyxin B, tetracycline, or chloramphenicol.

Selectable markers include, but are not limited to, ampicillin resistance, neomycin phosphotransferase, which confers resistance to the aminoglycosides neomycin, kanamycin and paromycin (Herrera-Estrella, EMBO J. 2:987-995, (1983)); dihydrofolate reductase, which confers resistance to methotrexate (Reiss, Plant Physiol. (Life Sci. Adv.) 13:143-149, (1994)); trpB, which allows cells to utilize indole in place of tryptophan; hisD, which allows cells to utilize histinol in place of histidine (Hartman, Proc. Natl. Acad. Sci., USA 85:8047, (1988)); mannose-6-phosphate isomerase which allows cells to utilize mannose (WO 94/20627); hygro, which confers resistance to hygromycin (Marsh, Gene 32:481-485, (1984)); ornithine decarboxylase, which confers resistance to the ornithine decarboxylase inhibitor, 2-(difluoromethyl)-DL-ornithine (DFMO; McConlogue, In: Current Communications in Molecular Biology, Cold Spring Harbor Laboratory ed., (1987)); deaminase from Aspergillus terreus, which confers resistance to Blasticidin S (Tamura, Biosci. Biotechnol. Biochem. 59:2336-2338, (1995)); phosphinothricin acetyltransferase gene, which confers resistance to phosphinothricin (White et al., Nucl. Acids Res. 18:1062, (1990); Spencer et al., Theor. Appl. Genet. 79:625-633, (1990)); a mutant acetolactate synthase, which confers imidazolione or sulfonylurea resistance (Lee et al., EMBO J. 7:1241-1248, (1988)), a mutant EPSPV-synthase, which confers glyphosate resistance (Hinchee et al., BioTechnology 91:915-922, (1998)); a mutant psbA, which confers resistance to atrazine (Smeda et al., Plant Physiol. 103:911-917, (1993)), a mutant protoporphyrinogen oxidase (see U.S. Pat. No. 5,767,373), or other markers conferring resistance to an herbicide such as glufosinate.

A marker protein can also be, for example, a fluorescence protein for easier monitoring of genome integration and expression, and to label or track particular cells.

A polynucleotide encoding a polypeptide as described herein can be operably linked to one or more marker polynucleotides.

### Vectors

Vectors for stable transformation of yeast can be obtained from commercial vendors or constructed from publicly available sequence information. Expression vectors can be engineered to produce heterologous and/or homologous protein(s) of interest (e.g., SEQ ID NO:209-236). Such vectors are useful for recombinantly producing a protein of interest and for modifying the natural phenotype of host cells.

If desired, polynucleotides (e.g., SEQ ID NO: 1-10, 193-208) can be cloned into an expression vector comprising expression control elements, including for example, origins of replication, promoters, enhancers, or other regulatory elements that drive expression of the polynucleotides in host cells. A vector can be, for example, a plasmid, such as YIp, YRp, YCp, YEp, and YLp.

To confirm the presence of recombinant polynucleotides or recombinant genes in transgenic cells, a polymerase chain reaction (PCR) amplification or Southern blot analysis can be performed using methods known to those skilled in the art. Expression products of the recombinant polynucleotides or recombinant genes can be detected in any of a variety of ways, and include for example, western blot and enzyme assay. Once recombinant organisms have been obtained, they may be grown in cell culture.

### Gene Disruptions and Mutations

A genetic mutation comprises a change or changes in a nucleotide sequence of a gene or related regulatory region or polynucleotide that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be 2, 3, 4, 5, 6, 7, 8, 9, 10, or more nucleotide changes. Mutations can occur within the coding region of the gene or polynucleotide as well as within the non-coding and regulatory elements of a gene. A genetic mutation can also include silent and conservative mutations within a coding region as well as changes which alter the amino acid sequence of the polypeptide encoded by the gene or polynucleotide. A genetic mutation can, for example, increase, decrease, or otherwise alter the activity (e.g., biological activity) of the polypeptide product. A genetic mutation in a regulatory element can increase, decrease, or otherwise alter the expression of sequences operably linked to the altered regulatory element.

A gene disruption is a genetic alteration in a polynucleotide or gene that renders an encoded gene product (e.g., PDC, GPD) attenuated (e.g., produced at a lower amount, or having lower biological activity than a gene that has not been disrupted). A gene disruption can include a disruption in a polynucleotide or gene that results in reduced expression of an encoded gene product (e.g., 10, 25, 50, 75% or less expression as compared to a gene that is not disrupted), or reduced or attenuated biological activity (e.g., 10, 25, 50, 75% or less biological activity) as compared to a gene that is not disrupted). The genetic alteration can be, for example, or addition or deletion of a regulatory element required for transcription or translation of the polynucleotide or gene, deletion or addition of a regulatory element required for transcription or translation or the polynucleotide or gene, addition of a different regulatory element required for transcription or translation or the gene or polynucleotide, deletion of a portion (e.g. 1, 2, 3, 6, 9, 21, 30, 60, 90, 120 or more nucleic acids) of the gene or polynucleotide, which results in an partially active gene product or a gene product with no biological activity, replacement of a gene's promoter with a weaker promoter or no promoter, replacement or insertion of one or more amino acids of the encoded protein to reduce its activity, stability, or concentration, or inactivation or activation of a gene's transactivating factor such as a regulatory protein. Zinc-finger nucleases (ZFNs), Talens, and CRISPR-Cas9 allow double strand DNA cleavage at specific sites in yeast chromosomes such that targeted gene insertion or deletion can be performed (Shukla et al. 2009, Nature 459:437-441; Townsend et al., 2009, Nature 459:442-445). This approach can be used to modify the promoter of endogenous genes or the endogenous genes themselves to modify expression of PDC and/or GPD which can be present in the genome of yeast of interest. ZFNs, Talens or CRISPR/Cas9 can be used to change the sequences regulating the expression of the polypeptides to decrease the expression or alter the timing of expression beyond that found in a non-engineered or wild-type yeast, or to delete the wild-type polynucleotide, or replace it with a deleted or mutated form to decrease the expression and/or activity of PDC and/or GPD.

### Recombinant Yeast

A “recombinant yeast” or “recombinant yeast cell” refers to a yeast cell that has been genetically modified from the native state. A recombinant yeast cell can have, for example, nucleotide insertions, nucleotide deletions, nucleotide rearrangements, gene disruptions, recombinant polynucleotides, heterologous polynucleotides, deleted polynucleotides, nucleotide modifications, or combinations thereof introduced into its DNA. These genetic modifications can be present in the chromosome of the yeast or yeast cell, or on a plasmid in the yeast or yeast cell. Recombinant yeast cells disclosed herein can comprise exogenous polynucleotides on plasmids. Alternatively, recombinant yeast cells can comprise exogenous polynucleotides stably incorporated into their chromosome.

A heterologous or exogenous polypeptide or polynucleotide refers to any polynucleotide or polypeptide that does not naturally occur or that is not present in the starting target microorganism. For example, a polynucleotide from bacteria that is transformed into a yeast cell that does naturally or otherwise comprise the bacterial polynucleotide is a heterologous or exogenous polynucleotide. A heterologous or exogenous polypeptide or polynucleotide can be a wild-type, synthetic, or mutated polypeptide or polynucleotide. In an aspect, a heterologous or exogenous polypeptide or polynucleotide is not naturally present in a starting target microorganism and is from a different genus or species than the starting target microorganism.

A homologous or endogenous polypeptide or polynucleotide refers to any polynucleotide or polypeptide that naturally occurs or that is otherwise present in a starting target microorganism. For example, a polynucleotide that is naturally present in a yeast cell is a homologous or endogenous polynucleotide. In an aspect, a homologous or endogenous polypeptide or polynucleotide is naturally present in a starting target microorganism.

A recombinant yeast can comprise one or more polynucleotides that are not present in a corresponding wild-type yeast, wherein the polynucleotides have been introduced into that yeast using recombinant DNA techniques, or which polynucleotides are not present in a wild-type yeast and is the result of one or more mutations.

A genetically modified or recombinant yeast can be, for example, a yeast (i.e., Ascomycota and Basidiomycota). Examples include Issatchenkia sp., e.g. I. orientalis, Saccharomyceraceae, such as Saccharomyces cerevisiae, Saccharomyces cerevisiae strain S8, Saccharomyces pastorianus, Saccharomyces beticus, Saccharomyces fermentati, Saccharomyces paradoxus, Saccharomyces uvarum and Saccharomyces bayanus; Schizosaccharomyces such as Schizosaccharomyces pombe, Schizosaccharomyces japonicus, Schizosaccharomyces octosporus and Schizosaccharomyces cryophilus; Torulaspora such as Torulaspora delbrueckii; Kluyveromyces such as Kluyveromyces marxianus; Pichia such as Pichia stipitis, Pichia pastoris or Pichia angusta, Zygosaccharomyces such as Zygosaccharomyces bailii; Brettanomyces such as Brettanomyces intermedius, Brettanomyces bruxellensis, Brettanomyces anomalus, Brettanomyces custersianus, Brettanomyces naardenensis, Brettanomyces nanus, Dekkera bruxellensis and Dekkera anomala; Metschmkowia, Issatchenkia, such as Issatchenkia orientalis, Kloeckera such as Kloeckera apiculata; Aureobasidium such as Aureobasidium pullulans.

In an aspect a recombinant yeast comprises one or more heterologous polynucleotides encoding 1, 2, 3, 4, 5, 6, 7, 8, 9, 10 or more copies of aspartate 1-decarboxylase (PAND) polypeptide. The one or more heterologous polynucleotides encoding PAND polypeptides can comprise 80, 85, 90, 91, 92, 93, 94, 95, 96, 97, 98, 99% or greater sequence identity to the sequences shown in Table 9. In an aspect, one or more PAND polypeptides can comprise 80, 85, 90, 91, 92, 93, 94, 95, 96, 97, 98, 99% or greater sequence identity to SEQ ID NO:209-217.

| | |
|---|---|
| MCSSKEGT------VVVEKL---SYKDLYpYNINGKAETRAFLLKVVSILLD---YVDEENDRTS-KILDFKMPEELEQI | 67 |
| MPASEASAERPRAdVVIEEIDgaSSKEAF-LSAPDKEIHGQFLHRVQQMLLD---HAFQSTHRES-KVTRYVDPDRLREI | 75 |
| MSSVTKTTATTQN--KVDEID----KIRL-----NLADDWKVLGSVYDLLKDgkaFCVSDADRDAvKIVDFKQPNEMKQL | 69 |
| LDLNLPD-KGLPLGDLLKDCRSTLKWQVKTGHPHFFNQLSSGLDIVSLAGEWLTSVANTNMFTYEIAPVFILMENVVLKK | 146 |
| IDMNLRK-EPASEEALLSYIRDTMKYSVKTGHPFFVNQLFSSLDPYALAGAWVTDSLNPSVYTYEVSPVLTLMEESVLKE | 154 |
| IDLSIDEnKTHTIAEIEDLCRLVIKYSVKINHGRFHNQLFGQMDPYGLMGQWITDALNASAYTYEVGPVFSLMETEIIRT | 149 |
| MREYLGFVHGDSILAPGGSISNMYAVMGARHKMFPNYKKLGLKALPQLVMYTSVDSHYSAMGAAASCGLGTDNVIQIGTD | 226 |
| MRSIVGWSDGDGIFAPGGSIANGYAISCARFHKIPDIKAKGLHGLPRLVLFTSEDAHYSIKKLAAFEGLGSDNVYPVKTD | 234 |
| VCQLAGYAEGDGIFSPGGSMSNMYGLVLARYSRHPQVKTAGLFGMRPLALFTSEESHYSFVKAAHWLGLGADNCVVVKTN | 229 |
| SMGRMRVDKLEEAVLATKAKGHVPFFVNCTCGTTVVGAFDPINPIADLCQKYGMWLHIDAAWGGGVLLSKKHRHLMAGVE | 306 |
| ARGKMDVLHLRELVQRAKAEGAVPFMVSATSGTTVLGAFDPLEAIHEVCEEEGMWLHVDAAWGGGALVSKKYRHLLRGIE | 314 |
| ERGQMLTNDLEAKILKAQESGCEPFFVNATAGTTVLGAFDDLNAIADVCEKYGLWLHVDACLGGSALLSYRNRHLLAGLD | 309 |
| RANSVTWNPHKLMGTHLQCSTIHFRQDGILLGCNQMCAEYLFQQDKHYDVSFDTGDKVPQCGRRNDIFKLWLMWRAKGTE | 386 |
| KADSVTWNPHKMLAAPQQCSTLLTKHKTILAECHSASAQYLFQKDKFYDTSFDTGDKHIQCGRRADVFKFWLMWKAKGTD | 394 |
| RTNSFAWNPHKSLGAPLQCSLFLTRERDLLTRCNSIQVNYLFQQDKFYDVSYDTGNKSVQCGRKIDAFKFWLMLKARGYG | 389 |
| GFEAHIDQLFDISQYLVKRIKEMPE--KFHLIQEPEMVNVLFWYIPQRLRDQPQSKERIAELGRITAQLKARMMNTGTLM | 464 |
| GLEKHVNRVYENAEYFTEKIRRRPG--FKLVLEEPECTNVTFWYVPPSIRGRDNEPEYNQKLHKVAPLIKERMTREGTML | 472 |
| SFGRLLDHAIDISKVLVEKIKQRGSehFRLVLEDNQYMNVCFWYIPKALRGLEETPEWRERLYTVAPKIKERMAYSGTVM | 469 |
| ITYQPL--RELPNFFRNIVSN-SAVTKDDIDELLAEMDRLGNDL-- | 505 P3 |
| | (SEQ ID NO: 211) |
| MTYQPL--KELPNFFRLVIQN-SALTTSDMDYIVSEFERLGRNL-- | 513 P5 |
| | (SEQ ID NO: 213) |
| IGYTPLqyRGLGNFFRMVFTCfPVLEEAELDFILNEIERLGEECey | 515 P7 |
| | (SEQ ID NO: 215) |

In an aspect, a PAND polynucleotide encodes a PAND polypeptide comprising 80, 85, 90, 91, 92, 93, 94, 95, 96, 97, 98, 99% or greater sequence identity to SEQ ID NO:213, 211, or 215, wherein the same amino acid that occurs in all three polypeptides in the same position (e.g., position 1 where all three sequences have an M) shown in the alignment above are retained. In an aspect, a PAND polynucleotide encodes a PAND polypeptide comprising 80, 85, 90, 91, 92, 93, 94, 95, 96, 97, 98, 99% or greater sequence identity to SEQ ID NO:213, 211, or 215, wherein one or more conservative amino acid mutations are present.

In an aspect, a recombinant yeast has its endogenous pyruvate decarboxylase (PDC) gene(s) inactivated. An inactivated PDC gene results in the amount of pyruvate decarboxylase expressed by the host recombinant yeast to be reduced by 50, 60, 70, 80, 90, 95, 98, 99, 99.5% or more as compared to a wild-type recombinant yeast of the same species of the host recombinant yeast. In an aspect, an inactivated PDC gene results in the expression of no pyruvate decarboxylase. In an aspect, a PDC gene has 85, 90, 91, 92, 93, 94, 95, 96, 97, 98, 99, 99.5% or more sequence identity to GenBank Accession Number KGK39951.1, XP_029319027.1, XP_019019447.1, KAG0673093.1, or TID19240.1.

In an aspect, a recombinant yeast has its endogenous glycerol-3-phosphate dehydrogenase (GPD) gene(s) inactivated. An inactivated GPD gene results in the amount of glycerol-3-phosphate dehydrogenase expressed by the host recombinant yeast to be reduced by 50, 60, 70, 80, 90, 95, 98, 99, 99.5% or more as compared to a wild-type recombinant yeast of the same species of the host recombinant yeast. In an aspect, an inactivated GPD gene results in the expression of no glycerol-3-phosphate dehydrogenase. In an aspect, a GPD gene has 85, 90, 91, 92, 93, 94, 95, 96, 97, 98, 99, 99.5% or more sequence identity to GenBank Accession Number KGK37046.1, XP_029323624.1, ABW38024.1, OUT22057.1, XP_019016990.1, GAV28552.1, TID22554.1, KAG0689929.1, or KAG0684433.1.

A recombinant yeast can comprise one or more heterologous polynucleotides encoding one or more copies of pyruvate carboxylase (PYC). In an aspect, the recombinant yeast can further comprise one or more heterologous polynucleotides encoding two or more copies (e.g., 1, 2, 3, 4, or more copies) of pyruvate carboxylase (PYC).

In an aspect, a recombinant yeast can comprise one or more (1, 2, 3, or more) heterologous polynucleotides encoding PYC polypeptides. In an aspect, a PYC polypeptide is shown in SEQ ID NO:235.

(SEQ ID NO: 235)
MNMKEKSFGPRQFKGNLQQHKQTILKTVLFLGLIWLFFCSYQSQGSSISKPSSKKFQKFFKKYFN
ELDKAIVHGDKLMFGTRANAEMENIYADVSETELKELEDFSPEIIKQLKVNHRKIVSFIPKEIHVE
GYCGSGYVTIGGGNNDYKSMLLIKQLRETGAKNPIEVIIPPFYEPNLNLCDNIFPSYDATCVFMNG
IFGKKNLQKLRYNSKLIGTLAIIASSFENALFISPDTIPLHNPDHLFEGKVISQFGLIVWPSVYRTSIT
PSYYEISDIQVENNPVRILNDRWVSPEDIHQHLQAKAQRDHENTQLSFHDLKGTSSDYAATSDLL
LINKRKNLDVLLLMLYYTNDGPSRYFPLLNAVSSKGNGDYIVAAAHYFKKPYYQLFKTADKIGE
EINSLTQVTSITGHYDPNEDYELLKGNLLSLFEQRRELKNDFKYDYFEKIRRPFNVFTSTPLFFHVE
ETLHPFKMKNSKVYKDNTGNQRRIFGNHIKFYGCDFELKLITSINELLCGAESTDFTFAGFENVD
VLCSDYIQPRMEFLQGNSKQFWNKYVFEKRPPNSVPQTSVSTIEKAIKEGFDRDVEYQKDEPARP
SILYL

In an aspect, a PYC polynucleotide encodes a PYC polypeptide comprising 80, 85, 90, 91, 92, 93, 94, 95, 96, 97, 98, 99% or greater sequence identity to SEQ ID NO:235. In an aspect, a PYC polynucleotide encodes a PYC polypeptide comprising 80, 85, 90, 91, 92, 93, 94, 95, 96, 97, 98, 99% or greater sequence identity to SEQ ID NO:235, wherein one or more conservative amino acid mutations are present. In an aspect, a PYC polynucleotide is derived from Issatchenkia sp., e.g., Issatchenkia orientalis.

A recombinant yeast can comprise one or more heterologous polynucleotides encoding one or more copies of aspartate aminotransferase (AAT). In an aspect, the recombinant yeast can further comprise one or more heterologous polynucleotides encoding two or more copies (e.g., 1, 2, 3, 4, or more copies) of aspartate aminotransferase (AAT).

SEQ ID NO: 236
MLSIYKSIRLAKPDPILGLSAAFLRDPSPSKINLGVGAYRDNNGNPWTLPSVALA
EEALARSHGNHEYVPILGSQKYIDSVQEFLFKEDKLGQLLREGKILTGQGLSGTGSLRVLGEFM
HSHLKIKTIHVSTPTWPNHVNVFTNSSLKVAKYPYYDPQTKGLAFHEMLDSLSKVPYGDAILLHS
CCHNPTGVDPTQDQWREILAIIKKRQLFPVIDMAYQGFAESLQKDMFVPQLTAKLGIPFILSQSF
AKNMGLYGERIGSFSIILNDESEFEPVKSQISIVARAMYSSPPKHGAEIVSYILTNDKLLAQWETE
VSIMRDRLANMRHSLYEELTRRTDNDWSHLIKQKGMFAFTGLNVNQVHKLREKNVYLTDNGRI
SIAGINNHNVNHLADAIAQVCK

In an aspect, the PYC and/or AAT genes, e.g. homologous or heterologous PYC or AAT polypeptides or genes can be overexpressed in a recombinant yeast described herein. The term “overexpression” or “overexpressed” refers to a level of polypeptide expression that is greater than what is measured in a wild-type cell of the same species as the host cell that has not been genetically altered. The overexpression of the polypeptides can be achieved by constructing inducible overexpression vectors encoding for the desired polypeptide. Strong promoters can be used to induce overexpression of a polypeptide as can the use of multiple copies of a polynucleotide in the recombinant microorganism. In an aspect a PAND, PYC, and/or AAT polypeptide or gene can be overexpressed. A strong promoter, as used herein, is a DNA sequence that controls a transcription rate of a gene, which is high for a strong promoter and low for a weak promoter. A strong promoter can have a transcription rate that is 10, 20, 30, 40, 50, 60, 70, 80, 90% or more as compared to a wild-type promoter of a gene. For all instances herein, a strong promoter can be, e.g., S. cerevisiae promoters TDH3 (GPD), TEF1, PGK1, ADH1, ENO1/ENO2, HXT7, or FBA1, synthetic promoters from the TEFmut-series (e.g., TEF1 mutants), pGPD or P_GPD variants, which are synthetic strengthened versions of TDH3/GPD), hybrid promoters such as CYC1-based hybrids, HAP1-independent promoters, Pichia pastoris GAP promoter (PGAP), Yarrowia lipolytica TEF promoter, and/or Schizosaccharomyces pombe adh1 promoter (pad1).

A recombinant yeast can comprise one or more heterologous polynucleotides encoding one or more copies of β-alanine-pyruvate aminotransferase (BAPAT). In an aspect, the recombinant yeast can further comprise one or more heterologous polynucleotides encoding two or more copies (e.g., 1, 2, 3, 4, or more copies) of β-alanine-pyruvate aminotransferase (BAPAT). In an aspect, the one or more heterologous polynucleotides encoding BAPAT comprise 80, 85, 90, 91, 92, 93, 94, 95, 96, 97, 98, 99, 99.5% or greater sequence identity to the sequences shown in Table 10. In an aspect, one or more BAPAT polypeptides can comprise 80, 85, 90, 91, 92, 93, 94, 95, 96, 97, 98, 99% or greater sequence identity to SEQ ID NO:209-217.

| | |
|---|---|
| MELMIVQVTEQTQSLKKTDEKYLW[5]AAPSPTNLIITKAEGAWVTD-IDGNRYLDGMSGLWCVNVGYGRKEL ARAA | 78 |
| ---MSSETISQSINVVHP-***** *******VTLSHGKNAEVWD-TEGKRYIDFVGGIGVLNLGHCNPRI VEAI | 57 |
| MENVKYQI--PPAEVLDTLGKYIL VDGFHTVIDLVKSHDNYIVDaVTGQEILDCYSYFASLPIGHNHPKL[5]LKAL | 77 |
| FE-QLEEMPYFPLTQS-HVPAIKLAEKLNEWLDDEYVI--FFSNSGSEANETAFKIARQYHQQKGDHGR YKFISRYR | 151 |
| RE-QATRLTHYAFNAAPHTPYLELMDRLTAFIPVSYPVSgMLTNSGAEAAENALKIVR------GATGR TAVIAFDG | 127 |
| FRaTIANPSNSDIYCPEYAWAVKTFAEFAK--PAEFKYM-FFVAGGAPAIGNALKVAFDWRTRKEMNAN[7]YGVLHLKE | 158 |
| AYHGNSMGALAATGQ-AQRKYKYEPLGQGFLHVAPPDT-YRNPEDVHTLASAEEIDRVMTWELS- QTVAGVIMEPII | 225 |
| AFHGRTLATLNLNGKVAPYKQKVGVLPGPVYHLPFPS----KDNGVTCAEALKAMDRLESVEIDV NDVACFIVEPVQ | 200 |
| AFHGRLGYTLSLTNT-DPVKTEYFP-KEDWPRITNPKIrFGKDGRISNATSVQRAEREAMIQIDR[4]QEIAALILEPIQ | 237 |
| TGGGILMPPDGYMEKVKEICEKHGALLICDEVICGFGRTGKPFGFMNYGVKPDIITMAKGITSAyLPLSATAVRREVYEA | 305 |
| GEAGFLAMDVEFAQALRRFCDEKNILLIADEIQSGFGRTGERFAFSRLGIEPDLILLAKSIAGG-VPLGAVVGRKSLL-- | 277 |
| GEGGDNHFRPCFFKALRQKCDDNDIMLIFDEVQTGVGLTGRMWAYEHFGVTPDILVFGKKTQ-----VCGIMATERVDEV | 312 |
| fvgSDDYDR--FRHVNTFGGNPAACALALKNLEIMENEKLIERSKELGERLLYELED---VKEHPNVGDVRGKGLLLGIE | 380 |
| ----DTLPK--GGLGGTYSGNPIACAAALATLDEMTDANLNAWGSQQEEAIVSRYEAwrVSKLSPYLGRLTGVGAMRGIE | 351 |
| ---PDNVFRksGRINSTWGANLADELRGARYLEIICEDNLVANAATVGKYFLDKLRA--EVYDPKHAQNIRGRGLMIAFD | 387 |
| LveDKQTKEPASIEKMNKVINACKEKGLIIGKNGDTVagyNNILQLAPPLSITEEDFTFIVKTMKECLAQ--HHHHHH | 456 B1 |
| | (SEQ ID |
| | NO: 237 |
| L--SNADGSPASAQ-LTQLLALARDAGLLLMPSGKS----RNIIRLLAPLTTEATVLEEGLDIFEACLKKItHHHHHH | 422 B2 |
| | SEQ ID |
| | NO: 229 |
| F-------KDAATR--NEFRRKAWDNGLATLSCGTYS------VRFRPPLTFTKENVDEAIRIIKKSL----HHHHHH | 446 B5 |
| | SEQ ID |
| | NO: 238 |

In an aspect, a BAPAT polynucleotide encodes a BAPAT polypeptide comprising 80, 85, 90, 91, 92, 93, 94, 95, 96, 97, 98, 99% or greater sequence identity to SEQ ID NO:228, 229, or 232, wherein the same amino acid that occurs at two or three polypeptides in the same position shown in the alignment above are retained. In an aspect, a BAPAT polynucleotide encodes a BAPAT polypeptide comprising 80, 85, 90, 91, 92, 93, 94, 95, 96, 97, 98, 99% or greater sequence identity to SEQ ID NO:213, 211, or 215, wherein one or more conservative amino acid mutations are present.

In an aspect, a BAPAT polynucleotide encodes a BAPAT polypeptide comprising 80, 85, 90, 91, 92, 93, 94, 95, 96, 97, 98, 99% or greater sequence identity to SEQ ID NO:230, 231, 233, or 234. Two or more polypeptides set forth in SEQ ID NO:228-234 can be aligned as demonstrated above to determine which amino acids can be retained and which can be substituted. In an aspect, BAPAT polynucleotide encodes a BAPAT polypeptide comprising 80, 85, 90, 91, 92, 93, 94, 95, 96, 97, 98, 99% or greater sequence identity to SEQ ID NO: 219, 221, 223, 225, or 226, wherein one or more conservative amino acid mutations are present.

In an aspect, a BAPAT polynucleotide is derived from Pseudomonas sp., Pichia sp., Burkholderia sp., Mesorhizobium sp., Falkowbacteria sp., and/or Klebsiella sp. In an aspect, a BAPAT polynucleotide is derived from Pseudomonas nunensis, Pichia kudriavzevii, Burkholderia sp. Bp9004, Mesorhizobium sp. Root552, candidatus Falkowbacteria bacterium RIFOXYA2_FULL_47_19, and/or Klebsiella michiganensis.

A recombinant yeast can comprise one or more heterologous YDFG polynucleotides encoding one or more copies of 3HP dehydrogenase (YDFG) polypeptides. In an aspect, the recombinant yeast can further comprise one or more heterologous polynucleotides encoding two or more copies (e.g., 1, 2, 3, 4, or more copies) of 3HP dehydrogenase (YDFG). In an aspect, the one or more heterologous polynucleotides encoding YDFG polypeptides comprise 80, 85, 90, 91, 92, 93, 94, 95, 96, 97, 98, 99, 99.5% or greater sequence identity to the sequences shown in Table 11. In an aspect, one or more YDFG polypeptides can comprise 80, 85, 90, 91, 92, 93, 94, 95, 96, 97, 98, 99% or greater sequence identity to SEQ ID NO:218-227.

| | | |
|---|---|---|
| 1 | --MIVLVTGATAGFGESITRRFVKNGHKVIATGRRQERLLELKEELGDN--LFTLQLDVRNRAAIEEALASLPAEWQHID | 76 |
| 1 | --MIVFVTGATAGFGESITRRFIAQGHKVVATGRRSERLQALKDELGEN--VYTLQLDVRNRAGIDAALHSLPAEWRDID | 76 |
| 1 | mtPTLFITGATSGFGEACARRFARDGWSLVITGRREDRLQALAKELSSQaeVLPITLDVRDRAAVAHAVDNLPAGEDKLR | 80 |
| 77 | ILVNNAGLALGMEPAHKANVDDWENMIDTNNKGLVYMTRAVLPGMVERNRG-HIINIGSTAGSWPYAGGNVYGATKAFVR | 155 |
| 77 | VLVNNAGLALGMAPAHQASIDDWEAMIDTNNKGLVEMTRAILPAMVERNVG-HIINIGSTAGNWPYAGGNVYGATKAFVR | 155 |
| 81 | ALINNAGLALGADPAQECDLDDWDTMVDTNIKGLMYCTRLLLPRLIAHGRGaSILNLGSVAGQWPYPGSHVYGATKAFVE | 160 |
| 156 | QFSLNLRTDLHKTAVRVTDIEPGMVGGTEFSNVRFKGDDDKAGKTYENANALTPEDVTEAVWWVATLPKHVNINTLEMMP | 235 |
| 156 | QFSLNLRTDLHGTALRVTDIEPGLVGGTEFSNVRFKGDSDKADSVYDGTEPLTAEDVTEAVFWVASLPKHVNINTLEMMP | 235 |
| 161 | QFSLSLRCDLQGTGVRVSNLEPGLCE-SEFSLVRFGGDQARYDKTYAGTEALQPEDIAETIHWIVNQPPHININSLEIMP | 239 |
| 236 | --QTSEDKKTMPLS[224] | 471 Y1 |
| | | SEQ ID |
| | | NO: 239 |
| 236 | VGQTLAGLKVHKA- | 248 Y3 |
| | | SEQ ID |
| | | NO: 220 |
| 240 | VSQAWAGFSVHRKG | 253 Y5 |
| | | SEQ ID |
| | | NO: 222 |

In an aspect, a YDFG polynucleotide encodes a YDFG polypeptide comprising 80, 85, 90, 91, 92, 93, 94, 95, 96, 97, 98, 99% or greater sequence identity to SEQ ID NO:218, 220, or 222, wherein the same amino acid that occurs in all three polypeptides in the same position (e.g., position 8 where all three sequences have a T) shown in the alignment above are retained. In an aspect, a YDFG polynucleotide encodes a YDFG polypeptide comprising 80, 85, 90, 91, 92, 93, 94, 95, 96, 97, 98, 99% or greater sequence identity to SEQ ID NO:218, 220, or 222, wherein one or more conservative amino acid mutations are present.

| | | |
|---|---|---|
| 1 | --MIVFVTGATAGFGESITRRFIAQGHKVVATGRRSERLQALKDELGENVYTLQLDVRNRAGIDAALHSLPAEWRDIDVL | 78 |
| 1 | MPLTVLVTGATAGFGHAIATRLVREGHRVIATGRRKERLEALKDELGESVLPYTLDMTDREALRALPAALPEEWQDIDVL | 80 |
| 1 | MKKTAFVTGATSGFGEAIARRLSKEGYKIVALARREDRLKKLASELGDT-HIIVADIRDKEAVFDAVDSLPENFKDIEVL | 79 |
| 79 | VNNAGLALGMAPAHQASIDDWEAMIDTNNKGLVEMTRAILPAMVERNVGHIINIGSTAGNWPYAGGNVYGATKAFVRQFS | 158 |
| 81 | VNNAGLALGMEPAQDAEPDNWESMIATNVSGVVELTRALLPGMVQRNTGYIIALGSTAGTYPYTGGNVYGATKAFVHQFT | 160 |
| 80 | VNNAGMALGLEKTIDAKVEDFEAMIDTNVKGLIYSTKAVLPLLYKQEKGYIFNIGSTAGSWPYPGSNVYGATKAFVKQFS | 159 |
| 159 | LNLRTDLHGTALRVTDIEPGLVGGTEFSNVRFKGDSDKADSVYDGTEPLTAEDVTEAVFWVASLPKHVNINTLEMMPVGQ | 238 |
| 161 | QNLRTDLLGTRIRVTNIEPGLCGGSEFSNVRL-GDDAKAAAVYKDTTPLTPEDIAGTVSWLIGLPWHVNVNYLEMMPVCQ | 239 |
| 160 | LNLRNDLVGTNIRVTNIEPGLCK-TEFSEVRERGDKAKADSLYENTNFITSEDIATILVNCLNMPGSVNINRVEVMANTQ | 238 |
| 239 | TLAGLKVHK--A | 248 Y3 |
| | | SEQ ID |
| | | NO: 220 |
| 240 | ASGGLAVNRHVG | 251 Y7 |
| | | SEQ ID |
| | | NO: 224 |
| 239 | TWAGLAIEKF-- | 248 Y10 |
| | | SEQ ID |
| | | NO: 227 |

In an aspect, a YDFG polynucleotide encodes a YDFG polypeptide comprising 80, 85, 90, 91, 92, 93, 94, 95, 96, 97, 98, 99% or greater sequence identity to SEQ ID NO:220, 224, or 227, wherein the same amino acid that occurs in all three polypeptides in the same position (e.g., position 7 where all three sequences have a V) shown in the alignment above are retained. In an aspect, a YDFG polynucleotide encodes a YDFG polypeptide comprising 80, 85, 90, 91, 92, 93, 94, 95, 96, 97, 98, 99% or greater sequence identity to SEQ ID NO:218, 220, or 222, wherein one or more conservative amino acid mutations are present.

In an aspect, a YDFG polynucleotide encodes a YDFG polypeptide comprising 80, 85, 90, 91, 92, 93, 94, 95, 96, 97, 98, 99% or greater sequence identity to SEQ ID NO:219, 221, 223, 225, or 226. Two or more polypeptides set forth in SEQ ID NO:218-227 can be aligned as demonstrated above to determine which amino acids can be retained and which can be substituted. In an aspect, a YDFG polynucleotide encodes a YDFG polypeptide comprising 80, 85, 90, 91, 92, 93, 94, 95, 96, 97, 98, 99% or greater sequence identity to SEQ ID NO: 219, 221, 223, 225, or 226, wherein one or more conservative amino acid mutations are present.

In an aspect, a YDFG polynucleotide can be derived from Beauveria sp., Plautia sp., Erwinia sp., Helicobacter sp., Pseudomonas sp., Acetobacter sp., Gluconobacter sp., Oleiagrimonas sp., Geobacter sp., and/or Campylobacter sp. In an aspect a YDFG polynucleotide can be derived from Beauveria bassiana D1-5. Plautia stali, Erwinia mallotivora, Helicobacter pylori, Pseudomonas citronellolis, Acetobacter sicerae, Gluconobacter oxydans NBRC 3293, Oleiagrimonas soli, Geobacter soli, and/or Campylobacter concisus.

The one or more heterologous polynucleotides encoding one or more copies of BAPAT or YDFG can be integrated into the recombinant yeast's chromosome.

The one or more heterologous polynucleotides encoding one or more copies of PAND, PYC, AAT, BAPAT and/or YDFG can be integrated into the recombinant yeast's chromosome.

In an aspect, a recombinant yeast can comprise one or more polynucleotides encoding 1, 2, 3, 4, or all 5 of PAND, PYC, AAT, YDFG, and BAPAT in any combination. Additionally, a PDC and/or GPD gene can be inactivated in the recombinant yeast. In an aspect, all PDC and/or all GPD genes can be inactivated in the recombinant yeast.

### Methods of Producing 3-Hydroxypropionic Acid (3HP)

Provided herein are methods of producing 3-hydroxypropionic acid (3HP). In an aspect one or more of the recombinant yeast strains disclosed herein are added to cell culture media. The recombinant yeast are cultivated to a desired cell density (e.g., about 10³, 10⁴, 10⁵, 10⁶, 10⁷, 10⁸, 10⁹, 10¹⁰ or more cells/mL) and the 3HP is harvested. In an aspect, the recombinant yeast can comprise one or more polynucleotides encoding 1, 2, 3, 4, or all 5 of PAND, PYC, AAT, YDFG, and BAPAT in any combination. Additionally, one or more of a PDC and/or one or more of a GPD gene can be inactivated in the recombinant yeast. In an aspect, the yeast is Issatchenkia sp., Issatchenkia orientalis, or other suitable yeast.

3HP can be produced at about 40, 50, 60, 70, 75, 80, 90, 100 g/L or more. The yield of 3HP can be about 0.2, 0.3, 0.4, 0.5, 0.6, 0.7, 0.8, 0.9 g/g glucose or more. In an aspect, the glucose concentration during fermentation is less than about 60, 50, 40, 30, 20, or 10 g/L. In an aspect, aeration during fermentation is about 0.2, 0.3, 0.4, 0.5, or 0.56 vvm. The pH of the cell culture media can be about 3.50, 3.75, 4.00, 4.25, 4.50, or more. The pH of the cell culture media can be about 4.50, 4.25, 4.00, 3.75, 3.50, or less. In an aspect, the dissolved oxygen of the cell culture media can be about 3, 4, 5, 6, 7% or more. In an aspect, CO₂ can be provided to the cell culture media during a 3HP production phase.

In an aspect, the cell culture media can be, for example, corn steep liquor, synthetic complete (SC) medium, synthetic complete dropout medium, yeast extract-peptone-dextrose (YPD) medium, or any other suitable medium.

In an aspect, less than about 0.2, 0.3, 0.4, 0.5, 0.6, 0.7, 0.8, 0.9, 1.0 g/L of pyruvate is produced during fermentation. The amount of pyruvate can be determined at, for example, 2, 3, 4, 5, 6, 7, or more days after inoculation of the cell culture media with the recombinant yeast.

In an aspect, a fermentation can be continuous, batch, or fed-batch fermentation. Fed-batch fermentation is a controlled bioprocessing method where a biological culture is initiated with a defined volume of medium and subsequently supplied with additional substrates during cultivation without removing a significant portion of the culture liquid. Fed-batch fermentation maintains a closed reactor volume but permits incremental or continuous feeding of selected components. In batch fermentation all nutrients are present from the outset. In continuous fermentation the culture is steadily added and removed. In an aspect, a fermentation process can begin in a batch mode and then and then changed to a fed-batch mode. The change over can occur, for example, when glucose limitation is observed.

In fed-batch fermentation, cells, enzymes, or other biological systems are cultivated under controlled environmental conditions (e.g., agitation, temperature, pH, and gas flow) while one or more limiting nutrients are introduced into the vessel according to a predetermined profile. This controlled addition modulates the metabolic state of the culture, allowing the operator to manage growth rate, reduce accumulation of inhibitory by-products, and influence the formation of desired metabolites, proteins, or biochemical intermediates.

The feed strategy can employ linear, exponential, or feedback-regulated delivery mechanisms. These mechanisms can be based on parameters such as dissolved oxygen trends, biomass-related signals, substrate concentration estimations, or other measurable indicators of culture status. By adjusting feed composition and timing, the process can be directed toward extended growth phases or enhanced production phases, depending on the target output.

Throughout the operation, the culture volume increases as feed is added, typically approaching but not exceeding the vessel's working capacity. Because no substantial effluent is removed, the system retains accumulated biomass and secreted products, enabling higher final product concentrations than are commonly achievable in batch mode. This mode also permits prolonged cultivation periods, providing operational flexibility for organisms or production systems that benefit from extended nutrient-limited or growth-controlled conditions.

### Methods of Isolating 3-HP

Currently, 3-HP is primarily synthesized from petroleum-based chemical routes that are both energy-intensive and environmentally detrimental, requiring operating temperatures of 260 to 370° C. and generating 3.3 to 7.7 kg CO₂ per kilogram of product. In contrast, microbial fermentation offers a more sustainable and economically favorable alternative, largely due to its ability to utilize renewable and waste-derived feedstocks such as crude glycerol and lignocellulosic residues, thereby reducing both raw material costs and net carbon emissions. However, downstream processing (DSP) remains one of the most challenging and cost-intensive steps in industrial biomanufacturing. For bulk fermentation products, DSP typically account for 30 to 40% of the total production cost, and for carboxylic acids, it can rise to as high as 60%. Although numerous studies have focused on enhancing 3-HP biosynthesis, the efficient recovery of purified 3-HP, particularly in solid form, from low-pH fermentation broth remains a key research challenge. Low-pH fermentation offers distinct advantages, including the elimination of gypsum and avoidance of costly acidification step during DSP, thereby simplifying the process and improving both its economic and environmental sustainability.

Methods of isolating sodium 3-hydroxypropionic acid from a fermentation broth are provided herein. After the completion of a fermentation as described herein yeast cells can be removed from the fermentation broth by, for example, centrifugation or other suitable method to form a cell-free fermentation broth. The cell-free fermentation broth can be decolorized to form a cell-free, decolorized fermentation broth using activated carbon or any other suitable method. In an aspect, activated carbon or charcoal can be used at about 1, 2, 3, 4, 5, 7, 10% w/v or more. Cations and anions can be removed from the cell-free, decolorized fermentation broth. In an aspect, NaOH can be added to form sodium 3-hydroxypropionic acid. The methods can further comprise evaporating and drying the sodium 3-hydroxypropionic acid. Optionally, the cell-free fermentation broth can be concentrated prior to the dying steps to achieve a 20-40% (w/v) 3-hydroxypropionic acid solution.

In an aspect, cations can be removed using a cation exchange resin (e.g., a strong acid cation exchange resin having sulfonic acid functional groups (R—SO3)) and anions can be removed using an anion exchange resin (e.g., a weak base anion exchange resin having tertiary amine functional groups). Any other suitable method for removal can also be used. In an aspect, 3-HP is produced under temperatures of about 20, 25, 30, 35° C. or more. In an aspect, the solution can be evaporated and dried to form solid sodium 3-hydroxypropionic acid. Optionally, the solution can be concentrated to achieve a 20-40% (w/v) 3-hydroxypropionic acid solution.

In an aspect, 3-HP can be produced by obtaining a cell-free fermentation broth using any of the yeast described herein. The cell-free fermentation broth can be treated with about 1, 2, 3, 4, 5, 7, 10% w/v or more of activated charcoal or carbon at about 30° C. The solution can be run through a strong cation exchange resin (e.g., a resin with sulfonic acid functional groups (R—SO3)). The pH can be adjusted to about 3.5, 4,0, 4.5 or 5.0 using NaOH. The solution can be run through a weak anion exchange resin (e.g., a resin with tertiary amine functional groups). The solution can be neutralized with 10 M NaOH.

The 3-HP produced using the methods can be about 60, 70, 80, 90, 95% w/w or more pure. The 3-HP can be recovered at about 50, 60, 70, 80, 90% w/w or more.

Therefore, this study develops and validates a simplified, fully solvent-free downstream processing (DSP) strategy for high-purity recovery of 3-hydroxypropionic acid (3-HP) from real fermentation broth containing 62.3 g/L of 3-HP. Optimized activated carbon treatment achieved 98% color removal, while Amberlite IRA-67 was operated at pH 4.5 and 30° C. to minimize product loss. This is the first integrated demonstration of a fully solvent-free DSP enabling recovery of bio-based 3-HP as both a solid sodium salt and a concentrated aqueous solution, supported by techno-economic analysis. At lab scale, the process achieved 77.3% recovery of sodium 3-HP with 83.2% (w/w) purity and produced a 30% (w/v) aqueous solution. Techno-economic analysis yielded minimum selling prices of $0.551/kg for the solution and $0.892/kg for the salt, both below target thresholds for cost-competitive bio-acrylic acid production. Overall, these results demonstrate an efficient, scalable, and economically viable industrial pathway for 3-HP recovery.

The procedures described herein employ, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, et al., Molecular Cloning, Cold Spring Harbor Laboratory Press, Cold Spring Harbor, N.Y. (1982); Sambrook et al., (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, NY (1992); Guthrie and Fink, Guide to Yeast Genetics and Molecular Biology, Academic Press, NY (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire, et al., RNA Interference Technology From Basic Science to Drug Development, Cambridge University Press, Cambridge (2005); Schepers, RNA Interference in Practice, Wiley-VCH (2005); Engelke, RNA Interference (RNAi): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004)).

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by aspects and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the aspects described in broad terms above.

### EXAMPLES

### Example 1: Materials

All the strains and plasmids are listed in Table 4 and Table 5, respectively. E. coli DH5α was used to maintain and amplify plasmids. I. orientalis SD108, and S. cerevisiae YSG50 were propagated in Yeast-Peptone-Adenine-Dextrose (YPAD) medium consisting of 1% yeast extract, 2% peptone, 0.01% adenine hemisulphate, and 2% glucose. Recombinant I. orientalis strains were grown in Synthetic Complete (SC) dropout medium lacking uracil (SC-URA). LB broth, bacteriological grade agar, yeast extract, peptone, yeast nitrogen base (w/o amino acid and ammonium sulfate), ammonium sulfate, and D-xylose were obtained from Difco (BD, Sparks, MD), while complete synthetic medium was purchased from MP Biomedicals (Solon, OH). All restriction endonucleases, Q5 DNA polymerase and Phusion polymerase were purchased from New England Biolabs (Ipswich, MA). The QIAprep spin mini-prep kit and RNA isolation mini kit were purchased from Qiagen (Valencia, CA), whereas Zymoclean Gel DNA Recovery Kit and Zymoprep Yeast Plasmid Miniprep Kits were purchased from Zymo Research (Irvine, CA). All other chemicals and consumables were purchased from Sigma (St. Louis, MO), VWR (Radnor, PA), and Fisher Scientific (Pittsburgh, PA). Oligonucleotides including gBlocks and primers (Tables 8, 9, 10, and 11) were all synthesized by Integrated DNA Technologies and Twist Biosciences (IDT, Coralville, IA; Twist Biosciences, San Francisco, CA). For plasmid construction, Gibson assembly or DNA assembly was used. For strain construction, the CRISPR method was used for gene integration/deletion and the lithium acetate-mediated method was used for transformation. Detailed plasmid construction, strain construction and gene selection are described in Examples 20, 21, and 22.

### Example 2: PAND Gene Screening Assay

Plasmids encoding different PAND genes were transformed into I. orientalis SD108/ura3Δ. Three colonies were picked into 2 mL SC-URA with 50 g/L of glucose and cultivated for one day and then transferred to 20 mL SC-URA and cultivated for two days at 30° C. with shaking at 250 rpm. All cells were collected and adjusted to OD of 100. The cells were disrupted by the glass beads method. For glass beads disruption, cells were lysed using FastPrep-24 5G for 20 s (6.0 m/s) and the lysed cycle was repeated three times. In each interval, the sample was placed on ice for 2 min cooling. The cell extract was derived by FMOC first and then subjected to amino acid analysis to quantify the β-alanine production. For sample preparation for amino acid analysis, 370 μL of 500 mM Borate (pH 10) and 20 μL of FMOC (2.5 mg/mL in acetonitrile) were added to the 110 μL sample. The mixture was vortexed and stood at room temperature for at least 20 mins, followed by centrifugation and filtering.

### Example 3: BAPAT Gene Screening Assay

Plasmids encoding different BAPAT genes were transformed into I. orientalis SD108/ura3Δ. Three colonies were picked into 2 mL SC-URA with 50 g/L of glucose and cultivated for one day and then transferred to 20 mL SC-URA and cultivated for three days at 30° C. with shaking at 250 rpm. All cells were collected and adjusted to OD₆₀₀ of 100. The concentrated cells were disrupted by the glass beads method. To set up the in vitro enzyme assay, in 1 mL reaction, 900 μL of solution containing 11.1 mM of pyruvate, 11.1 mM of β-alanine, 0.11 mM of PLP, and 55.6 mM of phosphate buffer (pH7) were mixed with 100 μL of cell extract, and the reaction mixtures were incubated at 37° C. for an hour. Then, 25% TCA was added to quench the reaction. To quantify the malonic semialdehyde production, 110 μL of the reaction mixture was mixed with 140 μL of 68 mM PurpaId (4-amino-3-hydrazino-5-mercapto-1,2,4-triazole), and the purple product was measured at 540 nm using a plate reader (Molecular Devices, San Jose, CA).

### Example 4 YDFG Gene Screening Assay

Plasmids encoding different YDFG genes were transformed into I. orientalis SD108/ura3Δ. Three colonies were picked into 2 mL SC-URA with 50 g/L of glucose for one day and then transferred to 20 mL SC-URA for three days at 30° C. with shaking at 250 rpm. All cells were collected and concentrated to OD₆₀₀ of 100. Then, the cells were disrupted by the glass beads method. To set up the in vitro enzyme assay, in 1 mL reaction, 800 μl of solution containing 37.5 of mM pyruvate, 37.5 mM of β-alanine, 0.125 mM of PLP, 6.25 U of GDH, 2.5 mM of NADP+, 125 mM of glucose and 62.5 mM of phosphate buffer (pH 7) were mixed with 100 μL of YDFG-containing cell lysate and 100 μL of B6 cell lysate containing the BAPAT6 protein from Methanosarcina sp. MTP4. The reaction mixture was incubated at 37° C. for 2 hours. Then, 25% TCA was added to quench the reaction. 3HP was measured by HPLC.

### Example 5 Tube, Shake-Flask Fermentation and DASbox Fed-Batch Fermentation

For tube fermentation (2 mL), three colonies were picked into SC-URA with 50 g/L glucose and cultured for 5 days. 3HP was quantified at day 5. For shake flask fermentation (20 mL) using plasmid-harboring strain, three colonies were picked into 2 mL SC-URA with 50 g/L of glucose for 3 days at 30° C. with shaking at 250 rpm. Then, the cells were subcultured to 20 mL SC-URA with 50 g/L of glucose and 10 g/L of CaCO₃ and cultured at 30° C. with shaking at 250 rpm. For shake flask fermentation using all-integrated strains (i.e., 1AP and 2AP strain), three colonies were picked in YPAD for 1 day. Then, the cells were subcultured into 20 mL CSM with 50 g/L of glucose and 10 g/L of CaCO₃. The initial OD₆₀₀ was 0.2 and, the samples were collected every 24 hours for HPLC analysis. For fed-batch fermentation in the DASbox bioreactors (DASbox Bioreactors, Eppendorf, Hamburg, Germany, 100 mL working volume, Table 6), single colonies of the I. orientalis strain were inoculated into the YPAD medium and cultured for 1 day. Then, 1 mL of cells was injected into 100 mL of CSL liquid medium in the bioreactor. The cells were cultivated at 30° C. and the DO was set to 5%. A cascade program was used to maintain DO by changing the stirrer speed between 400-1200 RPM, O₂ concentration in the gas mix between 21-100% pure O₂ and then O₂ flow rate between 1-3 vvm while maintaining the pH at 4 using 4 M HCl and 28% ammonium hydroxide. When the DO dropped below 5%, CO₂ was introduced at 0.2 vvm and conjointly aeration dropped to 0.2 vvm. Therefore, total aeration remained at 0.4 vvm and at this point DO was maintained using agitation only. The CO₂ supply could provide an ample amount of bicarbonate for 3HP production.

### Example 6 Analytic Methods

For quantification of glucose consumption, Agilent 1200 HPLC system equipped with a refractive index detector (Agilent Technologies, Wilmington, DE, USA) and Rezex ROA-Organic Acid H+(8%) column (Phenomenex, Torrance, CA, USA) were used. The column and the detector were operated at 50° C., and 0.005 N H₂SO₄ was used as the mobile phase at a flow rate of 0.6 mL/min. For quantification of 3HP production and pyruvate accumulation, Shimadzu HPLC with a UV-Vis detector and Aminex HPX-87H Column #1250140 (BioRad, USA) were used with injection of 50 μL sample. The column was operated at 60° C. with 0.005 N H₂SO₄ at a flow rate of 0.6 mL/min and 210 nm was used as the detection wavelength. For quantification of amino acids, Agilent 1260 HPLC system equipped with a DAD detector (262 nm was used as detecting wavelength) and Phenomenex Kinetex® 5 μm EVO C18 100A LC column (150×4.6 mm² Phenomenex) were used with injection of 5 μL sample. The column was run at room temperature with a gradient elution process using solvent A (20 mM sodium acetate) and solvent B (acetonitrile). The elution process runs the following program: 18% B to 57% B (linear gradient, 0-13 min), 57% to 100% (linear gradient, 13-13.3 min), 100% B (isocratic elution, 13.3 to 14.3 min), 100% to 18% (linear gradient, 14.3-15.8 min), 18% B (isocratic elution, 15.8 to 18 min).

### Example 7 Genome-Scale Metabolic Modeling, Flux Balance Analysis, and Thermodynamic Analysis

The genome-scale metabolic model used for I. orientalis SD108 is ilsor850²³ which follows nomenclature of the BiGG database³⁸. To evaluate 3HP production, the model was augmented to separately include seven different 3HP pathways²⁴, as well as the transport of 3HP from the cytosol to the extracellular environment and an exchange flux for extracellular 3HP. The BiGG database³⁸, MetaNetX³⁹, ChEBI⁴⁰, and PubChem⁴¹ were used for information about the metabolites and reactions for pathway intermediates. In general, BiGG nomenclature for metabolite and reaction ids was used when available, with compatible ids created otherwise, as noted in the model files; within BiGG 3-hydroypropionic acid (3HP) has the id 3hpp, which was used in the model and simulations. Marvin was used for characterizing chemical structures including formula and charge information (Marvin 23.17.0, Chemaxon). In agreement with conventions of flux balance modeling, these values for each reaction in each pathway were computed using pH 7.2 for each intracellular species. The reaction set was also updated at the malonic semialdehyde reductase step for YDFG to use NADPH as a cofactor instead of NADH (which was used in previous simulations for S. cerevisiae⁴²).

Flux balance analysis (FBA) was used for model predictions⁴³. For the simulation of a given pathway, only the reactions associated with that pathway were added to the model. The glucose substrate uptake rate was set to 10 mmol g/DW/h glucose during simulations. The maximum oxygen uptake rate was limited to no more than 18.18 mmol g/DW/h during all simulations, as this is the minimal oxygen uptake that does not impact the maximum growth rate for a 10 mmol glucose g/DW/h basis. By convention exchange fluxes assume negative values for uptake, thus these constraints were set as V_{EX_glc_D_e}=−10 and 0 V_{EX_o2_e}≥−18.18. Model simulations of growth phenotype or 3HP formation were obtained using FBA with the objective of maximizing the flux of the biomass reaction (V_{biomass}) or product exchange flux (V_{EX_3hyp_e}) in the context of the indicated pathway. Yields are presented as carbon yields by normalizing to the carbons from glucose. Specifically, for each pathway p yield is computed as

$${Y}_{3 hpp , glucose}^{p} = \frac{{v}_{EX \_ 3 hhp \_ e , p}}{- {v}_{EX \_ glc \_ D \_ e} \cdot {y}_{3 hpp , glc \_ D}^{c}} \qquad ( 1 )$$

$$where {y}_{3 hpp , glc \_ D}^{c}$$

is the number of carbons in 3hpp (i.e., three) divided by those in glucose (i.e., six). Since V_{EX_glc_D_e} is fixed to −10, for maximum yield this expression can be simplified to

$${Y}_{3 hpp}^{p , max} = \frac{{v}_{EX \_ 3 hhp \_ e , p}^{max}}{20} \qquad ( 2 )$$

FBA calculations were implemented using GAMS 33.2.0 using IBM ILOG CPLEX solver. Adding non-native reactions and data processing were performed using the COBRApy package (version 0.29.0)⁴⁴ and pandas⁴⁵ within Jupyter notebooks⁴⁶.

Thermodynamics analysis of 3HP metabolic pathways was performed by estimating the standard Gibbs energy change ΔᵣG′⁰) of enzymatic reactions using dGPredictor²⁶ and eQuilibrator²⁷, with SMILES from structures determined using Marvin for compounds not available within KEGG⁴⁷. The evaluations were performed at pH 7 to compare with previous studies and at pH 6, which corresponds as a lower estimate of the intracellular pH in I. orientalis using the value that S. cerevisiae can have in an external environment of pH 4²⁵.

### Example 8 3HP Biomanufacturing Design and Simulation

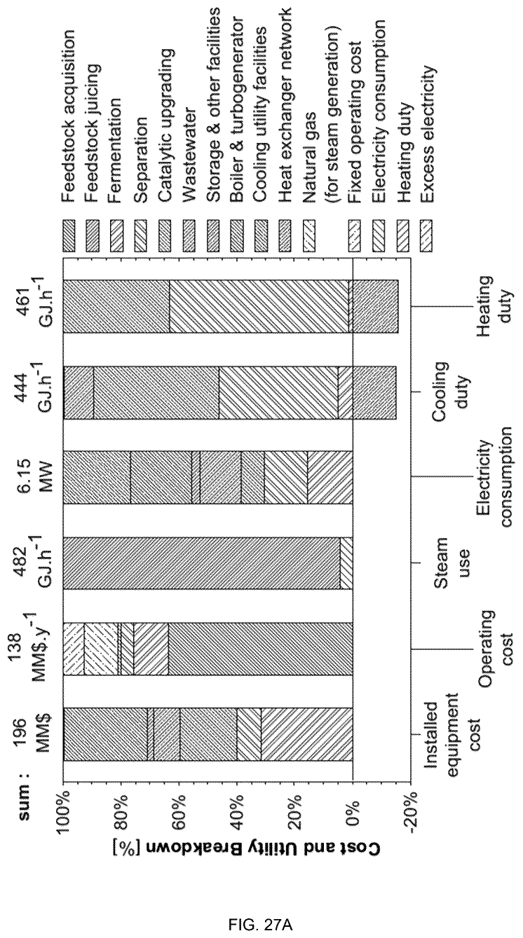

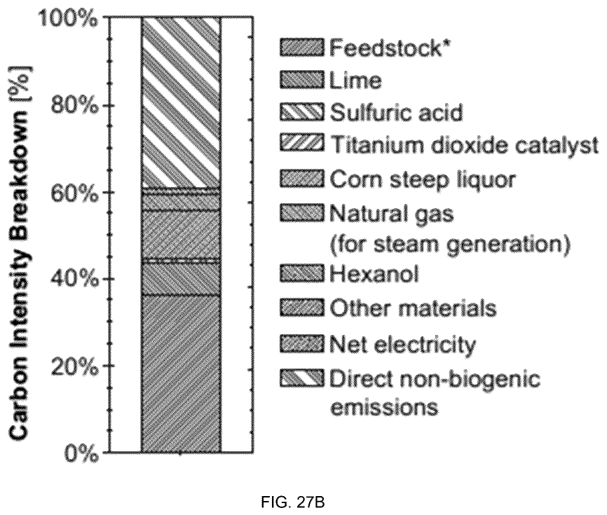

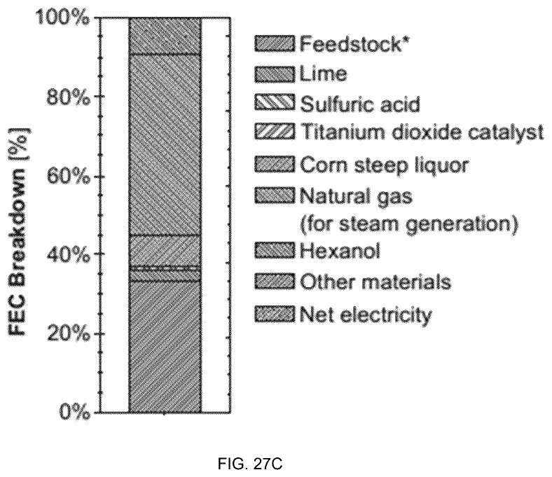

All 3HP biomanufacturing facilities discussed in this study were designed, simulated, and evaluated using BioSTEAM⁴⁸ and the thermodynamic package utilized was Thermosteam⁴⁹. Briefly, influent and effluent streams of each unit are simulated in BioSTEAM and coupled with operating parameters and equipment cost algorithms for unit design and cost calculations. Component equipment for major units (Table 18), breakdowns of system costs and environmental impacts (Tables 7 and 8, and FIG. 27), as well as baseline values and uncertainty distributions of key parameters (Table 19) are provided.

TABLE 3
List of major units and equipment included in the designed
and simulated 3HP biomanufacturing facilities.

| Process | ID | Unit | Equipment | Sources |
|---|---|---|---|---|
| corn dry grind | MH101 | Grain handling | grain handling | BioSTEAM; |
| pretreatment | | | | Zhang et al., 2023 |
| and | V102 | Corn storage | tank | BioSTEAM; |
| saccharification | | | | Zhang et al., 2023 |
| (300-L corn | MH103 | Cleaning | cleaning system | BioSTEAM; |
| scenario only) | | system | | Zhang et al., 2023 |
| | M104 | Hammer mill | hammer mill | BioSTEAM; |
| | | | | Zhang et al., 2023 |
| | V105 | Milled corn | tank | BioSTEAM; |
| | | surge tank | | Zhang et al., 2023 |
| | W106 | Milled corn | tank | BioSTEAM; |
| | | hopper | | Zhang et al., 2023 |
| | V107 | Milled corn | tank | BioSTEAM; |
| | | weigh tank | | Zhang et al., 2023 |
| | V301 | Alpha amylase | tank | BioSTEAM; |
| | | tank | | Zhang et al., 2023 |
| | P302 | Pump | pump, motor | BioSTEAM |
| | V305 | Lime hopper | tank | BioSTEAM; |
| | | | | Zhang et al., 2023 |
| | V307 | Slurry mix tank | tank | BioSTEAM; |
| | | | | Zhang et al., 2023 |
| | P311 | Pump | pump, motor | BioSTEAM |
| | E312 | Heat | floating head | BioSTEAM; |
| | | exchanger | | Zhang et al., 2023 |
| | E313 | Jet cooker | jet cooker | BioSTEAM; |
| | | | | Zhang et al., 2023 |
| | V314 | Cooked slurry | tank | BioSTEAM; |
| | | surge tank | | Zhang et al., 2023 |
| | P308 | Pump | pump, motor | BioSTEAM |
| | HX101 | Heat | floating head | BioSTEAM; |
| | | exchanger | | Zhang et al., 2023 |
| | V310 | Liquefaction | tank | BioSTEAM; |
| | | | | Zhang et al., 2023 |
| | V319 | Alpha amylase | tank | BioSTEAM; |
| | | tank | | Zhang et al., 2023 |
| | P320 | Pump | pump, motor | BioSTEAM |
| | V321 | Alpha amylase | tank | BioSTEAM; |
| | | tank | | Zhang et al., 2023 |
| | P322 | Pump | pump, motor | BioSTEAM |
| | S201 | Solids | centrifuges | BioSTEAM; |
| | | centrifuge | | Zhang et al., 2023 |
| sugarcane | U201 | Crushing mill | crushing mill | BioSTEAM; |
| juicing and | | | | Yu et al., 2022 |
| clarification | S201 | Vibrating | vibrating screen | BioSTEAM; |
| (300-L | | screen | | Yu et al., 2022 |
| sugarcane | M201 | Mixer | mixer | BioSTEAM |
| scenario only) | T202 | Storage tank | tank | BioSTEAM |
| | H201 | Heat | floating head | BioSTEAM |
| | | exchanger | | |
| | T203 | Mix tank | tank | BioSTEAM |
| | P201 | Pump | pump, motor | BioSTEAM |
| | U202 | Conveying belt | conveying belt | BioSTEAM; |
| | | | | Yu et al., 2022 |
| | T204 | Mix tank | tank | BioSTEAM |
| | T205 | Mix tank | tank | BioSTEAM |
| | P202 | Pump | pump, motor | BioSTEAM |
| | M202 | Mixer | mixer | BioSTEAM |
| | H202 | Heat | floating head | BioSTEAM |
| | | exchanger | | |
| | T206 | Mix tank | tank | BioSTEAM |
| | C201 | Clarifier | clarifier | BioSTEAM |
| | C202 | Rotary vacuum | vessels, vacuum | BioSTEAM; |
| | | filter | system - liquid-ring | Yu et al., 2022 |
| | | | pump, oil seal, | |
| | | | vacuum system | |
| | P203 | Pump | pump, motor | BioSTEAM |
| corn stover | H2SO4_storage | Sulfuric acid | pump, tank | BioSTEAM; |
| pretreatment | | storage tank | | Qin et al., 2024 |
| and | T201 | Sulfuric acid | pump, tank | BioSTEAM; |
| saccharification | | tank | | Qin et al., 2024 |
| | M201 | Sulfuric acid | mixer | BioSTEAM; |
| | | mixer | | Qin et al., 2024 |
| | M203 | Steam mixer | steam mixer | BioSTEAM; |
| | | | | Qin et al., 2024 |
| | R201 | Pretreatment | horizontal pressure | BioSTEAM; |
| | | reactor system | vessel, platform | Qin et al., 2024 |
| | | | and ladders, | |
| | | | pretreatment | |
| | | | reactor system | |
| | P201 | Blowdown | pump | BioSTEAM; |
| | | discharge | | Qin et al., 2024 |
| | | pump | | |
| | T202 | Oligomer | agitator, pump, | BioSTEAM; |
| | | conversion tank | tank | Qin et al., 2024 |
| | F201 | Pretreatment | agitator, pump, | BioSTEAM; |
| | | flash | tank, vapor | Qin et al., 2024 |
| | | | condenser, heat | |
| | | | exchanger, | |
| | | | vacuum system | |
| | Ammonia_storage | Ammonia | tank | BioSTEAM; |
| | | storage tank | | Qin et al., 2024 |
| | M205 | Ammonia mixer | mixer | BioSTEAM; |
| | | | | Qin et al., 2024 |
| | T203 | Ammonia | agitator, tank | BioSTEAM; |
| | | addition tank | | Qin et al., 2024 |
| | P202 | Hydrolysate | pump | BioSTEAM; |
| | | pump | | Qin et al., 2024 |
| | M301 | Enzyme | mixer | BioSTEAM; |
| | | hydrolysate | | Qin et al., 2024 |
| | | mixer | | |
| | H301 | Hydrolysate | heat exchanger | BioSTEAM; |
| | | heat exchanger | | Qin et al., 2024 |
| | R301 | Continuous | tanks, transfer | BioSTEAM; |
| | | presaccharification | pumps | Qin et al., 2024 |
| | R305 | Saccharification | heat exchangers, | BioSTEAM; |
| | | | reactors, agitators, | Qin et al., 2024 |
| | | | cleaning in place, | |
| | | | recirculation pumps | |
| | M204 | Mixer | mixer | BioSTEAM, ⁵ |
| | H201 | Waste vapor | heat exchanger | BioSTEAM; |
| | | condenser | | Qin et al., 2024 |
| fermentation (all | F301 | Multi-effect | evaporators, | BioSTEAM |
| scenarios) | | evaporator | condenser, mixer, | |
| | | | vacuum system | |
| | M304_P | Pump | pump, motor | BioSTEAM |
| | M304 | Mixer | mixer | BioSTEAM |
| | M304_H | Heat | floating head | BioSTEAM |
| | | exchanger | | |
| | S302 | Splitter | splitter | BioSTEAM |
| | R303 | Seed train | heat exchangers, | |
| | | | reactors, agitators, | |
| | | | cleaning in place, | |
| | | | recirculation pumps | |
| | T301 | Seed hold tank | pump, agitator, | BioSTEAM |
| | | | tank | |
| | S303 | Splitter | splitter | BioSTEAM |
| | K302 | Isothermal | compressor(s) | BioSTEAM |
| | | compressor | | |
| | V302 | Valve | valve | BioSTEAM |
| | R302 | Batch co | heat exchangers, | |
| | | fermentation | reactors, agitators, | |
| | | | cleaning in place, | |
| | | | recirculation pumps | |
| | M305 | Mixer | mixer | BioSTEAM |
| | A301 | Amine | makeup tank, | BioSTEAM |
| | | absorption | cooler, cross heat | |
| | | | exchanger, | |
| | | | reboiler, | |
| | | | condenser, pumps, | |
| | | | stripper, absorber | |
| | K301 | Isothermal | compressor(s) | BioSTEAM |
| | | compressor | | |
| | H302 | Heat | heat exchanger | BioSTEAM |
| | | exchanger | | |
| | P303 | Pump | pump, motor | BioSTEAM |
| | F301_P | Pump | pump, motor | BioSTEAM |
| separation (all | S401 | Solids | centrifuges | BioSTEAM |
| scenarios) | | centrifuge | | |
| | R401 | Acidulation | tank | BioSTEAM |
| | | reactor | | |
| | R401_H | Heat | floating head | BioSTEAM |
| | | exchanger | | |
| | R401_P | Pump | pump, motor | BioSTEAM |
| | S402 | Gypsum filter | centrifuges | BioSTEAM |
| | A410 | Adsorption | vertical pressure | BioSTEAM, this |
| | | | vessel, platform | study |
| | | | and ladders, | |
| | | | activated carbon, | |
| | | | heat exchanger | |
| | | | regeneration - | |
| | | | double pipe, heat | |
| | | | exchanger drying, | |
| | | | heat exchanger | |
| | | | regeneration | |
| | M420 | Mixer | mixer | BioSTEAM |
| | A420 | Cation | vertical pressure | BioSTEAM, this |
| | | exchange | vessel, platform | study |
| | | | and ladders, silica | |
| | | | gel, heat | |
| | | | exchanger | |
| | | | regeneration - | |
| | | | double pipe, heat | |
| | | | exchanger drying, | |
| | | | heat exchanger | |
| | | | regeneration | |
| | M430 | Mixer | mixer | BioSTEAM |
| | A430 | Anion | vertical pressure | BioSTEAM, this |
| | | exchange | vessel, platform | study |
| | | | and ladders, silica | |
| | | | gel, heat | |
| | | | exchanger | |
| | | | regeneration - | |
| | | | double pipe, heat | |
| | | | exchanger drying, | |
| | | | heat exchanger | |
| | | | regeneration | |
| | F403 | Multi-effect | evaporators, | BioSTEAM |
| | | evaporator | condenser - | |
| | | | floating head, | |
| | | | vacuum system - | |
| | | | liquid-ring pump, | |
| | | | one stage water | |
| | | | seal, evaporators, | |
| | | | condenser, mixer, | |
| | | | vacuum system | |
| | F403_P1 | Pump | pump, motor | BioSTEAM |
| | F403_P2 | Pump | pump, motor | BioSTEAM |
| separation of | M401 | Mixer | mixer | BioSTEAM |
| sodium 3- | F404 | Drum dryer | drum dryer | BioSTEAM |
| hydroxypropionate | | | | |
| (300-L | | | | |
| dextrose 3HP | | | | |
| salt scenario | | | | |
| only) | | | | |
| upgrading | M402 | Mixer | mixer | BioSTEAM |
| (DASbox | M402_P | Pump | pump, motor | BioSTEAM |
| dextrose, 10-L | R402 | Dehydration | vertical pressure | BioSTEAM; |
| dextrose, 300-L | | reactor | vessel, platform | Kim et al., 2020; |
| dextrose, 300-L | | | and ladders, tio2 | Wu et al., 2023 |
| corn, 300-L | | | catalyst | |
| sugarcane, and | R402_H | Heat | floating head | BioSTEAM |
| 300-L corn | | exchanger | | |
| stover | D408 | Divided | rectifier trays, | BioSTEAM |
| scenarios only) | | Distillation | stripper trays, | |
| | | Column | rectifier tower, | |
| | | | stripper platform | |
| | | | and ladders, | |
| | | | stripper tower, | |
| | | | rectifier platform | |
| | | | and ladders, | |
| | | | reboiler - floating | |
| | | | head, vacuum | |
| | | | system - liquid-ring | |
| | | | pump, one stage | |
| | | | water seal, | |
| | | | condenser - | |
| | | | floating head, | |
| | | | pump - pump, | |
| | | | pump - motor, | |
| | | | reboiler, reflux | |
| | | | drum, vacuum | |
| | | | system, top split, | |
| | | | bottoms split, | |
| | | | condenser, pump | |
| | D408_P | Pump | pump, motor | BioSTEAM |
| | D409 | Divided | rectifier trays, | BioSTEAM |
| | | Distillation | stripper trays, | |
| | | Column | rectifier tower, | |
| | | | stripper platform | |
| | | | and ladders, | |
| | | | stripper tower, | |
| | | | rectifier platform | |
| | | | and ladders, | |
| | | | reboiler - floating | |
| | | | head, vacuum | |
| | | | system - liquid-ring | |
| | | | pump, two stage | |
| | | | water seal, | |
| | | | condenser - | |
| | | | floating head, | |
| | | | pump - pump, | |
| | | | pump - motor, | |
| | | | reboiler, reflux | |
| | | | drum, vacuum | |
| | | | system, top split, | |
| | | | bottoms split, | |
| | | | condenser, pump | |
| | D409_bP | Pump | pump, motor | BioSTEAM |
| | D408_dP | Pump | pump, motor | BioSTEAM |
| | D409_dP | Pump | pump, motor | BioSTEAM |
| | D409_H | Heat | floating head | BioSTEAM |
| | | exchanger | | |
| | D409_P | Pump | pump, motor | BioSTEAM |
| wastewater | M501 | Mixer | mixer | BioSTEAM |
| treatment (all | M502 | Mixer | mixer | BioSTEAM |
| scenarios) | U501 | Internal | tank, sludge pump - | BioSTEAM; |
| | | circulation rx | pump, sludge | Liu et al., 2023 |
| | | | pump - motor, heat | |
| | | | exchanger - | |
| | | | floating head, | |
| | | | effluent pump - | |
| | | | pump, effluent | |
| | | | pump - motor, | |
| | | | sludge pump, heat | |
| | | | exchanger, effluent | |
| | | | pump | |
| | U502 | An MBR | reactor excavation, | BioSTEAM; |
| | | | wall concrete, slab | Liu et al., 2023 |
| | | | concrete, | |
| | | | membrane, gac, | |
| | | | pumps, pump | |
| | | | building, pump | |
| | | | excavation, air | |
| | | | pipes, blowers, | |
| | | | blower building, | |
| | | | degassing | |
| | | | membrane, heat | |
| | | | exchanger | |
| | U503 | Polishing filter | filter tank | BioSTEAM; |
| | | | excavation, wall | Liu et al., 2023 |
| | | | concrete, slab | |
| | | | concrete, packing | |
| | | | ldpe, packing | |
| | | | hdpe, pumps, | |
| | | | pump building, | |
| | | | pump excavation, | |
| | | | degassing | |
| | | | membrane, heat | |
| | | | exchanger - | |
| | | | floating head, heat | |
| | | | exchanger | |
| | S501 | Splitter | splitter | BioSTEAM |
| | U504 | Belt thickener | thickeners, sludge | BioSTEAM; |
| | | | pump - pump, | Liu et al., 2023 |
| | | | sludge pump - | |
| | | | motor, effluent | |
| | | | pump - pump, | |
| | | | effluent pump - | |
| | | | motor, sludge | |
| | | | pump, effluent | |
| | | | pump | |
| | C501 | Sludge | centrifuges, sludge | BioSTEAM; |
| | | centrifuge | pump - pump, | Liu et al., 2023 |
| | | | sludge pump - | |
| | | | motor, effluent | |
| | | | pump - pump, | |
| | | | effluent pump - | |
| | | | motor, sludge | |
| | | | pump, effluent | |
| | | | pump | |
| | M503 | Mixer | mixer | BioSTEAM |
| | M504 | Mixer | mixer | BioSTEAM |
| | U505 | Biogas | reactor | BioSTEAM; |
| | | upgrading | | Liu et al., 2023 |
| | U506 | Reverse | evaporator, reactor | BioSTEAM; |
| | | osmosis | | Liu et al., 2023 |
| | M510 | Mixer | mixer | BioSTEAM |
| boiler & | BT701 | Boiler | baghouse bags, | BioSTEAM |
| turbogenerator | | turbogenerator | boiler, deaerator, | |
| (all scenarios) | | | amine addition pkg, | |
| | | | hot process water | |
| | | | softener system, | |
| | | | turbogenerator | |
| cooling utility | CWP803 | Chilled water | chilled water | BioSTEAM |
| facilities (all | | package | package | |
| scenarios) | CWP802 | Chilled water | chilled water | BioSTEAM |
| | | package | package | |
| | CT801 | Cooling tower | cooling tower, | BioSTEAM |
| | | | cooling water pump | |
| heat exchanger | HXN1001 | Heat | heat exchangers | BioSTEAM |
| network (all | | exchanger | | |
| scenarios) | | network | | |
| storage & other | T603 | CSL storage | pump, agitator, | BioSTEAM |
| facilities (all | | tank | tank | |
| scenarios) | T604 | Lime storage | dust vent | BioSTEAM |
| | | tank | baghouse, | |
| | | | unloading blower, | |
| | | | feeder, storage bin | |
| | T609 | Magnesium | tank | BioSTEAM |
| | | chloride | | |
| | | storage tank | | |
| | T610 | Zinc sulfate | tank | BioSTEAM |
| | | storage tank | | |
| | T605 | Sulfuric acid | pump, tank | BioSTEAM |
| | | storage tank | | |
| | T611 | Sulfuric acid | tank | BioSTEAM |
| | | storage tank | | |
| | T612 | Caustics | tank | BioSTEAM |
| | | storage tank | | |
| | T608 | Sodium | tank | BioSTEAM |
| | | hydroxide | | |
| | | storage tank | | |
| | M901 | Mixer | mixer | BioSTEAM |
| | ADP902 | Air distribution | plant air | BioSTEAM |
| | | package | compressor, | |
| | | | instrument air | |
| | | | dryer, plant air | |
| | | | receiver | |
| | CIP901 | CIP Package | cip package | BioSTEAM |
| | FWT903 | Fire water tank | pump, tank | BioSTEAM |
| | PWC904 | Process water | tank, process water | BioSTEAM |
| | | center | pump, makeup | |
| | | | water pump | |
| product storage | T620 | Acrylic acid | tank | BioSTEAM |
| (DASbox | | storage tank | | |
| dextrose, 10-L | T620_P | Acrylic acid | pump, motor | BioSTEAM |
| dextrose, 300-L | | pump | | |
| dextrose, 300-L | | | | |
| corn, 300-L | | | | |
| sugarcane, and | | | | |
| 300-L corn | | | | |
| stover | | | | |
| scenarios only) | | | | |
| product storage | T620 | Sodium 3- | tank | BioSTEAM |
| (300-L dextrose | | hydroxypropionate | | |
| 3HP salt | | storage tank | | |
| scenario only) | T620_P | Sodium 3- | conveying belt | BioSTEAM |
| | | hydroxypropionate | | |
| | | conveying belt | | |

Simulations were run based on experimental fermentation performance at 300 mL and for multiple feedstocks (dextrose, corn, sugarcane, and corn stover). The dextrose-accepting biomanufacturing facilities in this study are comprised of three main (inside battery limits) processes (fermentation, separation, and catalytic upgrading) with outside-battery wastewater treatment and miscellaneous facilities (boiler, turbogenerator, cooling utility regeneration, heat exchanger network, clean-in-place, air distribution, fire water distribution, and process water distribution) with all design assumptions consistent with Bhagwat et al.¹⁶ other than for fermentation (based on experimental data obtained in this study). As done in previous studies that involved TEA and LCA to assess the production of platform chemicals,^{16,51,52} the designed biomanufacturing facilities included catalytic upgrading of the platform chemical (3HP) to a commodity chemical (acrylic acid) as no established market yet exists for 3H P. Including catalytic upgrading to acrylic acid in the designed biomanufacturing facilities enabled meaningful comparisons to market price ranges and environmental impacts from conventional production of acrylic acid (detailed under Techno-economic analysis and Life cycle assessment). Further descriptions of design and modeling assumptions for these processes (including catalytic upgrading) and facilities are detailed in Bhagwat et al.¹⁶, and all baseline values, references, and uncertainty distributions of key parameters are included in the Table 19.

TABLE 19
List of parameters included in uncertainty and sensitivity analyses for the 300-L dextrose scenario, and parameters for the
DASbox dextrose, 10-L dextrose, 300-L corn, 300-L sugarcane, and 300-L corn stover scenarios changed relative to the 300-L
dextrose scenario. Note all chemical prices were converted to the TEA year, 2019 (conversion m19thod detailed in the script⁸).

| | | | | | | | |
|---|---|---|---|---|---|---|---|
| Parameter name | Units | Baseline | Shape | Lower | Mode | Upper | References |

300-L dextrose scenario

| | | | | | | | |
|---|---|---|---|---|---|---|---|
| Plant annual | d | 330 | Triangular | 297 | 330 | 363 | baseline from |
| operating days | | | | | | | Borodina et al., |
| | | | | | | | 2015; bounds are |
| | | | | | | | baseline ±10% |
| Feedstock unit | $/wet-kg | 0.28 | Triangular | 0.23 | 0.27 | 0.34 | baseline is mean |
| price | | | | | | | of production |
| | | | | | | | costs reported in |
| | | | | | | | three studies |
| | | | | | | | (Zhang et al., |
| | | | | | | | 2022; Batista et |
| | | | | | | | al., 2024; Xiao |
| | | | | | | | et al., 2014) |
| | | | | | | | reviewed by |
| | | | | | | | Fatma et al., |
| | | | | | | | 2023; bounds are |
| | | | | | | | minimum and |
| | | | | | | | maximum values |
| Natural gas | $/kg | 0.2765 | Triangular | 0.2163 | 0.2765 | 0.3321 | minimum, mean, and |
| unit price | | | | | | | maximum price |
| | | | | | | | during 2010-2019 |
| | | | | | | | (Tran et al., 2019) |
| Electricity unit | $/kWh | 0.07 | Triangular | 0.067 | 0.07 | 0.074 | minimum, mean, and |
| price | | | | | | | maximum price |
| | | | | | | | during 2010-2019 |
| | | | | | | | (Tran et al., 2019) |
| CSL unit price | $/kg | 0.0747 | Uniform | 0.05976 | | 0.08964 | baseline from |
| | | | | | | | Tran et al., 2023; |
| | | | | | | | bounds are |
| | | | | | | | baseline ±20% |
| Magnesium chloride | $/kg | 0.38 | Uniform | 0.349 | | 0.411 | baseline and bounds |
| unit price | | | | | | | are mean, minimum, |
| | | | | | | | and maximum from |
| | | | | | | | Qin et al., 2024 |
| Zinc sulfate | $/kg | 0.795 | Uniform | 0.657 | | 0.931 | baseline and bounds |
| unit price | | | | | | | are mean, minimum, |
| | | | | | | | and maximum from |
| | | | | | | | Qin et al., 2024 |
| Lime unit price | $/kg | 0.286 | Triangular | 0.175 | 0.286 | 0.315 | baseline from Xiao |
| | | | | | | | et al., 2014; lower |
| | | | | | | | bound is 2015-2019 |
| | | | | | | | mean from Cao et |
| | | | | | | | al., 2020; upper |
| | | | | | | | bound is |
| | | | | | | | baseline +10% |
| Sulfuric acid | $/kg | 0.10355 | Triangular | 0.093195 | 0.10355 | 0.113905 | baseline from |
| unit price | | | | | | | Zhang et al., 2022, |
| | | | | | | | bounds are |
| | | | | | | | baseline ±10% |
| TiO2 catalyst | $/kg | 2.565 | Triangular | 2.3085 | 2.565 | 2.8215 | baseline is mean |
| unit price | | | | | | | price from ^{58.}, |
| | | | | | | | bounds are |
| | | | | | | | baseline ±10% |
| Gypsum (disposal) | $/kg | 0 | Uniform | −0.0288 | | 0.00776 | lower bound from ⁵⁶, |
| unit price | | | | | | | upper bound from |
| | | | | | | | Alsaleem et al., 2024 |
| Desired annual | pure metric | 134000 | Triangular | 107200 | 134000 | 160800 | based on projected |
| production | ton acrylic | | | | | | growth in U.S. |
| | acid/y | | | | | | market; Werpy et al., |
| | | | | | | | 2004 discussed in |
| | | | | | | | Analytical methods in |
| | | | | | | | the manuscript |
| Federal | % | 0.21 | Uniform | 0.15 | | 0.28 | baseline from Tran |
| corporate | | | | | | | et al., 2019; bounds |
| tax rate | | | | | | | based on Fatma et al, |
| | | | | | | | 2023; Suthers et al., |
| | | | | | | | 2020 |
| Internal rate | % | 0.1 | Uniform | 0.08 | | 0.12 | baseline for |
| of return | | | | | | | consistency with Kim |
| | | | | | | | et al., 2020; Qin |
| | | | | | | | et al., 2024; bounds |
| | | | | | | | are baseline ±20% |
| Fermentation | g/L | 23 | Uniform | 18.4 | | 27.6 | experimental design |
| CSL loading | | | | | | | in this study |
| Fermentation | g/L | 0.203 | Uniform | 0.01624 | | 0.2436 | |
| magnesium | | | | | | | |
| chloride | | | | | | | |
| loading | | | | | | | |
| Fermentation | g/L | 0.0066 | Uniform | 0.0053 | | 0.0079 | |
| zinc sulfate | | | | | | | |
| loading | | | | | | | |
| Fermentation | mol/L | 0.121 | Triangular | 0.097 | 0.121 | 0.145 | |
| lime loading | | | | | | | |
| Fermentation | % as | 2 | Uniform | 1 | | 3 | assumed |
| aeration rate | decimal | | | | | | |
| safety factor | | | | | | | |
| Fermentation | % as | 2 | Uniform | 1 | | 3 | assumed |
| CO2 sparging | decimal | | | | | | |
| rate safety | | | | | | | |
| factor | | | | | | | |
| Seed train | % as | 0.95 | Uniform | 0.9 | | 1 | baseline based on |
| fermentation | decimal | | | | | | Qin et al., 2024; |
| ratio | | | | | | | maximum is the |
| | | | | | | | theoretical limit |
| | | | | | | | (100%) and minimum |
| | | | | | | | mirrors the maximum |
| Inoculum ratio | % as | 0.01 | Uniform | 0.009 | | 0.011 | experimental design |
| | decimal | | | | | | in this study |
| Fermentation | % | 0.73 | Uniform | 0.584 | | 0.876 | experimental results |
| 3HP yield | theoretical | | | | | | from this study |
| Fermentation | g/L | 62.5 | Uniform | 50 | | 75 | |
| 3HP titer | | | | | | | |
| Fermentation | g/L/h | 0.326 | Uniform | 0.260 | | 0.391 | |
| 3HP productivity | | | | | | | |
| Fermentation I. | % | 0.245 | Uniform | 0.196 | | 0.294 | |
| orientalis cell | theoretical | | | | | | |
| mass yield | | | | | | | |
| Acidulation | h | 1 | Uniform | 0.9 | | 1.1 | baseline from |
| reaction time | | | | | | | BioSTEAM mix tank |
| Dehydration | wt % | 0.3 | Uniform | 0.27 | | 0.35 | baseline based on |
| feed HP weight | | | | | | | Jiang et al., 2009 |
| fraction | | | | | | | |
| Dehydration | % | 0.8 | Uniform | 0.72 | | 0.88 | baseline from Zallot |
| conversion | theoretical | | | | | | et al., 2019, upper |
| | | | | | | | bound based on Moussa |
| | | | | | | | et al., 2016, lower |
| | | | | | | | bound is baseline - |
| | | | | | | | 10% |
| Dehydration | h | 38.23 | Uniform | 34.41 | | 42.05 | baseline from Zallot |
| reaction time | | | | | | | et al., 2019, upper |
| | | | | | | | bound from Borodina |
| | | | | | | | et al., 2015, lower |
| | | | | | | | bound is baseline - |
| | | | | | | | 10% |
| Product acrylic | h | 168 | Triangular | 134.4 | 168 | 201.6 | baseline based on |
| acid storage | | | | | | | Kildegaard et al., |
| time | | | | | | | 2016; bounds are |
| | | | | | | | baseline ±20% |
| Boiler | % | 0.8 | Uniform | 0.72 | | 0.88 | baseline from Xiao |
| efficiency | | | | | | | et al., 2014; Ji et |
| | | | | | | | al., 2018 bounds are |
| | | | | | | | baseline ±10% based |
| | | | | | | | on Gao et al., 2013 |
| Turbogenerator | % | 0.85 | Uniform | 0.765 | | 0.935 | baseline from |
| efficiency | | | | | | | Borodina et al., |
| | | | | | | | 2015; bounds are |
| | | | | | | | baseline ±10% |

changed for the DASbox dextrose scenario

| | | | | | | | |
|---|---|---|---|---|---|---|---|
| Fermentation | g/L | 0.010 | Uniform | 0.008 | | 0.012 | experimental design |
| magnesium | | | | | | | in this study |
| chloride loading | | | | | | | |
| Fermentation | g/L | 0.010 | Uniform | 0.008 | | 0.012 | |
| zinc sulfate | | | | | | | |
| loading | | | | | | | |
| Fermentation | mol/L | 0.320 | Triangular | 0.300 | 0.320 | 0.355 | |
| lime loading | | | | | | | |
| Inoculum ratio | % as | 0.007 | Uniform | 0.0063 | | 0.0077 | |
| | decimal | | | | | | |
| Fermentation | % | 0.694 | Uniform | 0.555 | | 0.833 | experimental results |
| 3HP yield | theoretical | | | | | | from this study |
| Fermentation | g/L | 92 | Uniform | 73.6 | | 110.4 | |
| 3HP titer | | | | | | | |
| Fermentation | g/L/h | 0.548 | Uniform | 0.4384 | | 0.6576 | |
| 3HP | | | | | | | |
| productivity | | | | | | | |
| Fermentation I. | % | 0.15 | Uniform | 0.12 | | 0.18 | |
| orientalis cell | theoretical | | | | | | |
| mass yield | | | | | | | |

changed for the dextrose 10-L scenario

| | | | | | | | |
|---|---|---|---|---|---|---|---|
| Fermentation | g/L | 0.203 | Uniform | 0.162 | | 0.244 | experimental design |
| magnesium | | | | | | | in this study |
| chloride | | | | | | | |
| loading | | | | | | | |
| Fermentation | g/L | 0.0066 | Uniform | 0.0053 | | 0.0079 | |
| zinc sulfate | | | | | | | |
| loading | | | | | | | |
| Fermentation | mol/L | 0.211 | Triangular | 0.169 | 0.211 | 0.254 | |
| lime loading | | | | | | | |
| Inoculum ratio | % as | 0.05 | Uniform | 0.045 | | 0.055 | |
| | decimal | | | | | | |
| Fermentation | % | 0.747 | Uniform | 0.5976 | | 0.8964 | experimental results |
| 3HP yield | theoretical | | | | | | from this study |
| Fermentation | g/L | 74.3 | Uniform | 59.44 | | 89.16 | |
| 3HP titer | | | | | | | |
| Fermentation | g/L/h | 0.390 | Uniform | 0.312 | | 0.468 | |
| 3HP | | | | | | | |
| productivity | | | | | | | |
| Fermentation I. | % | 0.232 | Uniform | 0.186 | | 0.278 | |
| orientalis cell | theoretical | | | | | | |
| mass yield | | | | | | | |

changed for the 300-L corn scenario

| | | | | | | | |
|---|---|---|---|---|---|---|---|
| Feedstock unit | $/wet-kg | 0.139 | Triangular | 0.127 | 0.139 | 0.15 | baseline is mean |
| price | | | | | | | and bounds are 5th |
| | | | | | | | and 95th percentiles |
| | | | | | | | of 2015-2019 U.S. |
| | | | | | | | market prices |
| | | | | | | | reported by Valli |
| | | | | | | | et al., 2005 |

changed for the 300-L sugarcane scenario

| | | | | | | | |
|---|---|---|---|---|---|---|---|
| Plant annual | d | 180 | Triangular | 120 | 180 | 240 | baseline and bounds |
| operating days | | | | | | | based on Yu et al., |
| | | | | | | | 2022 |
| Feedstock unit | $/wet-kg | 0.036 | Triangular | 0.029 | 0.036 | 0.043 | baseline from Yu et |
| price | | | | | | | al., 2022, bounds are |
| | | | | | | | baseline ±10% |

changed for the 300-L corn stover scenario

| | | | | | | | |
|---|---|---|---|---|---|---|---|
| Plant annual | d | 330 | Triangular | 297 | 330 | 363 | baseline from Xiao |
| operating days | | | | | | | et al., 2014 and Ji |
| | | | | | | | et al., 2018; bounds |
| | | | | | | | are baseline ±10% |
| Feedstock unit | $/wet-kg | 0.069 | Triangular | 0.055 | 0.069 | 0.082 | baseline from Xiao |
| price | | | | | | | et al., 2014; bounds |
| | | | | | | | are baseline ±20% |

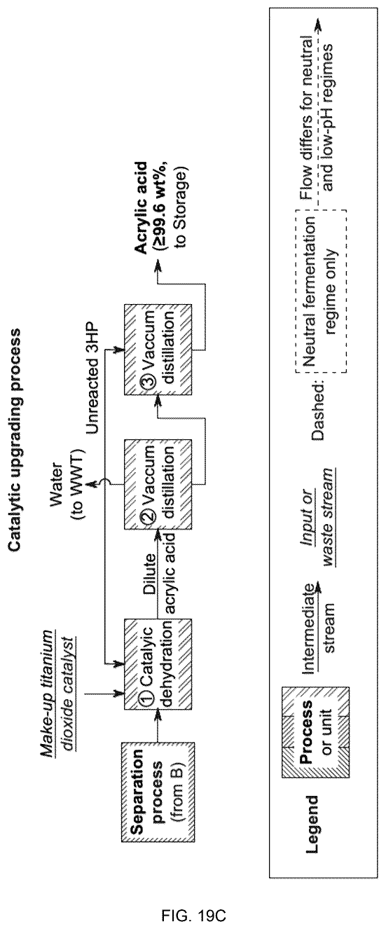

For biomanufacturing facilities in this study designed to accept biomass feedstocks, there were additional processes included inside battery limits: (i) for corn-accepting biomanufacturing facilities, design assumptions for the corn dry grind process were consistent with Cortes-Pena et al.⁴⁸; (ii) for sugarcane-accepting biomanufacturing facilities in this study, design assumptions for feedstock juicing and clarification were consistent with Bhagwat et al.⁵³; and (iii) for corn stover-accepting biomanufacturing facilities in this study, design assumptions for corn stover pretreatment and saccharification were consistent with Cortes-Pena et al.⁴⁸ For all biomanufacturing facilities, at each simulation, a heat exchanger network is newly synthesized using the Linnhoff-Hindmarsh pinch-based approach⁵⁴ to offset heating and cooling utility demands. The production capacity for all biomanufacturing facilities was set to 134,000 metric tons of acrylic acid annually in the baseline case to be consistent with a previous study.³⁵ The baseline production capacity was 2% of the global acrylic acid market size and 14% of U.S. capacity for glacial acrylic acid production in 2020, a fraction of the projected growth projected from 2021 to 2025 in the global market for acrylic acid (a growth of approximately 1,230,000 metric ton/y), and comparable to the projected growth in that 5-year period in the U.S. market (approximately 91,000 metric ton/y).³⁵ Further, a triangular distribution with bounds corresponding to ±20% of the baseline value was attributed to the annual production in the uncertainty analysis. Simplified block flow diagrams of the fermentation and separation processes are shown in FIG. 19 as well as baseline values and distributions for all parameters included in the uncertainty analysis in Table 19).⁵⁰

### Example 9 Techno-Economic Analysis (TEA)

We performed TEA for the designed and simulated biomanufacturing facilities using BioSTEAM's discounted cash flow rate of return analysis to calculate the minimum product selling price (MPSP, $/kg) of acrylic acid to achieve a net present value of zero with a 10% annual internal rate of return (a uniform distribution of 8-12% was assumed; a full list of baseline parameter values, uncertainty distributions, and literature references for the same are provided in Table 19. All costs and prices shown are presented in 2019 U.S. dollars. To benchmark the MPSP of the produced acrylic acid, we assumed a market price range of $1.40-1.65/kg from reported 2015-2019 market prices³⁵ to be consistent with Bhagwat et al.¹⁶ All biomanufacturing facilities were modeled as n^{th} plant designs (i.e., it is assumed a successful industry has been established with mature technologies). Key construction (e.g., warehouse, site development), fixed operating (e.g., labor burden, property insurance), and financial (e.g., depreciation, taxes) parameters, as well as all parameters used in the cash flow analysis, followed assumptions made by Bhagwat et al.¹⁶. Tabulated breakdowns of the estimated revenue and capital and operating expenditures, as well as additional details on the design, utility requirements, purchase costs, and installed equipment costs can be found online.⁵⁰

### Example 10 Life Cycle Assessment (LCA)

We performed LCA in Python using the simulated inventories for streams (input chemicals and output emissions) and utilities from BioSTEAM. The LCA scope included the operational phase of the biomanufacturing, including cradle-to-grave impacts for all raw materials, ancillary processes, and unit processes. The functional unit was set to 1 kg of produced acrylic acid to be consistent with the TEA. The sale of co-produced electricity was assumed to displace the impacts of marginal electricity production. Impacts resulting from infrastructure construction were excluded to be consistent with the U.S. renewable fuel standard (RFS)⁵⁵. Final characterization and discussion of environmental impacts focused on carbon intensity (CI; quantified as 100-year global warming potential, GWP₁₀₀) and fossil energy consumption (FEC). The impact assessment methodology used for Cl was Intergovernmental Panel on Climate Change (IPCC) 2013.⁵⁶ Life cycle inventory data were collected from ecoinvent 3.8 and some unit impacts were gathered from GREET 2020,^{36,37} and their sources were noted in the script.⁵⁰ The baseline impacts of feedstock farming (excluding credit for fixed carbon), harvest and collection, transportation, storage, and handling were considered. The amount of carbon fixed in the feedstock is equal to the sum of carbon in the product (acrylic acid) and in the biogenic portion of direct waste emissions from the biomanufacturing facility. To focus on the biomanufacturing facility, we assumed the end-of-life impacts associated with the product (acrylic acid) as well as non-gaseous wastes (e.g., unconsumed and non-combusted sugars and insoluble lignin in the brine, accounting for <0.0002% of the CI) would be exclusively from the oxidation of all carbon into CO₂.

### Example 11 Uncertainty and Sensitivity Analyses

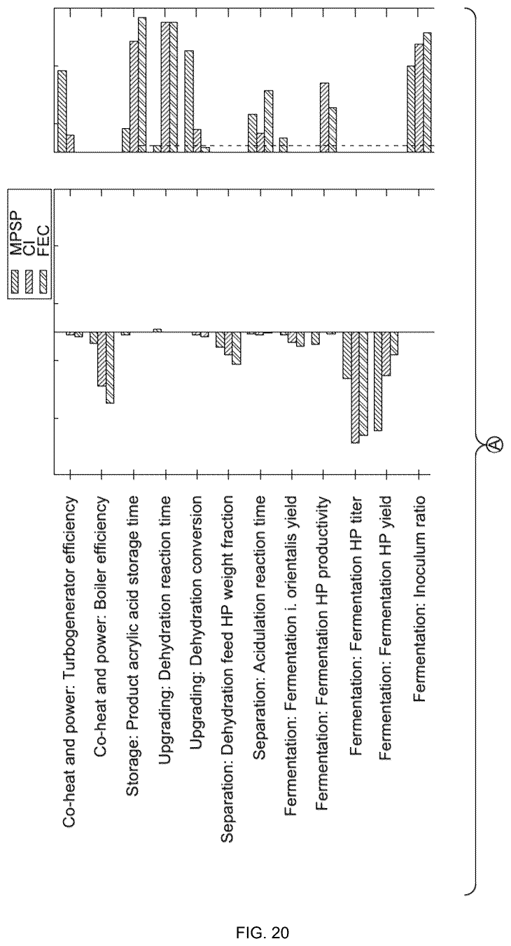

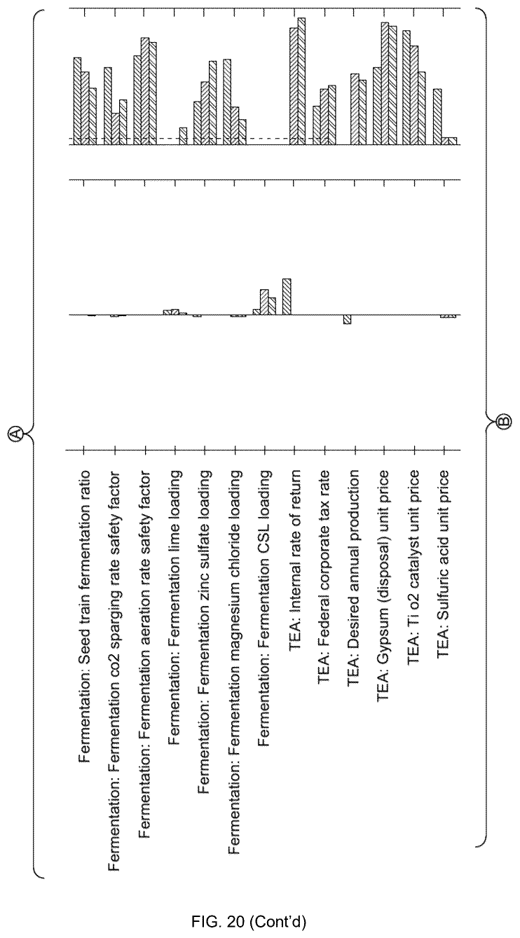

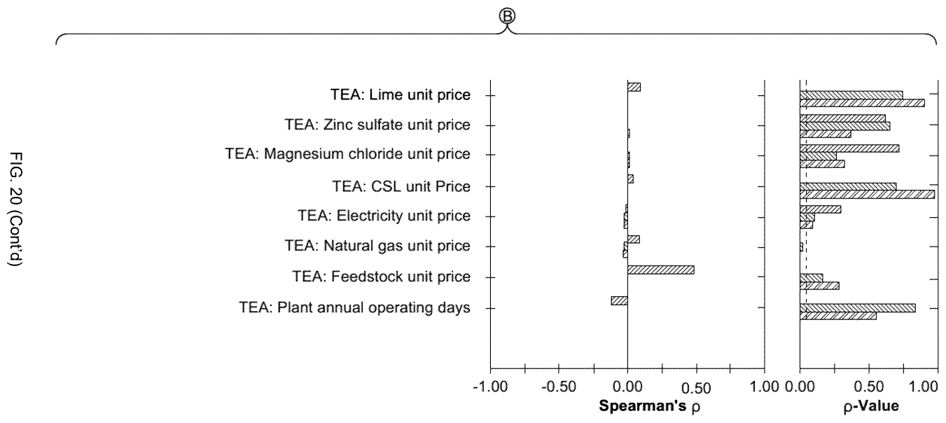

Uncertainty analyses were conducted using Monte Carlo simulation with Latin Hypercube Sampling (6,000 simulations) for 33 uncertain parameters each in the dextrose, corn, sugarcane, and corn stover scenarios. We assigned uncertainty distributions for parameters to be consistent with Bhagwat et al.¹⁶, except for the parameters describing the feedstock acquisition, juicing, and clarification processes (consistent with Bhagwat et al.⁵³) and the fermentation process (based on experimental data obtained in this study). The full list of the evaluated parameters, their uncertainty distributions, and literature references are provided in Table 19. The sensitivity of MPSP, Cl, and FEC to all uncertain inputs was determined via Spearman's rank order correlation coefficients (Spearman's p) using Monte Carlo simulation results, and only parameters with p-value <0.05 were discussed to ensure robustness (FIG. 20).

### Statistics & Reproducibility Example 12

No statistical method was used to predetermine sample size. Most experiments were performed with at least three independent biological replicates, and results are presented as mean±S.D. The 300 mL scale fed-batch fermentation was conducted with two biological replicates, whereas the 3 L scale fed-batch fermentation and qPCR analysis were performed with one biological replicate. No data were excluded from the analyses. The experiments were not randomized, and the investigators were not blinded to allocation during experiments and outcome assessment.

### Example 13 Selection of 3HP Biosynthetic Pathway in I. orientalis

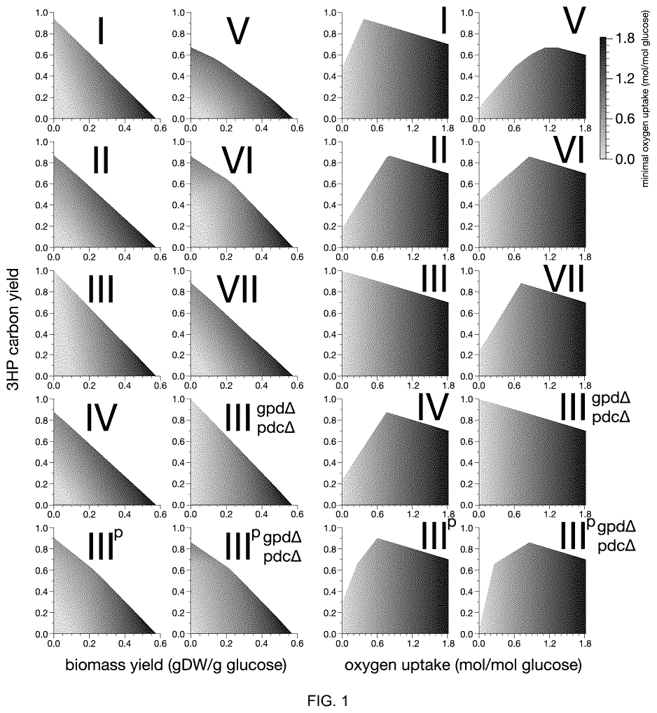

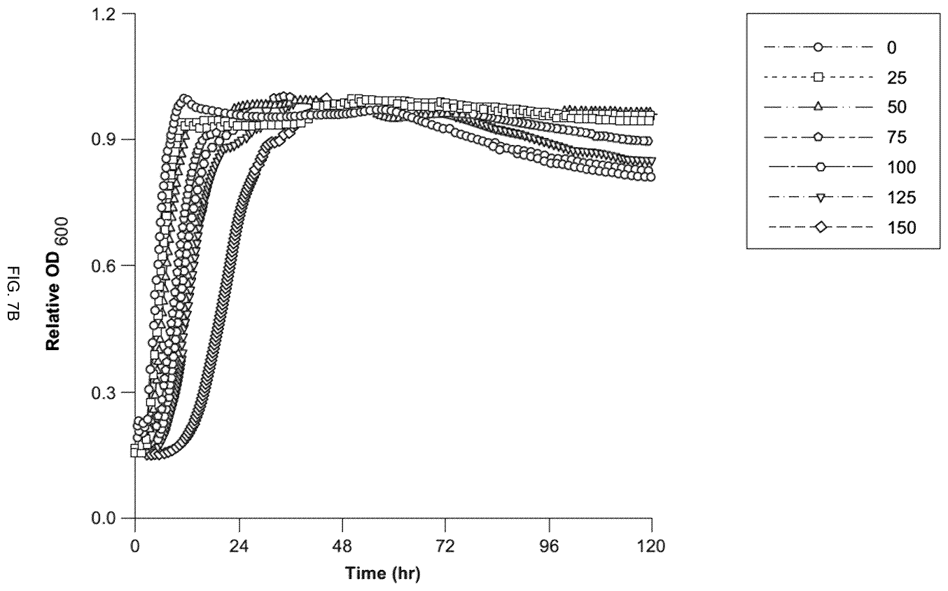

For host selection, we chose I. orientalis SD108 for 3HP production because of its high tolerance to 3HP (up to 125 g/L, FIG. 7 at initial pH of 5.6). For pathway selection, we employed a genome-scale metabolic (GSM) model of I. orientalis²³ augmented separately with reactions capable of producing 3HP by seven previously reported pathways²⁴, including β-alanine and malonyl-CoA pathways. Systematic evaluation revealed that in context of an I. orientalis background, the β-alanine pathway has both the highest maximum theoretical yield of 3HP (i.e., carbon yield from glucose of 99.7% or 0.986 g 3HP/g glucose) as well as the most favorable (i.e., lowest) minimal oxygen uptake requirements at high yield (i.e., only 0.018 mmol/mmol glucose) (FIG. 1). The reactions of this pathway, provided in Table 20, starting from the conversion of pyruvate requires three non-native enzymes: PAND (aspartate 1-decarboxylase), BAPAT (β-alanine-pyruvate aminotransferase), and YDFG (3HP dehydrogenase). Subsequently, we performed thermodynamics analysis of this pathway since external pH is known to affect the intracellular pH of Saccharomyces cerevisiae²⁵ and pH can impact thermodynamics. Table 20 gives the change in Gibbs free energy for each reaction step using dGPredictor, a recently developed moiety-based automated fragmentation tool that has improved goodness of fit over group contribution (GC) methods²⁶, at pH 7 and 6 and an assumed ionic strength of 0.1M (see Table 20 for computations using eQuilibrator²⁷). For pH 6, two non-native steps are substantially more negative (i.e., favorable) than at pH 7. Notably, though, for pH 6 the net change becomes more positive at the aspartate transaminase step and less negative at the pyruvate carboxylase step than they are at pH 7. By shifting the equilibrium through accumulation of reactants and fast removal of products (i.e., the latter by increased PAND levels), aspartate transaminase could nevertheless proceed in the desired direction. Therefore, we decided to assemble and express the β-alanine pathway in I. orientalis for 3HP production.

TABLE 20
Thermodynamic analysis of the β-alanine pathway

| | | ΔᵣG′°, pH 7 | ΔᵣG′°, pH 6 |
|---|---|---|---|
| Gene | Reaction Name | dGpredictor | |
| PYC | pyruvate carboxylase | −4.99 ± 0.10 | −1.72 ± 0.10 |
| AAT | aspartate transaminase | 3.01 ± 0.10 | 3.16 ± 0.10 |
| PAND | aspartate 1-decarboxylase^{a, b} | −22.49 ± 0.23 | −28.06 ± 0.23 |
| BAPAT | β-alanine-pyruvate aminotransferase^{a, c} | −3.28 ± 0.14 | −3.27 ± 0.14 |
| YDFG | malonic semialdehyde reductase^{a, d} | −23.89 ± 0.14 | −29.66 ± 0.14 |

^{a}non-native
^{b}catalyzes decarboxylation of aspartate to β-alanine
^{c}converts β-alanine into malonic semialdehyde (MSA)
^{d}converts MSA to 3HP
Data presents the estimated changes in Gibbs free energy, ΔᵣG′° (kJ/mole) ± standard error (kJ/mole) with both values calculated using the dGpredictor computational tool.

### Example 14 Construction of Efficient 3HP Biosynthetic Pathway in I. orientalis

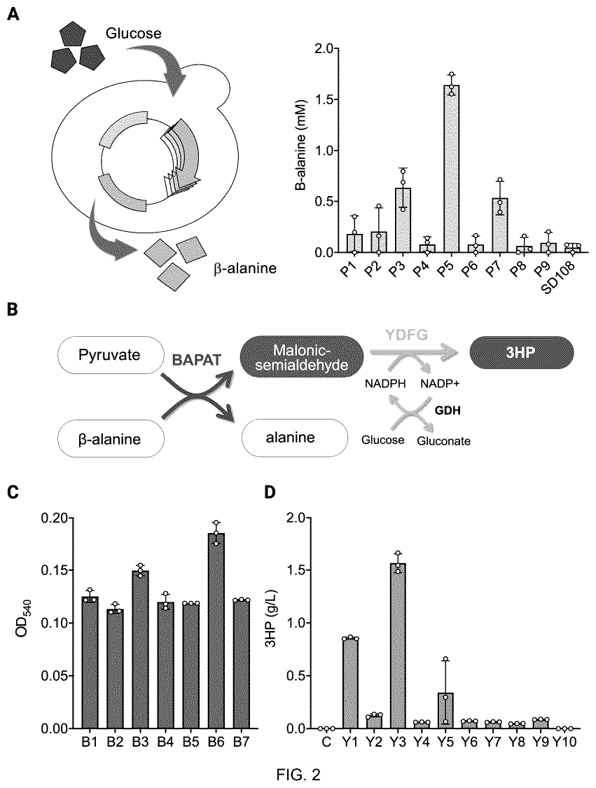

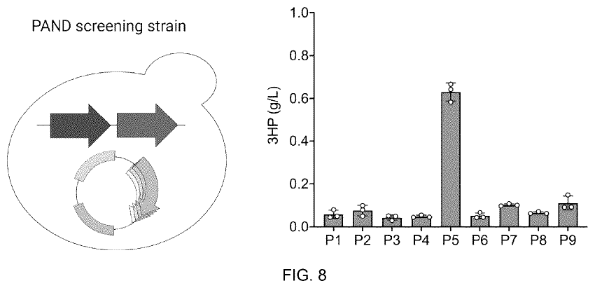

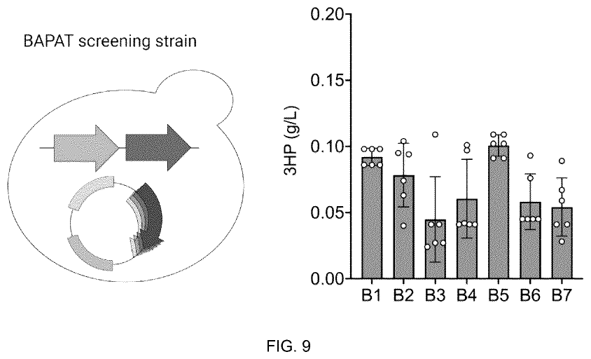

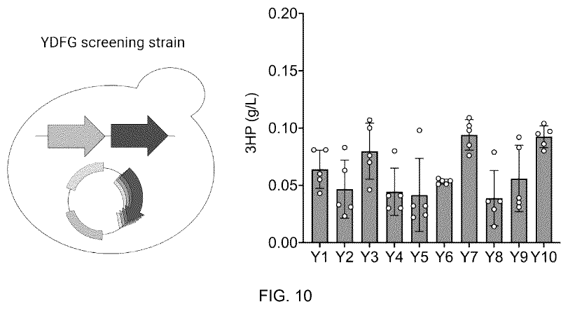

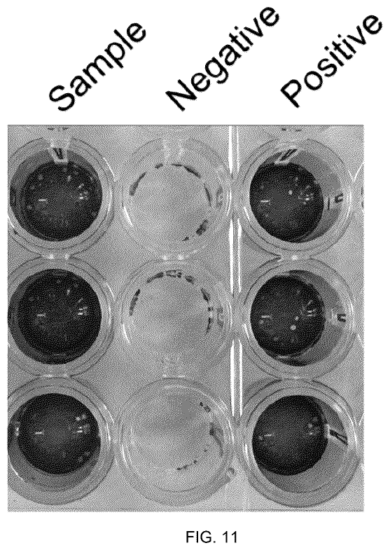

To design an optimal β-alanine pathway in I. orientalis, we used the sequence similarity network (SSN) analysis tool²⁸ to identify up to 10 different genes for each of the three genes in the pathway. The template PAND, BAPAT and YDFG genes were from a previous report in which S. cerevisiae was engineered to produce 3HP via the β-alanine pathway²⁹. At the beginning, we designed an in vivo strategy in which the screening strains were integrated with template BAPAT and template YDFG or template PAND and template YDFG or template PAND and template YDFG for screening candidate PAND or candidate BAPAT or candidate YDFG, respectively. Our first strategy can only allow us to effectively screen the best PAND, but not for BAPAT and YDFG (FIGS. 8, 9, and 10), which indicated that the first step was significantly rate-limiting due to limited supply of the precursors for BAPAT and YDFG. For the PAND gene, we also started another screening assay by directly measuring the intracellular β-alanine accumulation using the recombinant I. orientalis harboring different candidate PAND-encoding plasmids in SC-URA with 50 g/L glucose after two days. As shown in FIG. 2A, the hypothetical protein GE061_003755-Apolygus lucorum (i.e., P5), gave us the highest production of β-alanine, which was consistent with the result obtained from the in vivo test (FIG. 8). For characterizing the other two enzymes, BAPAT and YDFG, we chose an in vitro enzymatic reaction, in which the cell lysate with enzymes produced from I. orientalis were directly mixed with the enzyme substrate (FIG. 2B). For BAPAT, we adopted a previously developed assay by Borodina et al.²⁹, which used colorimetry to quantify the formation of an aldehyde product using PurpaId as a purple-color-forming agent (FIG. 11). We found that the protein aminotransferase class III-fold pyridoxal phosphate-dependent enzyme from Methanosarcina sp. MTP4 (i.e., B6), gave the best result (FIG. 2C). For YDFG, due to the lack of commercially available malonic semialdehyde and the high cost of NADPH, we decided to couple the best BAPAT enzyme and glucose dehydrogenase (GDH) to synthesize malonic semialdehyde and NADPH for the YDFG catalyzed reaction. The results shown in FIG. 2D indicate that the bifunctional NADP-dependent 3-hydroxy acid dehydrogenase/3-hydroxypropionate dehydrogenase YDFG from Erwinia mallotivora (i.e., Y3) produced the most 3HP.

### Example 15 Improving 3HP Production Via Metabolic Engineering

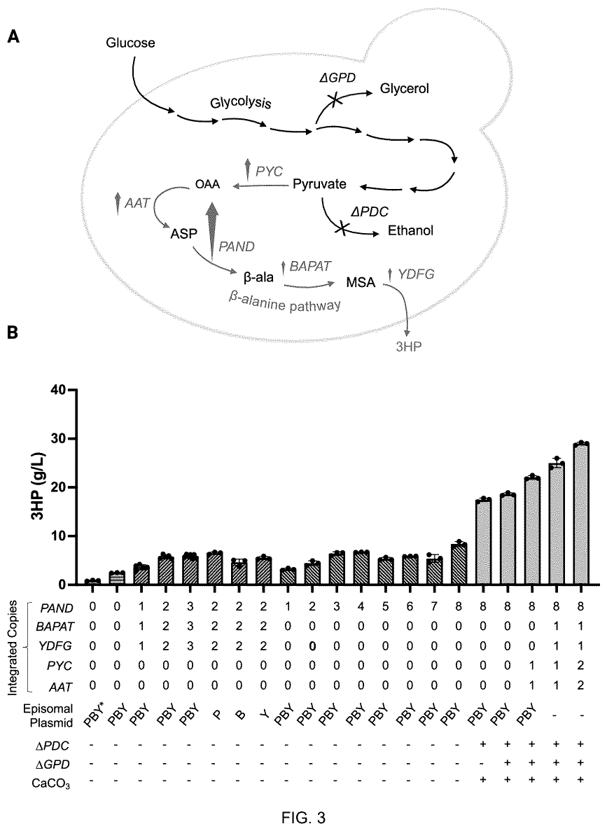

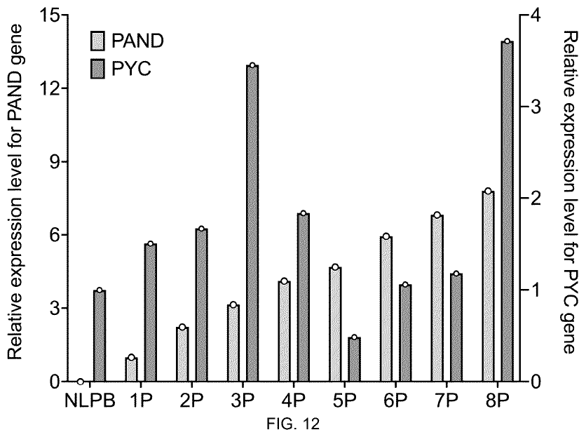

To improve the production of 3HP in I. orientalis, we explored various metabolic engineering strategies (FIG. 3). The strain containing an episomal plasmid, harboring β-alanine pathway comprising the best genes selected from the section of construction of efficient 3HP biosynthetic pathway in I. orientalis, referred to as PBY, produced 2.5 g/L of 3HP (FIG. 3B, column 2) compared to 0.86 g/L (FIG. 3B, column 1) from the strain harboring the β-alanine pathway consisting of the original PAND, BAPAT and YDFG genes. We posited that introducing more copies of the β-alanine pathway integrated into the genome along with an episomal plasmid harboring PBY would increase production. The resulting strain pPBY/PBY3/ura3Δ (3 copies of the pathway integrated in the genome with PBY on an episomal plasmid) led to an increase in 3HP production, reaching 5.81 g/L (FIG. 3B, column 5). To identify the rate-limiting step, we introduced individual episomal plasmids for the PAND gene, the BAPAT gene, the YDFG gene, and all three genes (PBY) into the strain PBY2/ura3Δ (FIG. 3B, column 6 to column 8). Only the strain harboring the plasmid with the PAND gene could produce 3HP as the strain harboring the plasmid with all three genes, indicating PAND was the rate-limiting step. We further investigated the effect of the copy number of the PAND gene on 3HP production by integrating up to eight copies into the LPB/Δura3 strain, our landing pad strain²² that allows multiple gene integrations at a time. This also circumvented generating redundant expression of the BAPAT and YDFG genes. This led to a substantial increase in the 3HP titer from 3.2 g/L for strain pPBY/1P/ura3Δ (1 copy of PAND, FIG. 3B, column 9) to 8.4 g/L for pPBY/8P/ura3Δ (8 copies of PAND, FIG. 3B, column 16), despite a drop to 5.5 g/L with five to seven copies of PAND. qPCR analysis (Example 18) confirmed that the expression level of PAND increased with increasing copy number (FIG. 12, left bars), but production did not continually increase. Further investigation of the PYC gene revealed its elevated expression levels at three and eight PAND copies (FIG. 12, right bars), suggesting that integration at the EG6 site activated PYC, boosting production. The data indicated that the native aspartate flux supported only three to four PAND copies.

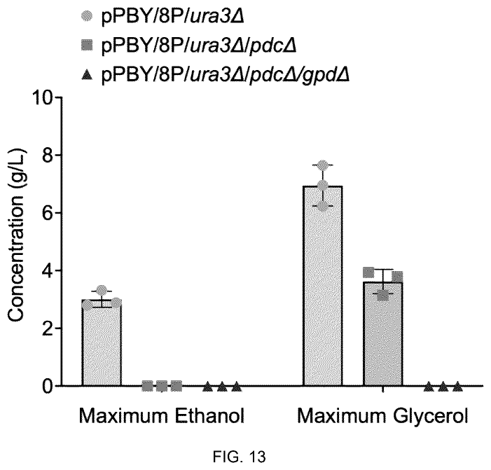

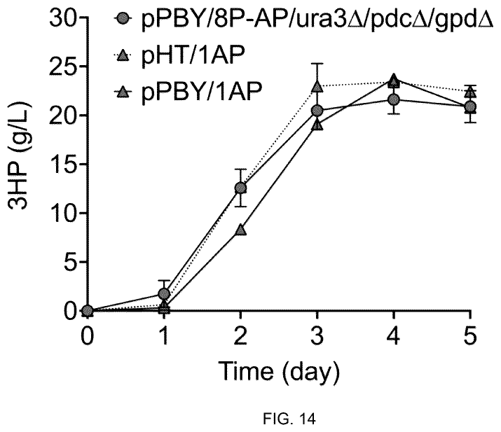

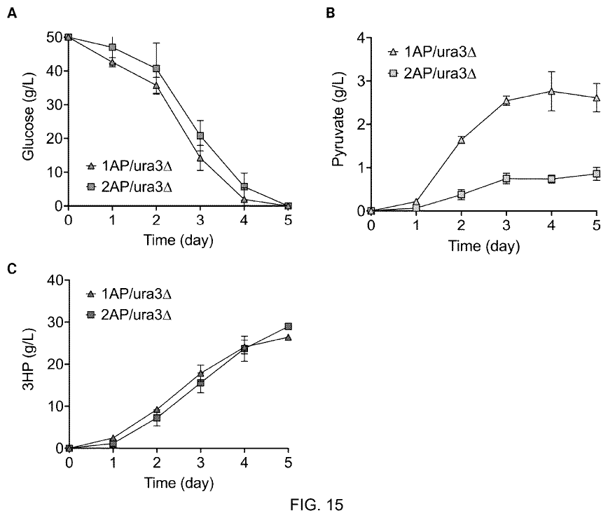

To further enhance 3HP production, we knocked out the PDC gene in the pPBY/8P/ura3Δ strain to generate pPBY/8P/ura3Δ/pdcΔ, and the 3HP titer significantly increased from 8.4 g/L to 17.4 g/L (FIG. 3B, column 17). Next, to further reduce the glycerol byproduct, we knocked out GPD (glycerol-3-phosphate dehydrogenase) in the PDC knock-out strain, leading to a production of 18.6 g/L (FIG. 3B, column 18). Both ethanol and glycerol were also analyzed for three strains, including pPBY/8P/ura3Δ, pPBY/8P/ura3Δ/pdcΔ, and pPBY/8P/ura3Δ/pdcΔ/gpdΔ. With PDC knockout, the maximum ethanol accumulation reduced from 3.0 g/L to 0 g/L and surprisingly the maximum glycerol accumulation also reduced from 6.9 g/L to 3.6 g/L, which explained the significant increase of 3HP titer after PDC knockout. With double knockout of PDC and GPD, there was no accumulation of ethanol and glycerol (FIG. 13). Predictions made using the GSM model to simultaneously knock out both GPD and PDC while using the β-alanine pathway had no decrease in the maximum theoretical carbon yield of 3HP or substantial change in oxygen uptake rate (FIG. 1). Based on the double knock-out strain, we introduced one copy of PYC-AAT to further enhance the carbon flux toward aspartate which is an important precursor in the β-alanine pathway. After integration of one copy of PYC-AAT, the strain pPBY/8P-AP/ura3Δ/pdcΔ/gpdA produced 22 g/L of 3HP (FIG. 3B, column 19). Next, considering that an industrially relevant strain should have all genes integrated into the chromosome to improve genetic stability, we decided to integrate the BAPAT and YDFG genes to the chromosome to generate the 1AP/ura3Δ strain and cultured it in synthetic complete medium (CSM medium), which produced 25 g/L of 3HP (FIG. 3B, column 20). Because the BAPAT and YDFG genes were provided from a two-copy episomal plasmid in the previous section, we transformed the episomal plasmid harboring the PBY genes and empty episomal plasmid back to the 1AP/ura3Δ strain to evaluate the gene copy number effect from BAPAT and YDFG. However, the results showed that there is no difference in production (FIG. 14). To further increase the yield, another copy of PYC-AAT was integrated, which resulted in a titer of 29 g/L (FIG. 3B, column 21). After integrating a second copy of PYC-AA T, the resulting strain, 2AP/ura3Δ, consumed glucose slightly slower than the 1AP/ura3Δ strain (FIG. 15A), but the pyruvate accumulation reduced from 2.6 g/L to 0.86 g/L (FIG. 15B).

### Example 16 Fed-Batch Fermentation

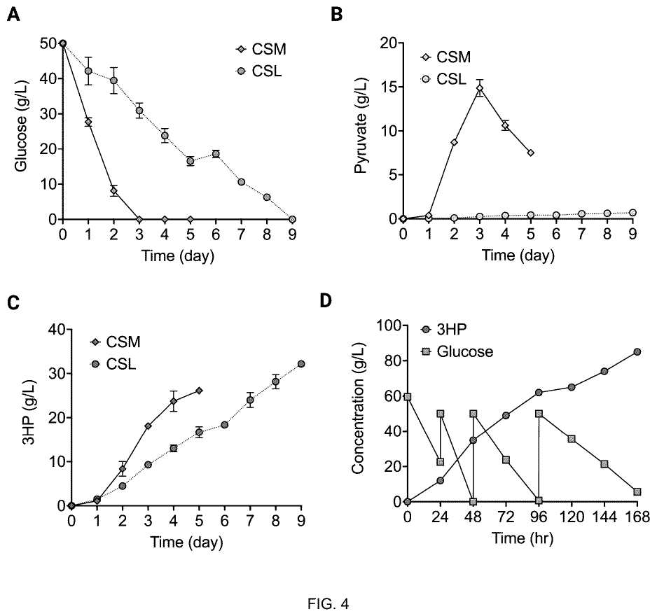

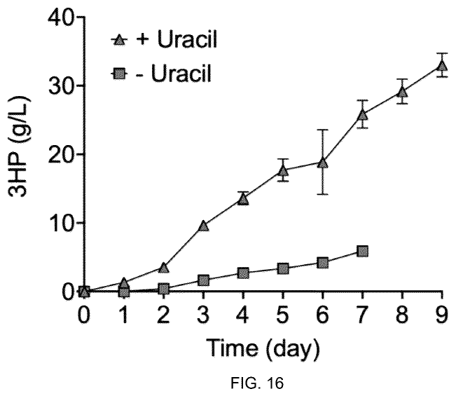

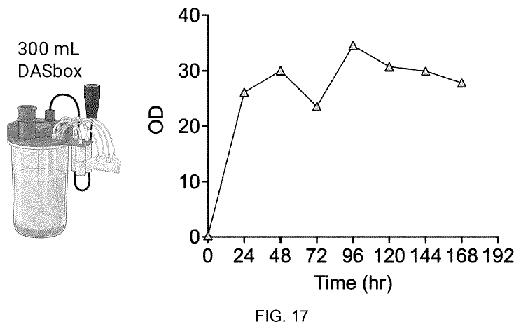

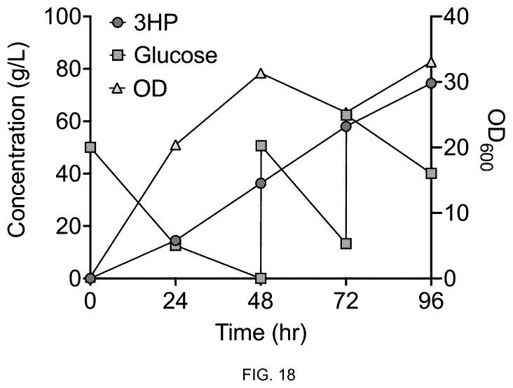

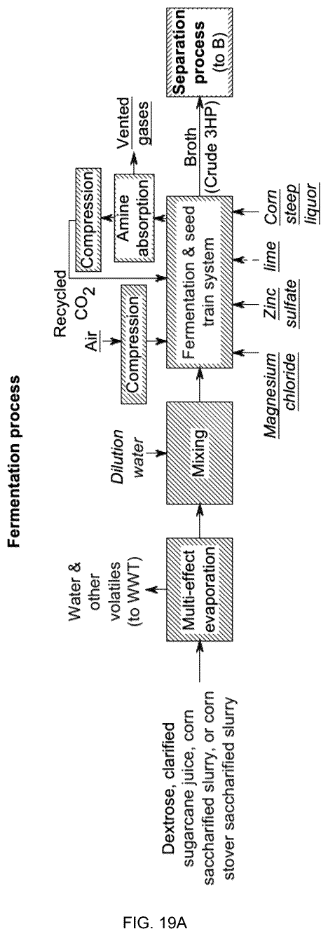

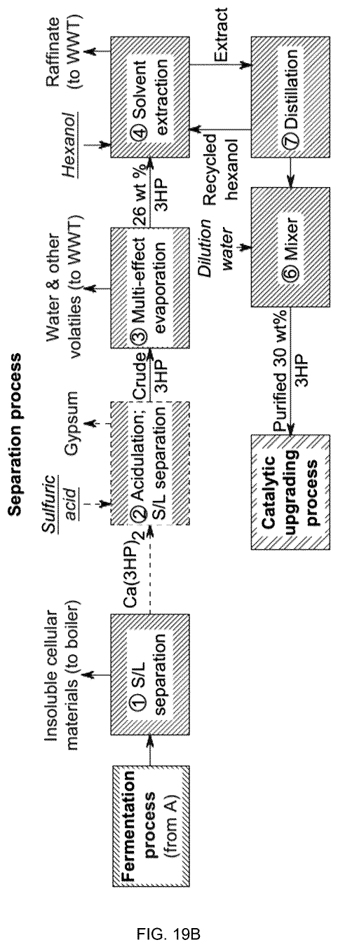

Next, we performed fed-batch fermentation to further increase the titer and yield of 3HP production. During fed-batch fermentation, a pH of 4 was maintained, which eliminated the acidification step for downstream processing. Moreover, dissolved oxygen (DO) was maintained at 5% to create a microaerobic environment, based on the computational modeling studies as illustrated in FIG. 1, to increase 3HP production. We utilized CSL (Corn Steep Liquor) as the fermentation media to make this fermentation industrially feasible and lower cost. To culture the strain in CSL, we recovered the ura3 gene in the 2AP/ura3Δ strain to generate the 2AP strain since the 2AP/ura3Δ strain grew very slowly in pure CSL medium but with uracil supplement, 2AP/ura3Δ could grow similarly as the 2AP strain in pure CSL (FIG. 16). The 2AP strain consumed 50 g/L of glucose, accumulated up to 15 g/L of pyruvate and produced 18 g/L of 3HP in CSM medium by day 3. From day 3 to day 5, the 2AP strain consumed pyruvate to produce 3HP, which reached 26 g/L and a yield of 0.52 g/g on day 5 (FIG. 4A to 4C). As for the CSL medium, the 2AP strain consumed 50 g/L glucose in 9 days and accumulated only 0.7 g/L pyruvate with a 3HP production of 32 g/L (FIG. 4A to 4C). Lower pyruvate accumulation in CSL explains why the 2AP strain produced more 3HP in CSL because pyruvate is an important precursor in the β-alanine pathway. Overall, although CSL medium reduced the glucose uptake rate, pyruvate did not accumulate and the 3HP yield was increased to 0.64 g/g glucose. The 2AP strain was used for all fed-batch fermentations. To demonstrate the maximum performance of 2AP, we performed the fed-batch fermentation in 300 mL DASbox system with CSL medium, DO set to 5%, pH of 4, and CO₂ gassing, which resulted in a titer, yield, and productivity of 92 g/L, 0.7 g/g, and 0.55 g/L/h, respectively (FIG. 4D). Feeding was performed manually at each scale in pulse mode and was dependent on the concentration of glucose at the time of sampling (Table 2). For the cell growth, the OD₆₀₀ (optical density) along with the fermentation time was shown in FIG. 17. Also, during the whole period of fermentation, no other metabolites at concentrations greater than 1 g/L were observed. We also attempted the preliminary scale-up study to 3 L scale and showed that the yield also reached 0.7 g/g with titer of 74.5 g/L. (FIG. 18).

### Example 17 Financial Viability and Environmental Benefits Under Uncertainty

We designed and simulated end-to-end biomanufacturing facilities for 3HP that could accept dextrose monohydrate as a feedstock (as well as corn, sugarcane, and corn stover), ferment to 3HP using I. orientalis, purify and concentrate the fermentation broth (to 30 wt % 3HP), catalytically upgrade 3HP to acrylic acid, and recover glacial acrylic acid of >99.5 wt % purity (FIG. 19) at an annual production capacity of 134,000 metric tons of acrylic acid. We also performed sensitivity analyses using Spearman's rank order correlation coefficients (Spearman's p) to identify key drivers of production costs and life cycle carbon intensity (CI), and designed and simulated the biomanufacturing facility across the theoretical fermentation space (i.e., 2,500 possible yield-titer combinations at the baseline productivity of 0.548 g/L/h as well as 100 possible productivity values at the baseline yield-titer combination of 0.694 g/g and 92 g/L) for each feedstock under both low-pH fermentation (i.e., fermentation pH of 4 and no acidulation required after fermentation) and neutral fermentation regimes (i.e., neutralization by base addition during fermentation and complete re-acidulation after fermentation) to prioritize research and development pathways to advance system sustainability.

For the simulated dextrose-based 3HP biomanufacturing based on experimental performance in the DASbox, the biomanufacturing facility could produce acrylic acid at an estimated minimum product selling price (MPSP) of $1.36/kg (baseline) with a range of $1.21-1.58/kg [5th-95th percentiles, hereafter in brackets] and a Cl of 4.43 kg CO₂-eq./kg [4.01-5.10 kg CO₂-eq./kg; FIG. 5A,B]. Integrating the end-to-end pipeline with an on-site corn dry-grind process would substantially improve the biomanufacturing's financial viability (MPSP of $1.13/kg [$1.01-1.27/kg], below the acrylic acid market price range in 100% of simulations) and reduce Cl (3.26 kg CO₂-eq./kg [2.78-3.92 kg CO₂-eq./kg], below the fossil-based acrylic acid Cl range in 98.5% of simulations). Biomanufacturing facilities designed to accept sugarcane could unlock even greater environmental benefits from the production of excess electricity by the turbogenerator, offsetting marginal grid impacts to achieve a Cl of 2.12 kg CO₂-eq./kg [1.49-2.76 kg CO₂-eq./kg].

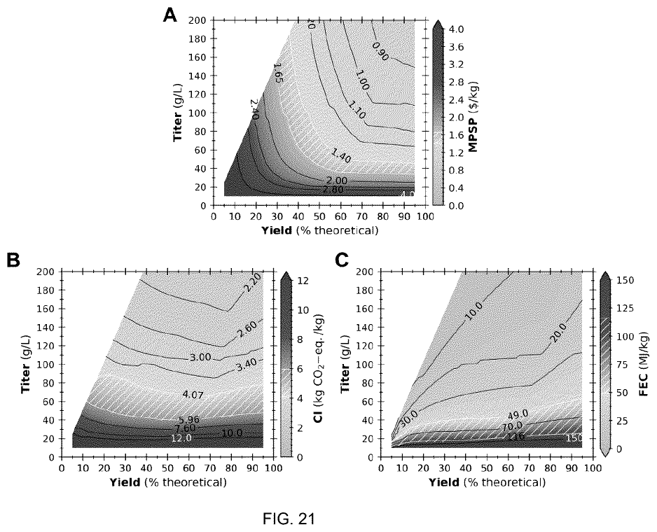

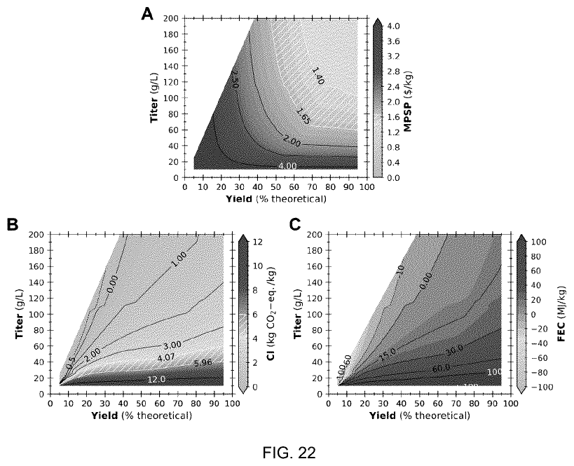

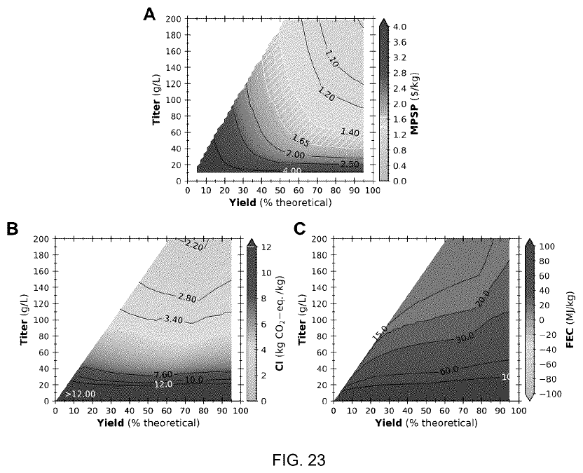

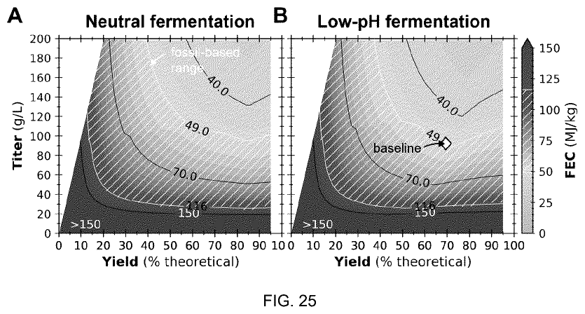

Simulations across the theoretical fermentation space demonstrated improving yield in low-pH fermentation will have the most direct benefit to MPSP (FIG. 5C,E), which was consistent with MPSP being most sensitive to fermentation yield among 33 uncertain parameters (Spearman's p of −0.69; FIG. 20). Regardless of the feedstock used, the biomanufacturing facility's MPSP benefited consistently from increased yield and titer values for both neutral and low-pH fermentation in the evaluated theoretical fermentation space (FIG. 5C,E; FIG. 21-23), with potential minimum values of $1.39/kg for neutral fermentation and $1.02/kg for low-pH fermentation for dextrose-accepting facilities at the baseline productivity of 0.548 g/L/h. The benefits of a higher titer (e.g., lower heating demands from separation units) generally outweighed the increased expenses (e.g., larger fermentation reactors at a fixed 3HP productivity), but the magnitude of the net benefits diminished with increasing titer. Similarly, at higher yield values, further improvements to yield had diminishing benefits for MPSP. The relative benefits to MPSP from comparable relative improvements to yield and titer depended on the location in the theoretical fermentation space. For example, at the baseline fermentation yield (69.4% of the theoretical maximum) and titer (92.0 g/L), improving yield by 6.9% (a 10% relative increase) would decrease the MPSP by $0.06/kg, while improving titer by 9.2 g/L (a 10% relative increase) would decrease the MPSP by only $0.03/kg (FIG. 5E). The biomanufacturing facility's MPSP, Cl, and fossil energy consumption (FEC) were relatively insensitive to potential improvements to fermentation productivity (e.g., decreasing by $0.09/kg, 0.01 kg CO₂-eq./kg, and 0.2 MJ/kg, respectively, with a 400% increase from the baseline productivity to 2.74 g/L/h; FIG. 24). While the biomanufacturing facility's Cl and FEC were most substantially sensitive to 3HP titer (Spearman's p of −0.78 and −0.73, respectively; FIG. 20) and consistently benefited from titer improvements (FIG. 5F, FIG. 25B), 3HP yield improvements at high yield values could actually increase Cl and FEC. This was because the yield of I. orientalis cell mass was assumed to decrease with 3HP yield improvements, resulting in lower-energy wastes being diverted to the boiler for combustion. The resulting decrease in steam production necessitated the purchase of larger amounts of natural gas to satisfy the biomanufacturing facility's heating, cooling, and power utility demands. For the simulated dextrose-based 3HP biomanufacturing based on experimental performance in the 3 L bioreactor (yield of 0.703 g/g, titer of 74.5 g/L, and productivity of 0.776 g/L/h), the MPSP did not change substantially ($1.39/kg) but the Cl and FEC increased to 4.87 kg CO₂-eq./kg and 54.06 MJ/kg, respectively, due to the decreased titer relative to that achieved in the DASbox (92.0 g/L). This demonstrates improving fermentation titer will have the most direct benefits to Cl and FEC. However, high 3HP yields will be necessary to unlock the high titers needed for environmental benefits, with potential minima of 2.96 kg CO₂-eq./kg and 31.8 MJ/kg for Cl and FEC, respectively, for low-pH fermentation in dextrose-accepting biomanufacturing facilities (FIG. 5F, FIG. 25 and Example 19).

### Example 18 qPCR Analysis

Engineered strains were inoculated in YPAD medium and grown at 30° C. with constant shaking at 200 rpm overnight. The cells were then subcultured into fresh YP medium with 20 g/L glucose with the initial OD600 of 0.2 and grown until the OD600 reached 1. Cells were collected from 1 mL of culture, and total RNA was extracted using the RNeasy mini kit from Qiagen (Valencia, CA). cDNA synthesis was performed using the iScript™ Reverse Transcription Supermix from Biorad, and iTaq Universal SYBR Green Supermix from Biorad was used for qPCR following the manufacturer's protocol. Primers for qPCR were designed using the IDT online tool (Primer Quest). The endogenous gene alg9, encoding a mannosyltransferase, was used as the internal control. Expression of the selected gene was normalized by the alg9 expression level. Raw data was analyzed using QuantStudio™ Real-time PCR software from Applied Biosystems. qPCR analysis was performed with technical triplicates.

### Example 19 Scenarios Simulated for Techno-Economic Analysis (TEA) and Life Cycle Assessment (LCA)

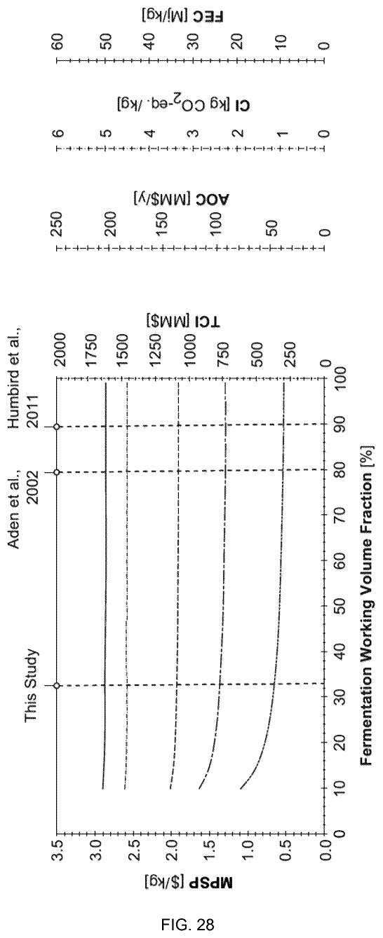

Dextrose-accepting biomanufacturing facilities were simulated under alternative fermentation scenarios with assumptions for yield, titer, and productivity corresponding to the fermentation performance achieved in this study in the 100 mL DASbox (DASbox dextrose scenario). A bioreactor working volume fraction of 33% was assumed in all scenarios based on the working volume fraction used in the DASbox experiments. The mass transfer properties and effects of gas sparging can be tightly coupled with reactor design parameters such as the reactor headspace volume.¹ Therefore, although this working volume fraction is substantially lower than bioreactor working volume fractions assumed in previous TEAs (e.g., 80%),² we wanted to use a working volume fraction consistent with that used in our experiments to conservatively represent the current state-of-technology under nth plant assumptions. Changing the fermentation working volume fraction to that assumed in previous TEAs (e.g., 80%-90%),^{2,3} would moderately change TEA results (MPSP, TCI, and AOC decreased by 4.8-5.3%, 16.8-18.2%, and 1.1-1.2%, respectively) and would not substantially influence LCA results (CI and FEC decreased by 0.2-0.2% and 0.3-0.3%, respectively, in the dextrose scenario; FIG. 28). Further, to assess the system sustainability implications of potential alternative feedstocks, we designed end-to-end biomanufacturing facilities that could accept corn, sugarcane, and corn stover as feedstocks, and simulated them under assumptions corresponding to the fermentation performance achieved in this study using dextrose in the DASbox (corn, sugarcane, and corn stover scenarios, respectively; assumed baseline values and distributions for all uncertain parameters in each scenario are reported in Table 19).

### Example 20 Plasmid Construction

Plasmids for gene overexpression were generated by the in vivo DNA assembly method in S. cerevisiae or Gibson assembly in E. coli. For DNA assembly, 50-100 ng of PCR-amplified fragments and restriction enzyme digested backbone were co-transformed into S. cerevisiae via lithium acetate-mediated method. All colonies formed on SC-URA plates were collected. Plasmids were extracted from S. cerevisiae and retransformed into E. coli. Plasmids from several E. coli colonies were extracted and verified by restriction digestion. Strong promoters and terminators identified previously were chosen for gene overexpression. To construct plasmids for gene deletion, gBlocks containing homology donors and spacers were synthesized by Integrated DNA Technologies. The homology donor contained two 50 bp homology arms flanking the Cas9 cutting site, and a restriction enzyme, such as XhoI, was in the middle of the two homology arms. The gBlocks were then cloned into the CRISPR/Cas9 plasmid by Golden Gate assembly. Detailed construction of each plasmid is described as following:

pTM-PAND(1-9). The primer pairs of P(1-9)PGK1t-F and ura3PGK1t-R were used to amplify for PGK1t, PGK1tura3p-F and TDH3pPDC1t-R for the backbone, PDC1tTDH3p-F and TDH3pP(1-9)-R for TDH3p, and for the PAND genes, PAND(1-9)-F and PAND(1-9)-R. DNA assembler was used to assemble the PCR products to generate the plasmids pTM_PAND(1-9). pTM-BAPAT(J-7). The primer pairs of B(1-7)Trp3t-F and Trp3tura3-R were used to amplify Trp3t, Trp3tura3-F and GPMJPDCJt-R for the backbone, GPMJPDCJt-F and GPMJB(1-7)-R for GPMJ, and for the BAPAT genes, BAPAT(J-7)-F and BAPATHIS-R. DNA assembler was used to assemble the PCR products to make pTM_BAPAT(J-7).

PTM_YDFG(J-J0). The primers pairs of Y(1-10)FBAJp-F and ura3FBAJp-R were used to amplify FBAJp, FBAJpura3-F and ENO2tPDCJt-R were used to amplify the backbone, PDCJtENO2t-F and Y(1-10)ENO2t-R were used to amplify ENO2t, and for the YDFG genes, YDFG(J-J0)-F and YDFG(J-J0)-R were used. DNA assembler was used to assemble the PCR products to generate the plasmids pTM_YDFG(J-J0).

pPBY. PAND5 gene cassette was amplified from pTM-PAND5 using primer pairs of LP10-TDH3p-F and GPMp-pTM-common-R. BAPAT6 gene cassette was amplified from pTM-BAPAT6 using primer pairs of pTM-GPMp-F and FBAp-Trp3t-R. YDFG3 gene cassette was amplified from pTM-YDFG3 using primer pairs of Trp3t-FBAp-F and LP10-ENO2t-R. The backbone pHT was linearized by 3HPBG-pZF-F and 3HPBG-pZF-R. After obtaining four fragments, DNA assembly was used to assemble fragments

pHT-AAT2. IoAAT2 was amplified from I. orientalis genomic DNA using primer pairs of GA-g963-F and GA-g693-R. pHT backbone containing GPM promoter and Eno2 terminator was linearized by primer pairs of GA-pZF-F and GA-pZF-R. After obtaining the two fragments, HIFI assembly was used to assemble fragments.

### Example 21 Strain Construction

For gene deletion, CRISPR/Cas9 plasmid was transformed to I. orientalis via a lithium acetate-mediated method, and transformants were plated on an SC-URA plate and incubated at 30° C. Several colonies were randomly picked and cultivated in liquid SC-URA medium. Then, colony PCR was performed or then restriction digestion was performed to verify the deletion. For gene overexpression, the cassette for gene overexpression was PCR-amplified to contain 40 bp to 42 bp homology arms flanking the integration site. The PCR product and CRISPR/Cas9 plasmid targeting the integration site were co-transformed to I. orientalis via the lithium acetate-mediated method. Transformants were plated on an SC-URA plate and incubated at 30° C. Several colonies were randomly picked and cultivated in liquid YPAD medium. Then, colony PCR was performed for verifying gene overexpression. Plasmid removal was performed using FOA selection. Detailed construction of each strain is described as following:

PBY1/ura3Δ. EG14-LHR-F and EG14-LHR-R were used to amplify the PBY expression cassette from pPBY. Amplified product and pVT36b-EG14 plasmid were then transformed into SD108/ura3Δ.

PBY2/ura3Δ. EG15-LHR-F and EG15-LHR-R were used to amplify the PBY expression cassette from pPBY. Amplified product and pVT36b-EG15 plasmid were then transformed into PBY1/ura3Δ.

PBY3/ura3Δ. EG2-LHR-F and EG15-LHR-R were used to amplify the PBY expression cassette from pPBY. Amplified product and pVT36b-EG2 plasmid were then transformed into PBY2/ura3Δ.

1P/ura3Δ. EG9-LHR-F and EG9-LHR-F were used amplified the PAND5 expression cassette from pTM-PAND5. Amplified product and pVT36b-EG9 plasmid were then transformed into LPB/ura3Δ.

1P/ura3Δ. EG8-LHR-F and EG8-LHR-F were used amplified the PAND5 expression cassette from pTM-PAND5. Amplified product and pVT36b-EG8 plasmid were then transformed into LPB/ura3Δ.

2P/ura3Δ. EG4-LHR-F and EG4-LHR-F were used amplified the PAND5 expression cassette from pTM-PAND5. Amplified product and pVT36b-EG4 plasmid were then transformed into LPB/ura3Δ.

3P/ura3Δ. EG6-LHR-F and EG6-LHR-F were used amplified the PAND5 expression cassette from pTM-PAND5. Amplified product and pVT36b-EG6 plasmid were then transformed into LPB/ura3Δ.

4P/ura3Δ. GD11-LHR-F and GD11-LHR-F were used amplified the PAND5 expression cassette from pTM-PAND5. Amplified product and pVT36b-GD11 plasmid were then transformed into LPB/ura3Δ.

5P/ura3Δ. GD27-LHR-F and GD27-LHR-F were used amplified the PAND5 expression cassette from pTM-PAND5. Amplified product and pVT36b-GD27 plasmid were then transformed into LPB/ura3Δ.

6P/ura3Δ. EG8-LHR-F and EG8-LHR-F were used amplified the PAND5 expression cassette from pTM-PAND5. Amplified product and pVT36b-EG8 plasmid were then transformed into 5P/ura3Δ.

7P/ura3Δ. EG4-LHR-F and EG4-LHR-F were used amplified the PAND5 expression cassette from pTM-PAND5. Amplified product and pVT36b-EG4 plasmid were then transformed into 5P/ura3Δ.

8P/ura3Δ. EG6-LHR-F and EG6-LHR-F were used amplified the PAND5 expression cassette from pTM-PAND5. Amplified product and pVT36b-EG6 plasmid were then transformed into 5P/ura3Δ.

8P/ura3Δ/pdcΔ. pVT36b-pcd which contained the homology arm targeting to pdc gene was transformed into 8P/ura3Δ.

8P/ura3Δ/pdcΔ/gpdΔ. The upstream and downstream sequences of gpd were amplified from I. orientalis genomic DNA. Primer pairs of GPD-seq-F and GPD-KO-Up-R were used for upstream sequence. Primer pairs of GPD-KO-Down-F and GPD-seq-R were used for downstream sequence. pVT36b-gpd, upstream sequence and downstream sequence were transformed into 8P/ura3Δ/pdcΔ.

8P-AP/ura3Δ/pdcΔ/gpdΔ. Primer pairs of EG8-FBAJp-IoPYC-F and GPMp-TEFt-IoPYC-R were used to amplify the IoPYC expression cassette from pRS416-SA-site1. Primers pairs of TEFJt-GPMp-F and EG8-ENO2t (pZF)-R were used to amplify the AAT2 expression cassette from pHT-AAT2. pVT36b-EG8 and fragments of the IoPYC expression cassette and the AAT2 expression cassette were transformed into 8P/ura3Δ/pdcΔ/gpdΔ.

1AP/ura3Δ. Primer pairs of EG16-LHR-F and EG16-LHR-F were used to amplify the BAPAT and YDFG expression cassette from pBY. Amplified product and pVT36b-EG16 plasmid were then transformed into 8P-AP/ura3Δ/pdcΔ/gpdΔ.

2AP/ura3Δ. Primer pairs of EG17-FBAJp-IoPYC-F and GPMp-TEFt-IoPYC-R were used to amplify the IoPYC expression cassette from pRS416-SA-site1. Primers pairs of TEFJt-GPMp-F and EG17-ENO2t (pZF)-R were used to amplify the AAT2 expression cassette from pHT-AAT2. pVT36b-EG17 and fragments of the IoPYC expression cassette and the AAT2 expression cassette were transformed into 1AP/ura3Δ

2AP. Primer pairs of ura3-F and ura3-R were used to amplify the ura3 expression cassette from pVT101a. The ura3 expression cassette was further transformed to 2AP/ura3Δ.

### Example 22. Sequence Similarity Network (SSN) Analysis

SSN analysis was performed for PAND, BAPAT and YDFG gene to get candidates of each gene (efi.igb.illinois.edu/efi-est/)⁴. For PAND gene, PAND gene from T. castaneum was input into the SSN analysis. For BAPAT gene, BAPAT from B. cereus. For YDFG gene, YDFG gene from E. coli was input into the SSN analysis. All the candidate genes were randomly picked in different cluster after SSN analysis.

TABLE 1
Thermodynamic analysis of the β-alanine pathway

| | | | ΔᵣG′°, pH 7 | ΔᵣG′°, pH 6 |
|---|---|---|---|---|
| RxnID | Gene | Reaction Name | eQuilibrator | |
| PC_c | PYC | pyruvate carboxylase | −5.90 ± 1.40 | −1.72 ± 1.40 |
| ASPTA_c | AAT | aspartate transaminase | 2.80 ± 1.10 | 3.16 ± 1.10 |
| ASP1DC_c | PAND | aspartate 1-decarboxylase | −17.30 ± 11.70 | −28.06 ± 11.70 |
| 3A2OA_c | BAPAT | beta-alanine-pyruvate | −3.90 ± 5.8 | −3.27 ± 5.80 |
| | | aminotransferase | | |
| MSAR_c | YDFG | malonic semialdehyde | −21.20 ± 5.70 | −29.66 ± 5.70 |
| | | reductase | | |

Reaction IDs (RxnID) and gene abbreviations are as in Table 20. Data presents the estimated changes in Gibbs free energy, ΔᵣG′° (kJ/mole) ± standard error (kJ/mole) with both values calculated using the eQuilibrator computational tool

TABLE 2
Feeding profile in the fed-batch fermentation 300 mL

| | |
|---|---|
| First feeding | 8.5 mL at 24 hr |
| Second feeding | 12.5 mL at 48 hr |
| Third feeding | 10 mL at 96 hr |
| Fourth feeding | N.A. |
| Final Titer (g/L) | 92.0 |
| Yield (g/g)* | 0.70 |
| Productivity | 0.548 |
| (g/L/hr) | |

P.S. Feeding solution for three scales is 590-600 g/L glucose.
*Yield = Sum of 3HP produced in each feeding/Total glucose consumed.

TABLE 3
Benchmark of 3HP production using different hosts and different pathways.

| | | Titer | Yield | Productivity | | pH | |
|---|---|---|---|---|---|---|---|
| Strain | Pathway | (g/L) | (g/g) | (g/L/hr) | Carbon | control | Refs |
| K. pneumoniae | Glycerol | 102.6 | 0.43 | 1.07 | MM with glycerol | pH 7.0 | ⁵ |
| | | | | | and yeast extract | | |
| E. coli | Glycerol | 76.2 | 0.46 | 1.89 | MM with glycerol | pH 7.0 | ⁶ |
| | | | | | and yeast extract | | |
| S. cerevisiae | Mitochondria Malonyl-CoA | 71.1 | 0.23 | 0.71 | MM with glucose | pH 5.0 | ⁷ |
| S. cerevisiae | Cytosol Malonyl-CoA | 56.5 | 0.31 | 0.53 | MM with glucose | pH 5.6 | ⁸ |
| S. cerevisiae | Cytosol Malonyl-CoA | 11.3 | 0.56 | N.A. | MM with Glucose | With Calcium | ⁹ |
| | | | | | | carbonate | |
| P. pastoris | Cytosol Malonyl-CoA | 48.2 | 0.23 | 0.16 | MM with Methanol | pH 5.6 | ¹⁰ |
| R. toruloides | Cytosol Malonyl-CoA | 45.2 | 0.11 | 0.44 | MM with Lignocellulosic | No | ¹¹ |
| | | | | | hydrolysate | | |
| O. polymorpha | Cytosol Malonyl-CoA | 79.6 | 0.35 | 0.40 | MM with xylose and | pH 5.6 | ¹² |
| | | | | | glucose | | |
| Y. lipolytica | Cytosol Malonyl-CoA | 100 | 0.2 | 0.48 | MM with glucose | pH 6.0 | ¹³ |
| S. cerevisiae | β-alanine | 13.7 | 0.14 | 0.17 | MM with glucose | pH 5.0 | ¹⁴ |
| E. coli | β-alanine | 72.2 | 0.6 | 1.64 | Semi-MM (15 g/L yeast | pH 6.5 | ¹⁵ |
| | | | | | extract) with glycerol | | |

TABLE 4

| Strains | Features | Sources |
|---|---|---|
| E. coli DH5α | Cloning host | NEB |
| SD108/ura3Δ | I. orientalis SD108 with uracil autotrophy | Xiao et al., |
| | | 2014 |
| LPB/ura3Δ | SD108/ura3Δ with landing pad integration | Fatma et al., |
| | | 2023 |
| pPBY/PBY1/ura3Δ | P5B6Y3 integrated to the EG14 site in SD108/ura3Δ | This study |
| | with pPBY plasmid | |
| pPBY/PBY2/ura3Δ | P5B6Y3 integrated to the EG15 site in PBY1/ura3Δ | This study |
| | with pPBY plasmid | |
| pPBY/PBY3/ura3Δ | P5B6Y3 integrated to the EG2 site in PBY2/ura3Δ | This study |
| | with pPBY plasmid | |
| pPBY/1P/ura3Δ | PAND5 integrated to LPB/ura3Δ at EG8 with pPBY | This study |
| | plasmid | |
| pPBY/2P/ura3Δ | PAND5 integrated to LPB/ura3Δ at EG4 with pPBY | This study |
| | plasmid | |
| pPBY/3P/ura3Δ | PAND5 integrated to LPB/ura3Δ at EG6 with pPBY | This study |
| | plasmid | |
| pPBY/4P/ura3Δ | PAND5 integrated to LPB/ura3Δ at GD11 with pPBY | This study |
| | plasmid | |
| pPBY/5P/ura3Δ | PAND5 integrated to LPB/ura3Δ at GD27 with pPBY | This study |
| | plasmid | |
| pPBY/6P/ura3Δ | PAND5 integrated to pPBY/5P/ura3Δ at EG8 with | This study |
| | pPBY plasmid | |
| pPBY/7P/ura3Δ | PAND5 integrated to pPBY/5P/ura3Δ at EG4 with | This study |
| | pPBY plasmid | |
| pPBY/8P/ura3Δ | PAND5 integrated to pPBY/5P/ura3Δ at EG6 with | This study |
| | pPBY plasmid | |
| pPBY/8P/ura3Δ/pdcΔ | pPBY/8P/ura3Δ with pdc knockout | This study |
| pPBY/8P/ura3Δ/pdcΔ/gpdΔ | pPBY/8P/ura3Δ/pdcΔ with gpd knockout | This study |
| pPBY/8P-AP/ | pPBY/8P/ura3Δ/pdcΔ/gpdΔ with PYC-AAT2 integrated | This study |
| ura3Δ/pdcΔ/gpdΔ | to EG8 | |
| 1AP/ura3Δ | 8P-AP/ura3Δ/pdcΔ/gpdΔ with BAPAT6 and YDFG3 | This study |
| | integrated to EG16 | |
| 2AP/ura3Δ | 1AP/ura3Δ with PYC-AAT2 integrated to EG17 | This study |
| 2AP | 2AP/ura3Δ with ura3 recovered | This study |

TABLE 5
Plasmids

| Plasmids | Features | Sources |
|---|---|---|
| pVT36b | I. orientalis plasmid for gene deletion and insertion | Tran et |
| | | al., 2019 |
| pVT36b-EG2 | pVT36b with insertion of spacer for integration at site EG2 | Fatma et |
| | | al., 2023 |
| pVT36b-EG4 | pVT36b with insertion of spacer for integration at site EG4 | Fatma et |
| | | al., 2023 |
| PVT36b-EG6 | pVT36b with insertion of spacer for integration at site EG6 | Fatma et |
| | | al., 2023 |
| pVT36b-EG8 | pVT36b with insertion of spacer for integration at site EG8 | Fatma et |
| | | al., 2023 |
| pVT36b-EG9 | pVT36b with insertion of spacer for integration at site EG9 | Fatma et |
| | | al., 2023 |
| pVT36b-EG14 | pVT36b with insertion of spacer for integration at site EG14 | Fatma et |
| | | al., 2023 |
| pVT36b-EG15 | pVT36b with insertion of spacer for integration at site EG15 | Fatma et |
| | | al., 2023 |
| pVT36b-EG16 | pVT36b with insertion of spacer for integration at site EG16 | Fatma et |
| | | al., 2023 |
| pVT36b-EG17 | pVT36b with insertion of spacer for integration at site EG17 | Fatma et |
| | | al., 2023 |
| pVT36b-GD5 | pVT36b with insertion of spacer for integration at site GD5 | Fatma et |
| | | al., 2023 |
| pVT36b-GD11 | pVT36b with insertion of spacer for integration at site GD11 | Fatma et |
| | | al., 2023 |
| pVT36b-GD21 | pVT36b with insertion of spacer for integration at site GD21 | Fatma et |
| | | al., 2023 |
| pVT36b-GD27 | pVT36b with insertion of spacer for integration at site GD27 | Fatma et |
| | | al., 2023 |
| pVT36b-pdc | pVT36b with insertion of spacer for deletion of pdc with 50 bp | Tran et |
| | upstream and downstream homology arm targeting to pdc | al., 2023 |
| | gene | |
| pVT36b-gpd | pVT36b with insertion of spacer for deletion of gpd | Tran et |
| | | al., 2023 |
| pRS416-SA- | pRS416 harboring reductive TCA pathway flanked with | Tran et |
| site1 | homology arms for integration to site 1 | al., 2023 |
| pVT101a | I. orientalis plasmid for gene deletion and insertion | Tran et |
| | | al., 2023 |
| pTM-PAND1 | pTM with insertion of TDH3p-PAND1-PGK1t | This |
| | | study |
| pTM-PAND2 | pTM with insertion of TDH3p-PAND2-PGK1t | This |
| | | study |
| pTM-PAND3 | pTM with insertion of TDH3p-PAND3-PGK1t | This |
| | | study |
| pTM-PAND4 | pTM with insertion of TDH3p-PAND4-PGK1t | This |
| | | study |
| pTM-PAND5 | pTM with insertion of TDH3p-PAND5-PGK1t | This |
| | | study |
| pTM-PAND6 | pTM with insertion of TDH3p-PAND6-PGK1t | This |
| | | study |
| pTM-PAND7 | pTM with insertion of TDH3p-PAND7-PGK1t | This |
| | | study |
| pTM-PAND8 | pTM with insertion of TDH3p-PAND8-PGK1t | This |
| | | study |
| pTM-BAPAT1 | pTM with insertion of GPMp-BAPAT1-Trp3t | This |
| | | study |
| pTM-BAPAT2 | pTM with insertion of GPMp-BAPAT2-Trp3t | This |
| | | study |
| pTM-BAPAT3 | pTM with insertion of GPMp-BAPAT3-Trp3t | This |
| | | study |
| pTM-BAPAT4 | pTM with insertion of GPMp-BAPAT4-Trp3t | This |
| | | study |
| pTM-BAPAT5 | pTM with insertion of GPMp-BAPAT5-Trp3t | This |
| | | study |
| pTM-BAPAT6 | pTM with insertion of GPMp-BAPAT6-Trp3t | This |
| | | study |
| pTM-BAPAT7 | pTM with insertion of GPMp-BAPAT7-Trp3t | This |
| | | study |
| pTM-YDFG1 | pTM with insertion of FBA1p-YDFG1-ENO2t | This |
| | | study |
| pTM-YDFG2 | pTM with insertion of FBA1p-YDFG2-ENO2t | This |
| | | study |
| pTM-YDFG3 | pTM with insertion of FBA1p-YDFG3-ENO2t | This |
| | | study |
| pTM-YDFG4 | pTM with insertion of FBA1p-YDFG4-ENO2t | This |
| | | study |
| pTM-YDFG5 | pTM with insertion of FBA1p-YDFG5-ENO2t | This |
| | | study |
| pTM-YDFG6 | pTM with insertion of FBA1p-YDFG6-ENO2t | This |
| | | study |
| pTM-YDFG7 | pTM with insertion of FBA1p-YDFG7-ENO2t | This |
| | | study |
| pTM-YDFG8 | pTM with insertion of FBA1p-YDFG8-ENO2t | This |
| | | study |
| pTM-YDFG9 | pTM with insertion of FBA1p-YDFG9-ENO2t | This |
| | | study |
| pTM-YDFG10 | pTM with insertion of FBA1p-YDFG10-ENO2t | This |
| | | study |
| pPBY | pHT with insertion of TDH3p-PAND5-PGK1t-GPMp-BAPAT6- | This |
| | Trp3t-FBA1p-YDFG3-ENO2t | study |
| pHT-AAT2 | pHT with insertion of GPMp-IoAAT2-ENO2t | This |
| | | study |

TABLE 6
Details of the DasBox reactor

| | Lab Scale |
|---|---|
| | Manufacturer |
| Parameter | Eppendorf DASbox |
| Nominal Volume (L) | 0.3 |
| Maximum Working Volume | |
| According to the Manufacture (L) | 0.25 |
| Actual Working Volume (L) | 0.1 |
| Tank Diameter (D_{T}, mm) | 90 |
| Tank Height (H_{T}, mm) | 360 |
| Impeller Type | 6 blade Rushton |
| Impeller Diameter (Dₗ, mm) | 30 |
| Number of impellers | 2 |
| H_{T}/D_{T} | 4 |
| Dₗ/D_{T} | 0.33 |
| Aeration Rate (vvm) | 2.4 sLPH |
| Stirrer Speed (RPM) | 400-1200 (Cascaded on DO) |

TABLE 7
Breakdown of the dextrose scenario baseline biomanufacturing
facility's total capital cost. ISBL and OSBL denote
inside and outside battery limits, respectively.

| | | |
|---|---|---|
| Cost category | Notes | Cost (MM$) |

Direct costs

| | | |
|---|---|---|
| ISBL installed equipment cost | — | 134.12 |
| OSBL installed equipment cost | — | 62.30 |
| Warehouse | 4.0% of ISBL | 5.36 |
| Site development | 9.0% of ISBL | 12.07 |
| Additional piping | 4.5% of ISBL | 6.04 |
| Total direct cost (TDC) | — | 219.88 |

Indirect costs

| | | |
|---|---|---|
| Pro-ratable costs | 10.0% of TDC | 21.99 |
| Field expenses | 10.0% of TDC | 21.99 |
| Construction | 20.0% of TDC | 43.98 |
| Contingency | 10.0% of TDC | 21.99 |
| Other indirect costs (start-up, | 10.0% of TDC | 21.99 |
| permits, etc.) | | |
| Total indirect cost (TIDC) | — | 131.93 |

Total capital investment

| | | |
|---|---|---|
| Fixed capital investment (FCI) | TDC + TIDC | 351.82 |
| Working capital (WC) | 5.0% of FCI | 17.59 |
| Total capital investment (TCI) | FCI + WC | 369.41 |

TABLE 8
Breakdown of the dextrose scenario baseline biomanufacturing
facility's fixed operating cost. ISBL and OSBL denote
inside and outside battery limits, respectively.

| Cost category | Notes | Cost (MM$/y) |
|---|---|---|
| Labor salary | — | 1.82 |
| Labor burden | 90% of labor salary | 1.64 |
| Maintenance | 3.0% of ISBL | 4.02 |
| Property insurance | 0.7% of ISBL | 2.46 |
| Total fixed operating cost (FOC) | — | 1.82 |

TABLE 8A
Primers used in this study.

| Primers | Sequences (5′ to 3′) |
|---|---|
| GA-pZF-F | tctagagccacctgggtc (SEQ ID NO: 11) |
| GA-pZF-R | cgtctgtagagtaaagaaacttctc (SEQ ID NO: 12) |
| GA-g963-F | gtatatcttaaaacacacacacaaaatgtccagaggcttctttact (SEQ ID NO: 13) |
| GA-g963-R | tatttagtaaaagcattagttagatctaaaggtctctcacgacag (SEQ ID NO: 14) |
| LP10-TDH3p-F | atgatacgcggtcgtgctattatggtttccaaaagtttaatgcttgatttaacctgatcc (SEQ ID |
| | NO: 15) |
| GPMp-pTM- | actatcggcctctttttctccgggtgtggtgcatttttcgttcggagattaccgaatc (SEQ ID |
| common-R | NO: 16) |
| pTM-GPMp-F | tcggtttctttgaaatttttttgattcggtaatctccgaacgaaaaatgcaccacacc (SEQ ID |
| | NO: 17) |
| FBAp-Trp3t-R | cacttaagacaatacacacttaatacaatacacatacaattgttcaaatgcttgtctg (SEQ |
| | ID NO: 18) |
| Trp3t-FBAp-F | agttctgcgcctcttcaccagacaagcatttgaacaattgtatgtgtattgtattaagtg (SEQ |
| | ID NO: 19) |
| LP10-ENO2t-R | tatattcacaatgattattttctacatgtatagatggacctaactggaacatgtttgtac (SEQ ID |
| | NO: 20) |
| 3HPBG-pZF-F | tgtagaaaataatcattgtgaatatatctagagccacctgggtc (SEQ ID NO: 21) |
| 3HPBG-pZF-R | ccataatagcacgaccgcgtatcatcgtctgtagagtaaagaaacttctc (SEQ ID |
| | NO: 22) |
| EG4-LHR-F | tgaagtgcaagtggagtgtggaagttgaggcggacacgtgcacttggctaaagaataaga |
| | (SEQ ID NO: 23) |
| EG4-RHR-R | acaaacaagagtcccacaggcagccatgccaacagtggcgcagacccaggtggctctaga |
| | (SEQ ID NO: 24) |
| EG6-LHR-F | tgtcacagcaaggcccgtctataccaacatgtgcagagcacacttggctaaagaataaga |
| | (SEQ ID NO: 25) |
| EG6-RHR-R | tgtgtgtgtatgttatatccacacacaggggcagccacccctgacccaggtggctctaga |
| | (SEQ ID NO: 26) |
| GD5-LHR-F | aggtggaatccagttgatttctatcaggttaaccacatacaacttggctaaagaataaga |
| | (SEQ ID NO: 27) |
| GD5-RHR-R | ctctgtatgtcgaaagttttaccttactgttacacaaaggttgacccaggtggctctaga (SEQ |
| | ID NO: 28) |
| GD11-LHR-F | tttatctcggacttgactcggcaccctcttgccgcggcaccacttggctaaagaataaga |
| | (SEQ ID NO: 29) |
| GD11-RHR-R | gtggaaatgacagaatattggttgttccgcggctcgagaggcgacccaggtggctctaga |
| | (SEQ ID NO: 30) |
| GD27-LHR-F | gcagcttacttacgtatacagattatcctctctatggactcacttggctaaagaataaga (SEQ |
| | ID NO: 31) |
| GD27-RHR-R | atcgtagctggacgttacggcgcatcacaaggtgccacgtacgacccaggtggctctaga |
| | (SEQ ID NO: 32) |
| EG9-LHR-F | ccatggcatgtataaataacggatgtgtgagttcaagatgcacttggctaaagaataaga |
| | (SEQ ID NO: 33) |
| EG9-RHR-R | ccatgtctattgtaaacaatagaagtacagacacaaacaaacgacccaggtggctctaga |
| | (SEQ ID NO: 34) |
| EG8-LHR-F | ataactaaattagactgtatttcaaatgagccgcctttagaacttggctaaagaataaga |
| | (SEQ ID NO: 35) |
| EG8-RHR-R | cttgaactcacacatccgttatttatacatgccatggaatttgacccaggtggctctaga (SEQ |
| | ID NO: 36) |
| EG2-LHR-F | taggtagacatgctacttttctctcttggttttaccacctaacttggctaaagaataaga (SEQ |
| | ID NO: 37) |
| EG2-RHR-R | tttccatatcattttatatgtagacaatttaatctacatacagacccaggtggctctaga (SEQ |
| | ID NO: 38) |
| EG14-LHR-F | tgatacgcggtcgtgctattatggtttccaaaagtttaatgacttggctaaagaataaga (SEQ |
| | ID NO: 39) |
| EG14-RHR-R | tatattcacaatgattattttctacatgtatagatggacctagacccaggtggctctaga (SEQ |
| | ID NO: 40) |
| EG15-LHR-F | aagaagtacctaagacggaaacctgaataatggagtgagttacttggctaaagaataaga |
| | (SEQ ID NO: 41) |
| EG15-RHR-R | caaaaggagatgctcgtgacttctattggcttgtggaatagtgacccaggtggctctaga |
| | (SEQ ID NO: 42) |
| EG17-FBA1p- | gtggcctaaacgttacttatgtactcagagatacgtatacgatcgattgtatgtgtattg (SEQ |
| loPYC-F | ID NO: 43) |
| EG17-ENO2t | taactcacttatagtatgaaccttctatttcggacatcaagtggctctagatggaacatg (SEQ |
| (pZF)-R | ID NO: 44) |
| EG8-FBA1p- | catggcatgtataaataacggatgtgtgagttcaagatgcgatcgattgtatgtgtattg (SEQ |
| loPYC-F | ID NO: 45) |
| EG8-ENO2t(pZF)- | atgtctattgtaaacaatagaagtacagacacaaacaaacgtggctctagatggaacatg |
| R | (SEQ ID NO: 46) |
| TEF1t-GPMp-F | gaaacttctcgttcatcttattctttagccaatacagacgcgaaaaatgc (SEQ ID NO: 47) |
| GPMp-TEFt- | cgggtgtggtgcatttttcgcgtctgtattggctaaagaataagatgaacg (SEQ ID |
| loPYC-R | NO: 48) |
| LP4-Check-F | ggtgagggtttgtaacaaag (SEQ ID NO: 49) |
| LP4-Check-R | tccgcctcagcatataataac (SEQ ID NO: 50) |
| EG4-Check-F | ccacaattccaaacaaggtg (SEQ ID NO: 51) |
| EG4-Check-R | gcttgtatccggagaaactc (SEQ ID NO: 52) |
| EG6-Check-F | gttagtggctcactatcgtg (SEQ ID NO: 53) |
| EG6-Check-R | gtttggacgtgctagacag (SEQ ID NO: 54) |
| GD5-Check-F | ggatcgacttgtcgttatc (SEQ ID NO: 55) |
| GD5-Check-R | gcatatcatgcaaacagcg (SEQ ID NO: 56) |
| GD11-Check-F | ccattttcggtcgaatgc (SEQ ID NO: 57) |
| GD11-Check-R | caatgcaggaggaaaacg (SEQ ID NO: 58) |
| GD27-Check-F | ttgcttatacagcagctgtg (SEQ ID NO: 59) |
| GD27-Check-R | gccaaactaacactgaggtc (SEQ ID NO: 60) |
| EG14-Check-F | tggactctctacgtgaatgt (SEQ ID NO: 61) |
| EG14-Check-R | tttccttctttctaccccttt (SEQ ID NO: 62) |
| EG15-Check-F | gtttccctgttttccttagat (SEQ ID NO: 63) |
| EG15-Check-R | tgtggcctcggagtcctt (SEQ ID NO: 64) |
| EG2-Check-F | agtgatgccctttgagatg (SEQ ID NO: 65) |
| EG2-Check-R | agacaaggcaaggattgtg (SEQ ID NO: 66) |
| Junction EG4/ | cacagaggccctcgcaac (SEQ ID NO: 67) |
| EG6-R | |
| Junction | ggatgcgtccacggaaac (SEQ ID NO: 68) |
| GD5/GD11-R | |
| Junction | caatctcaggactcacgggag (SEQ ID NO: 69) |
| GD11/GD21-R | |
| Junction EG4/ | gttgcgagggcctctgtg (SEQ ID NO: 70) |
| EG6-F | |
| Junction | gtttccgtggacgcatcc (SEQ ID NO: 71) |
| GD5/GD11-F | |
| Junction | ctcccgtgagtcctgagattg (SEQ ID NO: 72) |
| GD11/GD21-F | |
| qPCR-PYC-F | gaggcttatgcaatcggtaaga (SEQ ID NO: 73) |
| qPCR-PYC-R | acctggatggatcatggaaac (SEQ ID NO: 74) |
| qPCR-PAND5-F | gtcaagactgatgctagaggtaag (SEQ ID NO: 75) |
| qPCR-PAND5-R | ccagatgttgcagaaaccataaa (SEQ ID NO: 76) |
| GPD-seq-F | cccctgctgaaagattatctac (SEQ ID NO: 77) |
| GPD-KO-Up-R | gcagagacagaatgctgtgccatatatctaccatttcagtcaacttttcgcc (SEQ ID |
| | NO: 78) |
| GPD-KO-Down-F | gaattgattgaaggcgaaaagttgactgaaatggtagatatatggcacagc (SEQ ID |
| | NO: 79) |
| GPD-seq-R | tcttccacaggttcaagg (SEQ ID NO: 80) |
| PDC-seq-F | cattgttggaccacgtcaagg (SEQ ID NO: 81) |
| PDC-Seq-R | aacgagaatgattggtttttctg (SEQ ID NO: 82) |
| PAND1-F | acacaaacaaacacaattacaaaaaatgttcttggccatcaagtc (SEQ ID NO: 83) |
| PAND1-R | gattgggtaaggacttgtagatcaaacatagatcaacgtaatgaa (SEQ ID NO: 84) |
| PAND2-F | cacacaaacaaacacaattacaaaaaatgcctgctatctccaacg (SEQ ID NO: 85) |
| PAND2-R | tcgcaagagatttctttgtagatcaaacatagatcaacgtaatga (SEQ ID NO: 86) |
| PAND3-F | cacacaaacaaacacaattacaaaaaatgtgttcctcgaaggaag (SEQ ID NO: 87) |
| PAND3-R | atagattaggtaatgatctttagatcaaacatagatcaacgtaat (SEQ ID NO: 88) |
| PAND4-F | acaaacaaacacaattacaaaaaatgatttcctttcaaaccaaag (SEQ ID NO: 89) |
| PAND4-R | gctcgctgctgatttgtagatcaaacatagatcaacgtaatgaat (SEQ ID NO: 90) |
| PAND5-F | acacacaaaacacacaaacaaacacaattacaaaaaatgcctgcttctgaggctt (SEQ |
| | ID NO: 91) |
| PAND5-R | agattaggtagaaacctctagatcaaacatagatcaacgtaatga (SEQ ID NO: 92) |
| PAND6-F | acacaaacaaacacaattacaaaaaatgaacaatctccatgcaac (SEQ ID NO: 93) |
| PAND6-R | gacattgctatcgatatgtagatcaaacatagatcaacgtaatga (SEQ ID NO: 94) |
| PAND7-F | acacaaacaaacacaattacaaaaaatgtcctccgttactaagac (SEQ ID NO: 95) |
| PAND7-R | gagaagaatgtgagtactagatcaaacatagatcaacgtaatgaa (SEQ ID NO: 96) |
| PAND8-F | caaacaaacacaattacaaaaaatgcttactattttatttcgttc (SEQ ID NO: 97) |
| PAND8-R | aattgggcgaagatctttagatcaaacatagatcaacgtaatgaa (SEQ ID NO: 98) |
| PAND9-F | cacaaacaaacacaattacaaaaaatggaacgtcacattaatggt (SEQ ID NO: 99) |
| PAND9-R | ttcgttggctaacgcgtagatcaaacatagatcaacgtaatgaat (SEQ ID NO: 100) |
| BAPATHIS-R | catcatcatcatcatcattagaagcctctcttgaatcagctggttgtgtatgtat (SEQ ID |
| | NO: 101) |
| BAPAT1-F | gtatatcttaaaacacacacacaaaatggaacttatgatcgttcaag (SEQ ID NO: 102) |
| BAPAT2-F | gtatatcttaaaacacacacacaaaatgtcctcggaaaccattag (SEQ ID NO: 103) |
| BAPAT3-F | gtatatcttaaaacacacacacaaaatgtcgatctcagagaaatatt (SEQ ID NO: 104) |
| BAPAT4-F | gtatatcttaaaacacacacacaaaatgagtttgaacgacgatgc (SEQ ID NO: 105) |
| BAPAT5-F | gtatatcttaaaacacacacacaaaatggaaaacgtgaaataccaaa (SEQ ID |
| | NO: 106) |
| BAPAT6-F | gtatatcttaaaacacacacacaaaatggaagacaaaagaaaaggtg (SEQ ID |
| | NO: 107) |
| BAPAT7-F | gtatatcttaaaacacacacacaaaatgagtaaaagtgaaaacctct (SEQ ID |
| | NO: 108) |
| YDFG1-F | tatttagtaaaagcattagttagatttactcatcggaaaaccagt (SEQ ID NO: 109) |
| YDFG1-R | cacctgtaactaaaacaatcattgtgtttgtgttgggggtggtag (SEQ ID NO: 110) |
| YDFG2-F | atttagtaaaagcattagttagatttaacccttgtggacctttaa (SEQ ID NO: 111) |
| YDFG2-R | aaagataatgagcctggcattgtgtttgtgttgggggtggtagta (SEQ ID NO: 112) |
| YDFG3-F | tttagtaaaagcattagttagatttatgccttatgaaccttcaaa (SEQ ID NO: 113) |
| YDFG3-R | gcctgtaacaaagacaatcattgtgtttgtgttgggggtggtagtaatagtagta (SEQ ID |
| | NO: 114) |
| YDFG4-F | tttagtaaaagcattagttagatttatggattcttatgagtaggt (SEQ ID NO: 115) |
| YDFG4-R | tgagacaagaatatgagccattgtgtttgtgttgggggtggtagt (SEQ ID NO: 116) |
| YDFG5-F | atttagtaaaagcattagttagatttagccttttctgtgaacaga (SEQ ID NO: 117) |
| YDFG5-R | tgatgaataatgttggagtcattgtgtttgtgttgggggtggtag (SEQ ID NO: 118) |
| YDFG6-F | tatttagtaaaagcattagttagatttagtcgacatccctaacga (SEQ ID NO: 119) |
| YDFG6-R | aaccaaaacagtatcggtcattgtgtttgtgttgggggtggtagt (SEQ ID NO: 120) |
| YDFG7-F | atttagtaaaagcattagttagatttagccaacatgtctattaac (SEQ ID NO: 121) |
| YDFG7-R | accaagacggttaaaggcattgtgtttgtgttgggggtggtagta (SEQ ID NO: 122) |
| YDFG8-F | atttagtaaaagcattagttagatttaatctctgtggacttgcaa (SEQ ID NO: 123) |
| YDFG8-R | accatggctgttttggtcattgtgtttgtgttgggggtggtagta (SEQ ID NO: 124) |
| YDFG9-F | tatttagtaaaagcattagttagatttacgaagcaccaggtctat (SEQ ID NO: 125) |
| YDFG9-R | taataaaaacggttctggacattgtgtttgtgttgggggtggtag (SEQ ID NO: 126) |
| YDFG10-F | ttagtaaaagcattagttagatttagaatttttcaatagccaaac (SEQ ID NO: 127) |
| YDFG10-R | acaaatgcggtcttcttcattgtgtttgtgttgggggtggtagta (SEQ ID NO: 128) |
| P1PGK1t-F | aaagattgggtaaggacttgtagatcaaacatagatcaacgtaat (SEQ ID NO: 129) |
| P2PGK1t-F | tatcgcaagagatttctttgtagatcaaacatagatcaacgtaat (SEQ ID NO: 130) |
| P3PGK1t-F | atagattaggtaatgatctttagatcaaacatagatcaacgtaat (SEQ ID NO: 131) |
| P4PGK1t-F | agaagctcgctgctgatttgtagatcaaacatagatcaacgtaat (SEQ ID NO: 132) |
| P5PGK1t-F | cagaatttgaaagattaggtagaaacctctagatcaaacatagatcaacgtaatg (SEQ ID |
| | NO: 133) |
| P6PGK1t-F | ttgacattgctatcgatatgtagatcaaacatagatcaacgtaat (SEQ ID NO: 134) |
| P7PGK1t-F | taggagaagaatgtgagtactagatcaaacatagatcaacgtaat (SEQ ID NO: 135) |
| P8pgk1t-F | cgcaattgggcgaagatctttagatcaaacatagatcaacgtaat (SEQ ID NO: 136) |
| P9PGK1t-F | ctttttcgttggctaacgcgtagatcaaacatagatcaacgtaat (SEQ ID NO: 137) |
| ura3PGK1t-R | tattattattattattattattatcatattcaattcatcattttttttttattct (SEQ ID NO: 138) |
| PGK1tura3p-F | ttattattattattattattattatcatattcaattcatcattttttttttattc (SEQ ID NO: 139) |
| PDC1tTDH3p-R | tataattttataataaaaagtttaaacttgatttaacctgatccaaaaggggtat (SEQ ID |
| | NO: 140) |
| TDH3PDC1t-F | ataatatataattttataataaaaagtttaaacttgatttaacctgatccaaaag (SEQ ID |
| | NO: 141) |
| TDH3pP1-R | acaaacaaacacaattacaaaaaatgttcttggccatcaagtctt (SEQ ID NO: 142) |
| TDH3pP2-R | acaaacaaacacaattacaaaaaatgcctgctatctccaacggct (SEQ ID NO: 143) |
| TDH3pP3-R | cacacaaacaaacacaattacaaaaaatgtgttcctcgaaggaag (SEQ ID NO: 144) |
| TDH3pP4-R | cacacaaacaaacacaattacaaaaaatgatttcctttcaaacca (SEQ ID NO: 145) |
| TDH3pP5-R | cacaaacaaacacaattacaaaaaatgcctgcttctgaggcttccgccgaaaggc (SEQ |
| | ID NO: 146) |
| TDH3pP6-R | cacacaaacaaacacaattacaaaaaatgaacaatctccatgcaa (SEQ ID |
| | NO: 147) |
| TDH3pP7-R | cacacaaacaaacacaattacaaaaaatgtcctccgttactaaga (SEQ ID NO: 148) |
| TDH3pP8-R | cacacaaacaaacacaattacaaaaaatgcttactattttatttc (SEQ ID NO: 149) |
| TDH3pP9-R | cacacaaacaaacacaattacaaaaaatggaacgtcacattaatg (SEQ ID NO: 150) |
| PDC1tGPM1-F | agaataatatataattttataataaaaagtttaaaccgaaaaatgcaccacaccc (SEQ ID |
| | NO: 151) |
| B1GPM1-R | atatcttaaaacacacacacaaaatggaacttatgatcgttcaagtcacagagca (SEQ ID |
| | NO: 152) |
| B2GPM1-R | atatcttaaaacacacacacaaaatgtcctcggaaaccattagtcaatcaattaa (SEQ ID |
| | NO: 153) |
| B3GPM1-R | atatcttaaaacacacacacaaaatgtcgatctcagagaaatattttccacaaga (SEQ ID |
| | NO: 154) |
| B4GPM1-R | atatcttaaaacacacacacaaaatgagtttgaacgacgatgccacattttggcg (SEQ ID |
| | NO: 155) |
| B5GPM1-R | atatcttaaaacacacacacaaaatggaaaacgtgaaataccaaattccacctgc (SEQ |
| | ID NO: 156) |
| B6GPM1-R | atatcttaaaacacacacacaaaatggaagacaaaagaaaaggtgagagaaagcg |
| | (SEQ ID NO: 157) |
| B7GPM1-R | atatcttaaaacacacacacaaaatgagtaaaagtgaaaacctctataatgcagc (SEQ |
| | ID NO: 158) |
| GPM1PDC1t-R | tataattttataataaaaagtttaaaccgaaaaatgcaccacacccggagaaaaa (SEQ |
| | ID NO: 159) |
| Trp3tura3-F | tcttcaccagacaagcatttgaacattcaattcatcattttttttttatt (SEQ ID NO: 160) |
| Trp3tura3-R | cagacaagcatttgaaca (SEQ ID NO: 161) |
| B1Trp3t-F | aatgcttagcccaacatcatcatcatcatcattagaagcctctcttgaatcagct (SEQ ID |
| | NO: 162) |
| B2Trp3t-F | tgaagaagctcacgcatcatcatcatcatcattagaagcctctcttgaatcagct (SEQ ID |
| | NO: 163) |
| B3 Trp3t-F | cattttccaagatccatcatcatcatcatcattagaagcctctcttgaatcagct (SEQ ID |
| | NO: 164) |
| B4Trp3t-F | ttgaacgtgcacttcatcatcatcatcatcattagaagcctctcttgaatcagct (SEQ ID |
| | NO: 165) |
| B5 Trp3t-F | tcaagaagtccttacatcatcatcatcatcattagaagcctctcttgaatcagct (SEQ ID |
| | NO: 166) |
| B6 Trp3t-F | aacgtggtgttcgtcatcatcatcatcatcattagaagcctctcttgaatcagct (SEQ ID |
| | NO: 167) |
| B7 Trp3t-F | cctttgcaaaattacatcatcatcatcatcattagaagcctctcttgaatcagct (SEQ ID |
| | NO: 168) |
| FBA1pura3-F | atacacacttaatacaatacacatacaatttcaattcatcattttttttttattc (SEQ ID |
| | NO: 169) |
| ENO2tPDC1t-R | tataattttataataaaaagtttaaactggaacatgtttgtacaaatatttaagt (SEQ ID |
| | NO: 170) |
| ura3FBA1p-R | cacttaatacaatacacatacaatttcaattcatcattttttttttattcttttt (SEQ ID NO: 71) |
| Y1FBA1p-F | gaagcctgccgtggcacctgtaactaaaacaatcattgtgtttgtgttgggggtg (SEQ ID |
| | NO: 172) |
| Y2FBA1p-F | tttcgaaagtgttgcgaaagataatgagcctggcattgtgtttgtgttgggggtg (SEQ ID |
| | NO: 173) |
| Y3FBA1p-F | aaaacctgctgtggcgcctgtaacaaagacaatcattgtgtttgtgttgggggtg (SEQ ID |
| | NO: 174) |
| Y4FBA1p-F | tcccgaggtagcacctgagacaagaatatgagccattgtgtttgtgttgggggtg (SEQ ID |
| | NO: 175) |
| Y5FBA1p-F | agatgttgcaccggtgatgaataatgttggagtcattgtgtttgtgttgggggtg (SEQ ID |
| | NO: 176) |
| Y6FBA1p-F | agcagttgcacctgtaaccaaaacagtatcggtcattgtgtttgtgttgggggtg (SEQ ID |
| | NO: 177) |
| Y7FBA1p-F | agcggtagcacctgtgaccaagacggttaaaggcattgtgtttgtgttgggggtg (SEQ ID |
| | NO: 178) |
| Y8FBA1p-F | cgaagtagcaccggtaaccatggctgttttggtcattgtgtttgtgttgggggtg (SEQ ID |
| | NO: 179) |
| Y9FBA1p-F | tgcggatgcacctgtaataaaaacggttctggacattgtgtttgtgttgggggtg (SEQ ID |
| | NO: 180) |
| Y10FBA 1p-F | ggaggttgcgccagtgacaaatgcggtcttcttcattgtgtttgtgttgggggtg (SEQ ID |
| | NO: 181) |
| PDC1tENO2t-F | taatatataattttataataaaaagtttaaactggaacatgtttgtacaaatatt (SEQ ID |
| | NO: 182) |
| Y1ENO2t-R | atttagtaaaagcattagttagatttactcatcggaaaaccagtcttggttttct (SEQ ID |
| | NO: 183) |
| Y2ENO2t-R | atttagtaaaagcattagttagatttaacccttgtggacctttaaacccgccaag (SEQ ID |
| | NO: 184) |
| Y3ENO2t-R | attggctggtttgaaggttcataaggcataaatctaactaatgcttttactaaat (SEQ ID |
| | NO: 185) |
| Y4ENO2t-R | atttagtaaaagcattagttagatttatggattcttatgagtaggtaatggtgca (SEQ ID |
| | NO: 186) |
| Y5ENO2t-R | atttagtaaaagcattagttagatttagccttttctgtgaacagagaaaccggcc (SEQ ID |
| | NO: 187) |
| Y6ENO2t-R | atttagtaaaagcattagttagatttagtcgacatccctaacgacagacaaaccg (SEQ ID |
| | NO: 188) |
| Y7ENO2t-R | atttagtaaaagcattagttagatttagccaacatgtctattaacggccaaacct (SEQ ID |
| | NO: 189) |
| Y8ENO2t-R | atttagtaaaagcattagttagatttaatctctgtggacttgcaatcctgccgct (SEQ ID |
| | NO: 190) |
| Y9ENO2t-R | atttagtaaaagcattagttagatttacgaagcaccaggtctatgcacagcgaga (SEQ ID |
| | NO: 191) |
| Y10ENO2t-R | atttagtaaaagcattagttagatttagaatttttcaatagccaaaccagcccac (SEQ ID |
| | NO: 192) |

TABLE 9
PAND candidates

| PAND | |
|---|---|
| candidates | Sequence (5′ to 3′) |
| P1 from | ATGTTCTTGGCCATCAAGTCTTTGCAATTGTCTAGACAGAAACAAAAGATTGGTAGG |
| Terrapene | AGAAAGAAGTCGACTGATAGATGGTCCGACCTCTTAACAGATATCTTGCAAGGTATT |
| carolina | TTTCTCTGTTGCGCCGTCTTACAGCGTTCAATTGACATGGACTTCAGAACGGGCGC |
| triunguis | TAATAAAACCAATGAGCGTGTCCATCTCGAACCATCTCAGTTAAGAGAAAAGCATATT |
| | TCCTCGTTTCAGGGTGAATCGGAAGAGAAAGAAAAGGATACTAACGCAGAGATTTT |
| | GGTGGCTAACTTCTCCAATATCTACGCATCTGATTTGTTACCTACTAGAAATGGCGAG |
| | GAGTTCACCGATCAATTTTTGTTGGAGGTCGTTGAAATCCTCCTCAACTACATCAAG |
| | AAGACGTATGATGGTAAGTCTAAAGTTTTGGATTTTCACCACCCTCACCAATTATTGG |
| | AGGGTTTGGAAGGTTTTTCATTGGAATTGAGAGATCAACCTGAACCATTAGAGCAAA |
| | TCTTAGTTGACTGTCGTGATACCTTAAAATACGGTGTTAAGACTGGTCATCCAAGGTA |
| | CTTCAACCAATTGTCTTCCGGTTTAGACATTGTCGGTTTGGCTGGCGAGTGGTTGT |
| | CAGCTACTGCGAACACCAATATGTTCACATATGAGATTGCCCCAGTTTTCGTCGTGA |
| | TGGAAGAAATTTTGTTAAAGAAGATGCATGAAATGGTTGGATGGGGAGAAATGGAA |
| | GCAGACGGAATTTTCTCACCTGGTGGTACCATTTCTAACCTTTACTCTGTGTTAGTT |
| | GCAAGGTATAAGAGATTTCCAGAAATTAAAACCAAGGGTATGGCGGCTTTGCCTCAT |
| | ATCTCTTTGTTCGTCTCAGAACACTCCCATTACTCTGTCAAAAAGGCAGCAGCCGTT |
| | TTAGGTATCGGAACTGATAACGTTATTGCAGTTAAGTGTGACGAGAGAGGTAAGATG |
| | ATCCCATCAGATCTTGAAGCCAAAATCTTAAAGGTCAAGAGACAAGATCATATCCCT |
| | CTCTATGTCTCGGCCACTGCCGGCACTACAGTTTACGGTGCGTTCGACCCATTGGA |
| | TAATATCGCAGACATTTGTGAAAAGTATGGTTTGTGGATGCATGTTGATGCGGCTTG |
| | GGGTGGTGGCTTGTTAATGTCTAGAAAACACTCGTTCAAACTTAATGGTATTGAAAG |
| | AGCGAATTCGGTTACGTGGAACCCTCACAAGCTTATGGGAGTTCCATTACAATGTTC |
| | TGCATTTTTAGTTTGTGAAAAGGGTTTGTTACAAGCTTGCAATCAAATGTGTGCTGG |
| | CTATCTTTTCCAACCTGATAAACAGTATGATACCACATACGATACAGGTGATAAGACTA |
| | TTCAATGTGGTAGACATGTCGATGTTTTTAAGCTTTGGTTGATGTGGAAAGCCAAGG |
| | GAACTAGAGGATTCGAAATTCTCATCAACAAGTCCCTTGAATTGGCAGAGTATCTTT |
| | ACAAGGAATTAAACTCGCGTGAGAACTTTGAGTTAGTTTTTGATGCAGAACCTGAAT |
| | TAACAAATGTCTGTTTTTGGTACATTCCTCCTAATCTCAGAAGAATCCCAAATGGCCC |
| | AGAAAGGGAAAAATTATTACACCAGGTCGCACCAAAAATTAAGGCTCGTATGATTGA |
| | AGAAGGTACAACAATGGTTTCATACCAGCCACATGGTGATAAGGTGAATTTCTTTAG |
| | GATGGTGTTTTCCAACCCAGCTACCAGAAAGTCCGACGTGGACTTTTTATTGGAGG |
| | AAATTGAAAGATTGGGTAAGGACTTGTAG (SEQ ID NO: 193) |
| | PROTEIN |
| | MFLAIKSLQLSRQKQKIGRRKKSTDRWSDLLTDILQGIFLCCAVLQRSIDMDFRTGANKT |
| | NERVHLEPSQLREKHISSFQGESEEKEKDTNAEILVANFSNIYASDLLPTRNGEEFTDQF |
| | LLEVVEILLNYIKKTYDGKSKVLDFHHPHQLLEGLEGFSLELRDQPEPLEQILVDCRDTL |
| | KYGVKTGHPRYFNQLSSGLDIVGLAGEWLSATANTNMFTYEIAPVFVVMEEILLKKMHE |
| | MVGWGEMEADGIFSPGGTISNLYSVLVARYKRFPEIKTKGMAALPHISLFVSEHSHYSV |
| | KKAAAVLGIGTDNVIAVKCDERGKMIPSDLEAKILKVKRQDHIPLYVSATAGTTVYGAFD |
| | PLDNIADICEKYGLWMHVDAAWGGGLLMSRKHSFKLNGIERANSVTWNPHKLMGVPL |
| | QCSAFLVCEKGLLQACNQMCAGYLFQPDKQYDTTYDTGDKTIQCGRHVDVFKLWLM |
| | WKAKGTRGFEILINKSLELAEYLYKELNSRENFELVFDAEPELTNVCFWYIPPNLRRIPN |
| | GPEREKLLHQVAPKIKARMIEEGTTMVSYQPHGDKVNFFRMVFSNPATRKSDVDFLLE |
| | EIERLGKDL (SEQ ID NO: 209) |
| P2 from | ATGCCTGCTATCTCCAACGGCTACGTTGCAAACGGTAAGCCAGATTCACAATTAAAC |
| Trichoderma | CGTGCTGCTGAAACTGCCGATCTTTTGGAGTCAGTCCAACAGTTAATTATTCCTTTT |
| gamsii | ATTAAGGCTGCAGATGACGCAGCAGCCCACAGGGCATCTGGTAACCTCGGTTTAGA |
| | TGCAAATGGTGAGCCACAGAACGTTTTGATCGACTCCCAAAAGCCATCGGAGTTGG |
| | TCGCCAGACTCAAGTTCTTGTTACCAGAGGAGGGATTGGGTAAGGACGGCTTGCTT |
| | CAGTCTATTGACAAGTTGTTAAAATACTCTGTGAATACCTGGGATCAGGGTTTCATG |
| | GATAAGCTTTACGCATCTACCACACCTGTTGGCGTCATTGCCGATCTTATCCTCTCT |
| | GTCTTGAACACTAACGTCCACGTCTATCATGTGTCGCCAGCATTAACCGTGATTGAA |
| | AAGACAACCACGAAGGCTTTCGCTAATCTCTTTAATTTTGACGGTCCTCACGCGGG |
| | TGGTATTTCATGTCCAGGTGGTTCGGGTTCTAATCTTACTTCATTGATCGTCGCAAG |
| | AAACACATTGTACCCTGAAACCAAAACTAAGGGTAATGGTAAACGTGATTTTGTCGT |
| | TTTTACCTCCGCCCACGGTCATTACTCCGTTGAAAAGGCAGCTATGATTACTGGTTT |
| | AGGCTCGTCGGCGGTTTGGGCCGTTCCTGTTGATAATGAAGGTTCGATGAGGCCT |
| | CAAGCATTGAGAGAATTGGTTTTGTTGGCAAAAAAGCAAGGAAAGACTCCATTTTAC |
| | GTGAACTCCACGGCGGGAACAACAGTTATGGGTTCATTCGATCCTTTCGAAGAAAT |
| | TTCTAAGGTGTGTAAGGAATTCGGAATGTGGTTACATATCGATGCTTCCTGGGGGG |
| | ATCAGTCATCTTCTCGAAGGCTCAGTCCTGGAAGATGAAAGGTTCTCATCTTGCAG |
| | ACTCTTTAACTGTTAATCCACACAAAATGTTGAACGCGCCATGTACGTGTTCTTTGCT |
| | CTTGGGTCCAGATATGAGGATTTTCCAGAGAGCGAACTCTACAGCTGCCGGTTATC |
| | TCTTTCATCGTGGAGACGATGAAGAGGTTTGGGACTTGGCGGATCTTTCATTGCAA |
| | TGTGGTAGAAGAGGTGATGCCTTGAAGGTTGCCTTAGCTTGGTTATATTACGGAGCA |
| | AATGGATTTGAAAGACAAGTCGATCATGCATTTGATATGGCGTCTTATCTCTTCAGAT |
| | TATTGGAGGAATCCGGTGATTTCGTTATGGTTTCTTCGTATCCTCCACCTTGTTTGCA |
| | AGTTTGCTTTTATTCCGCTCCAGGTGGCAAGCTTTCATCTGACAAAAATGTCAATAC |
| | CTCCAGAACTCAAAGAATCGCCCATATGTTAATCGGTAGAGGTTTCATGGTCGACTA |
| | CGCTCCAGGTGATAAAGGCTCATTTTTGAGAGTGGTCGTTAATGTTCAAACTTTAGA |
| | AGGTACAGTTGAAGGTATCGTTAAGGCAATGGTTGAATTATCGCAAGAGATTTCTTT |
| | GTAG (SEQ ID NO: 194) |
| | PROTEIN |
| | MPAISNGYVANGKPDSQLNRAAETADLLESVQQLIIPFIKAADDAAAHRASGNLGLDAN |
| | GEPQNVLIDSQKPSELVARLKFLLPEEGLGKDGLLQSIDKLLKYSVNTWDQGFMDKLYA |
| | STTPVGVIADLILSVLNTNVHVYHVSPALTVIEKTTTKAFANLFNFDGPHAGGISCPGGS |
| | GSNLTSLIVARNTLYPETKTKGNGKRDFVVFTSAHGHYSVEKAAMITGLGSSAVWAVPV |
| | DNEGSMRPQALRELVLLAKKQGKTPFYVNSTAGTTVMGSFDPFEEISKVCKEFGMWL |
| | HIDASWGGSVIFSKAQSWKMKGSHLADSLTVNPHKMLNAPCTCSLLLGPDMRIFQRA |
| | NSTAAGYLFHRGDDEEVWDLADLSLQCGRRGDALKVALAWLYYGANGFERQVDHAF |
| | DMASYLFRLLEESGDFVMVSSYPPPCLQVCFYSAPGGKLSSDKNVNTSRTQRIAHMLI |
| | GRGFMVDYAPGDKGSFLRVVVNVQTLEGTVEGIVKAMVELSQEISL (SEQ ID NO: 210) |
| P3 from | ATGTGTTCCTCGAAGGAAGGTACGGTGGTCGTCGAGAAGCTTTCCTACAAGGATTT |
| Varroa | GTATCCATACAATATTAACGGAAAGGCGGAGACACGTGCTTTTTTATTGAAGGTTGT |
| destructor | GTCTATTCTCTTAGACTATGTTGATGAAGAAAACGACAGGACATCTAAGATTCTCGAC |
| | TTCAAGATGCCAGAAGAATTGGAACAGATTTTGGACCTTAACTTGCCTGATAAGGGT |
| | TTACCATTGGGAGATTTGTTAAAGGACTGCAGGTCGACGTTAAAATGGCAAGTTAAG |
| | ACCGGTCATCCACATTTCTTCAACCAATTGTCATCCGGATTAGATATTGTTTCGTTAG |
| | CCGGTGAGTGGTTGACCTCTGTTGCAAATACTAATATGTTCACCTACGAGATCGCAC |
| | CTGTTTTTATCTTGATGGAGAATGTTGTCTTGAAGAAGATGAGAGAATACCTTGGCTT |
| | CGTCCATGGTGATTCAATTTTGGCTCCTGGTGGTTCAATTTCAAACATGTACGCGGT |
| | TATGGGTGCAAGACATAAAATGTTCCCAAATTATAAGAAATTAGGATTGAAGGCCTTG |
| | CCTCAATTAGTCATGTACACATCTGTTGATTCACATTATTCCGCTATGGGTGCTGCTG |
| | CCTCGTGTGGTTTAGGTACTGACAATGTGATCCAAATTGGTACCGATTCCATGGGTC |
| | GTATGAGGGTTGACAAACTCGAAGAAGCAGTTTTGGCAACCAAAGCGAAAGGTCA |
| | CGTCCCATTCTTTGTCAATTGTACTTGTGGCACCACTGTTGTTGGTGCATTTGATCC |
| | AATTAACCCAATTGCTGATCTCTGCCAAAAATATGGTATGTGGTTACATATCGATGCC |
| | GCATGGGGTGGTGGTGTTTTGTTATCTAAGAAACACAGACATTTGATGGCAGGAGT |
| | CGAGAGAGCAAATTCCGTCACATGGAACCCTCACAAGCTTATGGGCACCCATCTTC |
| | AGTGTTCTACTATCCACTTTAGACAAGATGGCATTTTGTTAGGCTGTAATCAGATGTG |
| | TGCTGAATACTTGTTTCAACAAGACAAGCATTATGACGTCTCTTTCGATACTGGTGA |
| | CAAGGTCCCTCAATGTGGAAGAAGAAACGATATCTTCAAGCTTTGGTTAATGTGGAG |
| | AGCCAAGGGCACAGAAGGTTTTGAAGCACACATCGACCAGTTATTTGATATCTCCC |
| | AGTACCTCGTGAAAAGGATTAAGGAAATGCCTGAGAAATTCCACCTCATCCAAGAAC |
| | CAGAGATGGTGAATGTTTTGTTTTGGTATATCCCACAACGTTTAAGAGATCAGCCTC |
| | AATCTAAGGAACGTATCGCTGAATTAGGTAGAATTACGGCCCAACTCAAGGCGAGAA |
| | TGATGAACACTGGAACATTGATGATTACTTACCAGCCATTGAGAGAATTGCCAAATTT |
| | TTTTAGAAACATCGTTTCTAACTCGGCGGTTACAAAGGATGATATTGATTTCTTGTTA |
| | GCCGAGATGGATAGATTAGGTAATGATCTTTAG (SEQ ID NO: 195) |
| | Protein |
| | MCSSKEGTVWVEKLSYKDLYPYNINGKAETRAFLLKVVSILLDYVDEENDRTSKILDFKM |
| | PEELEQILDLNLPDKGLPLGDLLKDCRSTLKWQVKTGHPHFFNQLSSGLDIVSLAGEW |
| | LTSVANTNMFTYEIAPVFILMENVVLKKMREYLGFVHGDSILAPGGSISNMYAVMGARH |
| | KMFPNYKKLGLKALPQLVMYTSVDSHYSAMGAAASCGLGTDNVIQIGTDSMGRMRVD |
| | KLEEAVLATKAKGHVPFFVNCTCGTTVVGAFDPINPIADLCQKYGMWLHIDAAWGGGV |
| | LLSKKHRHLMAGVERANSVTWNPHKLMGTHLQCSTIHFRQDGILLGCNQMCAEYLFQ |
| | QDKHYDVSFDTGDKVPQCGRRNDIFKLWLMWRAKGTEGFEAHIDQLFDISQYLVKRIK |
| | EMPEKFHLIQEPEMVNVLFWYIPQRLRDQPQSKERIAELGRITAQLKARMMNTGTLMIT |
| | YQPLRELPNFFRNIVSNSAVTKDDIDFLLAEMDRLGNDL (SEQ ID NO: 211) |
| P4 from | ATGATTTCCTTTCAAACCAAAGGACTTTTTGGTTTACCTAGATTGGTTCTCTTCACCT |
| Anopheles | CTGAAGACGCCCACTACTCCATCAAAAAGTTGGCGTCGTTTATGGGTATTGGTTTCG |
| sinensis | ATAATGTTTTCGCAATCAAGACAGATGCCCGTGGCAAAATGTGTTTGACTGATCTTG |
| | AAGAACAAATTAAGCGTGCCTTAACGGAAGGAGCTCAGCCTTTCATGGTTTCTGCA |
| | ACTGCAGGTACTACAGTTTTGGGCGCCTTTGACCCATTGGAAGGTATTGCTGAGTTA |
| | TGTGCCAAGTATAAACTTTGGATGCACGTCGATGCCGCATGGGGTGGAGGTGCATT |
| | GATGTCCAAGAAGTATAGGCATCTTTTAAAAGGTATCGAGAGAGCTGATTCGGTCAC |
| | TTGGAATCCACATAAGCTCTTAACTGCACCACAACAATGTTCTACATTCTTGACCAG |
| | GCATGAGAATATCTTGTCACAGTGCAATTCCACAAACGCAACTTATCTCTTTCAAAAG |
| | GACAAGTTCTACGATACGAAGTACGATACGGGAGACAAGCATATCCAGTGCGGTAG |
| | AAGAGCTGATGTGTTAAAATTCTGGTTTATGTGGAGAGCAAAGGGTACCAAAGGTTT |
| | GGAACAACATGTTGACACAGCGTTCGATTGTGCAGAGTTTTTTACACAAACCATCAA |
| | GCAAAGACCTGGTTACAAGATGGTCGTTCATGAGCCAGAATGTACCAATGTCTGTTT |
| | TTGGTATCTTCCTCCATCATTACAATCAAGATCTGAACCAAAGACTGCTGAAGAAGC |
| | GACTGAATTTCGTGATGCTTTACACAGAGTGGCGCCTAAGGTCAAGGAAAGAATGA |
| | TGAAATCGGGTGCCATGATGATTACCTACCAGCCAATTCATGATAAGCCTAACTTCTT |
| | CAGATTGGTTATCCAGAACTCGGGCTTGAACCACTCTGACATGATTCACATTGCAGA |
| | CACAATTGAGAAGCTCGCTGCTGATTTGTAG (SEQ ID NO: 196) |
| | PROTEIN |
| | MISFQTKGLFGLPRLVLFTSEDAHYSIKKLASFMGIGFDNVFAIKTDARGKMCLTDLEEQ |
| | IKRALTEGAQPFMVSATAGTTVLGAFDPLEGIAELCAKYKLWMHVDAAWGGGALMSKK |
| | YRHLLKGIERADSVTWNPHKLLTAPQQCSTFLTRHENILSQCNSTNATYLFQKDKFYDT |
| | KYDTGDKHIQCGRRADVLKFWFMWRAKGTKGLEQHVDTAFDCAEFFTQTIKQRPGYK |
| | MVVHEPECTNVCFWYLPPSLQSRSEPKTAEEATEFRDALHRVAPKVKERMMKSGAM |
| | MITYQPIHDKPNFFRLVIQNSGLNHSDMIHIADTIEKLAADL (SEQ ID NO: 212) |
| P5 from | ATGCCTGCTTCTGAGGCTTCCGCCGAAAGGCCTAGAGCCGACGTTGTCATTGAGG |
| Apolygus | AAATTGACGGTGCATCCTCTAAGGAGGCCTTTTTATCGGCTCCAGACAAAGAAATCC |
| lucorum | ACGGACAATTCTTGCATAGAGTCCAACAGATGCTCTTGGATCACGCATTCCAATCAA |
| | CTCACAGAGAATCCAAGGTTACAAGATATGTTGACCCAGATCGTTTGAGAGAGATCA |
| | TCGATATGAATCTTAGAAAGGAACCAGCGTCCGAAGAGGCATTATTGTCATACATCA |
| | GAGATACTATGAAGTACTCGGTTAAAACCGGACATCCTTTTTTTGTCAATCAATTATTT |
| | TCGTCCCTCGACCCATACGCTTTAGCAGGTGCGTGGGTTACAGATTCTTTGAACCC |
| | ATCAGTTTACACGTATGAGGTTTCTCCAGTTTTAACATTGATGGAGGAATCAGTCTTA |
| | AAGGAAATGAGATCCATCGTTGGATGGTCAGACGGTGACGGTATCTTCGCTCCTGG |
| | TGGCTCTATCGCCAACGGCTATGCAATTTCGTGTGCGAGGTTCCATAAGATTCCAGA |
| | TATTAAGGCTAAAGGCTTGCATGGATTGCCAAGGCTCGTTTTGTTCACTTCGGAGGA |
| | TGCACACTATTCGATTAAGAAATTGGCGGCATTCGAGGGTTTGGGTTCTGATAATGT |
| | CTACCCAGTCAAGACTGATGCTAGAGGTAAGATGGACGTTTTGCATCTTAGGGAGTT |
| | GGTCCAACGTGCCAAAGCAGAAGGAGCCGTTCCTTTTATGGTTTCTGCAACATCTG |
| | GTACCACAGTCTTAGGTGCTTTTGATCCTTTGGAAGCCATTCATGAAGTGTGTGAAG |
| | AGGAAGGTATGTGGTTGCACGTCGATGCTGCATGGGGCGGTGGTGCATTAGTTTCC |
| | AAGAAGTACAGACACCTTTTGAGAGGCATTGAGAAGGCAGACTCTGTTACTTGGAA |
| | TCCTCACAAGATGTTGGCTGCACCACAGCAGTGCTCCACTCTOTTAACAAAGCATA |
| | AAACGATTTTAGCGGAATGTCATTCTGCCTCTGCTCAATACCTTTTCCAAAAAGACAA |
| | GTTCTATGATACCTCCTTTGATACCGGTGATAAGCATATCCAGTGTGGTAGACGTGC |
| | GGATGTGTTCAAGTTTTGGTTGATGTGGAAAGCGAAGGGTACCGATGGTTTAGAAA |
| | AGCATGTCAACCGTGTTTATGAAAATGCTGAATACTTCACCGAAAAGATTAGAAGAA |
| | GACCTGGATTCAAGCTTGTTTTAGAAGAGCCTGAATGTACTAACGTTACTTTTTGGTA |
| | CGTCCCACCTTCAATTAGAGGTAGAGATAATGAACCAGAATATAATCAAAAGTTGCAT |
| | AAGGTGGCACCTCTTATCAAAGAAAGGATGACTCGTGAAGGTACAATGTTGATGAC |
| | CTATCAACCATTAAAAGAATTACCTAACTTTTTTAGATTGGTGATCCAGAACTCTGCC |
| | CTCACAACGTCGGATATGGATTACATTGTGTCAGAATTTGAAAGATTAGGTAGAAAC |
| | CTCTAG (SEQ ID NO: 197) |
| | Protein |
| | MPASEASAERPRADVVIEEIDGASSKEAFLSAPDKEIHGQFLHRVQQMLLDHAFQSTH |
| | RESKVTRYVDPDRLREIIDMNLRKEPASEEALLSYIRDTMKYSVKTGHPFFVNQLFSSL |
| | DPYALAGAWVTDSLNPSVYTYEVSPVLTLMEESVLKEMRSIVGWSDGDGIFAPGGSIA |
| | NGYAISCARFHKIPDIKAKGLHGLPRLVLFTSEDAHYSIKKLAAFEGLGSDNVYPVKTDA |
| | RGKMDVLHLRELVQRAKAEGAVPFMVSATSGTTVLGAFDPLEAIHEVCEEEGMWLHV |
| | DAAWGGGALVSKKYRHLLRGIEKADSVTWNPHKMLAAPQQCSTLLTKHKTILAECHSA |
| | SAQYLFQKDKFYDTSFDTGDKHIQCGRRADVFKFWLMWKAKGTDGLEKHVNRVYEN |
| | AEYFTEKIRRRPGFKLVLEEPECTNVTFWYVPPSIRGRDNEPEYNQKLHKVAPLIKERM |
| | TREGTMLMTYQPLKELPNFFRLVIQNSALTTSDMDYIVSEFERLGRNL (SEQ ID |
| | NO: 213) |
| P6 from | ATGAACAATCTCCATGCAACCGATCTTTTGCCAGTCAATCAAGACGGATGGAAGGA |
| Strongyloides | GACAGAAACCTTTCTCAAGGAAGTTGTCGAAATTCTCTTGTCCTACATCAAGACGCA |
| ratti | GAATGACAGGTCGTCAAAGGTTTTAGAATTTCACCACCCACAGGATATGTTGAAGTT |
| | AATTGACCTCTCCATCCCTGATAAGCCAATGTCTTTAGATGATCTTTTGAACTCGTGC |
| | CAAGAAGTTTTAAAGTTGGGTGTGAAGACTGGTCATCCTAGATTTTTTAATCAAATCT |
| | CTTGCGGTTTAGATTTGGTTTGTATGGCCGGAGAGTGGCTTACAGCTACAGCGAAC |
| | ACCAACATGTTCACCTACGAAATTTCTCCAGTGTTCATTTTGATGGAAAAGGAGGTC |
| | ATGAGGAAGATGATTCAGTTGATCGGCTGGGGTGATGATGAAGGTGACGGTATTTT |
| | TTCACCTGGTGGTGCTATTGCTAATCTTTACGCAATGAATGCAGCGAGACATAATGC |
| | TTTCCCACGTTGTAAGCCTTTGGGTATGACTTCCGTCCCAACTTTGTGCTGTTTCAC |
| | ATCGATTGATTCGCACTATTCAATCAAGGGTGCTGCGGCGGTTACTGGAATCGGCA |
| | CTGATAATTGTTTTAACATTCCAACAGATAAGCTCGGTAGAATGATTCCAGAGGCCTT |
| | AGAGCAGAAGATCATCGAATGTAAAAAAGACGGTTTGTATCCTTTTTTCGTCGCAGC |
| | AACGGCCGGTACAACGGTTTATGGTGCTTTTGACCCTTTGATCCAAATCGCAGACAT |
| | TTGTGAAAAGTACGGTGTTTGGTTTCATGTTGACGCTGCCTGGGGCGGTGGTTTGT |
| | TGCTTTCTCCTGAGCATAAGTATAAACTTAGAGGTATCGAGAGAGCAAACTCGGTTA |
| | CTTGGAATCCACACAAACTTATGGGATCTTTATTACAATGTTCTGCATGTTTTATTAGG |
| | AAGGACGGTTTATTGTTCCAAACCAATCAGATGTCCGCAGATTATCTCTTCCAACAG |
| | GATAAGCCATACGATGTTTCATATGATACTGGCGATCGTTCAATTCAATGTGGCCGTA |
| | GAAATGACGTTTTAAAATTGTGGTTAATGTGGAGATCCAAAGGTATGGAAGGTTACC |
| | GTAAACAAATTAACAGGTTGTTGGACTTGGCACAATACTTCACATCCAAGATCAAAA |
| | GAACTGAGGGCTATGAAATGGTCATTGAAGATCCACAATTCTTAAACATTTGTTTTTG |
| | GTACGTGCCTAAGAGATTGAGAAATATGCCTCAAAAGGAAAAAATGGCCAGATTGGA |
| | AAAGATCGCCCCAAAGATTAAAGGAAAGATGATGGAAAGAGGAACCACTATGGTCG |
| | GTTACCAACCAGATAACAACACCCCTAACTTTTTCAGATTAATTATCAACAATCCTGC |
| | CACAACCGAAGAGGATCTTGATTTCTTAATCCAGGAAATTATTGACATTGCTATCGAT |
| | ATGTAG (SEQ ID NO: 198) |
| | PROTEIN |
| | MNNLHATDLLPVNQDGWKETETFLKEVVEILLSYIKTQNDRSSKVLEFHHPQDMLKLID |
| | LSIPDKPMSLDDLLNSCQEVLKLGVKTGHPRFFNQISCGLDLVCMAGEWLTATANTNM |
| | FTYEISPVFILMEKEVMRKMIQLIGWGDDEGDGIFSPGGAIANLYAMNAARHNAFPRCK |
| | PLGMTSVPTLCCFTSIDSHYSIKGAAAVTGIGTDNCFNIPTDKLGRMIPEALEQKIIECKK |
| | DGLYPFFVAATAGTTVYGAFDPLIQIADICEKYGVWFHVDAAWGGGLLLSPEHKYKLRG |
| | IERANSVTWNPHKLMGSLLQCSACFIRKDGLLFQTNQMSADYLFQQDKPYDVSYDTG |
| | DRSIQCGRRNDVLKLWLMWRSKGMEGYRKQINRLLDLAQYFTSKIKRTEGYEMVIEDP |
| | QFLNICFWYVPKRLRNMPQKEKMARLEKIAPKIKGKMMERGTTMVGYQPDNNTPNFF |
| | RLIINNPATTEEDLDFLIQEIIDIAIDM (SEQ ID NO: 214) |
| P7 from | ATGTCCTCCGTTACTAAGACTACTGCCACAACGCAAAATAAAGTTGACGAAATTGAT |
| Bactrocera | AAGATCAGACTTAACTTGGCCGATGATTGGAAGGTTTTAGGTTCAGTCTATGATTTG |
| dorsalis | TTGAAAGACGGTAAGGCTTTCTGTGTCTCAGATGCCGATAGAGATGCAGTTAAAATC |
| | GTTGATTTTAAACAGCCAAATGAGATGAAGCAATTAATTGACCTTTCCATCGACGAAA |
| | ATAAGACACATACTATCGCTGAAATCGAAGATCTTTGTAGATTGGTGATCAAGTACTC |
| | TGTCAAGACGAACCATGGTAGATTTCATAACCAGTTATTTGGTCAGATGGATCCATAC |
| | GGATTGATGGGCCAATGGATTACTGACGCGTTGAACGCTTCAGCTTATACATACGAA |
| | GTCGGTCCTGTCTTCTCCTTGATGGAAACTGAAATTATCAGAACAGTTTGCCAATTA |
| | GCGGGCTATGCAGAGGGTGATGGTATTTTCTCTCCTGGTGGATCGATGTCTAACAT |
| | GTACGGTTTAGTCCTCGCACGTTACTCCAGACACCCACAAGTTAAGACCGCGGGTT |
| | TGTTTGGTATGAGGCCTTTAGCGTTGTTCACCTCGGAAGAATCGCATTATTCCTTCG |
| | TTAAGGCAGCACATTGGTTAGGTTTAGGCGCGGACAATTGCGTTGTGGTTAAAACC |
| | AATGAGAGGGGTCAAATGCTCACAAATGACCTTGAAGCTAAGATTTTGAAGGCTCA |
| | AGAGTCTGGTTGTGAACCTTTTTTCGTTAATGCAACTGCAGGTACTACCGTTTTGGG |
| | TGCTTTCGATGATCTTAACGCAATTGCTGATGTGTGTGAGAAGTATGGTTTGTGGTT |
| | ACATGTCGATGCATGTTTAGGTGGATCGGCACTCTTATCGTATAGAAATAGACACCTT |
| | CTCGCCGGTTTGGACAGAACAAACTCTTTTGCGTGGAACCCACACAAGTCTTTAGG |
| | TGCCCCACTTCAATGTTCTTTGTTTTTAACCAGAGAGCGTGACCTCTTGACTCGTTG |
| | TAATTCAATCCAAGTCAACTACCTTTTCCAACAGGATAAGTTTTATGATGTTTCCTACG |
| | ATACCGGTAATAAGTCTGTCCAGTGTGGCAGAAAAATTGATGCTTTCAAGTTTTGGT |
| | TGATGTTGAAGGCAAGAGGTTACGGATCATTCGGACGTTTGTTAGATCACGCCATTG |
| | ATATTTCAAAAGTGCTCGTTGAAAAGATTAAGCAAAGGGGCTCGGAGCACTTCAGG |
| | CTCGTTTTGGAAGACAATCAGTACATGAACGTGTGTTTCTGGTACATTCCTAAAGCC |
| | TTAAGAGGTTTGGAGGAGACACCTGAATGGCGTGAAAGATTGTACACAGTCGCCCC |
| | AAAGATTAAAGAAAGAATGGCATATTCTGGAACCGTCATGATCGGTTACACCCCATT |
| | GCAATATAGAGGCTTAGGTAATTTTTTTAGAATGGTTTTTACGTGCTTTCCAGTTTTAG |
| | AAGAAGCTGAGTTGGACTTCATCTTGAACGAAATCGAAAGGTTAGGAGAAGAATGT |
| | GAGTACTAG (SEQ ID NO: 199) |
| | Protein |
| | MSSVTKTTATTQNKVDEIDKIRLNLADDWKVLGSVYDLLKDGKAFCVSDADRDAVKIVD |
| | FKQPNEMKQLIDLSIDENKTHTIAEIEDLCRLVIKYSVKTNHGRFHNQLFGQMDPYGLM |
| | GQWITDALNASAYTYEVGPVFSLMETEIIRTVCQLAGYAEGDGIFSPGGSMSNMYGLVL |
| | ARYSRHPQVKTAGLFGMRPLALFTSEESHYSFVKAAHWLGLGADNCVVVKTNERGQ |
| | MLTNDLEAKILKAQESGCEPFFVNATAGTTVLGAFDDLNAIADVCEKYGLWLHVDACLG |
| | GSALLSYRNRHLLAGLDRTNSFAWNPHKSLGAPLQCSLFLTRERDLLTRONSIQVNYLF |
| | QQDKFYDVSYDTGNKSVQCGRKIDAFKFWLMLKARGYGSFGRLLDHAIDISKVLVEKIK |
| | QRGSEHFRLVLEDNQYMNVCFWYIPKALRGLEETPEWRERLYTVAPKIKERMAYSGTV |
| | MIGYTPLQYRGLGNFFRMVFTCFPVLEEAELDFILNEIERLGEECEY (SEQ ID NO: 215) |
| P8 from | ATGCTTACTATTTTATTTCGTTCTTGGCCACATCTTTGGGTGTTAGTTCCAATTATGTG |
| Lutzomyia | TACTCAATACGTTCAAATGCATGCAGTCAAGTGGAAGTGGATTATTTTAGAATCCGAA |
| longipalpis | ATTTTGAGAGCCAAGTGTGAGGGTGGCCATCCTTTCATGGTTTCTGCAACTGCTGG |
| | TACTACAGTCTTGGGAGCTTTTGACCCTTTGACCGAAATCGCGAACTTGTGTGAAAA |
| | ATATCAATTGTGGTTTCATGTCGATGCAGCATGGGGTGGTGGTGCATTAGTTTCTCC |
| | AAAGTATAGAGCCTTGTTGGCCGGAATCGAGAGAGCTGATTCGGTGACCTGGAACC |
| | CTCATAAGCTCTTGGCTGCACCACAACAATGTTCGACTTTCTTAACGAGACACCCAA |
| | ACTTGTTGAAACAGTGTCATTCATGCAATGCATCTTACCTTTTTCAAAAGGATAAGTT |
| | TTACGATACTAAGTACGACACGGGCGACAAGCATATTCAGTGTGGTAGAAGGGCTG |
| | ATGTTTTCAAGTTTTGGCTCATGTGGAAGGCGAAAGGTACCTTGGGATTGCAAAGG |
| | CACGCCGAGAAGGTCTTCGAGATGGCAGAATTCTTCACCGAACAGATTAGACAGAG |
| | GGACGATTTTGAGATGGTTGTCGCGGAACCAGAATGCACAAATGTTTGCTTCTGGT |
| | ATCTCCCTCCTTCTCTTCGTTCCTGTCCAAGGGATGAAGAGTTCTTAACTAAGCTCC |
| | ACAGAGTGGCACCTAAGATCAAAGAAAGAATGATGAAGGAGGGTTCGATGATGATC |
| | ACGTACCAGCCTCTTCGTCAAAAACCAAACTTCTTCAGGTTAGTGTTGCAGAACTCA |
| | TCCTTGGAAAAGTCAGATATGCTCCATATCATTAACAAAATCGCCCAGTTAGGTGAA |
| | GACCTCGCAGATTCTGTTACATGGAATCCTCATAAACTTTTGGCTGCTCCTCAACAA |
| | TGTTCCACCTTTTTAACCAGACACCCAAATTTGTTGAAGCAGTGTCACTCATGTAAT |
| | GCCTCGTATCTCTTCCAAAAAGATAAGTTCTACGATACAAAGTACGATACAGGTGAC |
| | AAGCACATTCAATGTGGTAGACGTGCGGATGTCTTTAAGTTTTGGTTGATGTGGAAG |
| | GCTAAAGGAACATTAGGTTTACAAAGACATGCAGAGAAGGTTTTCGAAATGGCCGA |
| | ATTTTTTACTGAGCAAATTAGACAAAGAGACGATTTTGAAATGGTCGTTGCCGAACC |
| | TGAATGTACCAATGTTTGTTTCTGGTATCTTCCACCTTCTTTGAGATCGTGTCCAAGA |
| | GATGAGGAATTTTTAACTAAACTCCACAGAGTCGCTCCAAAGATTAAGGAACGTATG |
| | ATGAAAGAGGGTTCAATGATGATCACATACCAGCCTTTAAGACAAAAGCCAAACTTT |
| | TTCAGATTGGTTTTGCAAAATTCTTCCCTCGAAAAATCCGACATGTTACATATCATTAA |
| | TAAGATCGCGCAATTGGGCGAAGATCTTTAG (SEQ ID NO: 200) |
| | PROTEIN |
| | MLTILFRSWPHLWVLVPIMCTQYVQMHAVKWKWIILESEILRAKCEGGHPFMVSATAGT |
| | TVLGAFDPLTEIANLCEKYQLWFHVDAAWGGGALVSPKYRALLAGIERADSVTWNPHK |
| | LLAAPQQCSTFLTRHPNLLKQCHSCNASYLFQKDKFYDTKYDTGDKHIQCGRRADVFK |
| | FWLMWKAKGTLGLQRHAEKVFEMAEFFTEQIRQRDDFEMVVAEPECTNVCFWYLPP |
| | SLRSCPRDEEFLTKLHRVAPKIKERMMKEGSMMITYQPLRQKPNFFRLVLQNSSLEKS |
| | DMLHIINKIAQLGEDLADSVTWNPHKLLAAPQQCSTFLTRHPNLLKQCHSCNASYLFQK |
| | DKFYDTKYDTGDKHIQCGRRADVFKFWLMWKAKGTLGLQRHAEKVFEMAEFFTEQIR |
| | QRDDFEMVVAEPECTNVCFWYLPPSLRSCPRDEEFLTKLHRVAPKIKERMMKEGSMM |
| | ITYQPLRQKPNFFRLVLQNSSLEKSDMLHIINKIAQLGEDL (SEQ ID NO: 216) |
| P9 | ATGGAACGTCACATTAATGGTGTGAAGTCTTGTGAATCCCAAGCGCCTGTTGCCCA |
| Haplochromis | ACCAGAAAAGTGTTCTCCAGCGGGTTCATTTGACGTTTCGTCCTCAACTGCTGACG |
| burtoni | AACAGATTGCAGAATCGTTTATTCGTCAGGCCATGGAGATTATTGTGGACGAAGCCG |
| | TCAAGAAGGCCACCAATGTTCAAGAGAAGGTTTGTGAATGGCATTCACCTGAGCAA |
| | CTCAAGAAGTTGTTAGATCTTGAATTGAGAGATAGAGGCGAACCTGAGTCGGAGAT |
| | TTTGCAGAGATGCCGTGATGCCATCCGTTACTCGGTTAAGACCTCCCATCCTAGATT |
| | TTTTAACCAATTGTACGCTGGTATGGAACCATATTCTATGGTCGCTTCTTTCATTGCA |
| | GAAGCAGTTAAAACGAACCTCTACACTTATGAGGTCGCACCAGTTTTTACCTTGATG |
| | GAAGATGCCGTCTTAAGAAAGATGATTAAAGTCATCGGTTGGGAAGAAGGTGGCGA |
| | TGGAATCTTTAATCCAGGTGGATCTTTGTCAAATATCTACGCAGTCAATCTTGCAAGA |
| | TATAGGTACTGTCCAGACTTCAAAGAAGTCGGTATGTTCTCCGCACCTAGATTAGTG |
| | ATTTTGACTTCTCAAGAATGCCATTACTCTATCTCTAAGGGTGCTGCTTTGATGGGTA |
| | TCGGCACAAAGAATGTCTACACAGTTCCAACAGATGAGAGAGGTAGAATGATCCCT |
| | TCTGCATTAGAAGAAAAAATTAAGCTCGCAAAGTCGGAAGGAGCTGTCCCTTTTATG |
| | GTTAATGCCACTGCAGGTACAACTGTTTTAGGTGCGTTTGACCCAATCGACGAAATT |
| | GCCGATATTTGTGAGAAGCATAACTTGTGGTTGCACGTCGATGCTTGTTGGGGTGG |
| | TGCCGCAATTATGTCAAAGAAGCATAAACATCTTTTAAAGGGTATCCACAGAGTTGA |
| | CTCGGTTGCTTGGAACCCTCACAAGATGTTAATGGCGTCTTTACAATGTTCTGCTTT |
| | TTTAGTCAGAGATAAAACATCCTTGTTGCAGAGATGTCATTCGGCTAGAGCGTCCTA |
| | TTTGTTCCAACAGGATAAGTTCTATGATGTTTCTTACGACACGGGAGATAAGTCAGTT |
| | CAATGTTCTAGAAAGCCTGATGCTTTCAAAATCTGGTTGATGTGGAAGGCGTTGGG |
| | CTCCAGAGAGTTAGAGCAAAGGGTTGATCGTGCATTAGCAATGGCTAGATATCTTGC |
| | CGAGGAAATCAAAAAGAGAGAAGGTTTCAGGTTGGTGTTAGAGCCTGAATTCGCAA |
| | ACGTGTGTTTCTGGTATCTTCCACCATCTTTAAGAAATCTCCCAGAATCCCCAGAATT |
| | ATGGAAAAAATTGCACACCGTTAGGTCATCATCCTTGCAAAGGGTTATTTTAATGTAC |
| | TCCAACGCAGATATGAAGAGACTCTTCTCTTTTTCGTTGGCTAACGCGTAG (SEQ ID |
| | NO: 201) |
| | PROTEIN |
| | MERHINGVKSCESQAPVAQPEKCSPAGSFDVSSSTADEQIAESFIRQAMEIIVDEAVKK |
| | ATNVQEKVCEWHSPEQLKKLLDLELRDRGEPESEILQRCRDAIRYSVKTSHPRFFNQL |
| | YAGMEPYSMVASFIAEAVKTNLYTYEVAPVFTLMEDAVLRKMIKVIGWEEGGDGIFNPG |
| | GSLSNIYAVNLARYRYCPDFKEVGMFSAPRLVILTSQECHYSISKGAALMGIGTKNVYTV |
| | PTDERGRMIPSALEEKIKLAKSEGAVPFMVNATAGTTVLGAFDPIDEIADICEKHNLWLH |
| | VDACWGGAAIMSKKHKHLLKGIHRVDSVAWNPHKMLMASLQCSAFLVRDKTSLLQRC |
| | HSARASYLFQQDKFYDVSYDTGDKSVQCSRKPDAFKIWLMWKALGSRELEQRVDRAL |
| | AMARYLAEEIKKREGFRLVLEPEFANVCFWYLPPSLRNLPESPELWKKLHTVRSSSLQ |
| | RVILMYSNADMKRLFSFSLANA (SEQ ID NO: 217) |

TABLE 10
BAPAT candidates

| BAPAT | |
|---|---|
| candidates | Sequence (5′ to 3′) |
| B1 from | ATGGAACTTATGATCGTTCAAGTCACAGAGCAAACCCAGTCTTTAAAGAAGACT |
| Pseudomonas | GATGAAAAGTACTTGTGGCACGCAATGAGAGGTGCAGCGCCATCCCCTACAAA |
| nunensis | CTTGATCATTACCAAAGCGGAAGGCGCATGGGTCACAGATATTGACGGTAATAG |
| | ATATTTAGATGGAATGTCTGGATTGTGGTGTGTGAATGTTGGTTACGGTAGAAAG |
| | GAATTGGCAAGAGCGGCTTTCGAGCAGTTAGAAGAGATGCCATACTTCCCATTG |
| | ACCCAATCCCATGTCCCAGCCATCAAGTTGGCAGAAAAGTTAAATGAATGGTTA |
| | GATGATGAGTACGTCATCTTCTTCTCTAATTCGGGTTCTGAAGCTAATGAAACAG |
| | CGTTTAAGATCGCGAGACAATACCATCAACAAAAGGGTGACCACGGAAGATATA |
| | AGTTTATTTCAAGATACAGAGCGTATCATGGTAACTCCATGGGTGCATTAGCCGC |
| | CACTGGTCAGGCCCAAAGGAAGTATAAGTATGAGCCTTTGGGTCAAGGATTTCT |
| | TCACGTCGCGCCACCTGACACGTATAGGAATCCAGAAGACGTCCATACGTTGG |
| | CCTCGGCAGAGGAGATTGACAGAGTCATGACTTGGGAGCTCTCCCAGACAGT |
| | CGCAGGCGTCATTATGGAGCCAATCATCACCGGCGGCGGTATCCTCATGCCAC |
| | CTGATGGTTATATGGAAAAGGTGAAGGAAATCTGTGAAAAGCATGGTGCATTATT |
| | AATCTGTGATGAAGTCATCTGTGGATTCGGACGTACTGGCAAGCCTTTTGGTTT |
| | CATGAACTATGGCGTCAAACCAGACATTATTACTATGGCTAAGGGTATCACTTCC |
| | GCATATCTCCCTTTGTCTGCTACTGCTGTCCGTAGAGAGGTCTACGAAGCTTTC |
| | GTTGGTTCGGACGACTACGATAGATTCCGTCATGTTAACACATTCGGCGGCAAT |
| | CCTGCCGCCTGTGCACTCGCTTTGAAGAACTTGGAAATTATGGAAAATGAGAA |
| | GTTGATCGAGAGGTCCAAGGAATTGGGTGAAAGATTGCTCTATGAGTTGGAAG |
| | ACGTCAAAGAACACCCAAACGTTGGTGATGTTAGAGGTAAGGGTTTACTTCTTG |
| | GTATCGAGTTGGTCGAAGACAAACAAACCAAAGAACCAGCATCGATTGAGAAG |
| | ATGAATAAGGTTATTAATGCATGCAAGGAGAAAGGATTGATCATTGGTAAGAACG |
| | GCGATACTGTCGCCGGCTATAATAACATCTTGCAGTTAGCCCCACCATTGTCGAT |
| | TACTGAGGAAGATTTCACTTTTATCGTCAAGACAATGAAGGAATGCTTAGCCCAA |
| | CATCATCATCATCATCATTAG (SEQ ID NO: 202) |
| | Protein |
| | MELMIVQVTEQTQSLKKTDEKYLWHAMRGAAPSPTNLIITKAEGAWVTDIDGNRYL |
| | DGMSGLWCVNVGYGRKELARAAFEQLEEMPYFPLTQSHVPAIKLAEKLNEWLDD |
| | EYVIFFSNSGSEANETAFKIARQYHQQKGDHGRYKFISRYRAYHGNSMGALAATG |
| | QAQRKYKYEPLGQGFLHVAPPDTYRNPEDVHTLASAEEIDRVMTWELSQTVAGVI |
| | MEPIITGGGILMPPDGYMEKVKEICEKHGALLICDEVICGFGRTGKPFGFMNYGVK |
| | PDIITMAKGITSAYLPLSATAVRREVYEAFVGSDDYDRFRHVNTFGGNPAACALALK |
| | NLEIMENEKLIERSKELGERLLYELEDVKEHPNVGDVRGKGLLLGIELVEDKQTKEP |
| | ASIEKMNKVINACKEKGLIIGKNGDTVAGYNNILQLAPPLSITEEDFTFIVKTMKECLA |
| | QHHHHHH (SEQ ID NO: 228) |
| B2 from | ATGTCCTCGGAAACCATTAGTCAATCAATTAATGTGGTTCATCCTGTCACTCTTT |
| Pseudomonas | CACATGGTAAGAACGCCGAAGTCTGGGATACGGAAGGTAAGAGATACATTGATT |
| nunensis | TCGTCGGTGGAATTGGTGTTTTGAACCTTGGCCATTGTAATCCTAGGATTGTCG |
| | AAGCGATCAGAGAACAGGCAACAAGGTTGACTCATTACGCTTTCAACGCTGCT |
| | CCACACACTCCATATTTGGAATTGATGGACAGATTAACTGCCTTTATCCCAGTCT |
| | CATATCCAGTTTCTGGAATGTTAACAAATAGTGGCGCCGAAGCTGCCGAAAACG |
| | CATTGAAGATCGTGCGTGGTGCGACCGGTCGTACCGCAGTCATTGCATTCGAT |
| | GGTGCCTTTCACGGTAGAACTCTCGCTACACTCAATTTGAATGGTAAAGTTGCC |
| | CCTTACAAACAAAAGGTTGGTGTCTTACCAGGTCCTGTCTACCATTTACCATTTC |
| | CATCTAAGGATAACGGTGTGACATGTGCTGAAGCGCTCAAGGCAATGGACAGA |
| | TTATTTTCTGTTGAAATCGATGTCAATGACGTCGCATGTTTCATCGTCGAACCTG |
| | TGCAAGGCGAAGCCGGCTTCTTAGCAATGGATGTGGAATTTGCTCAAGCATTG |
| | AGAAGGTTCTGTGACGAAAAGAACATCTTGTTAATTGCGGATGAAATTCAAAGT |
| | GGTTTTGGTAGAACTGGTGAGCGTTTTGCTTTCTCCAGATTGGGAATCGAACCA |
| | GACTTAATCTTGTTGGCCAAGTCCATCGCTGGTGGTGTTCCTTTGGGCGCCGT |
| | GGTGGGTAGAAAGTCGTTATTGGATACTTTACCAAAGGGCGGTTTAGGTGGTAC |
| | TTATTCAGGTAACCCTATCGCTTGCGCGGCCGCTCTTGCAACCTTGGATGAAAT |
| | GACGGATGCTAATTTGAATGCTTGGGGTTCCCAACAGGAAGAAGCCATTGTCTC |
| | CCGTTATGAGGCATGGAGAGTGTCTAAGTTATCGCCATACTTAGGTCGTTTGAC |
| | GGGTGTTGGAGCAATGAGAGGTATCGAACTTTCCAATGCTGATGGTTCACCAG |
| | CTAGTGCGCAATTGACCCAACTCTTAGCCCTTGCAAGAGACGCAGGTTTACTTT |
| | TGATGCCTTCTGGTAAATCAAGAAACATTATTAGATTACTCGCCCCTTTGACGAC |
| | AGAAGCTACTGTTTTAGAAGAAGGTCTTGATATTTTCGAGGCGTGCTTGAAGAA |
| | GCTCACGCATCATCATCATCATCATTAG (SEQ ID NO: 203) |
| | Protein |
| | MSSETISQSINVVHPVTLSHGKNAEVWDTEGKRYIDFVGGIGVLNLGHCNPRIVEAI |
| | REQATRLTHYAFNAAPHTPYLELMDRLTAFIPVSYPVSGMLTNSGAEAAENALKIVR |
| | GATGRTAVIAFDGAFHGRTLATLNLNGKVAPYKQKVGVLPGPVYHLPFPSKDNGVT |
| | CAEALKAMDRLFSVEIDVNDVACFIVEPVQGEAGFLAMDVEFAQALRRFCDEKNIL |
| | LIADEIQSGFGRTGERFAFSRLGIEPDLILLAKSIAGGVPLGAVVGRKSLLDTLPKGG |
| | LGGTYSGNPIACAAALATLDEMTDANLNAWGSQQEEAIVSRYEAWRVSKLSPYLG |
| | RLTGVGAMRGIELSNADGSPASAQLTQLLALARDAGLLLMPSGKSRNIIRLLAPLTT |
| | EATVLEEGLDIFEACLKKLTHHHHHH (SEQ ID NO: 229) |
| B3 from | ATGTCGATCTCAGAGAAATATTTTCCACAAGAGCCCCAATCTCCAGAGTTAAAG |
| Pichia | ACTGCCATTCCTGGTCCACAATCTAAGGCTAAGTTGGAAGAATTGTCAGCCGTT |
| kudriavzevii | TACGATACTAAGGCCGCTTACTTTGTTACGGACTATTATAAATCCTTAGGTAACTA |
| | CATCGTCGACGCAGACGGAAACAAATTATTGGATTCCTACTGCCAGATTTCATC |
| | CATTGCTCTCGGTTACAACAATCCAGCGTTGCTCAAAGTTGCGCACTOGGATGA |
| | AATGGCGGTTGCTTTGTGCAACCGTCCAGCACTTGCTTGCTTCCCTTCCACCG |
| | ACTACTACGAGATCTTAAAGAAAGGTTTGCTTTCCGTTGCGCCTAAGGGTTTGG |
| | ACAAAGTCTGTACCGCCCACACAGGTTCGGATGCAAACGAGATGGCATTTAAG |
| | GCTGCATTTCTCTTTCAAGCCTCGAAGAAGAGAGGAGATAAGCCTTTTACTTCG |
| | GAAGAACTTGAATCAGTTATGGAAAATAAGTTGCCAGGAACATCAGATATGGTCA |
| | TCTTGTCATTTGAGAAAGGTTTCCATGGTAGATTGTTCGGTTCCTTGTCTACCAC |
| | CAGATCCAAGGCGATCCATAAGCTCGACATTCCAGCATTTGAATGGCCAAAAGC |
| | TCCTTTTCCCCAATTAAAGTATCCACTCGACCAATTTCAAGCAGAAAATAAGGCG |
| | GAAGAAGAGAGGTGTTTGAAGGCACTCGAAGAAATCATTGTCAACTCGCCTGC |
| | AAAGATTGCCGCGGCGATCATTGAACCTGTCCAGTCAGAAGGTGGTGACAATC |
| | ACGCGTCGCCTGAATTCTTCCAAGGTATCAGAGAAATCACGAAGAAGCACGGC |
| | GTTATCCTTATTGTCGATGAGGTTCAGACAGGCGGTGGTGCCAGTGGTAAAATG |
| | TGGTTGCATGAGCACTACGGCATCGTGCCTGACATTATGACTTTCTCTAAGAAA |
| | ATGCAGAATGCCGGATTCTTCTTTTCGGAAGCCGGTTTAGCAGGAGACCAGCC |
| | TTTCCGTCAATTTAACACCTGGTGTGGTGATCCATCGAAGGCTCTCATTGCTAG |
| | AACGATTATTGAGGAAATTAAGGACAAGAATTTACTCACTTCTGTTACAGAAACG |
| | GGAGACTACCTTTACTCCAAATTGGAAGCCATTTCTGCCAAGTACGACAAGATG |
| | ATCAACTTGAGAGGTAAGGGCAGGGGCTTCTTCATCGCGTTCGATGCTCCAAC |
| | ACCTGAACTTAGAAACAAATTCATTGCTGAGTGCAAGAAGCTTGGTTTAAATATC |
| | GGCGGTTGTGGTGAGCAGGGTGTCAGATTGCGTCCAGCTTTGGTCTTTGAAAA |
| | GAAACATGCCGATATTTTGGCATCTATCATTGATCAAGCATTTTCCAAGATCCATC |
| | ATCATCATCATCATTAG (SEQ ID NO: 204) |
| | Protein |
| | MSISEKYFPQEPQSPELKTAIPGPQSKAKLEELSAVYDTKAAYFVTDYYKSLGNYIV |
| | DADGNKLLDSYCQISSIALGYNNPALLKVAHSDEMAVALCNRPALACFPSTDYYEIL |
| | KKGLLSVAPKGLDKVCTAHTGSDANEMAFKAAFLFQASKKRGDKPFTSEELESVM |
| | ENKLPGTSDMVILSFEKGFHGRLFGSLSTTRSKAIHKLDIPAFEWPKAPFPQLKYPL |
| | DQFQAENKAEEERCLKALEEIIVNSPAKIAAAIIEPVQSEGGDNHASPEFFQGIREIT |
| | KKHGVILIVDEVQTGGGASGKMWLHEHYGIVPDIMTFSKKMQNAGFFFSEAGLAG |
| | DQPFRQFNTWCGDPSKALIARTIIEEIKDKNLLTSVTETGDYLYSKLEAISAKYDKMI |
| | NLRGKGRGFFIAFDAPTPELRNKFIAECKKLGLNIGGCGEQGVRLRPALVFEKKHA |
| | DILASIIDQAFSKIHHHHHH (SEQ ID NO: 230) |
| B4 from | ATGAGTTTGAACGACGATGCCACATTTTGGCGTAATGCAAGGCAACATTTGGTG |
| Burkholderia sp. | AGATATGGTGGTACCTTCGAACCTATGATTATTGAACGTGCGAAGGGTTCCTTT |
| Bp9004 | GTTTACGATGCAGATGGTAGGGCAATCTTGGATTTCACATCCGGTCAAATGTCC |
| | GCAGTCTTAGGCCACTGTCATCCAGAAATTGTCTCTGTCATTGGCGAATACGCA |
| | GGCAAGCTTGACCACTTATTTTCAGGTATGTTGTCGAGACCAGTTGTCGATTTG |
| | GCTACTAGACTCGCCGATATCACTCCAGATGGATTGGATAGAGCTCTTCTCCTC |
| | TCTACGGGTGCAGAATCGAACGAAGCAGCAATTAGAATGGCTAAGTTGGTTACA |
| | GGAAAGTACGAGATTGTCGGTTTTGCTCAAAGTTGGCACGGTATGACTGGTGC |
| | AGCAGCCTCAGCAACTTACTCAGCGGGTAGGAAAGGTGTTGGACCAGCTGCT |
| | GTCGGTTCTTTTGCAATTCCAGCCCCTTTTACTTACAGACCAAGATTCGAAAGA |
| | AATGGCGCCTATGACTATTTGGCTGAATTGGACTACGCATTCGATCTCATTGATA |
| | GACAATCATCGGGTAACTTAGCTGCATTCATTGCTGAACCCATTCTTTCTTCTGG |
| | TGGTATTATCGAATTGCCAGAAGGATATATGGCAGCATTAAAGAGAAAGTGTGAA |
| | GCCCGTGGTATGTTATTGATTTTGGATGAGGCACAAACGGGTGTCGGTCGTACT |
| | GGTACCATGTTTGCCTGCCAAAGAGACGGAGTCACTCCAGATATTCTCACTTTG |
| | TCGAAGACATTGGGTGCGGGTTTGCCTTTGGCCGCGATCGTTACTTCGGCAGC |
| | GATCGAAGAGAGGGCCCATGAGCTCGGTTATTTATTCTACACAACCCATGTTTC |
| | TGATCCTTTACCAGCAGCTGTCGGATTGAGAGTGTTGGATGTTGTTCAAAGAGA |
| | CGGATTGGTTGCTAGAGCAAACGTCATGGGAGACCGTTTGAGGAGAGGCTTAT |
| | TGGATTTGATGGAACAATTCGATTGTATTGGTGATGTCAGAGGTCGTGGTTTGT |
| | TGTTAGGTGTTGAGATCGTTAAGGATAGAAGAACAAAAGAACCAGCAGACGGT |
| | CTCGGCGCAAAGATCACTCGTGAATGTATGAACTTGGGCTTATCCATGAATATTG |
| | TTCAGTTGCCTGGAATGGGTGGTGTCTTTAGAATTGCACCACCTCTCACGGTCT |
| | CCGAAGACGAAATTGACTTAGGTTTATCTTTATTAGGACAGGCCATTGAACGTG |
| | CACTTCATCATCATCATCATCATTAG (SEQ ID NO: 205) |
| | Protein |
| | MSLNDDATFWRNARQHLVRYGGTFEPMIIERAKGSFVYDADGRAILDFTSGQMSA |
| | VLGHCHPEIVSVIGEYAGKLDHLFSGMLSRPVVDLATRLADITPDGLDRALLLSTGA |
| | ESNEAAIRMAKLVTGKYEIVGFAQSWHGMTGAAASATYSAGRKGVGPAAVGSFAI |
| | PAPFTYRPRFERNGAYDYLAELDYAFDLIDRQSSGNLAAFIAEPILSSGGIIELPEGY |
| | MAALKRKCEARGMLLILDEAQTGVGRTGTMFACQRDGVTPDILTLSKTLGAGLPLA |
| | AIVTSAAIEERAHELGYLFYTTHVSDPLPAAVGLRVLDVVQRDGLVARANVMGDRL |
| | RRGLLDLMEQFDCIGDVRGRGLLLGVEIVKDRRTKEPADGLGAKITRECMNLGLS |
| | MNIVQLPGMGGVFRIAPPLTVSEDEIDLGLSLLGQAIERALHHHHHH (SEQ ID |
| | NO: 231) |
| B5 from | ATGGAAAACGTGAAATACCAAATTCCACCTGCCGAGGTCTTGGACACATTGGGT |
| Mesorhizobium | AAATATATCTTGGTTGATGGTTTCCACACTGTGATCGATCTTGTTAAATCCCACG |
| sp. Root552 | ACAACTATATTGTGGATGCTGTCACGGGACAGGAGATTTTAGATTGTTACAGTTA |
| | TTTTGCATCATTGCCTATTGGTCATAATCACCCCAAACTTTTCTATGACGACTTCT |
| | TGAAGGCGCTCTTCAGAGCGACTATCGCTAATCCATCTAACTCTGACATTTACTG |
| | CCCAGAATACGCATGGGCCGTCAAGACATTCGCCGAATTCGCGAAGCCAGCTG |
| | AATTCAAGTATATGTTCTTTGTTGCGGGTGGTGCACCAGCCATTGGTAACGCCT |
| | TGAAGGTTGCATTCGACTGGAGAACACGTAAGGAAATGAACGCCAATTTTCTCC |
| | CACCTAAAGCGGGTTACGGTGTTCTCCACTTGAAGGAAGCGTTTCACGGCAGA |
| | CTCGGTTACACCTTGTCCTTGACTAATACGGATCCAGTTAAGACCGAGTATTTCC |
| | CTAAGTTCGATTGGCCAAGAATCACTAACCCTAAGATTAGATTTGGTAAGGATGG |
| | TAGAATCTCCAATGCCACTAGTGTGCAGAGAGCCGAGCGTGAAGCGATGATCC |
| | AGATTGACCGTGCTATGAGAAGACAGGAAATTGCTGCTTTGATCTTGGAACCAA |
| | TTCAAGGTGAAGGTGGTGATAACCATTTTAGACCATGCTTCTTCAAAGCGCTCA |
| | GACAAAAGTGTGATGACAACGATATCATGTTGATCTTCGATGAAGTGCAAACCG |
| | GTGTTGGCTTGACCGGACGTATGTGGGCTTACGAACACTTCGGCGTCACACCT |
| | GATATTCTCGTTTTCGGTAAGAAGACCCAGGTTTGTGGTATCATGGCAACCGAG |
| | AGAGTTGACGAGGTTCCCGACAACGTTTTCAGAAAATCAGGCAGGATTAATTCG |
| | ACTTGGGGAGCAAATTTGGCGGATTTCCTTAGAGGTGCACGTTATTTAGAAATTA |
| | TCTGTGAGGATAACTTAGTTGCCAACGCAGCAACCGTTGGTAAGTATTTCTTGG |
| | ATAAGTTACGTGCCGAAGTTTACGACCCTAAGCATGCACAAAACATCAGGGGAC |
| | GTGGCTTAATGATTGCCTTTGATTTCAAGGACGCGGCAACAAGAAACGAATTTA |
| | GAAGAAAGGCTTGGGACAACGGCTTAGCAACATTGTCATGCGGTACCTATTCTG |
| | TCCGTTTTAGACCACCACTCACGTTTACAAAGGAGAACGTGGATGAGGCGATT |
| | CGTATCATCAAGAAGTCCTTACATCATCATCATCATCATTAG (SEQ ID NO: 206) |
| | Protein |
| | MENVKYQIPPAEVLDTLGKYILVDGFHTVIDLVKSHDNYIVDAVTGQEILDCYSYFAS |
| | LPIGHNHPKLFYDDFLKALFRATIANPSNSDIYCPEYAWAVKTFAEFAKPAEFKYMFF |
| | VAGGAPAIGNALKVAFDWRTRKEMNANFLPPKAGYGVLHLKEAFHGRLGYTLSLT |
| | NTDPVKTEYFPKFDWPRITNPKIRFGKDGRISNATSVQRAEREAMIQIDRAMRRQE |
| | IAALILEPIQGEGGDNHFRPCFFKALRQKCDDNDIMLIFDEVQTGVGLTGRMWAYE |
| | HFGVTPDILVFGKKTQVCGIMATERVDEVPDNVFRKSGRINSTWGANLADFLRGA |
| | RYLEIICEDNLVANAATVGKYFLDKLRAEVYDPKHAQNIRGRGLMIAFDFKDAATRN |
| | EFRRKAWDNGLATLSCGTYSVRFRPPLTFTKENVDEAIRIIKKSLHHHHHH (SEQ ID |
| | NO: 232) |
| B6 Candidatus | ATGGAAGACAAAAGAAAAGGTGAGAGAAAGCGTGAGAGAAAGGAAGAACACG |
| Falkowbacteria | GAATGGGTACTGGAGATAAAATGAACCCTCAAATGCAAGAGATCGGTATGTTAG |
| bacterium | ATACCATCGGCCCTAGATCATTGGAAGTCATTGAAGAAGATTGTAACGTTATGTC |
| RIFOXYA2_FULL_ | AGCTTGTGAGACTAGACCATATCCACTCGTCGTCGATAGGGCGAAGGGATCCG |
| 47_19 | TCGTGACAGACTTGGACGGTAGAGAGTACATTGACTTTGTTGCAGGCATTGCA |
| | GTCTTGAATGCCGGTCATTCTAATCCAGCGGTTCAAGCAGCAATCAGTGCGCAA |
| | TTGGAGAAAATGACTCACTGTTGCTCAGCAGACTTCTTCGCCGAACCCCCATTA |
| | AAATTAGCCAGAAAGTTAAGAGAACTTTCGGGTTATAGTAAGGTTTTCTATTGTA |
| | ACTCTGGTACGGAAGCAGTTGAAGCCGCAATTAAGTTGGCCACTTGGAAGACC |
| | GGTAAGCAAAATTTGATTGGCTTCTACAACAGTTTCCATGGTAGGACTTTGGGT |
| | TCATTATCGTTGACTTCATCGAAAGCTAGACAGAAAGAGCACTTTCCAGGCATTA |
| | GAACAGTCCATACTCATTACGCATACTGCTACAGATGTCCATTGAACTTAGAATA |
| | CCCTGGCTGTGGAGTTGAGTGTGCTAAGCAAATCGAAAATTTGATCTTTAGAAA |
| | GGAATTGAACCCAGAAGATACAGCTGCCGTCTTCGTTGAGCCAATTCAGGGTG |
| | AAGGTGGTTACATCGTCCCACCACCTGAGTTTCACAAGGAGTTAAGAAAGATCT |
| | GTACCGATAACGACGTCCTTTTGGCTGCAGACGAAGTGCAAGCCGGTTGTTTC |
| | AGAACGGGTCCTTTCTTAGCTATGGAGAATTTCGGAGTCAAACCAGATATCTCC |
| | TGTCTTGCAAAGGCGCTTGGATCAGGTTTACCAATGGGTGCTATGTTGGCTGAT |
| | GCTAGTTTAATGGATTGGCCCCCAGGTGTTCATTCCAACACTTTTGGTGGTAAC |
| | TTGCTCGCATCCGCAGGCTCATTGGCCGCCTTAGAATTTATGGAAAAGGAGAAA |
| | ACAGGTGCCCACGCTAGGTCGTTAGGCTCACATATTCAACAAAGATTAAGGGAG |
| | TTGCAAGATTCGTACCCATGTATTGGAGATGTCAGAGGCTTGGGTCTCATGATC |
| | GGTGCTGAAATCGTGAAACCAGACAAATCTATCGATCCAGCCACCAGGAATAGA |
| | ATTGTTAGGGAAGGTTACAAGCAAGGAATCATGTTGTTAGGTTGTGGAGAAAGT |
| | GTGATTAGATTTTCCCCACCATTAGTCATGACGGAAGAGGAAGCAGATAAGGGC |
| | TTGGAGCGTTTTGAAGAAGCCGTTAAACGTGGTGTTCGTCATCATCATCATCAT |
| | CATTAG (SEQ ID NO: 207) |
| | Protein |
| | MEDKRKGERKRERKEEHGMGTGDKMNPQMQEIGMLDTIGPRSLEVIEEDCNVMS |
| | ACETRPYPLVVDRAKGSVVTDLDGREYIDFVAGIAVLNAGHSNPAVQAAISAQLEK |
| | MTHCCSADFFAEPPLKLARKLRELSGYSKVFYCNSGTEAVEAAIKLATWKTGKQNL |
| | IGFYNSFHGRTLGSLSLTSSKARQKEHFPGIRTVHTHYAYCYRCPLNLEYPGCGVE |
| | CAKQIENLIFRKELNPEDTAAVFVEPIQGEGGYIVPPPEFHKELRKICTDNDVLLAAD |
| | EVQAGCFRTGPFLAMENFGVKPDISCLAKALGSGLPMGAMLADASLMDWPPGVH |
| | SNTFGGNLLASAGSLAALEFMEKEKTGAHARSLGSHIQQRLRELQDSYPCIGDVR |
| | GLGLMIGAEIVKPDKSIDPATRNRIVREGYKQGIMLLGCGESVIRFSPPLVMTEEEA |
| | DKGLERFEEAVKRGVRHHHHHH (SEQ ID NO: 233) |
| B7 from | ATGAGTAAAAGTGAAAACCTCTATAATGCAGCTAGAGAACTCATTCCTGGTGGA |
| Klebsiella | GTTAATTCCCCTGTTAGAGCCTTTACAGGTGTTGGCGGTACACCTTTGTTCATT |
| michiganensis | GAAAGGGCAGATGGAGCTTACTTGTACGATGTCGACGGAAAAGCATACATTGA |
| | CTACGTGGGTTCATGGGGTCCTATGGTCTTGGGCCATAACCATCCTGCCATCAG |
| | AAACGCTGTCATCGAAGCCGCAGAGAGAGGCCTTTCGTTTGGCGCGCCAACA |
| | GAAATGGAAGTTAAGATGGCAGCTTTAGTTACGGAGTTGGTTCCAACAATGGAT |
| | ATGGTTCGTATGGTCAACTCAGGTACAGAAGCTACCATGTCTGCAATCCGTTTG |
| | GCCCGTGGTTTCACTGGTAGAGACAAAATCATCAAGTTTGAAGGTTGTTACCAT |
| | GGTCACGCAGACTGCTTGTTGGTTAAGGCTGGTTCAGGTGCATTAACTTTGGG |
| | TCAACCCAATAGTCCAGGTGTTCCAGCGGATTTCGCTAAGCATACCCTTACTTG |
| | TACATATAATGACTTGTCTTCCGTCCGTGCAGCCTTTGAACAGTACCCACAAGAT |
| | ATCGCGTGTATTATTGTTGAGCCAGTCGCAGGCAATATGAATTGCATTCCCCCTT |
| | TGCCTGAATTCCTTCCAGGTCTTAGAGCTTTATGTGACGAGTTTGGAGCGCTTT |
| | TAATCATCGATGAAGTGATGACTGGTTTTAGAGTGGCCCTTGCAGGTGCTCAAG |
| | ATTACTACGGAGTGGTGCCAGATCTCACATGTTTGGGAAAGATCATTGGTGGTG |
| | GTATGCCAGTTGGCGCCTTTGGTGGAAGAAGAGAGGTTATGGATGCATTGGCA |
| | CCAACTGGCCCTGTTTATCAAGCAGGTACGTTGTCAGGTAACCCAATCGCGATG |
| | GCCGCTGGTTTTGCCTGCTTGACAGAAGTCGCCCAACCTGGCGTCCATGAAAC |
| | CCTCACAGAGTTGACCAATCAGTTGGCACAGGGTTTATTAGACGCAGCAAGAG |
| | ATGCAGGTATCCCATTGGTTGTTAACAATGTCGGTGGAATGTTCGGTATCTTCTT |
| | TACCGATGCTCCAACAGTGACCTGTTACCAAGATGTTGTCAAGTGTGATGTTGA |
| | AAGGTTTAAAAGATTCTTCCACTTAATGTTGCAAGAAGGTGTGTATTTAGCACCA |
| | TCCGCATTTGAAGCCGGTTTTATGAGTATTGCCCACTCGGAAGAAGACATCGAT |
| | AACACTGTGGCTGCCGCTAGAAACGCCTTTGCAAAATTACATCATCATCATCATC |
| | ATTAG (SEQ ID NO: 208) |
| | Protein |
| | MSKSENLYNAARELIPGGVNSPVRAFTGVGGTPLFIERADGAYLYDVDGKAYIDYV |
| | GSWGPMVLGHNHPAIRNAVIEAAERGLSFGAPTEMEVKMAALVTELVPTMDMVR |
| | MVNSGTEATMSAIRLARGFTGRDKIIKFEGCYHGHADCLLVKAGSGALTLGQPNSP |
| | GVPADFAKHTLTCTYNDLSSVRAAFEQYPQDIACIIVEPVAGNMNCIPPLPEFLPGL |
| | RALCDEFGALLIIDEVMTGFRVALAGAQDYYGVVPDLTCLGKIIGGGMPVGAFGGR |
| | REVMDALAPTGPVYQAGTLSGNPIAMAAGFACLTEVAQPGVHETLTELTNQLAQG |
| | LLDAARDAGIPLVVNNVGGMFGIFFTDAPTVTCYQDVVKCDVERFKRFFHLMLQE |
| | GVYLAPSAFEAGFMSIAHSEEDIDNTVAAARNAFAKLHHHHHH (SEQ ID NO: 234) |

TABLE 11
YDFG candidates

| YDFG candidates | Sequence (5′ to 3′) |
|---|---|
| Y1 from | ATGATTGTTTTAGTTACAGGTGCCACGGCAGGCTTCGGAGAATCTATCACAAGAA |
| Beauveria | GATTTGTCAAGAACGGTCATAAAGTTATTGCAACAGGTAGAAGGCAGGAAAGGT |
| bassiana D1-5 | TATTAGAATTAAAGGAGGAATTGGGTGATAACCTTTTCACGTTGCAGTTAGATGTC |
| | CGTAATCGTGCAGCCATCGAGGAAGCCTTAGCTTCTTTACCTGCTGAATGGCAA |
| | CACATTGATATTCTTGTTAATAATGCTGGATTGGCATTGGGAATGGAGCCTGCACA |
| | CAAGGCAAACGTGGATGACTGGGAAAACATGATCGACACTAATAACAAGGGTTT |
| | GGTCTACATGACCCGTGCGGTTTTGCCTGGCATGGTTGAGAGAAATAGAGGTCA |
| | TATTATCAATATTGGTTCAACTGCTGGTTCTTGGCCATACGCGGGTGGCAACGTG |
| | TACGGTGCGACCAAAGCATTTGTTAGACAATTCTCTTTGAACTTGAGAACCGACC |
| | TCCATAAGACAGCGGTCCGTGTTACCGATATTGAACCAGGTATGGTCGGTGGTA |
| | CTGAGTTTTCAAATGTTAGGTTTAAGGGTGATGACGACAAGGCAGGTAAAACTTA |
| | TGAAAATGCAAACGCATTGACCCCAGAAGATGTGACCGAAGCAGTGTGGTGGG |
| | TCGCTACTCTCCCAAAGCACGTCAATATCAACACTTTAGAAATGATGCCACAAAC |
| | CTCGGAAGACAAAAAGACGATGCCTTTGTCCCGTGAAATCAATCCAACACAACC |
| | AGTTAATCAGCAAATCTACCATATTTTAAGAAGAGAGATCGTTAGATGTCAAATTG |
| | CTCCAGGCACACCTTTGTCGGAGAAGGAAGTTTCGGGTAGATTTGATGTTTCCA |
| | GACAGCCTGTTAGAGAAGCGTTTATTAAACTTGCAGAGACAGGATTGGTCCAGA |
| | TCAGACCACAGAGGGGTACTTGGGTGTCGAAGATTTCAGTTTCTCACGTTAGAA |
| | ACGGATGTTTCATCAGAGAAGCTATCGAAAGAGCTGTCGTTAAGCGTGCGGCCG |
| | AGAAGGTCAATGATTCAGATCTTTACGCCTTAGAACATCTCTTAAACCAACAAAGA |
| | TTAGCAATTACTGACTCCCAAATCGATGATTTTTTCTCGCTCGATGATAAGTTCCA |
| | TAGGCAATTAGCCTTAATTGCCGACTGCTCCTTGGCTTGGGACACCGTTGAAAAT |
| | ATCAAAGCAACAATGGATAGAGTCAGATATATGTCATTGGATCATGTTTCCCCTCC |
| | TGCTATGTTGTTGGAACAACATTATGCTATTTTCAACGCCCTCAAGGCGCATGAT |
| | GTCGCTGAAGTCGATACTGCCATGCACCTTCACTTGCACGAGATTTCTCAGTCTA |
| | TTTTGTTAATCAGACAAGAAAACCAAGACTGGTTTTCCGATGAGTAA (SEQ ID |
| | NO: 1) |
| | Protein |
| | MIVLVTGATAGFGESITRRFVKNGHKVIATGRRQERLLELKEELGDNLFTLQLDVRN |
| | RAAIEEALASLPAEWQHIDILVNNAGLALGMEPAHKANVDDWENMIDTNNKGLVYM |
| | TRAVLPGMVERNRGHIINIGSTAGSWPYAGGNVYGATKAFVRQFSLNLRTDLHKTAV |
| | RVTDIEPGMVGGTEFSNVRFKGDDDKAGKTYENANALTPEDVTEAVWWWVATLPKH |
| | VNINTLEMMPQTSEDKKTMPLSREINPTQPVNQQIYHILRREIVRCQIAPGTPLSEKE |
| | VSGRFDVSRQPVREAFIKLAETGLVQIRPQRGTWVSKISVSHVRNGCFIREAIERAV |
| | VKRAAEKVNDSDLYALEHLLNQQRLAITDSQIDDFFSLDDKFHRQLALIADCSLAWD |
| | TVENIKATMDRVRYMSLDHVSPPAMLLEQHYAIFNALKAHDVAEVDTAMHLHLHEIS |
| | QSILLIRQENQDWFSDE (SEQ ID NO: 218) |
| Y2 from | ATGCCAGGCTCATTATCTTTCGCAACACTTTCGAAACAGCAAGCTGAATCAGGTT |
| Plautia stali | ACGAGTTAAACACATCTCATCGTCAACCAGACTCCACTAGACAAGCAAGAAACT |
| | CTGAAATGATCATCTTTGTTACTGGTGCTACCGCTGGTTTTGGTGAATCGATTAC |
| | GAGAAGGTTCATTGCTAACGGACACAAGGTCATCGCATCTGGTAGACGTGCTGA |
| | AAGATTAAAAGCCTTGAAGGAGGAATTGGGTGCAAACCTTTATACAGTCCAGTTG |
| | GATGTTAGAAACAGAGCAGCCATTGACGAAGCTATCGCCGCGTTGCCAGCTGAA |
| | TGGCGTGAGATCGATGTTCTCGTGAATAATGCAGGTTTAGCCTTGGGTATTGAAC |
| | CAGCTCATAAGGCCAACATTGAGGATTGGGAAAACATGATTGACACCAATAATAA |
| | AGGCTTAGTCTATATGACTAGAGCGGTTCTCCCTGCAATGGTTGAAAGGAATGTT |
| | GGTCATATTGTTAATATTGGATCCATCGCAGGTTCATGGCCATATCAAGGAGGTAA |
| | TGTTTACGGAGCCACCAAGGCATTTGTGAGACAATTTTCGTTGAACTTGAGAACA |
| | GATCTTCATGGTACTGCATTGAGAGTCACAGATATTGAGCCTGGTTTAGTTGGTG |
| | GTACCGAATTCTCCAACGTCAGGTTCAAGGGCGACGATGGAAGAGCGGATGCA |
| | GTTTACGAGGGCACCACTGCCTTGACTGCCGAGGATGTCACAGAAGCTGTCTA |
| | CTGGGTTACTACGTTACCTAAGCACGTTAATATCAATACTCTCGAAATTATGCCTG |
| | TGTCCCAGACCTTGGCGGGTTTAAAGGTCCACAAGGGTTAA (SEQ ID NO: 2) |
| | PROTEIN |
| | MPGSLSFATLSKQQAESGYELNTSHRQPDSTRQARNSEMIIFVTGATAGFGESITRR |
| | FIANGHKVIASGRRAERLKALKEELGANLYTVQLDVRNRAAIDEAIAALPAEWREIDV |
| | LVNNAGLALGIEPAHKANIEDWENMIDTNNKGLVYMTRAVLPAMVERNVGHIVNIGSI |
| | AGSWPYQGGNVYGATKAFVRQFSLNLRTDLHGTALRVTDIEPGLVGGTEFSNVRFK |
| | GDDGRADAVYEGTTALTAEDVTEAVYWVTTLPKHVNINTLEIMPVSQTLAGLKVHK |
| | (SEQ ID NO: 219) |
| Y3 | ATGATTGTCTTTGTTACAGGCGCCACAGCAGGTTTTGGTGAATCCATCACTAGAA |
| Erwinia | GGTTTATTGCTCAAGGACATAAGGTTGTTGCAACAGGTAGACGTTCGGAGAGGC |
| mallotivora | TTCAAGCCTTGAAGGATGAACTCGGTGAAAACGTTTACACTCTCCAGTTAGACG |
| | TCAGAAACAGAGCGGGTATTGATGCAGCTTTACATTCGTTGCCAGCGGAGTGGA |
| | GAGACATTGATGTTCTTGTCAATAATGCTGGTTTGGCCTTGGGTATGGCCCCAGC |
| | CCACCAAGCTTCAATTGACGATTGGGAAGCAATGATCGATACCAATAATAAAGGT |
| | TTAGTCTTTATGACAAGAGCAATCTTACCTGCAATGGTGGAAAGGAACGTCGGTC |
| | ATATCATCAATATTGGCTCTACTGCCGGAAACTGGCCTTATGCGGGTGGCAACGT |
| | GTATGGTGCCACCAAAGCTTTCGTTAGACAATTCTCCTTAAACTTGCGTACTGAT |
| | CTTCACGGAACTGCATTGAGAGTCACCGATATTGAACCTGGATTAGTTGGTGGC |
| | ACTGAATTTTCAAATGTTAGATTCAAGGGTGACTCTGATAAGGCGGATTCCGTCT |
| | ACGACGGTACGGAACCATTGACCGCAGAGGATGTCACGGAAGCTGTTTTCTGG |
| | GTTGCTTCTCTCCCAAAACACGTGAACATCAATACCTTGGAGATGATGCCAGTTG |
| | GTCAGACATTGGCTGGTTTGAAGGTTCATAAGGCATAA (SEQ ID NO: 3) |
| | Protein |
| | MIVFVTGATAGFGESITRRFIAQGHKVVATGRRSERLQALKDELGENVYTLQLDVRN |
| | RAGIDAALHSLPAEWRDIDVLVNNAGLALGMAPAHQASIDDWEAMIDTNNKGLVFM |
| | TRAILPAMVERNVGHIINIGSTAGNWPYAGGNVYGATKAFVRQFSLNLRTDLHGTAL |
| | RVTDIEPGLVGGTEFSNVRFKGDSDKADSVYDGTEPLTAEDVTEAVFWVASLPKHV |
| | NINTLEMMPVGQTLAGLKVHKA (SEQ ID NO: 220) |
| Y4 | ATGGCTCATATTCTTGTCTCAGGTGCTACCTCGGGATTCGGATTAGAGATCGCCA |
| Helicobacter | AGGCATTCCTCCAAAAGAATCACGTCGTCTTTGGTACAGGTAGAAGAAAGGAGA |
| pylori | ACCTTCAAGAATTGCAGCTCGCGTATCCAAAAAGATTCATTCCTTTGTGTTTTGA |
| | CCTCCAAAACAAGCTCGAAACGAAAAGAGCTATCGAAACAATCTTTTCTATGACA |
| | GACAGAATTGATGCCTTAATCAATAATGCTGGTTTGGCATTGGGTTTGAACAAGG |
| | CGTATGAATGTGAATTGGACGATTGGGAAGTTATGATTGATACTAACATCAAGGGT |
| | TTATTGTACCTTACGAGGTTAATTTTGCCATCCATGATCGAACATGATCAAGGCAC |
| | TATTATTAATCTTGGTTCTATTGCCGGTACCTACGCATATCCAGGTGGCAACGTTT |
| | ACGGCGCTTCCAAGGCGTTTGTTAAGCAATTCTCTTTGAACTTACGTGCAGATTT |
| | GGCCGGTACTAACATCAGAGTGTCCAATGTTGAGCCTGGTTTATGCGGAGAGAC |
| | TGAATTCTCAATGGTTAGGTTTAAGGGTGATAAAATCAAAGCACAGTCGGTCTAT |
| | GAGAATACATTATACTTGAAACCACAGGACATCGCAAACATTGTTTTGTGGATCTA |
| | CGAACAACCTTCGCACGTGAATATTAATCGTATTGAAATTATGCCTACCTCACAAA |
| | CCTTTGCACCATTACCTACTCATAAGAATCCATAA (SEQ ID NO: 4) |
| | PROTEIN |
| | MAHILVSGATSGFGLEIAKAFLQKNHVVFGTGRRKENLQELQLAYPKRFIPLCFDLQ |
| | NKLETKRAIETIFSMTDRIDALINNAGLALGLNKAYECELDDWEVMIDTNIKGLLYLTR |
| | LILPSMIEHDQGTIINLGSIAGTYAYPGGNVYGASKAFVKQFSLNLRADLAGTNIRVS |
| | NVEPGLCGETEFSMVRFKGDKIKAQSVYENTLYLKPQDIANIVLWIYEQPSHVNINRI |
| | EIMPTSQTFAPLPTHKNP (SEQ ID NO: 221) |
| Y5 from | ATGACTCCAACATTATTCATCACCGGTGCAACATCTGGTTTTGGTGAGGCATGTG |
| Pseudomonas | CGAGAAGATTTGCTAGAGATGGTTGGTCCTTGGTTATTACCGGTAGAAGAGAAG |
| citronellolis | ACAGGTTGCAAGCATTGGCCAAGGAGTTGTCGTCTCAAGCCGAAGTTTTACCTA |
| | TCACATTGGATGTTAGAGATAGAGCGGCAGTCGCACACGCAGTGGATAATCTTC |
| | CTGCGGGTTTTGACAAGCTTAGAGCTCTCATTAATAATGCAGGTTTGGCTTTAGG |
| | TGCTGATCCAGCCCAAGAATGTGATCTCGATGATTGGGACACTATGGTCGATACG |
| | AACATTAAGGGTTTGATGTACTGCACTAGGTTGTTGTTACCACGTCTCATCGCTC |
| | ATGGAAGGGGAGCCTCGATCTTGAACTTAGGATCTGTCGCGGGTCAATGGCCTT |
| | ACCCTGGATCGCATGTTTATGGTGCCACTAAAGCTTTCGTTGAACAGTTTTCCTT |
| | ATCATTGCGTTGTGACCTCCAGGGCACCGGTGTTAGAGTTTCTAATCTTGAACCA |
| | GGTTTGTGTGAGTCAGAATTTTCCCTTGTCAGATTCGGCGGTGACCAAGCTCGT |
| | TATGACAAGACTTACGCAGGCACAGAAGCGTTACAACCTGAAGATATTGCTGAAA |
| | CCATTCATTGGATTGTCAATCAGCCACCACATATCAACATTAACTCCTTAGAGATC |
| | ATGCCAGTGTCACAAGCATGGGCCGGTTTCTCTGTTCACAGAAAAGGCTAA |
| | (SEQ ID NO: 5) |
| | Protein |
| | MTPTLFITGATSGFGEACARRFARDGWSLVITGRREDRLQALAKELSSQAEVLPITL |
| | DVRDRAAVAHAVDNLPAGFDKLRALINNAGLALGADPAQECDLDDWDTMVDTNIKG |
| | LMYCTRLLLPRLIAHGRGASILNLGSVAGQWPYPGSHVYGATKAFVEQFSLSLRCD |
| | LQGTGVRVSNLEPGLCESEFSLVRFGGDQARYDKTYAGTEALQPEDIAETIHWIVN |
| | QPPHININSLEIMPVSQAWAGFSVHRKG (SEQ ID NO: 222) |
| Y6 from | ATGACCGATACTGTTTTGGTTACAGGTGCAACTGCTGGTTTCGGTAGAGCAATCT |
| Acetobacter | CCGAACGTCTTGTTAAGGACGGTTATAGAGTCATCGGTACGGGTCGTAGGGCTG |
| sicerae | AAAGATTGGACGCGTTACAGGCATCTTTGGGTGACGCTTTTCTCCCATTGGCCT |
| | TGGATATGTTGGATAAAGAAGCCTTAAGAGGTTTACCAACCGAATTACCAGAGGG |
| | TTGGAGAGAGGTTGATGTTTTGATTAACAATGCGGGCTTAGCTTTGGGCATGGAT |
| | CCAGCTCAAACTACAAACATCGATGACTGGGAACGTATGATCGGAACTAATGTGT |
| | CGGGTATGGTGGAGATTACGAGGGCGCTCTTACCTGGTATGGTTGAACGTAATC |
| | ATGGACACGTTGTCTCGCTTGGTTCCACAGCTGGCACCTACCCTTACAAGGGTG |
| | GCAACGTTTATGGAGCCTCAAAGGCGTTTGTTTCCCAATTCATGAGAAATCTTAG |
| | AACAGACCTTTTGGGTACCAGAGTTAGAGCAACTAACCTCGAACCAGGATTGTG |
| | TGGTGGTTCAGAATTTTCTAACGTGAGACTCGGAGATGAGGCAAAAGCCGCAGA |
| | TGTCTACAGAGGTACTAAGCCTATTTTGCCTGAAGATATTGCAGAGACAGTCGCC |
| | TGGGTCTTGGGTTTACCAGAACATGTCAATATTAATAGAATTGAAATGATGCCTGT |
| | TTGTCAAGCAGCTGGCGGTTTGTCTGTCGTTAGGGATGTCGACTAA (SEQ ID |
| | NO: 6) |
| | PROTEIN |
| | MTDTVLVTGATAGFGRAISERLVKDGYRVIGTGRRAERLDALQASLGDAFLPLALDM |
| | LDKEALRGLPTELPEGWREVDVLINNAGLALGMDPAQTTNIDDWERMIGTNVSGM |
| | VEITRALLPGMVERNHGHVVSLGSTAGTYPYKGGNVYGASKAFVSQFMRNLRTDL |
| | LGTRVRATNLEPGLCGGSEFSNVRLGDEAKAADVYRGTKPILPEDIAETVAWVLGLP |
| | EHVNINRIEMMPVCQAAGGLSVVRDVD (SEQ ID NO: 223) |
| Y7 from | ATGCCTTTAACCGTCTTGGTCACAGGTGCTACCGCTGGTTTTGGCCACGCCATT |
| Gluconobacter | GCCACGAGATTAGTTAGAGAAGGTCATAGGGTTATTGCCACAGGTCGTAGAAAG |
| oxydans NBRC | GAGAGATTGGAAGCTTTAAAGGACGAGTTAGGTGAATCAGTGCTCCCATACACC |
| 3293 | CTTGATATGACTGATAGAGAAGCTTTGCGTGCATTACCAGCTGCGCTCCCTGAAG |
| | AATGGCAGGATATTGATGTGCTCGTCAATAACGCAGGTTTGGCGTTGGGTATGG |
| | AACCAGCGCAAGATGCAGAACCTGATAATTGGGAGTCTATGATCGCTACTAACGT |
| | TTCCGGTGTTGTCGAGTTGACTAGAGCCTTGTTACCTGGAATGGTTCAGCGTAAT |
| | ACTGGTTACATCATCGCATTGGGTTCGACTGCCGGTACGTATCCTTATACTGGCG |
| | GAAACGTTTACGGTGCAACTAAAGCTTTTGTCCACCAATTCACACAAAACCTTAG |
| | AACAGACCTTTTGGGTACCAGAATTAGAGTTACCAACATCGAACCAGGTTTGTGT |
| | GGCGGATCTGAGTTCTCAAATGTTAGGTTAGGTGATGACGCAAAAGCTGCAGCG |
| | GTGTATAAGGACACAACTCCATTGACACCAGAAGATATTGCAGGAACCGTTTCGT |
| | GGCTCATTGGTTTACCATGGCATGTCAATGTCAACTACCTTGAAATGATGCCTGT |
| | CTGTCAAGCATCCGGAGGTTTGGCCGTTAATAGACATGTTGGCTAA (SEQ ID |
| | NO: 7) |
| | PROTEIN |
| | MPLTVLVTGATAGFGHAIATRLVREGHRVIATGRRKERLEALKDELGESVLPYTLDMT |
| | DREALRALPAALPEEWQDIDVLVNNAGLALGMEPAQDAEPDNWESMIATNVSGVV |
| | ELTRALLPGMVQRNTGYIIALGSTAGTYPYTGGNVYGATKAFVHQFTQNLRTDLLGT |
| | RIRVTNIEPGLCGGSEFSNVRLGDDAKAAAVYKDTTPLTPEDIAGTVSWLIGLPWHV |
| | NVNYLEMMPVCQASGGLAVNRHVG (SEQ ID NO: 224) |
| Y8 from | ATGACCAAAACAGCCATGGTTACCGGTGCTACTTCGGGCTTTGGTGCAGCCATT |
| Oleiagrimonas | GCAGAGAGATTGGTTAAGGACGGCTGGAGAGTTGTGATCTGTGGTAGACGTGA |
| soli | AGAACGTTTACAGACCATGGTGGATAGGTTAGGTGCAGACAAGGCTCACGCAGC |
| | AGCTTTCGACATTAGAGATGCAGAGGCGATGTCTGAAGCGTTGGATGCCTTGCC |
| | AGCAGCGTTTGCGGATATCGATTTGTTAGTTAATAACGCTGGTTTAGCCTTGGGT |
| | ACCGAACCAGCACAATTCGCCGACCTTGATGAGTGGAGGCAGATGATCGATACT |
| | AACGTCACTGCTTTGGTCACACTCACTCATGCCTTGTTGCCTGCCTTAATTGAGA |
| | GACGTGGCGCTATCGTTAATATCTCCTCGATTTCAGGTTCTTATCCTTACAAAGGC |
| | GGAAACGTTTATGGTGGTACGAAGGCATTTGTTACTCAATTTTCGCTTGGATTAA |
| | GAACAGATCTTCACGGTACAGGTGTCAGAGTCACTTCCGTGGAACCTGGTATGG |
| | CAGAGACTGAATTCACATTAGTTAGAACCGGAGGTGACCAAGCTGCTTCTGATAA |
| | GCTCTACACAGGTGCCCATCCTATTACGGCGGAAGACATTGCAGAAACCGTTGC |
| | TTGGATTGCTCATCTCCCACCACATTTGAATGTCAATAGAATCGAAGTTATGCCAA |
| | CTTCCCAATCAGCGGCAGGATTGCAAGTCCACAGAGATTAA (SEQ ID NO: 8) |
| | PROTEIN |
| | MTKTAMVTGATSGFGAAIAERLVKDGWRVVICGRREERLQTMVDRLGADKAHAAA |
| | FDIRDAEAMSEALDALPAAFADIDLLVNNAGLALGTEPAQFADLDEWRQMIDTNVTA |
| | LVTLTHALLPALIERRGAIVNISSISGSYPYKGGNVYGGTKAFVTQFSLGLRTDLHGT |
| | GVRVTSVEPGMAETEFTLVRTGGDQAASDKLYTGAHPITAEDIAETVAWIAHLPPHL |
| | NVNRIEVMPTSQSAAGLQVHRD (SEQ ID NO: 225) |
| Y9 from | ATGTCCAGAACCGTTTTTATTACAGGTGCATCCGCAGGATTTGGCGCGGCATGT |
| Geobacter soli | GCAAGATTGTTCGCCGACAGAGGAGATAGATTGGTTTTAGCAGCCCGTAGGGAA |
| | GAGCCATTGTTGGCTTTAAGGGATGAATTGGCTCCACAAGCAGAAGTCCACACT |
| | GTCACCCTCGATGTCAGAAGAAGAAAGGCAGTTCAAGAAGCCGTTGAGGGTTTA |
| | CCAGACGCCTTTTCCCGTATTGACATTTTGGTCAACAATGCAGGCCTCGCTTTAG |
| | GTTTGGAACCAGCTCATGAAACCAACCCTGATGATTGGGATGCGATGGTCGATA |
| | CGAACATCAAGGGTTTGATCTACTGCACTAGGTTTTTATTGCCAGGTATGGTTGC |
| | GAGAAACAGAGGTCATATTGTGAATATTGGTTCTGTGGCGGGATCATGGCCATAT |
| | CCTGGTGGTAACGTTTATGGCGGTACTAAGGCCTTCGTCGAACAATTCTCGCGT |
| | AATCTTAGAGCCGACCTTTTGGGCAAAAGAGTCCGTGTTACATGTTTAGCACCTG |
| | GTATGGCAGAAACCGAGTTCTCAATGGTTAGGTTTAAAGGAGATGCAGAAAGAG |
| | CTGCTCAAGTGTACGCCGGTACACAGGCCTTAACAGCAGAGGACATCGCGGGT |
| | ATTGTCGCTTGGGTTACTGAATTACCACCTCACGTTAATGTTAATGCTCTTGAGGT |
| | TATGTCTATCTTCCAGTCTTGGGGTCCTCTCGCTGTGCATAGACCTGGTGCTTCG |
| | TAA (SEQ ID NO: 9) |
| | PROTEIN |
| | MSRTVFITGASAGFGAACARLFADRGDRLVLAARREEPLLALRDELAPQAEVHTVTL |
| | DVRRRKAVQEAVEGLPDAFSRIDILVNNAGLALGLEPAHETNPDDWDAMVDTNIKG |
| | LIYCTRFLLPGMVARNRGHIVNIGSVAGSWPYPGGNVYGGTKAFVEQFSRNLRADL |
| | LGKRVRVTCLAPGMAETEFSMVRFKGDAERAAQVYAGTQALTAEDIAGIVAWVTEL |
| | PPHVNVNALEVMSIFQSWGPLAVHRPGAS (SEQ ID NO: 226) |
| Y10 from | ATGAAGAAGACCGCATTTGTCACTGGCGCAACCTCCGGTTTTGGTGAGGCCATT |
| Campylobacter | GCAAGGAGATTATCGAAAGAAGGTTACAAGATTGTTGCTTTGGCAAGACGTGAG |
| concisus | GATAGATTGAAAAAGCTCGCATCAGAATTAGGTGATACGCATATTATCGTTGCGGA |
| | TATTAGAGATAAAGAAGCAGTTTTCGACGCCGTGGATTCATTGCCTGAAAACTTC |
| | AAGGACATTGAAGTTTTGGTGAATAACGCCGGCATGGCCTTGGGTTTAGAAAAG |
| | ACAATCGATGCTAAGGTTGAAGACTTCGAAGCTATGATCGATACAAACGTCAAGG |
| | GATTGATCTACTCTACAAAGGCCGTCTTGCCTTTGTTATACAAGCAGGAAAAAGG |
| | ATATATTTTTAATATCGGTTCTACTGCGGGTTCTTGGCCATACCCAGGTTCAAACG |
| | TCTATGGTGCGACTAAGGCTTTTGTCAAACAATTTTCCCTCAACCTCAGGAATGA |
| | CCTTGTTGGCACAAATATTAGAGTGACCAATATCGAACCAGGTCTTTGTAAGACC |
| | GAGTTCTCGGAGGTTAGATTTCGTGGTGACAAGGCAAAGGCAGATTCCCTTTAT |
| | GAGAACACTAACTTCATTACTTCGGAGGATATCGCGACAATTTTAGTTAATTGTTT |
| | AAACATGCCAGGATCTGTTAATATCAATAGAGTTGAAGTCATGGCTAATACTCAAA |
| | CGTGGGCTGGTTTGGCTATTGAAAAATTCTAA (SEQ ID NO: 10) |
| | PROTEIN |
| | MKKTAFVTGATSGFGEAIARRLSKEGYKIVALARREDRLKKLASELGDTHIIVADIRDK |
| | EAVFDAVDSLPENFKDIEVLVNNAGMALGLEKTIDAKVEDFEAMIDTNVKGLIYSTKA |
| | VLPLLYKQEKGYIFNIGSTAGSWPYPGSNVYGATKAFVKQFSLNLRNDLVGTNIRVT |
| | NIEPGLCKTEFSEVRFRGDKAKADSLYENTNFITSEDIATILVNCLNMPGSVNINRVE |
| | VMANTQTWAGLAIEKF (SEQ ID NO:: 227) |

TABLE 12
Seed culture inoculation and growth.

| | Primary | Primary Seed |
|---|---|---|
| | Flask: 250 mL | for 300 L |
| Flask | OD 600 | OD 600 |
| 1 | 5.16 | 2.71 |
| 2 | 6.02 | 2.7 |
| 3 | 6.02 | 2.97 |
| 4 | 7.08 | 2.58 |
| 5 | 4.18 | 2.68 |
| 6 | 4.58 | 3.4 |
| 7 | 5.04 | 2.64 |
| 8 | 7.56 | 2.69 |

TABLE 13
Start-up conditions, and parameters applied
during 10 L and 300 L fermentation.

| Parameters | 10 L fermenter | 300 L fermenter |
|---|---|---|
| Temperature (C.) | 30 | 30 |
| pH | 4 | 4 |
| Agitation (RPM) | cascaded | cascaded |
| Aeration (vvm) | 0.4 vvm | 0.4 vvm |
| DO set point | 10% in metabolite | 10% in metabolite |
| (% saturation) | accumulation phase | accumulation phase |
| Inoculation Volume | 5 | 5 |
| (% v/v) | | |

TABLE 14
Details of the reactor at 100 mL, 10 L, and 300 L scale.

| | Lab Scale | Pilot Scale | |
|---|---|---|---|
| | Manufacturer | | |
| | | | New Brunswick |
| | Eppendorf | B-DCU | Scientific, Edison, |
| Parameter | DasBox | Sartorius | NJ, USA |
| Nominal Volume (L) | 0.3 | 10 L | 300 L |
| Maximum Working Volume | 0.25 | 10 | 240 |
| According to the Manufacture (L) | | | |
| Actual Working Volume (L) | 0.1 | 4 | 100 |
| Tank Diameter (D_{T}, mm) | 90 | 190 | 1420 |
| Tank Height (H_{T}, mm) | 360 | 470 | 3120 |
| Impeller Type | 6 blade | 6 blade | 6 blade |
| | Rushton | Rushton | Rushton |
| Impeller Diameter (Dₗ, mm) | 30 | 75 | 554 |
| Number of impellers | 2 | 2 | 3 |
| H_{T}/D_{T} | 4 | 2.5 | 2.2 |
| Dₗ/D_{T} | 0.33 | 0.39 | 0.39 |
| Aeration Rate (vvm) | 2.4 sLPH | Max. 20 sLPM | |
| Stirrer Speed (RPM) | 400-1200 | 275-900 | 45-450 |
| | (Cascaded | (Cascaded | (Cascaded |
| | on DO) | on DO) | on DO) |

TABLE 15
Breakdown of the 300-L dextrose scenario baseline biomanufacturing
facility's total capital cost. ISBL and OSBL denote
inside and outside battery limits, respectively.

| | | |
|---|---|---|
| Cost category | Notes | Cost [MM$] |

Direct costs

| | | |
|---|---|---|
| ISBL installed equipment | — | 166.13 |
| cost | | |
| OSBL installed equipment | — | 70.83 |
| cost | | |
| Warehouse | 4.0% of ISBL | 6.64 |
| Site development | 9.0% of ISBL | 14.95 |
| Additional piping | 4.5% of ISBL | 7.48 |
| Total direct cost (TDC) | — | 266.03 |

Indirect costs

| | | |
|---|---|---|
| Pro-ratable costs | 10.0% of TDC | 26.603 |
| Field expenses | 10.0% of TDC | 26.603 |
| Construction | 20.0% of TDC | 53.21 |
| Contingency | 10.0% of TDC | 26.60 |
| Other indirect costs (start-up, | 10.0% of TDC | 26.60 |
| permits, etc.) | | |
| Total indirect cost (TIDC) | — | 159.62 |
| Fixed capital investment | TDC + TIDC | 425.65 |
| (FCI) | | |
| Working capital (WC) | 5.0% of FCI | 21.28 |
| Total capital investment | FCI + WC | 446.93 |
| (TCI) | | |

TABLE 16
Breakdown of the 300-L dextrose scenario baseline biomanufacturing
facility's fixed operating cost. ISBL and OSBL denote
inside and outside battery limits, respectively.

| Cost category | Notes | Cost [MM$ · y⁻¹] |
|---|---|---|
| Labor salary | — | 7.01 |
| Labor burden | 90% of labor salary | 6.31 |
| Maintenance | 3.0% of ISBL | 4.98 |
| Property insurance | 0.7% of ISBL | 1.16 |
| Total fixed operating cost (FOC) | — | 19.46 |

TABLE 17
Increment in 3HP titer and yield observed
between every batch/fed-batch mode

| | 10 L | | 300 L | |
|---|---|---|---|---|
| | Increment in | Yield | Increment in | Yield |
| Fermentation | 3-HP titer | (g/g | 3HP titer | (g/g |
| span | (g/L) | glucose) | (g/L) | glucose) |
| Batch | 22.39 | 0.455 | 20.40 | 0.520 |
| Fed-batch 1 | 12.08 | 0.416 | 26.45 | 0.892 |
| Fed-batch 2 | 11.28 | 0.443 | 12.40 | 0.422 |
| Fed-batch 3 | 12.16 | 0.518 | 16.90 | 0.454 |
| Fed-batch 4 | 36.20 | 0.812 | 24.70 | 0.479 |

| | | |
|---|---|---|
| Overall Yield | 0.744 | 0.750 |

### Example 23 Isolation of 3-HP

A simplified downstream processing (DSP) strategy to recover 3-hydroxypropionic acid (3-HP) in both solid (salt) form and as a 30% (w/v) aqueous solution is provided. Following cell removal by centrifugation, activated charcoal was used to remove color impurities. Subsequently, cation- and anion-exchange resins were employed to capture positively and negatively charged ions, respectively. The solid Na(3-HP) salt offers advantages in storage and transport, while the 3-HP solution is well-suited for direct conversion to acrylic acid in an integrated production system.

The purification process utilized two types of activated charcoal powders (Darco® Activated G-60, Sigma-Aldrich, USA and Darco® 100 mesh, Sigma-Aldrich, USA) for preliminary decolorization, followed by ion exchange employing a strong acid cation exchange resin and a weak base anion exchange resin. Specifically, a strong acid cation exchanger, such as Dowex® G26 H₂ form (Sigma-Aldrich, USA), was utilized, which is typically based on a sulfonic acid functional group (R—SO3-) on a styrene-divinylbenzene (DVB) matrix for the efficient removal of residual metal ions (e.g., Na+, NH4+). For the highly selective separation of the 3-HP anion, the weak base anion exchanger Amberlite™ IRA-67 (Sigma-Aldrich, USA) was chosen, which possesses tertiary amine functional groups on an acrylic matrix; this resin is recognized for its effectiveness under acidic conditions and its resistance to fouling by organic matter, making it ideal for purifying complex organic acid fermentation broths.

In general, the binding preference for the free base form of this weak base resin is driven by acid strength. In acidic conditions, the binding preference of Amberlite™ IRA-67 (Sigma-Aldrich, USA), a weak base anion exchange resin, is primarily determined by the acid strength of the anions. The resin, which has acrylic tertiary amine functional groups, is protonated at low pH and acts as an ion exchanger. It binds stronger to anions of stronger acids than to anions of weaker acids. Based on the relative acid strengths, the binding preference for the given anions is: sulfate>chloride>carboxylic acid anions.

The fermentation broth containing 3-HP was collected from a previously operated 300 L fermenter run in fed-batch mode. Briefly, the process began in batch mode with a high-performance 3-HP-producing strain in a corn steep liquor (CSL)-based medium with 55 g/L glucose. The initial batch phase was maintained for 44.0 h with an aeration rate of 0.4 vvm. Dissolved oxygen (DO) was consistently controlled at 10% of saturation by introducing carbon dioxide (CO₂) and cascading aeration and agitation. The fed-batch operation was initiated after a DO spike signaled glucose limitation, at which point a pulse feeding profile was implemented. Throughout the process, the reactor was pressurized to 10 psig, and a one-way pH control strategy maintained the pH at 4.0 using 28-30 wt % ammonium hydroxide (NH₄OH). After 230 h of operation, the collected broth was centrifuged at 10,000 rpm to separate the cell mass and the supernatant was characterized for 3-HP, glucose and major impurities, such as ammonium and sulfate. No metabolite other than 3-HP and unused glucose was present in the fermentation broth. The primary characteristics of the fermentation broth are listed in Table 21. The broth was centrifuged at 10,000 rpm for 15 min and the supernatant was used in the present study.

TABLE 21
Specific information of components in
fermentation broth used in this study.

| Components | Unit | Concentration | Reference |
|---|---|---|---|
| Glucose | g/L | 4 ± 1.2 | Experimental value |
| 3-HP | g/L | 62.3 ± 5 | Experimental value |
| Biomass | g/L | 15 ± 1.5 | Experimental value |
| Ammonium | mg/L | 3700 ± 650 | Experimental value |
| Sulfate | mg/L | 2800 ± 430 | Experimental value |
| Color | mg/L | 50 | (Alexandri et al., Sep. Purif. |
| impurities | | | Technol., 209: 666 (2019)) |
| Other | Mass % | 0.4 | Assumption |
| impurities | | | |

Before developing the complete DSP flowsheet, a series of preliminary adsorption studies were conducted to optimize key purification steps for this study. For color removal, two different types of activated charcoal powders (charcoal 1: Darco® Activated G-60, charcoal 2: Darco® 100 mesh) were examined, with their color efficiency measured by the optical density (OD) at 240 nm. The experiments for both charcoals were conducted with a 3% w/v activated charcoal at 30° C. and 200 rpm for 2 h (Law et al, J Memb Sci, 583 (2019), pp. 139-151. The charcoal with the highest color removal efficiency was selected for the subsequent experiments. The color removal efficiency was estimated using Eq. (1) from Law et al., 2019.

Furthermore, preliminary studies were performed on Amberlite IRA-67, a weak base anion exchange resin, to minimize 3-HP loss while selectively removing anionic impurities. The binding preference of the resin at low pH is primarily determined by the acid strength of the anions. As a weak base resin with acrylic tertiary amine functional groups, it is protonated at low pH and preferentially binds to anions of stronger acids, such as sulfate and chloride, more strongly than to the carboxylic acid anion of 3-HP. To optimize this selective removal, various pH and temperature (below 35° C. as recommended by manufacturer) conditions were tested for 30 min batch. Subsequently, the adsorption kinetics of anion exchange were investigated to determine the optimal batch time, ensuring minimal 3-HP loss and maximizing the removal of impurities. All preliminary studies were conducted in 250 mL Erlenmeyer flasks with a working volume of 100 mL under continuous shaking at 200 rpm. In the present study, the resin dosage (10% w/v) was selected based on preliminary saturation and adsorption kinetics experiments, supported by literature, indicating sufficient active sites for effective impurity removal while minimizing resin overuse and non-specific adsorption of 3-HP. Adsorption times for activated charcoal and cation exchange were adopted from literature, while the adsorption time for the anion exchange resin was further refined through kinetic studies, which showed that impurity uptake reaches near-equilibrium within the first 5-10 min, extended contact primarily increases 3-HP loss without improving selectivity.

The fermentation broth containing 3-HP (62.3 g/L), collected from a 300 L fermenter, was centrifuged at 10,000 rpm for 15 min to separate the cell mass from the liquid. In the fermentation broth, 3-HP was present predominantly in its acid form. Cell-free 1 L broth was then decolorized with 3% w/v activated charcoal (Darco® Activated G-60, Sigma-Aldrich, USA) at 30° C. and 200 rpm for 2 h. Impurities in the form of cations (ammonium and metal ions) and anions (sulfate, phosphate, and nitrate) were removed using cation and anion exchange resins, respectively. Before application, the resins were conditioned with deionized water overnight, washed until the effluent pH reached neutral, and then dried at 30-35° C. A strong cation exchanger (Dowex® G26 H₂ form) was used to remove cations at 10% w/v dose, pH 4.5, 30° C., and 200 rpm for 1 h. After separating the resins by vacuum filtration, the pH of the permeate was adjusted to 4.5 using 10 M NaOH solution. A weak anion exchanger (Amberlite® IRA-67 free base) was employed to remove anions at 10% w/v dose, pH 4.5, 30° C., and 200 rpm for 10 min. Next, the 3-HP-rich liquid obtained after anion exchange chromatography, in which 3-HP remained in the acid form, was neutralized using 10 M NaOH. Total approx. 111 mL of 10 M NaOH was required to neutralize 100 g of 3-HP based on the stoichiometry. Neutralization was calculated assuming stoichiometric conversion of 3-HP to sodium 3-hydroxypropionate, requiring one mole of NaOH per mole of 3-HP, thereby ensuring complete salt formation while avoiding excess base addition and the introduction of additional inorganic impurities. Following neutralization, water was evaporated to obtain the Na(3-HP) salt precipitate, which was subsequently dried using a lyophilizer. In addition, 30% w/v 3-HP solution was obtained by concentrating the liquid obtained after anion exchange chromatography.

This section describes the industrial-scale replication of the experimentally validated lab-scale DSP presented above, using the same sequence of unit operations for techno-economic evaluation. The industry-relevant DSP scheme proposed was for recovering 3-HP in high purity from the fermentation broth as (A) sodium 3-HP salt and (B) 30% w/v 3-HP aqueous solution. The obtained fermentation broth was clarified using a disk-stack centrifuge (S/L separation). The clarified broth was processed to remove coloring impurities by passing through an activated carbon column. The column operates in batch/semi-continuous mode and cycles independently of the main recipe to ensure continuous processing of the broth. There is always one column being loaded, and another one is being regenerated. Then, the clarified and decolorized broth was processed through a cation followed by anion exchange column to remove the unfermented nutrients and salts. This sequential operation can retain both all cations, including the ammonium cations (NH₄⁺) of the 3-hydroxy propionate salts, and all salt related to anions (SO₄²⁺). The columns were operated in batch mode, staggered and cycles independently of the main recipe. Later, the resulting broth was sent to the neutralizer tank. Wherein, sodium hydroxide (NaOH) was added stoichiometrically, and the sodium salt of 3-HP was formed (efficiency 77.3%). The resulting slurry is sent to a basket centrifuge for separating the sodium 3-HP salt crystals from the mother liquor. The basket centrifugation unit operates in batch mode and cycling independently from the main. The separated sodium 3-HP crystals were finally dried in a rotary drying procedure. The crystals exit the dryer with a sodium 3-HP salt purity 83.2%. The mother liquor and the wash drain from the basket centrifuge contain a significant amount of dissolved 3-HP and can be recycled back to the salting out section.

To produce a 30% w/v 3-HP aqueous solution, a single-effect evaporator was added after an ion exchange (cation/anion) column. While the neutralization, basket centrifuge, and rotary evaporator were removed.

### Results and Discussion

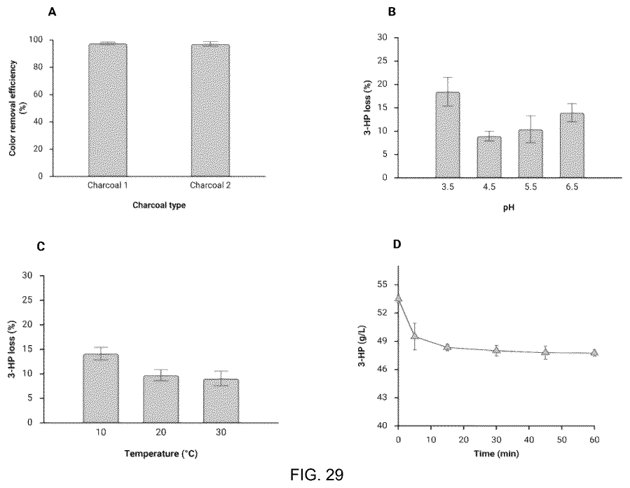

Preliminary adsorption studies were conducted to optimize decolorization and minimize 3-HP loss during subsequent ion-exchange steps. Furthermore, the optimal contact time for adsorption on the Amberlite IRA-67 weak base anion exchange resin was evaluated. The results are summarized in FIG. 29. The decolorization efficiencies of the two tested activated charcoal powders (Charcoal 1: Darco® Activated G-60; Charcoal 2: Darco® 100 mesh) were nearly identical. As shown in FIG. 29A, both Charcoal 1 and Charcoal 2 achieved a color removal efficiency of approximately 98%. It is noteworthy to mention that the residual glucose in the broth was almost completely removed (>92% removal efficiency). Since the differences in performance were statistically insignificant between two charcoals, the charcoal type was selected based on the ease of separation. Charcoal 1 (Darco® Activated G-60) was selected for subsequent studies because the Darco® Activated G-60 is coarser, providing greater flow and lower pressure drop for column operation.

Subsequently, the relationship between solution pH and 3-HP loss (adsorption) onto the Amberlite™ IRA-67 weak base anion exchange resin was investigated for selective removal, see FIG. 29B. The study revealed a clear trend: 3-HP loss was highest at pH 3.5 (~20%) and decreased sharply to a minimum loss of approximately 9% at pH 4.5. This behavior, which is initially counter-intuitive to classical ion exchange principles, confirms a mechanism shift at low pH. Below the pKa of 3-HP (~4.0 to 4.5), the molecule is primarily in its neutral molecular form, which is readily captured by the resin's matrix via physical adsorption, resulting in the high loss observed at pH 3.5. Under these conditions, non-specific interactions between neutral 3-HP molecules and the resin surface, including hydrophobic and van der Waals interactions with the polymeric matrix, contribute significantly to physical adsorption and apparent product loss. This is due to the dominated interactions of hydrophobic forces at pH below the pKa value of the acids. Conversely, at pH 4.5, product loss was minimized because the low concentration of neutral 3-HP molecules reduced physical adsorption. At the same time, the declining positive charge on the weak base resin suppressed electrostatic ion exchange. Consequently, pH 4.5 was identified as the optimal operating condition for minimizing 3-HP loss.

Furthermore, the influence of temperature on 3-HP loss was assessed under optimal pH conditions. FIG. 29C. The results showed that 3-HP loss was minimal at 30° C. (~8%). Notably, the loss increased to nearly 15% when the temperature was lowered to 10° C. This indicates that the adsorption process is exothermic, as decreasing the temperature favors the forward (adsorption) reaction, according to Le Chatelier's principle.

Finally, the adsorption kinetics study confirmed that most anion adsorption occurred in the first 5 min. FIG. 29D. Because the Amberlite IRA-67 weak base resin exhibits a high binding affinity for stronger acid anions (e.g., sulfate and chloride) over the weaker carboxylic acid anion of 3-HP, the rapid initial adsorption is attributed to the swift removal of these highly favored impurities. Therefore, the onset of measurable 3-HP adsorption was used as an indicator of the near-complete saturation of the resin's binding sites by these preferred anionic impurities.

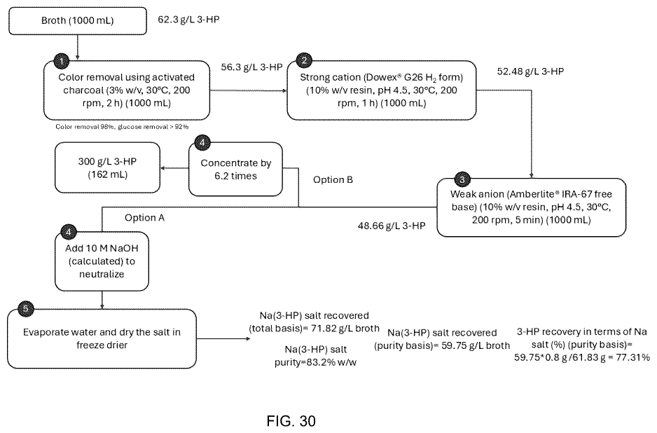

The simplified downstream purification (DSP) flowsheet for recovering 3-HP from the fermentation broth is summarized in FIG. 30, which presents the sequence of unit operations, operating conditions, and the stepwise mass balance of 3-HP across each processing stage.

Decolorization using activated carbon resulted in 98% color removal, with 8.9% loss in 3-HP concentration. The activated carbon also absorbed more than 92% of the glucose present in the supernatant. The predominant cations and anions in the broth were NH₄⁺ and SO₄²⁻, respectively, with other ions either below the detection limit or present at concentrations below 100 mg/L. Cation-exchange resin, Dowex® G26 H₂ form, effectively removed 96% of the ammonium ions, whereas more than 95% of the sulfate ions were removed by the anion-exchange resin, Amberlite® IRA-67 free base. Although the anion exchange resin has lower selectivity for carboxylate anions, a reduction in 3-HP concentration from 52.48 g/L to 48.66 g/L was observed in the weak anion exchange chromatography. After neutralization, 3-HP was recovered in the form of sodium salt with 77.31% 3-HP recovery and 83.2% purity of the salt crystals. Apart from the solid form of Na(3-HP), the purified liquid after the anion exchange step was concentrated 6.2 times to obtain a 30% w/v 3-HP solution. The remaining impurity is likely dominated by co-retained trace inorganic salts from the broth and processing chemicals, minor amount of glucose, and residual mother liquor/entrained moisture. Higher purity could be achieved by implementing additional crystal washing (to remove adhering mother liquor), recrystallization/fractional crystallization, and/or a polishing desalting step prior to salting-out.

Moreover, the Na(3-HP) salt samples were qualitatively and quantitatively compared with the standard salt using NMR spectroscopy. The spectrum showed identical peaks with a purity of approx. 85%, which was consistent with the HPLC data. The structural integrity and high purity of the recovered Na(3-HP) salt were confirmed by comparing it with the predicted structure and a commercial standard using both ¹H and ¹³C NMR spectroscopy. The ¹H NMR spectra verified the two expected methylene proton environments of 3-HP. Specifically, the spectra of the recovered product, standard, and prediction showed characteristic resonance signals (δ(H)) at 3.78-3.96 ppm, corresponding to the CH₂ protons adjacent to the hydroxyl (—OH) group, and another multiplet at 2.46-2.65 ppm, confirming the CH₂ protons near the carboxylate ion (COO—). This correlation confirms the assigned structure and expected chemical environments of the hydrogen atoms within the 3-HP molecule. Furthermore, the ¹³C NMR spectra verified the presence of three distinct carbon atoms in the C3 backbone. All samples exhibited signals (5(C)) near 180.66 ppm (assigned to the C═O of the carboxylate), 58.90 ppm (assigned to the CH₂—OH carbon), and 39.99 ppm (assigned to the intermediate CH₂ group). This precise agreement between the recovered sample and the standard across both ¹H and ¹³C NMR spectroscopy verifies the successful synthesis and high purity of the 3-HP sodium salt.

This Example demonstrates the successful development and validation of a novel, simplified, solvent-free downstream processing (DSP) flowsheet for the recovery of 3-Hydroxypropionic acid (3-HP) at an industrially relevant scale. Preliminary studies were conducted to minimize the 3-HP loss. The integrated scheme efficiently removed major cationic and anionic impurities, providing 3-HP in two valuable, high-purity forms: a solid Na(3-HP) salt and a 30% w/v aqueous solution. Techno-economic analysis (TEA) confirmed that the minimum selling prices (MSP) for both forms of 3-HP are comparatively below the target range required for 3-HP to be cost-competitive with petroleum-derived precursors for acrylic acid synthesis. This developed DSP could be an economically sustainable and scalable solution for overcoming the critical DSP bottleneck in bio-based 3-HP production.

### REFERENCES

1. Nielsen, J. & Keasling, J. D. Engineering cellular metabolism. Cell 164, 1185-1197 (2016).
2. Lee, S. Y., Kim, H. U., Chae, T. U., Cho, J. S., Kim, J. W., Shin, J. H., Kim, D. I., Ko, Y. S., Jang, W. D., & Jang, Y. S. A comprehensive metabolic map for production of bio-based chemicals. Nat. Catal. 2, 18-33 (2019).
3. Volk, M. J., Tran, V. G., Tan, S. I., Mishra, S., Fatma, Z., Boob, A., Li, H., Xue, P., Martin, T. A., & Zhao, H. Metabolic engineering: methodologies and applications. Chem. Rev. 123, 5521-5570 (2023).
4. Werpy, T. & Petersen, G. Top value added chemicals from biomass: volume I—results of screening for potential candidates from sugars and synthesis gas. osti.gov/biblio/15008859 (2004). doi:10.2172/15008859.
5. Bozell, J. J. & Petersen, G. R. Technology development for the production of biobased products from biorefinery carbohydrates—the US Department of Energy's “Top 10” revisited. Green Chem. 12, 539-554 (2010).
6. Vidra, A. & Nemeth, A. Bio-based 3-hydroxypropionic acid: a review. Period. Polytech. Chem. Eng. 62, 156-166 (2018).
7. Kumar, V., Ashok, S. & Park, S. Recent advances in biological production of 3-hydroxypropionic acid. Biotechnol. Adv. 31, 945-961 (2013).
8. Kildegaard, K. R., Jensen, N. B., Schneider, K., Czarnotta, E., Ozdemir, E., Klein, T., Maury, J., Ebert, B. E., Christensen, H. B., Chen, Y., Kim, I. K., Herrgard, M. J., Blank, L. M., Forster, J., Nielsen, J., & Borodina, I. Engineering and systems-level analysis of Saccharomyces cerevisiae for production of 3-hydroxypropionic acid via malonyl-CoA reductase-dependent pathway. Microb. Cell Factories 15, 53 (2016).
9. Moussa, M., Burge, G., Chemarin, F., Bounader, R., Saulou-Berion, C., Allais, F., Spinnler, H.-E., & Athes, V. Reactive extraction of 3-hydroxypropionic acid from model aqueous solutions and real bioconversion media. Comparison with its isomer 2-hydroxypropionic (lactic) acid: Reactive extraction of 3-HP acid from aqueous solutions and bioconversion broths. J. Chem. Technol. Biotechnol. 91, 2276-2285 (2016).
10. Rathnasingh, C., Raj, S. M., Jo, J.-E. & Park, S. Development and evaluation of efficient recombinant Escherichia coli strains for the production of 3-hydroxypropionic acid from glycerol. Biotechnol. Bioeng. 104, 729-739 (2009).
11. IndustrialSectorPMR, A. Acrylic Acid: Growth of Coatings Industry—Key Influencer 2029. Site Title marketreserchfmi.wordpress.com/2021/10/01/acrylic-acid-growth-of-coatings-industry-key-influencer-2029/(2021).
12. Dishisha, T., Pyo, S.-H. & Hatti-Kaul, R. Bio-based 3-hydroxypropionic- and acrylic acid production from biodiesel glycerol via integrated microbial and chemical catalysis. Microb. Cell Factories 14, 200 (2015).
13. Abraham, T. W., Allen, E., Hahn, J. J., Tsobanakis, P., Bohnert, E. C., & Frank, C. L. U.S. Pat. No. 11,236,036. Washington, DC: U.S. Patent and Trademark Office. (2022).
14. Wang, X., Cui, Z., Sun, X., Wang, Z. & Chen, T. Production of 3-hydroxypropionic acid from renewable substrates by metabolically engineered microorganisms: a review. Molecules 28, 1888 (2023).
15. Batista, R. S., Chaves, G. L., Oliveira, D. B., Pantaleão, V. L., dos Santos Neves, J. D., & da Silva, A. J. Glycerol as substrate and NADP+-dependent glyceraldehyde-3-phosphate dehydrogenase enable higher production of 3-hydroxypropionic acid through the β-alanine pathway in E. coli. Bioresour. Technol. 393, 130142 (2024).
16. Bhagwat, S. S., Li, Y., Cortes-Pena, Y. R., Brace, E. C., Martin, T. A., Zhao, H., & Guest, J. S. Sustainable production of acrylic acid via 3-hydroxypropionic acid from lignocellulosic biomass. ACS Sustain. Chem. Eng. 9, 16659-16669 (2021).
17. Kang, F., Gu, F., Zhong, Y., Cui, Z., Liang, Q., & Qi, Q. Expanding the genetic toolkit of Yarrowia lipolytica: dynamic promoter engineering enables high-titer biosynthesis of 3-hydroxypropionic acid. Bioresour. Technol. (2025).
18. Qin, N., Li, L., Wan, X., Ji, X., Chen, Y., Li, C., Liu, P., Zhang, Y., Yang, W., Jiang, J., Xia, J., Shi, S., Tan, T., Nielsen, J., Chen, Y., & Liu, Z. Increased CO₂ fixation enables high carbon-yield production of 3-hydroxypropionic acid in yeast. Nat. Commun. 15, 1591 (2024).
19. Xiao, H., Shao, Z., Jiang, Y., Dole, S. & Zhao, H. Exploiting Issatchenkia orientalis SD108 for succinic acid production. Microb. Cell Factories 13, 121 (2014).
20. Cao, M., Fatma, Z., Song, X., Hsieh, P. H., Tran, V. G., Lyon, W. L., Sayadi, M., Shao, Z., Yoshikuni, Y., & Zhao, H. A genetic toolbox for metabolic engineering of Issatchenkia orientalis. Metab. Eng. 59, 87-97 (2020).
21. Tran, V., Fatma, Z., Song, X. & Zhao, H. Development of a CRISPR/Cas9-based tool for gene deletion in Issatchenkia orientalis. mSphere 4, (2019).
22. Fatma, Z., Tan, S.-I., Boob, A. G. & Zhao, H. A landing pad system for multicopy gene integration in Issatchenkia orientalis. Metab. Eng. 78, 200-208 (2023).
23. Suthers, P. F., Dinh, H. V., Fatma, Z., Shen, Y., Chan, S. H. J., Rabinowitz, J. D., Zhao, H., & Maranas, C. D. Genome-scale metabolic reconstruction of the non-model yeast Issatchenkia orientalis SD108 and its application to organic acids production. Metab. Eng. Commun. 11, e00148 (2020).
24. Jiang, X., Meng, X. & Xian, M. Biosynthetic pathways for 3-hydroxypropionic acid production. Appl. Microbiol. Biotechnol. 82, 995-1003 (2009).
25. Valli, M., Sauer, M., Branduardi, P., Borth, N., Porro, D., & Mattanovich, D. Intracellular pH distribution in Saccharomyces cerevisiae cell populations, analyzed by flow cytometry. Appl. Environ. Microbiol. 71, 1515-1521 (2005).
26. Wang, L., Upadhyay, V. & Maranas, C. D. dGPredictor: automated fragmentation method for metabolic reaction free energy prediction and de novo pathway design. PLOS Comput. Biol. 17, e1009448 (2021).
27. Beber, M. E., Gollub, M. G., Mozaffari, D., Shebek, K. M., Flamholz, A. I., Milo, R., & Noor, E. eQuilibrator 3.0: a database solution for thermodynamic constant estimation. Nucleic Acids Res. 50, D603-D609 (2022).
28. Zallot, R., Oberg, N. & Gerlt, J. A. The EFI web resource for genomic enzymology tools: leveraging protein, genome, and metagenome databases to discover novel enzymes and metabolic pathways. Biochemistry 58, 4169-4182 (2019).
29. Borodina, I., Kildegaard, K. R., Jensen, N. B., Blicher, T. H., Maury, J., Sherstyk, S., Schneider, K., Lamosa, P., Herrgard, M. J., Rosenstand, I., Öberg, F., Forster, J., & Nielsen, J. Establishing a synthetic pathway for high-level production of 3-hydroxypropionic acid in Saccharomyces cerevisiae via β-alanine. Metab. Eng. 27, 57-64 (2015).
30. Tran, V. G., Mishra, S., Bhagwat, S. S., Shafaei, S., Shen, Y., Allen, J. L., Crosly, B. A., Tan, S. I., Fatma, Z., Rabinowitz, J. D., Guest, J. S., Singh, V., & Zhao, H. An end-to-end pipeline for succinic acid production at an industrially relevant scale using Issatchenkia orientalis. Nat. Commun. 14, 6152 (2023).
31. Tran, V. G., Tan, S. I., Xu, H., Weilandt, D. R., Li, X., Bhagwat, S. S., Zhu, Z., Guest, J. S., Rabinowitz, J. D., & Zhao, H. Decompartmentalization of the yeast mitochondrial metabolism to improve chemical production in Issatchenkia orientalis. Nat. Commun. 16, 7110 (2025).
32. Lee, Y. G., Kang, N. K., Kim, C., Tran, V. G., Cao, M., Yoshikuni, Y., Zhao, H., & Jin, Y. S. Self-buffering system for cost-effective production of lactic acid from glucose and xylose using acid-tolerant Issatchenkia orientalis. Bioresour. Technol. 399, 130641 (2024).
33. Tan, S. I., Liu, Z., Tran, V. G., Martin, T. A. & Zhao, H. Issatchenkia orientalis as a platform organism for cost-effective production of organic acids. Metab. Eng. 89, 12-21 (2025).
34. Matsakas, L., Hrůzová, K., Rova, U. & Christakopoulos, P. Biological production of 3-hydroxypropionic acid: an update on the current status. Fermentation 4, 13 (2018).
35. IHS Markit. Chemical Economics Handbook—Acrylic Acid and Esters. IHS Markit, London, England. (2020).
36. Wang, M., Elgowainy, A., Lu, Z., Bafana, A., Benavides, P. T., Burnham, A., Cai, H., Dai, Q., Gracida, U., Hawkins, T. R., Jaquez, P. V., Kelly, J. C., Kwon, H., Lee, U., Liu, X., Ou, L., Sun, P., Winjobi, O., Xu, H., Yoo, E., Zaimes, G. G., & Zang, G. Greenhouse gases, regulated emissions, and energy use in technologies model. Computer Software. USDOE Office of Energy Efficiency and Renewable Energy (EERE). (2020).
37. Wernet, G., Bauer, C., Steubing, B., Reinhard, J., Moreno-Ruiz, E., & Weidema, B. The ecoinvent database version 3 (part 1): overview and methodology. The Int. J. Life Cycle Assess. 21, 1218-1230 (2016).
38. Norsigian, C. J., Pusarla, N., McConn, J. L., Yurkovich, J. T., Dräger, A., Palsson, B. O., & King, Z. BiGG Models 2020: multi-strain genome-scale models and expansion across the phylogenetic tree. Nucleic Acids Res. 48, D402-D406 (2020).
39. Moretti, S., Tran, V. D. T., Mehl, F., Ibberson, M. & Pagni, M. MetaNetX/MNXref: unified namespace for metabolites and biochemical reactions in the context of metabolic models. Nucleic Acids Res. 49, D570-D574 (2021).
40. Hastings, J., Owen, G., Dekker, A., Ennis, M., Kale, N., Muthukrishnan, V., Turner, S., Swainston, N., Mendes, P., & Steinbeck, C. ChEBI in 2016: Improved services and an expanding collection of metabolites. Nucleic Acids Res. 44, D1214-D1219 (2016).
41. Kim, S., Chen, J., Cheng, T., Gindulyte, A., He, J., He, S., Li, Q., Shoemaker, B. A., Thiessen, P. A., Yu, B., Zaslavsky, L., Zhang, J., & Bolton, E. E. PubChem 2023 update. Nucleic Acids Res. 51, D1373-D1380 (2023).
42. Ji, R. Y., Ding, Y., Shi, T. Q., Lin, L., Huang, H., Gao, Z., & Ji, X. J. Metabolic engineering of yeast for the production of 3-hydroxypropionic acid. Front. Microbiol. 9, 2185 (2018).
43. Orth, J. D., Thiele, I. & Palsson, B. Ø. What is flux balance analysis? Nat. Biotechnol. 28, 245-248 (2010).
44. Ebrahim, A., Lerman, J. A., Palsson, B. O. & Hyduke, D. R. COBRApy: COnstraints-Based Reconstruction and Analysis for Python. BMC Syst. Biol. 7, 74 (2013).
45. McKinney, W. Data structures for statistical computing in Python. scipy, 445(1), 51-56 (2010).
46. Granger, B. E. & Pérez, F. Jupyter: Thinking and storytelling with code and data. Comput. Sci. Eng. 23, 7-14 (2021).
47. Kanehisa, M., Furumichi, M., Sato, Y., Ishiguro-Watanabe, M. & Tanabe, M. KEGG: integrating viruses and cellular organisms. Nucleic Acids Res. 49, D545-D551 (2021).
48. Cortes-Pena, Y., Kumar, D., Singh, V. & Guest, J. S. BioSTEAM: a fast and flexible platform for the design, simulation, and techno-economic analysis of biorefineries under uncertainty. ACS Sustain. Chem. Eng. 8, 3302-3310 (2020).
49. Cortes-Pena, Y. Thermosteam: BioSTEAM's Premier Thermodynamic Engine. J. Open Source Softw. 5, 2814 (2020).
50. BioSTEAM Development Group. 3-Hydroxypropionic Acid Biorefineries. github.com/BioSTEAMDevelopmentGroup/Bioindustrial-Park/tree/master/biorefineries/HP (2024).
51. Lee, J. W., Bhagwat, S. S., Kuanyshev, N., Cho, Y. B., Sun, L., Lee, Y. G., Cortes-Pena, Y. R., Li, Y., Rao, C. V., Guest, J. S.& Jin, Y. S. Rewiring yeast metabolism for producing 2,3-butanediol and two downstream applications: techno-economic analysis and life cycle assessment of methyl ethyl ketone (MEK) and agricultural biostimulant production. Chem. Eng. J. 451, 138886 (2023).
52. Kim, M. S., Bhagwat, S. S., Santiago-Martinez, L., Shi, X., Choi, K., Guest, J. S., & Huber, G. W. Sustainable potassium sorbate production from triacetic acid lactone in food-grade solvents. Green Chem. 27(21), 6087-6104 (2025).
53. Bhagwat, S. S., Dell'Anna, M. N., Li, Y., Cao, M., Brace, E. C., Bhagwat, S. S., Huber, G. W., Zhao, H., & Guest, J. S. Sustainable triacetic acid lactone production from sugarcane and sweet sorghum by fermentation and fractional crystallization. ChemRxiv. (2024).
54. Seider, W. D., Lewin, D. R., Seader, J. D., Widagdo, S., Gani, R., & Ng, K. M. Product and process design principles: synthesis, analysis and evaluation. John Wiley & Sons. (2016).
55. U.S. EPA. Lifecycle Analysis of Greenhouse Gas Emissions under the Renewable Fuel Standard.epa.gov/renewable-fuel-standard-program/lifecycle-analysis-greenhouse-gas-emissions-under-renewable-fuel. (2025).
56. W G, I. The physical science basis. Contrib. Work. Group Fifth Assess. Rep. Intergov. Panel Clim. Change 1535, (2013).

1. A recombinant yeast comprising one or more heterologous polynucleotides encoding 2, 3, 4, 5, 6, 7, or 8 copies of an aspartate 1-decarboxylase (PAND) polypeptide.

2. The recombinant yeast of claim 1, wherein the one or more heterologous polynucleotides encoding one or more copies of an PAND polypeptide are derived from Apolygus lucorum.

3. The recombinant yeast of claim 1, wherein the one or more heterologous polynucleotides encode a PAND polypeptide comprising 90% or greater sequence identity to the sequence set forth in SEQ ID NO:213.

4. The recombinant yeast of claim 1, wherein a pyruvate decarboxylase (PDC) gene of the recombinant yeast is inactivated.

5. The recombinant yeast of claim 1, wherein a glycerol-3-phosphate dehydrogenase (GPD) gene of the recombinant yeast is inactivated.

6. The recombinant yeast of claim 1, wherein the recombinant yeast further comprises one or more heterologous polynucleotides encoding one or more copies of a pyruvate carboxylase (PYC) polypeptide, or wherein a PYC polypeptide is overexpressed.

7. The recombinant yeast of claim 1, wherein the recombinant yeast further comprises one or more heterologous polynucleotides encoding two or more copies of a pyruvate carboxylase (PYC) polypeptide, or wherein a PYC polypeptide is overexpressed.

8. The recombinant yeast of claim 1, wherein the recombinant yeast further comprises one or more heterologous polynucleotides encoding one or more copies of an aspartate aminotransferase (AAT) polypeptide or wherein an ATT polypeptide is overexpressed.

9. The recombinant yeast of claim 1, wherein the recombinant yeast further comprises one or more heterologous polynucleotides encoding two or more copies of an aspartate aminotransferase (AAT) polypeptide or wherein an ATT polypeptide is overexpressed.

10. The recombinant yeast of claim 1, wherein the recombinant yeast further comprises one or more heterologous polynucleotides encoding one or more copies of a β-alanine-pyruvate aminotransferase (BAPAT) polypeptide.

11. The recombinant yeast of claim 10, wherein the one or more heterologous polynucleotides encode a BAPAT polypeptide comprising 90% or greater sequence identity to the sequence set forth in SEQ ID NO:228-234.

12. (canceled)

13. The recombinant yeast of claim 1, wherein the recombinant yeast further comprises one or more heterologous polynucleotides encoding one or more copies of a 3HP dehydrogenase (YDFG) polypeptide.

14. The recombinant yeast of claim 13, wherein the one or more heterologous polynucleotides encoding a YDFG polypeptide comprise 90% or greater sequence identity to the sequence set forth in SEQ ID NO:218-227.

15. (canceled)

16. (canceled)

17. (canceled)

18. (canceled)

19. (canceled)

20. A method of producing 3-hydroxypropionic acid (3HP) comprising adding the recombinant yeast of claim 1 to cell culture media, cultivating the recombinant yeast to a desired cell density, and harvesting the 3HP.

21. The method of claim 20, wherein the 3HP is produced at 75 g/L or more, or at about 90 g/L or more.

22. The method of claim 20, wherein a yield of the 3HP is 0.5 g/g glucose or more, or 0.7 g/g glucose or more.

23. (canceled)

24. (canceled)

25. (canceled)

26. The method of claim 20, wherein less than 0.7 g/L of pyruvate is produced.

27. The method of claim 20, further comprising providing CO₂ to the cell culture media during a 3HP production phase.

28. A method of isolating 3-hydroxypropionic acid from a fermentation broth comprising:
(a) removing cells from the fermentation broth to form a cell-free fermentation broth;
(b) decolorizing the cell-free fermentation broth to form a cell-free, decolorized fermentation broth;
(c) removing cations and anions from the cell-free, decolorized fermentation broth; and
(d) adding NaOH to the product of step (c) to form 3-hydroxypropionic acid.

29. (canceled)

30. (canceled)

31. (canceled)

32. (canceled)

33. (canceled)

34. (canceled)

35. (canceled)

36. (canceled)

37. A polynucleotide molecule encoding a polypeptide as set forth in SEQ ID NO:1-10 or 193-208, wherein the polynucleotide molecule is operably linked to a heterologous yeast promoter or wherein the polynucleotide molecule is operably linked to a marker polynucleotide.

38. (canceled)

39. (canceled)


SEQUENCE LISTING (239 sequences)

SEQ ID NO: 1 - DNA, 1416 bp, unidentified
  source 1..1416: mol_type = genomic DNA; note = Beauveria bassiana; strain = D1-5
atgattgttttagttacaggtgccacggcaggcttcggagaatctatcacaagaagatttgtcaagaacggtcataaagttattgcaacaggtagaaggcaggaaaggttattagaattaaaggaggaattgggtgataaccttttcacgttgcagttagatgtccgtaatcgtgcagccatcgaggaagccttagcttctttacctgctgaatggcaacacattgatattcttgttaataatgctggattggcattgggaatggagcctgcacacaaggcaaacgtggatgactgggaaaacatgatcgacactaataacaagggtttggtctacatgacccgtgcggttttgcctggcatggttgagagaaatagaggtcatattatcaatattggttcaactgctggttcttggccatacgcgggtggcaacgtgtacggtgcgaccaaagcatttgttagacaattctctttgaacttgagaaccgacctccataagacagcggtccgtgttaccgatattgaaccaggtatggtcggtggtactgagttttcaaatgttaggtttaagggtgatgacgacaaggcaggtaaaacttatgaaaatgcaaacgcattgaccccagaagatgtgaccgaagcagtgtggtgggtcgctactctcccaaagcacgtcaatatcaacactttagaaatgatgccacaaacctcggaagacaaaaagacgatgcctttgtcccgtgaaatcaatccaacacaaccagttaatcagcaaatctaccatattttaagaagagagatcgttagatgtcaaattgctccaggcacacctttgtcggagaaggaagtttcgggtagatttgatgtttccagacagcctgttagagaagcgtttattaaacttgcagagacaggattggtccagatcagaccacagaggggtacttgggtgtcgaagatttcagtttctcacgttagaaacggatgtttcatcagagaagctatcgaaagagctgtcgttaagcgtgcggccgagaaggtcaatgattcagatctttacgccttagaacatctcttaaaccaacaaagattagcaattactgactcccaaatcgatgattttttctcgctcgatgataagttccataggcaattagccttaattgccgactgctccttggcttgggacaccgttgaaaatatcaaagcaacaatggatagagtcagatatatgtcattggatcatgtttcccctcctgctatgttgttggaacaacattatgctattttcaacgccctcaaggcgcatgatgtcgctgaagtcgatactgccatgcaccttcacttgcacgagatttctcagtctattttgttaatcagacaagaaaaccaagactggttttccgatgagtaa

SEQ ID NO: 2 - DNA, 861 bp, Plautia stali
  source 1..861: mol_type = genomic DNA
atgccaggctcattatctttcgcaacactttcgaaacagcaagctgaatcaggttacgagttaaacacatctcatcgtcaaccagactccactagacaagcaagaaactctgaaatgatcatctttgttactggtgctaccgctggttttggtgaatcgattacgagaaggttcattgctaacggacacaaggtcatcgcatctggtagacgtgctgaaagattaaaagccttgaaggaggaattgggtgcaaacctttatacagtccagttggatgttagaaacagagcagccattgacgaagctatcgccgcgttgccagctgaatggcgtgagatcgatgttctcgtgaataatgcaggtttagccttgggtattgaaccagctcataaggccaacattgaggattgggaaaacatgattgacaccaataataaaggcttagtctatatgactagagcggttctccctgcaatggttgaaaggaatgttggtcatattgttaatattggatccatcgcaggttcatggccatatcaaggaggtaatgtttacggagccaccaaggcatttgtgagacaattttcgttgaacttgagaacagatcttcatggtactgcattgagagtcacagatattgagcctggtttagttggtggtaccgaattctccaacgtcaggttcaagggcgacgatggaagagcggatgcagtttacgagggcaccactgccttgactgccgaggatgtcacagaagctgtctactgggttactacgttacctaagcacgttaatatcaatactctcgaaattatgcctgtgtcccagaccttggcgggtttaaaggtccacaagggttaa

SEQ ID NO: 3 - DNA, 747 bp, Erwinia mallotivora
  source 1..747: mol_type = genomic DNA
atgattgtctttgttacaggcgccacagcaggttttggtgaatccatcactagaaggtttattgctcaaggacataaggttgttgcaacaggtagacgttcggagaggcttcaagccttgaaggatgaactcggtgaaaacgtttacactctccagttagacgtcagaaacagagcgggtattgatgcagctttacattcgttgccagcggagtggagagacattgatgttcttgtcaataatgctggtttggccttgggtatggccccagcccaccaagcttcaattgacgattgggaagcaatgatcgataccaataataaaggtttagtctttatgacaagagcaatcttacctgcaatggtggaaaggaacgtcggtcatatcatcaatattggctctactgccggaaactggccttatgcgggtggcaacgtgtatggtgccaccaaagctttcgttagacaattctccttaaacttgcgtactgatcttcacggaactgcattgagagtcaccgatattgaacctggattagttggtggcactgaattttcaaatgttagattcaagggtgactctgataaggcggattccgtctacgacggtacggaaccattgaccgcagaggatgtcacggaagctgttttctgggttgcttctctcccaaaacacgtgaacatcaataccttggagatgatgccagttggtcagacattggctggtttgaaggttcataaggcataa

SEQ ID NO: 4 - DNA, 753 bp, Helicobacter pylori
  source 1..753: mol_type = genomic DNA
atggctcatattcttgtctcaggtgctacctcgggattcggattagagatcgccaaggcattcctccaaaagaatcacgtcgtctttggtacaggtagaagaaaggagaaccttcaagaattgcagctcgcgtatccaaaaagattcattcctttgtgttttgacctccaaaacaagctcgaaacgaaaagagctatcgaaacaatcttttctatgacagacagaattgatgccttaatcaataatgctggtttggcattgggtttgaacaaggcgtatgaatgtgaattggacgattgggaagttatgattgatactaacatcaagggtttattgtaccttacgaggttaattttgccatccatgatcgaacatgatcaaggcactattattaatcttggttctattgccggtacctacgcatatccaggtggcaacgtttacggcgcttccaaggcgtttgttaagcaattctctttgaacttacgtgcagatttggccggtactaacatcagagtgtccaatgttgagcctggtttatgcggagagactgaattctcaatggttaggtttaagggtgataaaatcaaagcacagtcggtctatgagaatacattatacttgaaaccacaggacatcgcaaacattgttttgtggatctacgaacaaccttcgcacgtgaatattaatcgtattgaaattatgcctacctcacaaacctttgcaccattacctactcataagaatccataa

SEQ ID NO: 5 - DNA, 762 bp, Pseudomonas citronellolis
  source 1..762: mol_type = genomic DNA
atgactccaacattattcatcaccggtgcaacatctggttttggtgaggcatgtgcgagaagatttgctagagatggttggtccttggttattaccggtagaagagaagacaggttgcaagcattggccaaggagttgtcgtctcaagccgaagttttacctatcacattggatgttagagatagagcggcagtcgcacacgcagtggataatcttcctgcgggttttgacaagcttagagctctcattaataatgcaggtttggctttaggtgctgatccagcccaagaatgtgatctcgatgattgggacactatggtcgatacgaacattaagggtttgatgtactgcactaggttgttgttaccacgtctcatcgctcatggaaggggagcctcgatcttgaacttaggatctgtcgcgggtcaatggccttaccctggatcgcatgtttatggtgccactaaagctttcgttgaacagttttccttatcattgcgttgtgacctccagggcaccggtgttagagtttctaatcttgaaccaggtttgtgtgagtcagaattttcccttgtcagattcggcggtgaccaagctcgttatgacaagacttacgcaggcacagaagcgttacaacctgaagatattgctgaaaccattcattggattgtcaatcagccaccacatatcaacattaactccttagagatcatgccagtgtcacaagcatgggccggtttctctgttcacagaaaaggctaa

SEQ ID NO: 6 - DNA, 756 bp, Acetobacter sp.
  source 1..756: mol_type = genomic DNA; note = Acetobacter sicerae
atgaccgatactgttttggttacaggtgcaactgctggtttcggtagagcaatctccgaacgtcttgttaaggacggttatagagtcatcggtacgggtcgtagggctgaaagattggacgcgttacaggcatctttgggtgacgcttttctcccattggccttggatatgttggataaagaagccttaagaggtttaccaaccgaattaccagagggttggagagaggttgatgttttgattaacaatgcgggcttagctttgggcatggatccagctcaaactacaaacatcgatgactgggaacgtatgatcggaactaatgtgtcgggtatggtggagattacgagggcgctcttacctggtatggttgaacgtaatcatggacacgttgtctcgcttggttccacagctggcacctacccttacaagggtggcaacgtttatggagcctcaaaggcgtttgtttcccaattcatgagaaatcttagaacagaccttttgggtaccagagttagagcaactaacctcgaaccaggattgtgtggtggttcagaattttctaacgtgagactcggagatgaggcaaaagccgcagatgtctacagaggtactaagcctattttgcctgaagatattgcagagacagtcgcctgggtcttgggtttaccagaacatgtcaatattaatagaattgaaatgatgcctgtttgtcaagcagctggcggtttgtctgtcgttagggatgtcgactaa

SEQ ID NO: 7 - DNA, 756 bp, Gluconobacter oxydans
  source 1..756: mol_type = genomic DNA; strain = NBRC 3293
atgcctttaaccgtcttggtcacaggtgctaccgctggttttggccacgccattgccacgagattagttagagaaggtcatagggttattgccacaggtcgtagaaaggagagattggaagctttaaaggacgagttaggtgaatcagtgctcccatacacccttgatatgactgatagagaagctttgcgtgcattaccagctgcgctccctgaagaatggcaggatattgatgtgctcgtcaataacgcaggtttggcgttgggtatggaaccagcgcaagatgcagaacctgataattgggagtctatgatcgctactaacgtttccggtgttgtcgagttgactagagccttgttacctggaatggttcagcgtaatactggttacatcatcgcattgggttcgactgccggtacgtatccttatactggcggaaacgtttacggtgcaactaaagcttttgtccaccaattcacacaaaaccttagaacagaccttttgggtaccagaattagagttaccaacatcgaaccaggtttgtgtggcggatctgagttctcaaatgttaggttaggtgatgacgcaaaagctgcagcggtgtataaggacacaactccattgacaccagaagatattgcaggaaccgtttcgtggctcattggtttaccatggcatgtcaatgtcaactaccttgaaatgatgcctgtctgtcaagcatccggaggtttggccgttaatagacatgttggctaa

SEQ ID NO: 8 - DNA, 750 bp, unidentified
  source 1..750: mol_type = genomic DNA; note = Oleiagrimonas soli
atgaccaaaacagccatggttaccggtgctacttcgggctttggtgcagccattgcagagagattggttaaggacggctggagagttgtgatctgtggtagacgtgaagaacgtttacagaccatggtggataggttaggtgcagacaaggctcacgcagcagctttcgacattagagatgcagaggcgatgtctgaagcgttggatgccttgccagcagcgtttgcggatatcgatttgttagttaataacgctggtttagccttgggtaccgaaccagcacaattcgccgaccttgatgagtggaggcagatgatcgatactaacgtcactgctttggtcacactcactcatgccttgttgcctgccttaattgagagacgtggcgctatcgttaatatctcctcgatttcaggttcttatccttacaaaggcggaaacgtttatggtggtacgaaggcatttgttactcaattttcgcttggattaagaacagatcttcacggtacaggtgtcagagtcacttccgtggaacctggtatggcagagactgaattcacattagttagaaccggaggtgaccaagctgcttctgataagctctacacaggtgcccatcctattacggcggaagacattgcagaaaccgttgcttggattgctcatctcccaccacatttgaatgtcaatagaatcgaagttatgccaacttcccaatcagcggcaggattgcaagtccacagagattaa

SEQ ID NO: 9 - DNA, 765 bp, Geobacter sp.
  source 1..765: mol_type = genomic DNA; note = Geobacter soli
atgtccagaaccgtttttattacaggtgcatccgcaggatttggcgcggcatgtgcaagattgttcgccgacagaggagatagattggttttagcagcccgtagggaagagccattgttggctttaagggatgaattggctccacaagcagaagtccacactgtcaccctcgatgtcagaagaagaaaggcagttcaagaagccgttgagggtttaccagacgccttttcccgtattgacattttggtcaacaatgcaggcctcgctttaggtttggaaccagctcatgaaaccaaccctgatgattgggatgcgatggtcgatacgaacatcaagggtttgatctactgcactaggtttttattgccaggtatggttgcgagaaacagaggtcatattgtgaatattggttctgtggcgggatcatggccatatcctggtggtaacgtttatggcggtactaaggccttcgtcgaacaattctcgcgtaatcttagagccgaccttttgggcaaaagagtccgtgttacatgtttagcacctggtatggcagaaaccgagttctcaatggttaggtttaaaggagatgcagaaagagctgctcaagtgtacgccggtacacaggccttaacagcagaggacatcgcgggtattgtcgcttgggttactgaattaccacctcacgttaatgttaatgctcttgaggttatgtctatcttccagtcttggggtcctctcgctgtgcatagacctggtgcttcgtaa

SEQ ID NO: 10 - DNA, 747 bp, Campylobacter concisus
  source 1..747: mol_type = genomic DNA
atgaagaagaccgcatttgtcactggcgcaacctccggttttggtgaggccattgcaaggagattatcgaaagaaggttacaagattgttgctttggcaagacgtgaggatagattgaaaaagctcgcatcagaattaggtgatacgcatattatcgttgcggatattagagataaagaagcagttttcgacgccgtggattcattgcctgaaaacttcaaggacattgaagttttggtgaataacgccggcatggccttgggtttagaaaagacaatcgatgctaaggttgaagacttcgaagctatgatcgatacaaacgtcaagggattgatctactctacaaaggccgtcttgcctttgttatacaagcaggaaaaaggatatatttttaatatcggttctactgcgggttcttggccatacccaggttcaaacgtctatggtgcgactaaggcttttgtcaaacaattttccctcaacctcaggaatgaccttgttggcacaaatattagagtgaccaatatcgaaccaggtctttgtaagaccgagttctcggaggttagatttcgtggtgacaaggcaaaggcagattccctttatgagaacactaacttcattacttcggaggatatcgcgacaattttagttaattgtttaaacatgccaggatctgttaatatcaatagagttgaagtcatggctaatactcaaacgtgggctggtttggctattgaaaaattctaa

SEQ ID NO: 11 - DNA, 18 bp, synthetic construct
  source 1..18: mol_type = other DNA
tctagagccacctgggtc

SEQ ID NO: 12 - DNA, 25 bp, synthetic construct
  source 1..25: mol_type = other DNA
cgtctgtagagtaaagaaacttctc

SEQ ID NO: 13 - DNA, 46 bp, synthetic construct
  source 1..46: mol_type = other DNA
gtatatcttaaaacacacacacaaaatgtccagaggcttctttact

SEQ ID NO: 14 - DNA, 45 bp, synthetic construct
  source 1..45: mol_type = other DNA
tatttagtaaaagcattagttagatctaaaggtctctcacgacag

SEQ ID NO: 15 - DNA, 60 bp, synthetic construct
  source 1..60: mol_type = other DNA
atgatacgcggtcgtgctattatggtttccaaaagtttaatgcttgatttaacctgatcc

SEQ ID NO: 16 - DNA, 58 bp, synthetic construct
  source 1..58: mol_type = other DNA
actatcggcctctttttctccgggtgtggtgcatttttcgttcggagattaccgaatc

SEQ ID NO: 17 - DNA, 58 bp, synthetic construct
  source 1..58: mol_type = other DNA
tcggtttctttgaaatttttttgattcggtaatctccgaacgaaaaatgcaccacacc

SEQ ID NO: 18 - DNA, 58 bp, synthetic construct
  source 1..58: mol_type = other DNA
cacttaagacaatacacacttaatacaatacacatacaattgttcaaatgcttgtctg

SEQ ID NO: 19 - DNA, 60 bp, synthetic construct
  source 1..60: mol_type = other DNA
agttctgcgcctcttcaccagacaagcatttgaacaattgtatgtgtattgtattaagtg

SEQ ID NO: 20 - DNA, 60 bp, synthetic construct
  source 1..60: mol_type = other DNA
tatattcacaatgattattttctacatgtatagatggacctaactggaacatgtttgtac

SEQ ID NO: 21 - DNA, 44 bp, synthetic construct
  source 1..44: mol_type = other DNA
tgtagaaaataatcattgtgaatatatctagagccacctgggtc

SEQ ID NO: 22 - DNA, 50 bp, synthetic construct
  source 1..50: mol_type = other DNA
ccataatagcacgaccgcgtatcatcgtctgtagagtaaagaaacttctc

SEQ ID NO: 23 - DNA, 60 bp, synthetic construct
  source 1..60: mol_type = other DNA
tgaagtgcaagtggagtgtggaagttgaggcggacacgtgcacttggctaaagaataaga

SEQ ID NO: 24 - DNA, 60 bp, synthetic construct
  source 1..60: mol_type = other DNA
acaaacaagagtcccacaggcagccatgccaacagtggcgcagacccaggtggctctaga

SEQ ID NO: 25 - DNA, 60 bp, synthetic construct
  source 1..60: mol_type = other DNA
tgtcacagcaaggcccgtctataccaacatgtgcagagcacacttggctaaagaataaga

SEQ ID NO: 26 - DNA, 60 bp, synthetic construct
  source 1..60: mol_type = other DNA
tgtgtgtgtatgttatatccacacacaggggcagccacccctgacccaggtggctctaga

SEQ ID NO: 27 - DNA, 60 bp, synthetic construct
  source 1..60: mol_type = other DNA
aggtggaatccagttgatttctatcaggttaaccacatacaacttggctaaagaataaga

SEQ ID NO: 28 - DNA, 60 bp, synthetic construct
  source 1..60: mol_type = other DNA
ctctgtatgtcgaaagttttaccttactgttacacaaaggttgacccaggtggctctaga

SEQ ID NO: 29 - DNA, 60 bp, synthetic construct
  source 1..60: mol_type = other DNA
tttatctcggacttgactcggcaccctcttgccgcggcaccacttggctaaagaataaga

SEQ ID NO: 30 - DNA, 60 bp, synthetic construct
  source 1..60: mol_type = other DNA
gtggaaatgacagaatattggttgttccgcggctcgagaggcgacccaggtggctctaga

SEQ ID NO: 31 - DNA, 60 bp, synthetic construct
  source 1..60: mol_type = other DNA
gcagcttacttacgtatacagattatcctctctatggactcacttggctaaagaataaga

SEQ ID NO: 32 - DNA, 60 bp, synthetic construct
  source 1..60: mol_type = other DNA
atcgtagctggacgttacggcgcatcacaaggtgccacgtacgacccaggtggctctaga

SEQ ID NO: 33 - DNA, 60 bp, synthetic construct
  source 1..60: mol_type = other DNA
ccatggcatgtataaataacggatgtgtgagttcaagatgcacttggctaaagaataaga

SEQ ID NO: 34 - DNA, 60 bp, synthetic construct
  source 1..60: mol_type = other DNA
ccatgtctattgtaaacaatagaagtacagacacaaacaaacgacccaggtggctctaga

SEQ ID NO: 35 - DNA, 60 bp, synthetic construct
  source 1..60: mol_type = other DNA
ataactaaattagactgtatttcaaatgagccgcctttagaacttggctaaagaataaga

SEQ ID NO: 36 - DNA, 60 bp, synthetic construct
  source 1..60: mol_type = other DNA
cttgaactcacacatccgttatttatacatgccatggaatttgacccaggtggctctaga

SEQ ID NO: 37 - DNA, 60 bp, synthetic construct
  source 1..60: mol_type = other DNA
taggtagacatgctacttttctctcttggttttaccacctaacttggctaaagaataaga

SEQ ID NO: 38 - DNA, 60 bp, synthetic construct
  source 1..60: mol_type = other DNA
tttccatatcattttatatgtagacaatttaatctacatacagacccaggtggctctaga

SEQ ID NO: 39 - DNA, 60 bp, synthetic construct
  source 1..60: mol_type = other DNA
tgatacgcggtcgtgctattatggtttccaaaagtttaatgacttggctaaagaataaga

SEQ ID NO: 40 - DNA, 60 bp, synthetic construct
  source 1..60: mol_type = other DNA
tatattcacaatgattattttctacatgtatagatggacctagacccaggtggctctaga

SEQ ID NO: 41 - DNA, 60 bp, synthetic construct
  source 1..60: mol_type = other DNA
aagaagtacctaagacggaaacctgaataatggagtgagttacttggctaaagaataaga

SEQ ID NO: 42 - DNA, 60 bp, synthetic construct
  source 1..60: mol_type = other DNA
caaaaggagatgctcgtgacttctattggcttgtggaatagtgacccaggtggctctaga

SEQ ID NO: 43 - DNA, 60 bp, synthetic construct
  source 1..60: mol_type = other DNA
gtggcctaaacgttacttatgtactcagagatacgtatacgatcgattgtatgtgtattg

SEQ ID NO: 44 - DNA, 60 bp, synthetic construct
  source 1..60: mol_type = other DNA
taactcacttatagtatgaaccttctatttcggacatcaagtggctctagatggaacatg

SEQ ID NO: 45 - DNA, 60 bp, synthetic construct
  source 1..60: mol_type = other DNA
catggcatgtataaataacggatgtgtgagttcaagatgcgatcgattgtatgtgtattg

SEQ ID NO: 46 - DNA, 60 bp, synthetic construct
  source 1..60: mol_type = other DNA
atgtctattgtaaacaatagaagtacagacacaaacaaacgtggctctagatggaacatg

SEQ ID NO: 47 - DNA, 50 bp, synthetic construct
  source 1..50: mol_type = other DNA
gaaacttctcgttcatcttattctttagccaatacagacgcgaaaaatgc

SEQ ID NO: 48 - DNA, 51 bp, synthetic construct
  source 1..51: mol_type = other DNA
cgggtgtggtgcatttttcgcgtctgtattggctaaagaataagatgaacg

SEQ ID NO: 49 - DNA, 20 bp, synthetic construct
  source 1..20: mol_type = other DNA
ggtgagggtttgtaacaaag

SEQ ID NO: 50 - DNA, 21 bp, synthetic construct
  source 1..21: mol_type = other DNA
tccgcctcagcatataataac

SEQ ID NO: 51 - DNA, 20 bp, synthetic construct
  source 1..20: mol_type = other DNA
ccacaattccaaacaaggtg

SEQ ID NO: 52 - DNA, 20 bp, synthetic construct
  source 1..20: mol_type = other DNA
gcttgtatccggagaaactc

SEQ ID NO: 53 - DNA, 20 bp, synthetic construct
  source 1..20: mol_type = other DNA
gttagtggctcactatcgtg

SEQ ID NO: 54 - DNA, 19 bp, synthetic construct
  source 1..19: mol_type = other DNA
gtttggacgtgctagacag

SEQ ID NO: 55 - DNA, 19 bp, synthetic construct
  source 1..19: mol_type = other DNA
ggatcgacttgtcgttatc

SEQ ID NO: 56 - DNA, 19 bp, synthetic construct
  source 1..19: mol_type = other DNA
gcatatcatgcaaacagcg

SEQ ID NO: 57 - DNA, 18 bp, synthetic construct
  source 1..18: mol_type = other DNA
ccattttcggtcgaatgc

SEQ ID NO: 58 - DNA, 18 bp, synthetic construct
  source 1..18: mol_type = other DNA
caatgcaggaggaaaacg

SEQ ID NO: 59 - DNA, 20 bp, synthetic construct
  source 1..20: mol_type = other DNA
ttgcttatacagcagctgtg

SEQ ID NO: 60 - DNA, 20 bp, synthetic construct
  source 1..20: mol_type = other DNA
gccaaactaacactgaggtc

SEQ ID NO: 61 - DNA, 20 bp, synthetic construct
  source 1..20: mol_type = other DNA
tggactctctacgtgaatgt

SEQ ID NO: 62 - DNA, 21 bp, synthetic construct
  source 1..21: mol_type = other DNA
tttccttctttctaccccttt

SEQ ID NO: 63 - DNA, 21 bp, synthetic construct
  source 1..21: mol_type = other DNA
gtttccctgttttccttagat

SEQ ID NO: 64 - DNA, 18 bp, synthetic construct
  source 1..18: mol_type = other DNA
tgtggcctcggagtcctt

SEQ ID NO: 65 - DNA, 19 bp, synthetic construct
  source 1..19: mol_type = other DNA
agtgatgccctttgagatg

SEQ ID NO: 66 - DNA, 19 bp, synthetic construct
  source 1..19: mol_type = other DNA
agacaaggcaaggattgtg

SEQ ID NO: 67 - DNA, 18 bp, synthetic construct
  source 1..18: mol_type = other DNA
cacagaggccctcgcaac

SEQ ID NO: 68 - DNA, 18 bp, synthetic construct
  source 1..18: mol_type = other DNA
ggatgcgtccacggaaac

SEQ ID NO: 69 - DNA, 21 bp, synthetic construct
  source 1..21: mol_type = other DNA
caatctcaggactcacgggag

SEQ ID NO: 70 - DNA, 18 bp, synthetic construct
  source 1..18: mol_type = other DNA
gttgcgagggcctctgtg

SEQ ID NO: 71 - DNA, 18 bp, synthetic construct
  source 1..18: mol_type = other DNA
gtttccgtggacgcatcc

SEQ ID NO: 72 - DNA, 21 bp, synthetic construct
  source 1..21: mol_type = other DNA
ctcccgtgagtcctgagattg

SEQ ID NO: 73 - DNA, 22 bp, synthetic construct
  source 1..22: mol_type = other DNA
gaggcttatgcaatcggtaaga

SEQ ID NO: 74 - DNA, 21 bp, synthetic construct
  source 1..21: mol_type = other DNA
acctggatggatcatggaaac

SEQ ID NO: 75 - DNA, 24 bp, synthetic construct
  source 1..24: mol_type = other DNA
gtcaagactgatgctagaggtaag

SEQ ID NO: 76 - DNA, 23 bp, synthetic construct
  source 1..23: mol_type = other DNA
ccagatgttgcagaaaccataaa

SEQ ID NO: 77 - DNA, 22 bp, synthetic construct
  source 1..22: mol_type = other DNA
cccctgctgaaagattatctac

SEQ ID NO: 78 - DNA, 52 bp, synthetic construct
  source 1..52: mol_type = other DNA
gcagagacagaatgctgtgccatatatctaccatttcagtcaacttttcgcc

SEQ ID NO: 79 - DNA, 51 bp, synthetic construct
  source 1..51: mol_type = other DNA
gaattgattgaaggcgaaaagttgactgaaatggtagatatatggcacagc

SEQ ID NO: 80 - DNA, 18 bp, synthetic construct
  source 1..18: mol_type = other DNA
tcttccacaggttcaagg

SEQ ID NO: 81 - DNA, 21 bp, synthetic construct
  source 1..21: mol_type = other DNA
cattgttggaccacgtcaagg

SEQ ID NO: 82 - DNA, 23 bp, synthetic construct
  source 1..23: mol_type = other DNA
aacgagaatgattggtttttctg

SEQ ID NO: 83 - DNA, 45 bp, synthetic construct
  source 1..45: mol_type = other DNA
acacaaacaaacacaattacaaaaaatgttcttggccatcaagtc

SEQ ID NO: 84 - DNA, 45 bp, synthetic construct
  source 1..45: mol_type = other DNA
gattgggtaaggacttgtagatcaaacatagatcaacgtaatgaa

SEQ ID NO: 85 - DNA, 45 bp, synthetic construct
  source 1..45: mol_type = other DNA
cacacaaacaaacacaattacaaaaaatgcctgctatctccaacg

SEQ ID NO: 86 - DNA, 45 bp, synthetic construct
  source 1..45: mol_type = other DNA
tcgcaagagatttctttgtagatcaaacatagatcaacgtaatga

SEQ ID NO: 87 - DNA, 45 bp, synthetic construct
  source 1..45: mol_type = other DNA
cacacaaacaaacacaattacaaaaaatgtgttcctcgaaggaag

SEQ ID NO: 88 - DNA, 45 bp, synthetic construct
  source 1..45: mol_type = other DNA
atagattaggtaatgatctttagatcaaacatagatcaacgtaat

SEQ ID NO: 89 - DNA, 45 bp, synthetic construct
  source 1..45: mol_type = other DNA
acaaacaaacacaattacaaaaaatgatttcctttcaaaccaaag

SEQ ID NO: 90 - DNA, 45 bp, synthetic construct
  source 1..45: mol_type = other DNA
gctcgctgctgatttgtagatcaaacatagatcaacgtaatgaat

SEQ ID NO: 91 - DNA, 55 bp, synthetic construct
  source 1..55: mol_type = other DNA
acacacaaaacacacaaacaaacacaattacaaaaaatgcctgcttctgaggctt

SEQ ID NO: 92 - DNA, 45 bp, synthetic construct
  source 1..45: mol_type = other DNA
agattaggtagaaacctctagatcaaacatagatcaacgtaatga

SEQ ID NO: 93 - DNA, 45 bp, synthetic construct
  source 1..45: mol_type = other DNA
acacaaacaaacacaattacaaaaaatgaacaatctccatgcaac

SEQ ID NO: 94 - DNA, 45 bp, synthetic construct
  source 1..45: mol_type = other DNA
gacattgctatcgatatgtagatcaaacatagatcaacgtaatga

SEQ ID NO: 95 - DNA, 45 bp, synthetic construct
  source 1..45: mol_type = other DNA
acacaaacaaacacaattacaaaaaatgtcctccgttactaagac

SEQ ID NO: 96 - DNA, 45 bp, synthetic construct
  source 1..45: mol_type = other DNA
gagaagaatgtgagtactagatcaaacatagatcaacgtaatgaa

SEQ ID NO: 97 - DNA, 45 bp, synthetic construct
  source 1..45: mol_type = other DNA
caaacaaacacaattacaaaaaatgcttactattttatttcgttc

SEQ ID NO: 98 - DNA, 45 bp, synthetic construct
  source 1..45: mol_type = other DNA
aattgggcgaagatctttagatcaaacatagatcaacgtaatgaa

SEQ ID NO: 99 - DNA, 45 bp, synthetic construct
  source 1..45: mol_type = other DNA
cacaaacaaacacaattacaaaaaatggaacgtcacattaatggt

SEQ ID NO: 100 - DNA, 45 bp, synthetic construct
  source 1..45: mol_type = other DNA
ttcgttggctaacgcgtagatcaaacatagatcaacgtaatgaat

SEQ ID NO: 101 - DNA, 55 bp, synthetic construct
  source 1..55: mol_type = other DNA
catcatcatcatcatcattagaagcctctcttgaatcagctggttgtgtatgtat

SEQ ID NO: 102 - DNA, 47 bp, synthetic construct
  source 1..47: mol_type = other DNA
gtatatcttaaaacacacacacaaaatggaacttatgatcgttcaag

SEQ ID NO: 103 - DNA, 45 bp, synthetic construct
  source 1..45: mol_type = other DNA
gtatatcttaaaacacacacacaaaatgtcctcggaaaccattag

SEQ ID NO: 104 - DNA, 47 bp, synthetic construct
  source 1..47: mol_type = other DNA
gtatatcttaaaacacacacacaaaatgtcgatctcagagaaatatt

SEQ ID NO: 105 - DNA, 45 bp, synthetic construct
  source 1..45: mol_type = other DNA
gtatatcttaaaacacacacacaaaatgagtttgaacgacgatgc

SEQ ID NO: 106 - DNA, 47 bp, synthetic construct
  source 1..47: mol_type = other DNA
gtatatcttaaaacacacacacaaaatggaaaacgtgaaataccaaa

SEQ ID NO: 107 - DNA, 47 bp, synthetic construct
  source 1..47: mol_type = other DNA
gtatatcttaaaacacacacacaaaatggaagacaaaagaaaaggtg

SEQ ID NO: 108 - DNA, 47 bp, synthetic construct
  source 1..47: mol_type = other DNA
gtatatcttaaaacacacacacaaaatgagtaaaagtgaaaacctct

SEQ ID NO: 109 - DNA, 45 bp, synthetic construct
  source 1..45: mol_type = other DNA
tatttagtaaaagcattagttagatttactcatcggaaaaccagt

SEQ ID NO: 110 - DNA, 45 bp, synthetic construct
  source 1..45: mol_type = other DNA
cacctgtaactaaaacaatcattgtgtttgtgttgggggtggtag

SEQ ID NO: 111 - DNA, 45 bp, synthetic construct
  source 1..45: mol_type = other DNA
atttagtaaaagcattagttagatttaacccttgtggacctttaa

SEQ ID NO: 112 - DNA, 45 bp, synthetic construct
  source 1..45: mol_type = other DNA
aaagataatgagcctggcattgtgtttgtgttgggggtggtagta

SEQ ID NO: 113 - DNA, 45 bp, synthetic construct
  source 1..45: mol_type = other DNA
tttagtaaaagcattagttagatttatgccttatgaaccttcaaa

SEQ ID NO: 114 - DNA, 55 bp, synthetic construct
  source 1..55: mol_type = other DNA
gcctgtaacaaagacaatcattgtgtttgtgttgggggtggtagtaatagtagta

SEQ ID NO: 115 - DNA, 45 bp, synthetic construct
  source 1..45: mol_type = other DNA
tttagtaaaagcattagttagatttatggattcttatgagtaggt

SEQ ID NO: 116 - DNA, 45 bp, synthetic construct
  source 1..45: mol_type = other DNA
tgagacaagaatatgagccattgtgtttgtgttgggggtggtagt

SEQ ID NO: 117 - DNA, 45 bp, synthetic construct
  source 1..45: mol_type = other DNA
atttagtaaaagcattagttagatttagccttttctgtgaacaga

SEQ ID NO: 118 - DNA, 45 bp, synthetic construct
  source 1..45: mol_type = other DNA
tgatgaataatgttggagtcattgtgtttgtgttgggggtggtag

SEQ ID NO: 119 - DNA, 45 bp, synthetic construct
  source 1..45: mol_type = other DNA
tatttagtaaaagcattagttagatttagtcgacatccctaacga

SEQ ID NO: 120 - DNA, 45 bp, synthetic construct
  source 1..45: mol_type = other DNA
aaccaaaacagtatcggtcattgtgtttgtgttgggggtggtagt

SEQ ID NO: 121 - DNA, 45 bp, synthetic construct
  source 1..45: mol_type = other DNA
atttagtaaaagcattagttagatttagccaacatgtctattaac

SEQ ID NO: 122 - DNA, 45 bp, synthetic construct
  source 1..45: mol_type = other DNA
accaagacggttaaaggcattgtgtttgtgttgggggtggtagta

SEQ ID NO: 123 - DNA, 45 bp, synthetic construct
  source 1..45: mol_type = other DNA
atttagtaaaagcattagttagatttaatctctgtggacttgcaa

SEQ ID NO: 124 - DNA, 45 bp, synthetic construct
  source 1..45: mol_type = other DNA
accatggctgttttggtcattgtgtttgtgttgggggtggtagta

SEQ ID NO: 125 - DNA, 45 bp, synthetic construct
  source 1..45: mol_type = other DNA
tatttagtaaaagcattagttagatttacgaagcaccaggtctat

SEQ ID NO: 126 - DNA, 45 bp, synthetic construct
  source 1..45: mol_type = other DNA
taataaaaacggttctggacattgtgtttgtgttgggggtggtag

SEQ ID NO: 127 - DNA, 45 bp, synthetic construct
  source 1..45: mol_type = other DNA
ttagtaaaagcattagttagatttagaatttttcaatagccaaac

SEQ ID NO: 128 - DNA, 45 bp, synthetic construct
  source 1..45: mol_type = other DNA
acaaatgcggtcttcttcattgtgtttgtgttgggggtggtagta

SEQ ID NO: 129 - DNA, 45 bp, synthetic construct
  source 1..45: mol_type = other DNA
aaagattgggtaaggacttgtagatcaaacatagatcaacgtaat

SEQ ID NO: 130 - DNA, 45 bp, synthetic construct
  source 1..45: mol_type = other DNA
tatcgcaagagatttctttgtagatcaaacatagatcaacgtaat

SEQ ID NO: 131 - DNA, 45 bp, synthetic construct; identical to SEQ ID NO: 88
  source 1..45: mol_type = other DNA

SEQ ID NO: 132 - DNA, 45 bp, synthetic construct
  source 1..45: mol_type = other DNA
agaagctcgctgctgatttgtagatcaaacatagatcaacgtaat

SEQ ID NO: 133 - DNA, 55 bp, synthetic construct
  source 1..55: mol_type = other DNA
cagaatttgaaagattaggtagaaacctctagatcaaacatagatcaacgtaatg

SEQ ID NO: 134 - DNA, 45 bp, synthetic construct
  source 1..45: mol_type = other DNA
ttgacattgctatcgatatgtagatcaaacatagatcaacgtaat

SEQ ID NO: 135 - DNA, 45 bp, synthetic construct
  source 1..45: mol_type = other DNA
taggagaagaatgtgagtactagatcaaacatagatcaacgtaat

SEQ ID NO: 136 - DNA, 45 bp, synthetic construct
  source 1..45: mol_type = other DNA
cgcaattgggcgaagatctttagatcaaacatagatcaacgtaat

SEQ ID NO: 137 - DNA, 45 bp, synthetic construct
  source 1..45: mol_type = other DNA
ctttttcgttggctaacgcgtagatcaaacatagatcaacgtaat

SEQ ID NO: 138 - DNA, 55 bp, synthetic construct
  source 1..55: mol_type = other DNA
tattattattattattattattatcatattcaattcatcattttttttttattct

SEQ ID NO: 139 - DNA, 55 bp, synthetic construct
  source 1..55: mol_type = other DNA
ttattattattattattattattatcatattcaattcatcattttttttttattc

SEQ ID NO: 140 - DNA, 55 bp, synthetic construct
  source 1..55: mol_type = other DNA
tataattttataataaaaagtttaaacttgatttaacctgatccaaaaggggtat

SEQ ID NO: 141 - DNA, 55 bp, synthetic construct
  source 1..55: mol_type = other DNA
ataatatataattttataataaaaagtttaaacttgatttaacctgatccaaaag

SEQ ID NO: 142 - DNA, 45 bp, synthetic construct
  source 1..45: mol_type = other DNA
acaaacaaacacaattacaaaaaatgttcttggccatcaagtctt

SEQ ID NO: 143 - DNA, 45 bp, synthetic construct
  source 1..45: mol_type = other DNA
acaaacaaacacaattacaaaaaatgcctgctatctccaacggct

SEQ ID NO: 144 - DNA, 45 bp, synthetic construct; identical to SEQ ID NO: 87
  source 1..45: mol_type = other DNA

SEQ ID NO: 145 - DNA, 45 bp, synthetic construct
  source 1..45: mol_type = other DNA
cacacaaacaaacacaattacaaaaaatgatttcctttcaaacca

SEQ ID NO: 146 - DNA, 55 bp, synthetic construct
  source 1..55: mol_type = other DNA
cacaaacaaacacaattacaaaaaatgcctgcttctgaggcttccgccgaaaggc

SEQ ID NO: 147 - DNA, 45 bp, synthetic construct
  source 1..45: mol_type = other DNA
cacacaaacaaacacaattacaaaaaatgaacaatctccatgcaa

SEQ ID NO: 148 - DNA, 45 bp, synthetic construct
  source 1..45: mol_type = other DNA
cacacaaacaaacacaattacaaaaaatgtcctccgttactaaga

SEQ ID NO: 149 - DNA, 45 bp, synthetic construct
  source 1..45: mol_type = other DNA
cacacaaacaaacacaattacaaaaaatgcttactattttatttc

SEQ ID NO: 150 - DNA, 45 bp, synthetic construct
  source 1..45: mol_type = other DNA
cacacaaacaaacacaattacaaaaaatggaacgtcacattaatg

SEQ ID NO: 151 - DNA, 55 bp, synthetic construct
  source 1..55: mol_type = other DNA
agaataatatataattttataataaaaagtttaaaccgaaaaatgcaccacaccc

SEQ ID NO: 152 - DNA, 55 bp, synthetic construct
  source 1..55: mol_type = other DNA
atatcttaaaacacacacacaaaatggaacttatgatcgttcaagtcacagagca

SEQ ID NO: 153 - DNA, 55 bp, synthetic construct
  source 1..55: mol_type = other DNA
atatcttaaaacacacacacaaaatgtcctcggaaaccattagtcaatcaattaa

SEQ ID NO: 154 - DNA, 55 bp, synthetic construct
  source 1..55: mol_type = other DNA
atatcttaaaacacacacacaaaatgtcgatctcagagaaatattttccacaaga

SEQ ID NO: 155 - DNA, 55 bp, synthetic construct
  source 1..55: mol_type = other DNA
atatcttaaaacacacacacaaaatgagtttgaacgacgatgccacattttggcg

SEQ ID NO: 156 - DNA, 55 bp, synthetic construct
  source 1..55: mol_type = other DNA
atatcttaaaacacacacacaaaatggaaaacgtgaaataccaaattccacctgc

SEQ ID NO: 157 - DNA, 55 bp, synthetic construct
  source 1..55: mol_type = other DNA
atatcttaaaacacacacacaaaatggaagacaaaagaaaaggtgagagaaagcg

SEQ ID NO: 158 - DNA, 55 bp, synthetic construct
  source 1..55: mol_type = other DNA
atatcttaaaacacacacacaaaatgagtaaaagtgaaaacctctataatgcagc

SEQ ID NO: 159 - DNA, 55 bp, synthetic construct
  source 1..55: mol_type = other DNA
tataattttataataaaaagtttaaaccgaaaaatgcaccacacccggagaaaaa

SEQ ID NO: 160 - DNA, 50 bp, synthetic construct
  source 1..50: mol_type = other DNA
tcttcaccagacaagcatttgaacattcaattcatcattttttttttatt

SEQ ID NO: 161 - DNA, 18 bp, synthetic construct
  source 1..18: mol_type = other DNA
cagacaagcatttgaaca

SEQ ID NO: 162 - DNA, 55 bp, synthetic construct
  source 1..55: mol_type = other DNA
aatgcttagcccaacatcatcatcatcatcattagaagcctctcttgaatcagct

SEQ ID NO: 163 - DNA, 55 bp, synthetic construct
  source 1..55: mol_type = other DNA
tgaagaagctcacgcatcatcatcatcatcattagaagcctctcttgaatcagct

SEQ ID NO: 164 - DNA, 55 bp, synthetic construct
  source 1..55: mol_type = other DNA
cattttccaagatccatcatcatcatcatcattagaagcctctcttgaatcagct

SEQ ID NO: 165 - DNA, 55 bp, synthetic construct
  source 1..55: mol_type = other DNA
ttgaacgtgcacttcatcatcatcatcatcattagaagcctctcttgaatcagct

SEQ ID NO: 166 - DNA, 55 bp, synthetic construct
  source 1..55: mol_type = other DNA
tcaagaagtccttacatcatcatcatcatcattagaagcctctcttgaatcagct

SEQ ID NO: 167 - DNA, 55 bp, synthetic construct
  source 1..55: mol_type = other DNA
aacgtggtgttcgtcatcatcatcatcatcattagaagcctctcttgaatcagct

SEQ ID NO: 168 - DNA, 55 bp, synthetic construct
  source 1..55: mol_type = other DNA
cctttgcaaaattacatcatcatcatcatcattagaagcctctcttgaatcagct

SEQ ID NO: 169 - DNA, 55 bp, synthetic construct
  source 1..55: mol_type = other DNA
atacacacttaatacaatacacatacaatttcaattcatcattttttttttattc

SEQ ID NO: 170 - DNA, 55 bp, synthetic construct
  source 1..55: mol_type = other DNA
tataattttataataaaaagtttaaactggaacatgtttgtacaaatatttaagt

SEQ ID NO: 171 - DNA, 55 bp, synthetic construct
  source 1..55: mol_type = other DNA
cacttaatacaatacacatacaatttcaattcatcattttttttttattcttttt

SEQ ID NO: 172 - DNA, 55 bp, synthetic construct
  source 1..55: mol_type = other DNA
gaagcctgccgtggcacctgtaactaaaacaatcattgtgtttgtgttgggggtg

SEQ ID NO: 173 - DNA, 55 bp, synthetic construct
  source 1..55: mol_type = other DNA
tttcgaaagtgttgcgaaagataatgagcctggcattgtgtttgtgttgggggtg

SEQ ID NO: 174 - DNA, 55 bp, synthetic construct
  source 1..55: mol_type = other DNA
aaaacctgctgtggcgcctgtaacaaagacaatcattgtgtttgtgttgggggtg

SEQ ID NO: 175 - DNA, 55 bp, synthetic construct
  source 1..55: mol_type = other DNA
tcccgaggtagcacctgagacaagaatatgagccattgtgtttgtgttgggggtg

SEQ ID NO: 176 - DNA, 55 bp, synthetic construct
  source 1..55: mol_type = other DNA
agatgttgcaccggtgatgaataatgttggagtcattgtgtttgtgttgggggtg

SEQ ID NO: 177 - DNA, 55 bp, synthetic construct
  source 1..55: mol_type = other DNA
agcagttgcacctgtaaccaaaacagtatcggtcattgtgtttgtgttgggggtg

SEQ ID NO: 178 - DNA, 55 bp, synthetic construct
  source 1..55: mol_type = other DNA
agcggtagcacctgtgaccaagacggttaaaggcattgtgtttgtgttgggggtg

SEQ ID NO: 179 - DNA, 55 bp, synthetic construct
  source 1..55: mol_type = other DNA
cgaagtagcaccggtaaccatggctgttttggtcattgtgtttgtgttgggggtg

SEQ ID NO: 180 - DNA, 55 bp, synthetic construct
  source 1..55: mol_type = other DNA
tgcggatgcacctgtaataaaaacggttctggacattgtgtttgtgttgggggtg

SEQ ID NO: 181 - DNA, 55 bp, synthetic construct
  source 1..55: mol_type = other DNA
ggaggttgcgccagtgacaaatgcggtcttcttcattgtgtttgtgttgggggtg

SEQ ID NO: 182 - DNA, 55 bp, synthetic construct
  source 1..55: mol_type = other DNA
taatatataattttataataaaaagtttaaactggaacatgtttgtacaaatatt

SEQ ID NO: 183 - DNA, 55 bp, synthetic construct
  source 1..55: mol_type = other DNA
atttagtaaaagcattagttagatttactcatcggaaaaccagtcttggttttct

SEQ ID NO: 184 - DNA, 55 bp, synthetic construct
  source 1..55: mol_type = other DNA
atttagtaaaagcattagttagatttaacccttgtggacctttaaacccgccaag

SEQ ID NO: 185 - DNA, 55 bp, synthetic construct
  source 1..55: mol_type = other DNA
attggctggtttgaaggttcataaggcataaatctaactaatgcttttactaaat

SEQ ID NO: 186 - DNA, 55 bp, synthetic construct
  source 1..55: mol_type = other DNA
atttagtaaaagcattagttagatttatggattcttatgagtaggtaatggtgca

SEQ ID NO: 187 - DNA, 55 bp, synthetic construct
  source 1..55: mol_type = other DNA
atttagtaaaagcattagttagatttagccttttctgtgaacagagaaaccggcc

SEQ ID NO: 188 - DNA, 55 bp, synthetic construct
  source 1..55: mol_type = other DNA
atttagtaaaagcattagttagatttagtcgacatccctaacgacagacaaaccg

SEQ ID NO: 189 - DNA, 55 bp, synthetic construct
  source 1..55: mol_type = other DNA
atttagtaaaagcattagttagatttagccaacatgtctattaacggccaaacct

SEQ ID NO: 190 - DNA, 55 bp, synthetic construct
  source 1..55: mol_type = other DNA
atttagtaaaagcattagttagatttaatctctgtggacttgcaatcctgccgct

SEQ ID NO: 191 - DNA, 55 bp, synthetic construct
  source 1..55: mol_type = other DNA
atttagtaaaagcattagttagatttacgaagcaccaggtctatgcacagcgaga

SEQ ID NO: 192 - DNA, 55 bp, synthetic construct
  source 1..55: mol_type = other DNA
atttagtaaaagcattagttagatttagaatttttcaatagccaaaccagcccac

SEQ ID NO: 193 - DNA, 1797 bp, Terrapene sp.
  source 1..1797: mol_type = genomic DNA; note = Terrapene carolina triunguis
atgttcttggccatcaagtctttgcaattgtctagacagaaacaaaagattggtaggagaaagaagtcgactgatagatggtccgacctcttaacagatatcttgcaaggtatttttctctgttgcgccgtcttacagcgttcaattgacatggacttcagaacgggcgctaataaaaccaatgagcgtgtccatctcgaaccatctcagttaagagaaaagcatatttcctcgtttcagggtgaatcggaagagaaagaaaaggatactaacgcagagattttggtggctaacttctccaatatctacgcatctgatttgttacctactagaaatggcgaggagttcaccgatcaatttttgttggaggtcgttgaaatcctcctcaactacatcaagaagacgtatgatggtaagtctaaagttttggattttcaccaccctcaccaattattggagggtttggaaggtttttcattggaattgagagatcaacctgaaccattagagcaaatcttagttgactgtcgtgataccttaaaatacggtgttaagactggtcatccaaggtacttcaaccaattgtcttccggtttagacattgtcggtttggctggcgagtggttgtcagctactgcgaacaccaatatgttcacatatgagattgccccagttttcgtcgtgatggaagaaattttgttaaagaagatgcatgaaatggttggatggggagaaatggaagcagacggaattttctcacctggtggtaccatttctaacctttactctgtgttagttgcaaggtataagagatttccagaaattaaaaccaagggtatggcggctttgcctcatatctctttgttcgtctcagaacactcccattactctgtcaaaaaggcagcagccgttttaggtatcggaactgataacgttattgcagttaagtgtgacgagagaggtaagatgatcccatcagatcttgaagccaaaatcttaaaggtcaagagacaagatcatatccctctctatgtctcggccactgccggcactacagtttacggtgcgttcgacccattggataatatcgcagacatttgtgaaaagtatggtttgtggatgcatgttgatgcggcttggggtggtggcttgttaatgtctagaaaacactcgttcaaacttaatggtattgaaagagcgaattcggttacgtggaaccctcacaagcttatgggagttccattacaatgttctgcatttttagtttgtgaaaagggtttgttacaagcttgcaatcaaatgtgtgctggctatcttttccaacctgataaacagtatgataccacatacgatacaggtgataagactattcaatgtggtagacatgtcgatgtttttaagctttggttgatgtggaaagccaagggaactagaggattcgaaattctcatcaacaagtcccttgaattggcagagtatctttacaaggaattaaactcgcgtgagaactttgagttagtttttgatgcagaacctgaattaacaaatgtctgtttttggtacattcctcctaatctcagaagaatcccaaatggcccagaaagggaaaaattattacaccaggtcgcaccaaaaattaaggctcgtatgattgaagaaggtacaacaatggtttcataccagccacatggtgataaggtgaatttctttaggatggtgttttccaacccagctaccagaaagtccgacgtggactttttattggaggaaattgaaagattgggtaaggacttgtag

SEQ ID NO: 194 - DNA, 1536 bp, unidentified
  source 1..1536: mol_type = genomic DNA; note = Trichoderma gamsii
atgcctgctatctccaacggctacgttgcaaacggtaagccagattcacaattaaaccgtgctgctgaaactgccgatcttttggagtcagtccaacagttaattattccttttattaaggctgcagatgacgcagcagcccacagggcatctggtaacctcggtttagatgcaaatggtgagccacagaacgttttgatcgactcccaaaagccatcggagttggtcgccagactcaagttcttgttaccagaggagggattgggtaaggacggcttgcttcagtctattgacaagttgttaaaatactctgtgaatacctgggatcagggtttcatggataagctttacgcatctaccacacctgttggcgtcattgccgatcttatcctctctgtcttgaacactaacgtccacgtctatcatgtgtcgccagcattaaccgtgattgaaaagacaaccacgaaggctttcgctaatctctttaattttgacggtcctcacgcgggtggtatttcatgtccaggtggttcgggttctaatcttacttcattgatcgtcgcaagaaacacattgtaccctgaaaccaaaactaagggtaatggtaaacgtgattttgtcgtttttacctccgcccacggtcattactccgttgaaaaggcagctatgattactggtttaggctcgtcggcggtttgggccgttcctgttgataatgaaggttcgatgaggcctcaagcattgagagaattggttttgttggcaaaaaagcaaggaaagactccattttacgtgaactccacggcgggaacaacagttatgggttcattcgatcctttcgaagaaatttctaaggtgtgtaaggaattcggaatgtggttacatatcgatgcttcctggggcggatcagtcatcttctcgaaggctcagtcctggaagatgaaaggttctcatcttgcagactctttaactgttaatccacacaaaatgttgaacgcgccatgtacgtgttctttgctcttgggtccagatatgaggattttccagagagcgaactctacagctgccggttatctctttcatcgtggagacgatgaagaggtttgggacttggcggatctttcattgcaatgtggtagaagaggtgatgccttgaaggttgccttagcttggttatattacggagcaaatggatttgaaagacaagtcgatcatgcatttgatatggcgtcttatctcttcagattattggaggaatccggtgatttcgttatggtttcttcgtatcctccaccttgtttgcaagtttgcttttattccgctccaggtggcaagctttcatctgacaaaaatgtcaatacctccagaactcaaagaatcgcccatatgttaatcggtagaggtttcatggtcgactacgctccaggtgataaaggctcatttttgagagtggtcgttaatgttcaaactttagaaggtacagttgaaggtatcgttaaggcaatggttgaattatcgcaagagatttctttgtag

SEQ ID NO: 195 - DNA, 1518 bp, Varroa destructor
  source 1..1518: mol_type = genomic DNA
atgtgttcctcgaaggaaggtacggtggtcgtcgagaagctttcctacaaggatttgtatccatacaatattaacggaaaggcggagacacgtgcttttttattgaaggttgtgtctattctcttagactatgttgatgaagaaaacgacaggacatctaagattctcgacttcaagatgccagaagaattggaacagattttggaccttaacttgcctgataagggtttaccattgggagatttgttaaaggactgcaggtcgacgttaaaatggcaagttaagaccggtcatccacatttcttcaaccaattgtcatccggattagatattgtttcgttagccggtgagtggttgacctctgttgcaaatactaatatgttcacctacgagatcgcacctgtttttatcttgatggagaatgttgtcttgaagaagatgagagaataccttggcttcgtccatggtgattcaattttggctcctggtggttcaatttcaaacatgtacgcggttatgggtgcaagacataaaatgttcccaaattataagaaattaggattgaaggccttgcctcaattagtcatgtacacatctgttgattcacattattccgctatgggtgctgctgcctcgtgtggtttaggtactgacaatgtgatccaaattggtaccgattccatgggtcgtatgagggttgacaaactcgaagaagcagttttggcaaccaaagcgaaaggtcacgtcccattctttgtcaattgtacttgtggcaccactgttgttggtgcatttgatccaattaacccaattgctgatctctgccaaaaatatggtatgtggttacatatcgatgccgcatggggtggtggtgttttgttatctaagaaacacagacatttgatggcaggagtcgagagagcaaattccgtcacatggaaccctcacaagcttatgggcacccatcttcagtgttctactatccactttagacaagatggcattttgttaggctgtaatcagatgtgtgctgaatacttgtttcaacaagacaagcattatgacgtctctttcgatactggtgacaaggtccctcaatgtggaagaagaaacgatatcttcaagctttggttaatgtggagagccaagggcacagaaggttttgaagcacacatcgaccagttatttgatatctcccagtacctcgtgaaaaggattaaggaaatgcctgagaaattccacctcatccaagaaccagagatggtgaatgttttgttttggtatatcccacaacgtttaagagatcagcctcaatctaaggaacgtatcgctgaattaggtagaattacggcccaactcaaggcgagaatgatgaacactggaacattgatgattacttaccagccattgagagaattgccaaatttttttagaaacatcgtttctaactcggcggttacaaaggatgatattgatttcttgttagccgagatggatagattaggtaatgatctttag

SEQ ID NO: 196 - DNA, 999 bp, Anopheles sinensis
  source 1..999: mol_type = genomic DNA
atgatttcctttcaaaccaaaggactttttggtttacctagattggttctcttcacctctgaagacgcccactactccatcaaaaagttggcgtcgtttatgggtattggtttcgataatgttttcgcaatcaagacagatgcccgtggcaaaatgtgtttgactgatcttgaagaacaaattaagcgtgccttaacggaaggagctcagcctttcatggtttctgcaactgcaggtactacagttttgggcgcctttgacccattggaaggtattgctgagttatgtgccaagtataaactttggatgcacgtcgatgccgcatggggtggaggtgcattgatgtccaagaagtataggcatcttttaaaaggtatcgagagagctgattcggtcacttggaatccacataagctcttaactgcaccacaacaatgttctacattcttgaccaggcatgagaatatcttgtcacagtgcaattccacaaacgcaacttatctctttcaaaaggacaagttctacgatacgaagtacgatacgggagacaagcatatccagtgcggtagaagagctgatgtgttaaaattctggtttatgtggagagcaaagggtaccaaaggtttggaacaacatgttgacacagcgttcgattgtgcagagttttttacacaaaccatcaagcaaagacctggttacaagatggtcgttcatgagccagaatgtaccaatgtctgtttttggtatcttcctccatcattacaatcaagatctgaaccaaagactgctgaagaagcgactgaatttcgtgatgctttacacagagtggcgcctaaggtcaaggaaagaatgatgaaatcgggtgccatgatgattacctaccagccaattcatgataagcctaacttcttcagattggttatccagaactcgggcttgaaccactctgacatgattcacattgcagacacaattgagaagctcgctgctgatttgtag

SEQ ID NO: 197 - DNA, 1542 bp, unidentified
  source 1..1542: mol_type = genomic DNA; note = Apolygus lucorum
atgcctgcttctgaggcttccgccgaaaggcctagagccgacgttgtcattgaggaaattgacggtgcatcctctaaggaggcctttttatcggctccagacaaagaaatccacggacaattcttgcatagagtccaacagatgctcttggatcacgcattccaatcaactcacagagaatccaaggttacaagatatgttgacccagatcgtttgagagagatcatcgatatgaatcttagaaaggaaccagcgtccgaagaggcattattgtcatacatcagagatactatgaagtactcggttaaaaccggacatcctttttttgtcaatcaattattttcgtccctcgacccatacgctttagcaggtgcgtgggttacagattctttgaacccatcagtttacacgtatgaggtttctccagttttaacattgatggaggaatcagtcttaaaggaaatgagatccatcgttggatggtcagacggtgacggtatcttcgctcctggtggctctatcgccaacggctatgcaatttcgtgtgcgaggttccataagattccagatattaaggctaaaggcttgcatggattgccaaggctcgttttgttcacttcggaggatgcacactattcgattaagaaattggcggcattcgagggtttgggttctgataatgtctacccagtcaagactgatgctagaggtaagatggacgttttgcatcttagggagttggtccaacgtgccaaagcagaaggagccgttccttttatggtttctgcaacatctggtaccacagtcttaggtgcttttgatcctttggaagccattcatgaagtgtgtgaagaggaaggtatgtggttgcacgtcgatgctgcatggggcggtggtgcattagtttccaagaagtacagacaccttttgagaggcattgagaaggcagactctgttacttggaatcctcacaagatgttggctgcaccacagcagtgctccactctcttaacaaagcataaaacgattttagcggaatgtcattctgcctctgctcaataccttttccaaaaagacaagttctatgatacctcctttgataccggtgataagcatatccagtgtggtagacgtgcggatgtgttcaagttttggttgatgtggaaagcgaagggtaccgatggtttagaaaagcatgtcaaccgtgtttatgaaaatgctgaatacttcaccgaaaagattagaagaagacctggattcaagcttgttttagaagagcctgaatgtactaacgttactttttggtacgtcccaccttcaattagaggtagagataatgaaccagaatataatcaaaagttgcataaggtggcacctcttatcaaagaaaggatgactcgtgaaggtacaatgttgatgacctatcaaccattaaaagaattacctaacttttttagattggtgatccagaactctgccctcacaacgtcggatatggattacattgtgtcagaatttgaaagattaggtagaaacctctag

SEQ ID NO: 198 - DNA, 1491 bp, unidentified
  source 1..1491: mol_type = genomic DNA; note = Strongyloides ratti
atgaacaatctccatgcaaccgatcttttgccagtcaatcaagacggatggaaggagacagaaacctttctcaaggaagttgtcgaaattctcttgtcctacatcaagacgcagaatgacaggtcgtcaaaggttttagaatttcaccacccacaggatatgttgaagttaattgacctctccatccctgataagccaatgtctttagatgatcttttgaactcgtgccaagaagttttaaagttgggtgtgaagactggtcatcctagattttttaatcaaatctcttgcggtttagatttggtttgtatggccggagagtggcttacagctacagcgaacaccaacatgttcacctacgaaatttctccagtgttcattttgatggaaaaggaggtcatgaggaagatgattcagttgatcggctggggtgatgatgaaggtgacggtattttttcacctggtggtgctattgctaatctttacgcaatgaatgcagcgagacataatgctttcccacgttgtaagcctttgggtatgacttccgtcccaactttgtgctgtttcacatcgattgattcgcactattcaatcaagggtgctgcggcggttactggaatcggcactgataattgttttaacattccaacagataagctcggtagaatgattccagaggccttagagcagaagatcatcgaatgtaaaaaagacggtttgtatccttttttcgtcgcagcaacggccggtacaacggtttatggtgcttttgaccctttgatccaaatcgcagacatttgtgaaaagtacggtgtttggtttcatgttgacgctgcctggggcggtggtttgttgctttctcctgagcataagtataaacttagaggtatcgagagagcaaactcggttacttggaatccacacaaacttatgggatctttattacaatgttctgcatgttttattaggaaggacggtttattgttccaaaccaatcagatgtccgcagattatctcttccaacaggataagccatacgatgtttcatatgatactggcgatcgttcaattcaatgtggccgtagaaatgacgttttaaaattgtggttaatgtggagatccaaaggtatggaaggttaccgtaaacaaattaacaggttgttggacttggcacaatacttcacatccaagatcaaaagaactgagggctatgaaatggtcattgaagatccacaattcttaaacatttgtttttggtacgtgcctaagagattgagaaatatgcctcaaaaggaaaaaatggccagattggaaaagatcgccccaaagattaaaggaaagatgatggaaagaggaaccactatggtcggttaccaaccagataacaacacccctaactttttcagattaattatcaacaatcctgccacaaccgaagaggatcttgatttcttaatccaggaaattattgacattgctatcgatatgtag

SEQ ID NO: 199 - DNA, 1548 bp, Bactrocera dorsalis
  source 1..1548: mol_type = genomic DNA
atgtcctccgttactaagactactgccacaacgcaaaataaagttgacgaaattgataagatcagacttaacttggccgatgattggaaggttttaggttcagtctatgatttgttgaaagacggtaaggctttctgtgtctcagatgccgatagagatgcagttaaaatcgttgattttaaacagccaaatgagatgaagcaattaattgacctttccatcgacgaaaataagacacatactatcgctgaaatcgaagatctttgtagattggtgatcaagtactctgtcaagacgaaccatggtagatttcataaccagttatttggtcagatggatccatacggattgatgggccaatggattactgacgcgttgaacgcttcagcttatacatacgaagtcggtcctgtcttctccttgatggaaactgaaattatcagaacagtttgccaattagcgggctatgcagagggtgatggtattttctctcctggtggatcgatgtctaacatgtacggtttagtcctcgcacgttactccagacacccacaagttaagaccgcgggtttgtttggtatgaggcctttagcgttgttcacctcggaagaatcgcattattccttcgttaaggcagcacattggttaggtttaggcgcggacaattgcgttgtggttaaaaccaatgagaggggtcaaatgctcacaaatgaccttgaagctaagattttgaaggctcaagagtctggttgtgaaccttttttcgttaatgcaactgcaggtactaccgttttgggtgctttcgatgatcttaacgcaattgctgatgtgtgtgagaagtatggtttgtggttacatgtcgatgcatgtttaggtggatcggcactcttatcgtatagaaatagacaccttctcgccggtttggacagaacaaactcttttgcgtggaacccacacaagtctttaggtgccccacttcaatgttctttgtttttaaccagagagcgtgacctcttgactcgttgtaattcaatccaagtcaactaccttttccaacaggataagttttatgatgtttcctacgataccggtaataagtctgtccagtgtggcagaaaaattgatgctttcaagttttggttgatgttgaaggcaagaggttacggatcattcggacgtttgttagatcacgccattgatatttcaaaagtgctcgttgaaaagattaagcaaaggggctcggagcacttcaggctcgttttggaagacaatcagtacatgaacgtgtgtttctggtacattcctaaagccttaagaggtttggaggagacacctgaatggcgtgaaagattgtacacagtcgccccaaagattaaagaaagaatggcatattctggaaccgtcatgatcggttacaccccattgcaatatagaggcttaggtaatttttttagaatggtttttacgtgctttccagttttagaagaagctgagttggacttcatcttgaacgaaatcgaaaggttaggagaagaatgtgagtactag

SEQ ID NO: 200 - DNA, 1512 bp, Lutzomyia sp.
  source 1..1512: mol_type = genomic DNA; note = Lutzomyia longipalpis
atgcttactattttatttcgttcttggccacatctttgggtgttagttccaattatgtgtactcaatacgttcaaatgcatgcagtcaagtggaagtggattattttagaatccgaaattttgagagccaagtgtgagggtggccatcctttcatggtttctgcaactgctggtactacagtcttgggagcttttgaccctttgaccgaaatcgcgaacttgtgtgaaaaatatcaattgtggtttcatgtcgatgcagcatggggtggtggtgcattagtttctccaaagtatagagccttgttggccggaatcgagagagctgattcggtgacctggaaccctcataagctcttggctgcaccacaacaatgttcgactttcttaacgagacacccaaacttgttgaaacagtgtcattcatgcaatgcatcttacctttttcaaaaggataagttttacgatactaagtacgacacgggcgacaagcatattcagtgtggtagaagggctgatgttttcaagttttggctcatgtggaaggcgaaaggtaccttgggattgcaaaggcacgccgagaaggtcttcgagatggcagaattcttcaccgaacagattagacagagggacgattttgagatggttgtcgcggaaccagaatgcacaaatgtttgcttctggtatctccctccttctcttcgttcctgtccaagggatgaagagttcttaactaagctccacagagtggcacctaagatcaaagaaagaatgatgaaggagggttcgatgatgatcacgtaccagcctcttcgtcaaaaaccaaacttcttcaggttagtgttgcagaactcatccttggaaaagtcagatatgctccatatcattaacaaaatcgcccagttaggtgaagacctcgcagattctgttacatggaatcctcataaacttttggctgctcctcaacaatgttccacctttttaaccagacacccaaatttgttgaagcagtgtcactcatgtaatgcctcgtatctcttccaaaaagataagttctacgatacaaagtacgatacaggtgacaagcacattcaatgtggtagacgtgcggatgtctttaagttttggttgatgtggaaggctaaaggaacattaggtttacaaagacatgcagagaaggttttcgaaatggccgaattttttactgagcaaattagacaaagagacgattttgaaatggtcgttgccgaacctgaatgtaccaatgtttgtttctggtatcttccaccttctttgagatcgtgtccaagagatgaggaatttttaactaaactccacagagtcgctccaaagattaaggaacgtatgatgaaagagggttcaatgatgatcacataccagcctttaagacaaaagccaaactttttcagattggttttgcaaaattcttccctcgaaaaatccgacatgttacatatcattaataagatcgcgcaattgggcgaagatctttag

SEQ ID NO: 201 - DNA, 1473 bp, Haplochromis sp.
  source 1..1473: mol_type = genomic DNA; note = Haplochromis burtoni
atggaacgtcacattaatggtgtgaagtcttgtgaatcccaagcgcctgttgcccaaccagaaaagtgttctccagcgggttcatttgacgtttcgtcctcaactgctgacgaacagattgcagaatcgtttattcgtcaggccatggagattattgtggacgaagccgtcaagaaggccaccaatgttcaagagaaggtttgtgaatggcattcacctgagcaactcaagaagttgttagatcttgaattgagagatagaggcgaacctgagtcggagattttgcagagatgccgtgatgccatccgttactcggttaagacctcccatcctagattttttaaccaattgtacgctggtatggaaccatattctatggtcgcttctttcattgcagaagcagttaaaacgaacctctacacttatgaggtcgcaccagtttttaccttgatggaagatgccgtcttaagaaagatgattaaagtcatcggttgggaagaaggtggcgatggaatctttaatccaggtggatctttgtcaaatatctacgcagtcaatcttgcaagatataggtactgtccagacttcaaagaagtcggtatgttctccgcacctagattagtgattttgacttctcaagaatgccattactctatctctaagggtgctgctttgatgggtatcggcacaaagaatgtctacacagttccaacagatgagagaggtagaatgatcccttctgcattagaagaaaaaattaagctcgcaaagtcggaaggagctgtcccttttatggttaatgccactgcaggtacaactgttttaggtgcgtttgacccaatcgacgaaattgccgatatttgtgagaagcataacttgtggttgcacgtcgatgcttgttggggtggtgccgcaattatgtcaaagaagcataaacatcttttaaagggtatccacagagttgactcggttgcttggaaccctcacaagatgttaatggcgtctttacaatgttctgcttttttagtcagagataaaacatccttgttgcagagatgtcattcggctagagcgtcctatttgttccaacaggataagttctatgatgtttcttacgacacgggagataagtcagttcaatgttctagaaagcctgatgctttcaaaatctggttgatgtggaaggcgttgggctccagagagttagagcaaagggttgatcgtgcattagcaatggctagatatcttgccgaggaaatcaaaaagagagaaggtttcaggttggtgttagagcctgaattcgcaaacgtgtgtttctggtatcttccaccatctttaagaaatctcccagaatccccagaattatggaaaaaattgcacaccgttaggtcatcatccttgcaaagggttattttaatgtactccaacgcagatatgaagagactcttctctttttcgttggctaacgcgtag

SEQ ID NO: 202 - DNA, 1371 bp, Pseudomonas sp.
  source 1..1371: mol_type = genomic DNA; note = Pseudomonas nunensis
atggaacttatgatcgttcaagtcacagagcaaacccagtctttaaagaagactgatgaaaagtacttgtggcacgcaatgagaggtgcagcgccatcccctacaaacttgatcattaccaaagcggaaggcgcatgggtcacagatattgacggtaatagatatttagatggaatgtctggattgtggtgtgtgaatgttggttacggtagaaaggaattggcaagagcggctttcgagcagttagaagagatgccatacttcccattgacccaatcccatgtcccagccatcaagttggcagaaaagttaaatgaatggttagatgatgagtacgtcatcttcttctctaattcgggttctgaagctaatgaaacagcgtttaagatcgcgagacaataccatcaacaaaagggtgaccacggaagatataagtttatttcaagatacagagcgtatcatggtaactccatgggtgcattagccgccactggtcaggcccaaaggaagtataagtatgagcctttgggtcaaggatttcttcacgtcgcgccacctgacacgtataggaatccagaagacgtccatacgttggcctcggcagaggagattgacagagtcatgacttgggagctctcccagacagtcgcaggcgtcattatggagccaatcatcaccggcggcggtatcctcatgccacctgatggttatatggaaaaggtgaaggaaatctgtgaaaagcatggtgcattattaatctgtgatgaagtcatctgtggattcggacgtactggcaagccttttggtttcatgaactatggcgtcaaaccagacattattactatggctaagggtatcacttccgcatatctccctttgtctgctactgctgtccgtagagaggtctacgaagctttcgttggttcggacgactacgatagattccgtcatgttaacacattcggcggcaatcctgccgcctgtgcactcgctttgaagaacttggaaattatggaaaatgagaagttgatcgagaggtccaaggaattgggtgaaagattgctctatgagttggaagacgtcaaagaacacccaaacgttggtgatgttagaggtaagggtttacttcttggtatcgagttggtcgaagacaaacaaaccaaagaaccagcatcgattgagaagatgaataaggttattaatgcatgcaaggagaaaggattgatcattggtaagaacggcgatactgtcgccggctataataacatcttgcagttagccccaccattgtcgattactgaggaagatttcacttttatcgtcaagacaatgaaggaatgcttagcccaacatcatcatcatcatcattag

SEQ ID NO: 203 - DNA, 1269 bp, Pseudomonas sp.
  source 1..1269: mol_type = genomic DNA; note = Pseudomonas nunensis
atgtcctcggaaaccattagtcaatcaattaatgtggttcatcctgtcactctttcacatggtaagaacgccgaagtctgggatacggaaggtaagagatacattgatttcgtcggtggaattggtgttttgaaccttggccattgtaatcctaggattgtcgaagcgatcagagaacaggcaacaaggttgactcattacgctttcaacgctgctccacacactccatatttggaattgatggacagattaactgcctttatcccagtctcatatccagtttctggaatgttaacaaatagtggcgccgaagctgccgaaaacgcattgaagatcgtgcgtggtgcgaccggtcgtaccgcagtcattgcattcgatggtgcctttcacggtagaactctcgctacactcaatttgaatggtaaagttgccccttacaaacaaaaggttggtgtcttaccaggtcctgtctaccatttaccatttccatctaaggataacggtgtgacatgtgctgaagcgctcaaggcaatggacagattattttctgttgaaatcgatgtcaatgacgtcgcatgtttcatcgtcgaacctgtgcaaggcgaagccggcttcttagcaatggatgtggaatttgctcaagcattgagaaggttctgtgacgaaaagaacatcttgttaattgcggatgaaattcaaagtggttttggtagaactggtgagcgttttgctttctccagattgggaatcgaaccagacttaatcttgttggccaagtccatcgctggtggtgttcctttgggcgccgtggtgggtagaaagtcgttattggatactttaccaaagggcggtttaggtggtacttattcaggtaaccctatcgcttgcgcggccgctcttgcaaccttggatgaaatgacggatgctaatttgaatgcttggggttcccaacaggaagaagccattgtctcccgttatgaggcatggagagtgtctaagttatcgccatacttaggtcgtttgacgggtgttggagcaatgagaggtatcgaactttccaatgctgatggttcaccagctagtgcgcaattgacccaactcttagcccttgcaagagacgcaggtttacttttgatgccttctggtaaatcaagaaacattattagattactcgcccctttgacgacagaagctactgttttagaagaaggtcttgatattttcgaggcgtgcttgaagaagctcacgcatcatcatcatcatcattag

SEQ ID NO: 204 - DNA, 1422 bp, Pichia sp.
  source 1..1422: mol_type = genomic DNA; note = Pichia kudriavzevii
atgtcgatctcagagaaatattttccacaagagccccaatctccagagttaaagactgccattcctggtccacaatctaaggctaagttggaagaattgtcagccgtttacgatactaaggccgcttactttgttacggactattataaatccttaggtaactacatcgtcgacgcagacggaaacaaattattggattcctactgccagatttcatccattgctctcggttacaacaatccagcgttgctcaaagttgcgcactcggatgaaatggcggttgctttgtgcaaccgtccagcacttgcttgcttcccttccaccgactactacgagatcttaaagaaaggtttgctttccgttgcgcctaagggtttggacaaagtctgtaccgcccacacaggttcggatgcaaacgagatggcatttaaggctgcatttctctttcaagcctcgaagaagagaggagataagccttttacttcggaagaacttgaatcagttatggaaaataagttgccaggaacatcagatatggtcatcttgtcatttgagaaaggtttccatggtagattgttcggttccttgtctaccaccagatccaaggcgatccataagctcgacattccagcatttgaatggccaaaagctccttttccccaattaaagtatccactcgaccaatttcaagcagaaaataaggcggaagaagagaggtgtttgaaggcactcgaagaaatcattgtcaactcgcctgcaaagattgccgcggcgatcattgaacctgtccagtcagaaggtggtgacaatcacgcgtcgcctgaattcttccaaggtatcagagaaatcacgaagaagcacggcgttatccttattgtcgatgaggttcagacaggcggtggtgccagtggtaaaatgtggttgcatgagcactacggcatcgtgcctgacattatgactttctctaagaaaatgcagaatgccggattcttcttttcggaagccggtttagcaggagaccagcctttccgtcaatttaacacctggtgtggtgatccatcgaaggctctcattgctagaacgattattgaggaaattaaggacaagaatttactcacttctgttacagaaacgggagactacctttactccaaattggaagccatttctgccaagtacgacaagatgatcaacttgagaggtaagggcaggggcttcttcatcgcgttcgatgctccaacacctgaacttagaaacaaattcattgctgagtgcaagaagcttggtttaaatatcggcggttgtggtgagcagggtgtcagattgcgtccagctttggtctttgaaaagaaacatgccgatattttggcatctatcattgatcaagcattttccaagatccatcatcatcatcatcattag

SEQ ID NO: 205 - DNA, 1320 bp, Burkholderia sp.
  source 1..1320: mol_type = genomic DNA; strain = Bp9004
atgagtttgaacgacgatgccacattttggcgtaatgcaaggcaacatttggtgagatatggtggtaccttcgaacctatgattattgaacgtgcgaagggttcctttgtttacgatgcagatggtagggcaatcttggatttcacatccggtcaaatgtccgcagtcttaggccactgtcatccagaaattgtctctgtcattggcgaatacgcaggcaagcttgaccacttattttcaggtatgttgtcgagaccagttgtcgatttggctactagactcgccgatatcactccagatggattggatagagctcttctcctctctacgggtgcagaatcgaacgaagcagcaattagaatggctaagttggttacaggaaagtacgagattgtcggttttgctcaaagttggcacggtatgactggtgcagcagcctcagcaacttactcagcgggtaggaaaggtgttggaccagctgctgtcggttcttttgcaattccagccccttttacttacagaccaagattcgaaagaaatggcgcctatgactatttggctgaattggactacgcattcgatctcattgatagacaatcatcgggtaacttagctgcattcattgctgaacccattctttcttctggtggtattatcgaattgccagaaggatatatggcagcattaaagagaaagtgtgaagcccgtggtatgttattgattttggatgaggcacaaacgggtgtcggtcgtactggtaccatgtttgcctgccaaagagacggagtcactccagatattctcactttgtcgaagacattgggtgcgggtttgcctttggccgcgatcgttacttcggcagcgatcgaagagagggcccatgagctcggttatttattctacacaacccatgtttctgatcctttaccagcagctgtcggattgagagtgttggatgttgttcaaagagacggattggttgctagagcaaacgtcatgggagaccgtttgaggagaggcttattggatttgatggaacaattcgattgtattggtgatgtcagaggtcgtggtttgttgttaggtgttgagatcgttaaggatagaagaacaaaagaaccagcagacggtctcggcgcaaagatcactcgtgaatgtatgaacttgggcttatccatgaatattgttcagttgcctggaatgggtggtgtctttagaattgcaccacctctcacggtctccgaagacgaaattgacttaggtttatctttattaggacaggccattgaacgtgcacttcatcatcatcatcatcattag

SEQ ID NO: 206 - DNA, 1341 bp, Mesorhizobium sp.
  source 1..1341: mol_type = genomic DNA; strain = Root552
atggaaaacgtgaaataccaaattccacctgccgaggtcttggacacattgggtaaatatatcttggttgatggtttccacactgtgatcgatcttgttaaatcccacgacaactatattgtggatgctgtcacgggacaggagattttagattgttacagttattttgcatcattgcctattggtcataatcaccccaaacttttctatgacgacttcttgaaggcgctcttcagagcgactatcgctaatccatctaactctgacatttactgcccagaatacgcatgggccgtcaagacattcgccgaattcgcgaagccagctgaattcaagtatatgttctttgttgcgggtggtgcaccagccattggtaacgccttgaaggttgcattcgactggagaacacgtaaggaaatgaacgccaattttctcccacctaaagcgggttacggtgttctccacttgaaggaagcgtttcacggcagactcggttacaccttgtccttgactaatacggatccagttaagaccgagtatttccctaagttcgattggccaagaatcactaaccctaagattagatttggtaaggatggtagaatctccaatgccactagtgtgcagagagccgagcgtgaagcgatgatccagattgaccgtgctatgagaagacaggaaattgctgctttgatcttggaaccaattcaaggtgaaggtggtgataaccattttagaccatgcttcttcaaagcgctcagacaaaagtgtgatgacaacgatatcatgttgatcttcgatgaagtgcaaaccggtgttggcttgaccggacgtatgtgggcttacgaacacttcggcgtcacacctgatattctcgttttcggtaagaagacccaggtttgtggtatcatggcaaccgagagagttgacgaggttcccgacaacgttttcagaaaatcaggcaggattaattcgacttggggagcaaatttggcggatttccttagaggtgcacgttatttagaaattatctgtgaggataacttagttgccaacgcagcaaccgttggtaagtatttcttggataagttacgtgccgaagtttacgaccctaagcatgcacaaaacatcaggggacgtggcttaatgattgcctttgatttcaaggacgcggcaacaagaaacgaatttagaagaaaggcttgggacaacggcttagcaacattgtcatgcggtacctattctgtccgttttagaccaccactcacgtttacaaaggagaacgtggatgaggcgattcgtatcatcaagaagtccttacatcatcatcatcatcattag

SEQ ID NO: 207 - DNA, 1407 bp, unidentified
  source 1..1407: mol_type = genomic DNA; note = Candidatus Falkowbacteria bacterium RIFOXYA2_FULL_47_19
atggaagacaaaagaaaaggtgagagaaagcgtgagagaaaggaagaacacggaatgggtactggagataaaatgaaccctcaaatgcaagagatcggtatgttagataccatcggccctagatcattggaagtcattgaagaagattgtaacgttatgtcagcttgtgagactagaccatatccactcgtcgtcgatagggcgaagggatccgtcgtgacagacttggacggtagagagtacattgactttgttgcaggcattgcagtcttgaatgccggtcattctaatccagcggttcaagcagcaatcagtgcgcaattggagaaaatgactcactgttgctcagcagacttcttcgccgaacccccattaaaattagccagaaagttaagagaactttcgggttatagtaaggttttctattgtaactctggtacggaagcagttgaagccgcaattaagttggccacttggaagaccggtaagcaaaatttgattggcttctacaacagtttccatggtaggactttgggttcattatcgttgacttcatcgaaagctagacagaaagagcactttccaggcattagaacagtccatactcattacgcatactgctacagatgtccattgaacttagaataccctggctgtggagttgagtgtgctaagcaaatcgaaaatttgatctttagaaaggaattgaacccagaagatacagctgccgtcttcgttgagccaattcagggtgaaggtggttacatcgtcccaccacctgagtttcacaaggagttaagaaagatctgtaccgataacgacgtccttttggctgcagacgaagtgcaagccggttgtttcagaacgggtcctttcttagctatggagaatttcggagtcaaaccagatatctcctgtcttgcaaaggcgcttggatcaggtttaccaatgggtgctatgttggctgatgctagtttaatggattggcccccaggtgttcattccaacacttttggtggtaacttgctcgcatccgcaggctcattggccgccttagaatttatggaaaaggagaaaacaggtgcccacgctaggtcgttaggctcacatattcaacaaagattaagggagttgcaagattcgtacccatgtattggagatgtcagaggcttgggtctcatgatcggtgctgaaatcgtgaaaccagacaaatctatcgatccagccaccaggaatagaattgttagggaaggttacaagcaaggaatcatgttgttaggttgtggagaaagtgtgattagattttccccaccattagtcatgacggaagaggaagcagataagggcttggagcgttttgaagaagccgttaaacgtggtgttcgtcatcatcatcatcatcattag

SEQ ID NO: 208 - DNA, 1299 bp, Klebsiella sp.
  source 1..1299: mol_type = genomic DNA; note = Klebsiella michiganensis
atgagtaaaagtgaaaacctctataatgcagctagagaactcattcctggtggagttaattcccctgttagagcctttacaggtgttggcggtacacctttgttcattgaaagggcagatggagcttacttgtacgatgtcgacggaaaagcatacattgactacgtgggttcatggggtcctatggtcttgggccataaccatcctgccatcagaaacgctgtcatcgaagccgcagagagaggcctttcgtttggcgcgccaacagaaatggaagttaagatggcagctttagttacggagttggttccaacaatggatatggttcgtatggtcaactcaggtacagaagctaccatgtctgcaatccgtttggcccgtggtttcactggtagagacaaaatcatcaagtttgaaggttgttaccatggtcacgcagactgcttgttggttaaggctggttcaggtgcattaactttgggtcaacccaatagtccaggtgttccagcggatttcgctaagcatacccttacttgtacatataatgacttgtcttccgtccgtgcagcctttgaacagtacccacaagatatcgcgtgtattattgttgagccagtcgcaggcaatatgaattgcattccccctttgcctgaattccttccaggtcttagagctttatgtgacgagtttggagcgcttttaatcatcgatgaagtgatgactggttttagagtggcccttgcaggtgctcaagattactacggagtggtgccagatctcacatgtttgggaaagatcattggtggtggtatgccagttggcgcctttggtggaagaagagaggttatggatgcattggcaccaactggccctgtttatcaagcaggtacgttgtcaggtaacccaatcgcgatggccgctggttttgcctgcttgacagaagtcgcccaacctggcgtccatgaaaccctcacagagttgaccaatcagttggcacagggtttattagacgcagcaagagatgcaggtatcccattggttgttaacaatgtcggtggaatgttcggtatcttctttaccgatgctccaacagtgacctgttaccaagatgttgtcaagtgtgatgttgaaaggtttaaaagattcttccacttaatgttgcaagaaggtgtgtatttagcaccatccgcatttgaagccggttttatgagtattgcccactcggaagaagacatcgataacactgtggctgccgctagaaacgcctttgcaaaattacatcatcatcatcatcattag

SEQ ID NO: 209 - protein, 598 aa, Terrapene sp.
  source 1..598: note = Terrapene carolina triunguis
MFLAIKSLQLSRQKQKIGRRKKSTDRWSDLLTDILQGIFLCCAVLQRSIDMDFRTGANKTNERVHLEPSQLREKHISSFQGESEEKEKDTNAEILVANFSNIYASDLLPTRNGEEFTDQFLLEVVEILLNYIKKTYDGKSKVLDFHHPHQLLEGLEGFSLELRDQPEPLEQILVDCRDTLKYGVKTGHPRYFNQLSSGLDIVGLAGEWLSATANTNMFTYEIAPVFVVMEEILLKKMHEMVGWGEMEADGIFSPGGTISNLYSVLVARYKRFPEIKTKGMAALPHISLFVSEHSHYSVKKAAAVLGIGTDNVIAVKCDERGKMIPSDLEAKILKVKRQDHIPLYVSATAGTTVYGAFDPLDNIADICEKYGLWMHVDAAWGGGLLMSRKHSFKLNGIERANSVTWNPHKLMGVPLQCSAFLVCEKGLLQACNQMCAGYLFQPDKQYDTTYDTGDKTIQCGRHVDVFKLWLMWKAKGTRGFEILINKSLELAEYLYKELNSRENFELVFDAEPELTNVCFWYIPPNLRRIPNGPEREKLLHQVAPKIKARMIEEGTTMVSYQPHGDKVNFFRMVFSNPATRKSDVDFLLEEIERLGKDL

SEQ ID NO: 210 - protein, 511 aa, unidentified
  source 1..511: note = Trichoderma gamsii
MPAISNGYVANGKPDSQLNRAAETADLLESVQQLIIPFIKAADDAAAHRASGNLGLDANGEPQNVLIDSQKPSELVARLKFLLPEEGLGKDGLLQSIDKLLKYSVNTWDQGFMDKLYASTTPVGVIADLILSVLNTNVHVYHVSPALTVIEKTTTKAFANLFNFDGPHAGGISCPGGSGSNLTSLIVARNTLYPETKTKGNGKRDFVVFTSAHGHYSVEKAAMITGLGSSAVWAVPVDNEGSMRPQALRELVLLAKKQGKTPFYVNSTAGTTVMGSFDPFEEISKVCKEFGMWLHIDASWGGSVIFSKAQSWKMKGSHLADSLTVNPHKMLNAPCTCSLLLGPDMRIFQRANSTAAGYLFHRGDDEEVWDLADLSLQCGRRGDALKVALAWLYYGANGFERQVDHAFDMASYLFRLLEESGDFVMVSSYPPPCLQVCFYSAPGGKLSSDKNVNTSRTQRIAHMLIGRGFMVDYAPGDKGSFLRVVVNVQTLEGTVEGIVKAMVELSQEISL

SEQ ID NO: 211 - protein, 505 aa, Varroa destructor
MCSSKEGTVVVEKLSYKDLYPYNINGKAETRAFLLKVVSILLDYVDEENDRTSKILDFKMPEELEQILDLNLPDKGLPLGDLLKDCRSTLKWQVKTGHPHFFNQLSSGLDIVSLAGEWLTSVANTNMFTYEIAPVFILMENVVLKKMREYLGFVHGDSILAPGGSISNMYAVMGARHKMFPNYKKLGLKALPQLVMYTSVDSHYSAMGAAASCGLGTDNVIQIGTDSMGRMRVDKLEEAVLATKAKGHVPFFVNCTCGTTVVGAFDPINPIADLCQKYGMWLHIDAAWGGGVLLSKKHRHLMAGVERANSVTWNPHKLMGTHLQCSTIHFRQDGILLGCNQMCAEYLFQQDKHYDVSFDTGDKVPQCGRRNDIFKLWLMWRAKGTEGFEAHIDQLFDISQYLVKRIKEMPEKFHLIQEPEMVNVLFWYIPQRLRDQPQSKERIAELGRITAQLKARMMNTGTLMITYQPLRELPNFFRNIVSNSAVTKDDIDFLLAEMDRLGNDL

SEQ ID NO: 212 - protein, 332 aa, Anopheles sinensis
MISFQTKGLFGLPRLVLFTSEDAHYSIKKLASFMGIGFDNVFAIKTDARGKMCLTDLEEQIKRALTEGAQPFMVSATAGTTVLGAFDPLEGIAELCAKYKLWMHVDAAWGGGALMSKKYRHLLKGIERADSVTWNPHKLLTAPQQCSTFLTRHENILSQCNSTNATYLFQKDKFYDTKYDTGDKHIQCGRRADVLKFWFMWRAKGTKGLEQHVDTAFDCAEFFTQTIKQRPGYKMVVHEPECTNVCFWYLPPSLQSRSEPKTAEEATEFRDALHRVAPKVKERMMKSGAMMITYQPIHDKPNFFRLVIQNSGLNHSDMIHIADTIEKLAADL

SEQ ID NO: 213 - protein, 513 aa, unidentified
  source 1..513: note = Apolygus lucorum
MPASEASAERPRADVVIEEIDGASSKEAFLSAPDKEIHGQFLHRVQQMLLDHAFQSTHRESKVTRYVDPDRLREIIDMNLRKEPASEEALLSYIRDTMKYSVKTGHPFFVNQLFSSLDPYALAGAWVTDSLNPSVYTYEVSPVLTLMEESVLKEMRSIVGWSDGDGIFAPGGSIANGYAISCARFHKIPDIKAKGLHGLPRLVLFTSEDAHYSIKKLAAFEGLGSDNVYPVKTDARGKMDVLHLRELVQRAKAEGAVPFMVSATSGTTVLGAFDPLEAIHEVCEEEGMWLHVDAAWGGGALVSKKYRHLLRGIEKADSVTWNPHKMLAAPQQCSTLLTKHKTILAECHSASAQYLFQKDKFYDTSFDTGDKHIQCGRRADVFKFWLMWKAKGTDGLEKHVNRVYENAEYFTEKIRRRPGFKLVLEEPECTNVTFWYVPPSIRGRDNEPEYNQKLHKVAPLIKERMTREGTMLMTYQPLKELPNFFRLVIQNSALTTSDMDYIVSEFERLGRNL

SEQ ID NO: 214 - protein, 496 aa, unidentified
  source 1..496: note = Strongyloides ratti
MNNLHATDLLPVNQDGWKETETFLKEVVEILLSYIKTQNDRSSKVLEFHHPQDMLKLIDLSIPDKPMSLDDLLNSCQEVLKLGVKTGHPRFFNQISCGLDLVCMAGEWLTATANTNMFTYEISPVFILMEKEVMRKMIQLIGWGDDEGDGIFSPGGAIANLYAMNAARHNAFPRCKPLGMTSVPTLCCFTSIDSHYSIKGAAAVTGIGTDNCFNIPTDKLGRMIPEALEQKIIECKKDGLYPFFVAATAGTTVYGAFDPLIQIADICEKYGVWFHVDAAWGGGLLLSPEHKYKLRGIERANSVTWNPHKLMGSLLQCSACFIRKDGLLFQTNQMSADYLFQQDKPYDVSYDTGDRSIQCGRRNDVLKLWLMWRSKGMEGYRKQINRLLDLAQYFTSKIKRTEGYEMVIEDPQFLNICFWYVPKRLRNMPQKEKMARLEKIAPKIKGKMMERGTTMVGYQPDNNTPNFFRLIINNPATTEEDLDFLIQEIIDIAIDM

SEQ ID NO: 215 - protein, 515 aa, Bactrocera dorsalis
MSSVTKTTATTQNKVDEIDKIRLNLADDWKVLGSVYDLLKDGKAFCVSDADRDAVKIVDFKQPNEMKQLIDLSIDENKTHTIAEIEDLCRLVIKYSVKTNHGRFHNQLFGQMDPYGLMGQWITDALNASAYTYEVGPVFSLMETEIIRTVCQLAGYAEGDGIFSPGGSMSNMYGLVLARYSRHPQVKTAGLFGMRPLALFTSEESHYSFVKAAHWLGLGADNCVVVKTNERGQMLTNDLEAKILKAQESGCEPFFVNATAGTTVLGAFDDLNAIADVCEKYGLWLHVDACLGGSALLSYRNRHLLAGLDRTNSFAWNPHKSLGAPLQCSLFLTRERDLLTRCNSIQVNYLFQQDKFYDVSYDTGNKSVQCGRKIDAFKFWLMLKARGYGSFGRLLDHAIDISKVLVEKIKQRGSEHFRLVLEDNQYMNVCFWYIPKALRGLEETPEWRERLYTVAPKIKERMAYSGTVMIGYTPLQYRGLGNFFRMVFTCFPVLEEAELDFILNEIERLGEECEY

SEQ ID NO: 216 - protein, 503 aa, Lutzomyia sp.
  source 1..503: note = Lutzomyia longipalpis
MLTILFRSWPHLWVLVPIMCTQYVQMHAVKWKWIILESEILRAKCEGGHPFMVSATAGTTVLGAFDPLTEIANLCEKYQLWFHVDAAWGGGALVSPKYRALLAGIERADSVTWNPHKLLAAPQQCSTFLTRHPNLLKQCHSCNASYLFQKDKFYDTKYDTGDKHIQCGRRADVFKFWLMWKAKGTLGLQRHAEKVFEMAEFFTEQIRQRDDFEMVVAEPECTNVCFWYLPPSLRSCPRDEEFLTKLHRVAPKIKERMMKEGSMMITYQPLRQKPNFFRLVLQNSSLEKSDMLHIINKIAQLGEDLADSVTWNPHKLLAAPQQCSTFLTRHPNLLKQCHSCNASYLFQKDKFYDTKYDTGDKHIQCGRRADVFKFWLMWKAKGTLGLQRHAEKVFEMAEFFTEQIRQRDDFEMVVAEPECTNVCFWYLPPSLRSCPRDEEFLTKLHRVAPKIKERMMKEGSMMITYQPLRQKPNFFRLVLQNSSLEKSDMLHIINKIAQLGEDL

SEQ ID NO: 217 - protein, 490 aa, Haplochromis sp.
  source 1..490: note = Haplochromis burtoni
MERHINGVKSCESQAPVAQPEKCSPAGSFDVSSSTADEQIAESFIRQAMEIIVDEAVKKATNVQEKVCEWHSPEQLKKLLDLELRDRGEPESEILQRCRDAIRYSVKTSHPRFFNQLYAGMEPYSMVASFIAEAVKTNLYTYEVAPVFTLMEDAVLRKMIKVIGWEEGGDGIFNPGGSLSNIYAVNLARYRYCPDFKEVGMFSAPRLVILTSQECHYSISKGAALMGIGTKNVYTVPTDERGRMIPSALEEKIKLAKSEGAVPFMVNATAGTTVLGAFDPIDEIADICEKHNLWLHVDACWGGAAIMSKKHKHLLKGIHRVDSVAWNPHKMLMASLQCSAFLVRDKTSLLQRCHSARASYLFQQDKFYDVSYDTGDKSVQCSRKPDAFKIWLMWKALGSRELEQRVDRALAMARYLAEEIKKREGFRLVLEPEFANVCFWYLPPSLRNLPESPELWKKLHTVRSSSLQRVILMYSNADMKRLFSFSLANA

SEQ ID NO: 218 - protein, 471 aa, unidentified
  source 1..471: note = Beauveria bassiana
MIVLVTGATAGFGESITRRFVKNGHKVIATGRRQERLLELKEELGDNLFTLQLDVRNRAAIEEALASLPAEWQHIDILVNNAGLALGMEPAHKANVDDWENMIDTNNKGLVYMTRAVLPGMVERNRGHIINIGSTAGSWPYAGGNVYGATKAFVRQFSLNLRTDLHKTAVRVTDIEPGMVGGTEFSNVRFKGDDDKAGKTYENANALTPEDVTEAVWWVATLPKHVNINTLEMMPQTSEDKKTMPLSREINPTQPVNQQIYHILRREIVRCQIAPGTPLSEKEVSGRFDVSRQPVREAFIKLAETGLVQIRPQRGTWVSKISVSHVRNGCFIREAIERAVVKRAAEKVNDSDLYALEHLLNQQRLAITDSQIDDFFSLDDKFHRQLALIADCSLAWDTVENIKATMDRVRYMSLDHVSPPAMLLEQHYAIFNALKAHDVAEVDTAMHLHLHEISQSILLIRQENQDWFSDE

SEQ ID NO: 219 - protein, 285 aa, Plautia stali
MPGSLSFATLSKQQAESGYELNTSHRQPDSTRQARNSEMIIFVTGATAGFGESITRRFIANGHKVIASGRRAERLKALKEELGANLYTVQLDVRNRAAIDEAIAALPAEWREIDVLVNNAGLALGIEPAHKANIEDWENMIDTNNKGLVYMTRAVLPAMVERNVGHIVNIGSIAGSWPYQGGNVYGATKAFVRQFSLNLRTDLHGTALRVTDIEPGLVGGTEFSNVRFKGDDGRADAVYEGTTALTAEDVTEAVYWVTTLPKHVNINTLEIMPVSQTLAGLKVHK

SEQ ID NO: 220 - protein, 248 aa, Erwinia mallotivora
MIVFVTGATAGFGESITRRFIAQGHKVVATGRRSERLQALKDELGENVYTLQLDVRNRAGIDAALHSLPAEWRDIDVLVNNAGLALGMAPAHQASIDDWEAMIDTNNKGLVFMTRAILPAMVERNVGHIINIGSTAGNWPYAGGNVYGATKAFVRQFSLNLRTDLHGTALRVTDIEPGLVGGTEFSNVRFKGDSDKADSVYDGTEPLTAEDVTEAVFWVASLPKHVNINTLEMMPVGQTLAGLKVHKA

SEQ ID NO: 221 - protein, 250 aa, Helicobacter pylori
MAHILVSGATSGFGLEIAKAFLQKNHVVFGTGRRKENLQELQLAYPKRFIPLCFDLQNKLETKRAIETIFSMTDRIDALINNAGLALGLNKAYECELDDWEVMIDTNIKGLLYLTRLILPSMIEHDQGTIINLGSIAGTYAYPGGNVYGASKAFVKQFSLNLRADLAGTNIRVSNVEPGLCGETEFSMVRFKGDKIKAQSVYENTLYLKPQDIANIVLWIYEQPSHVNINRIEIMPTSQTFAPLPTHKNP

SEQ ID NO: 222 - protein, 253 aa, Pseudomonas citronellolis
MTPTLFITGATSGFGEACARRFARDGWSLVITGRREDRLQALAKELSSQAEVLPITLDVRDRAAVAHAVDNLPAGFDKLRALINNAGLALGADPAQECDLDDWDTMVDTNIKGLMYCTRLLLPRLIAHGRGASILNLGSVAGQWPYPGSHVYGATKAFVEQFSLSLRCDLQGTGVRVSNLEPGLCESEFSLVRFGGDQARYDKTYAGTEALQPEDIAETIHWIVNQPPHININSLEIMPVSQAWAGFSVHRKG

SEQ ID NO: 223 - protein, 251 aa, Acetobacter sp.
  source 1..251: note = Acetobacter sicerae
MTDTVLVTGATAGFGRAISERLVKDGYRVIGTGRRAERLDALQASLGDAFLPLALDMLDKEALRGLPTELPEGWREVDVLINNAGLALGMDPAQTTNIDDWERMIGTNVSGMVEITRALLPGMVERNHGHVVSLGSTAGTYPYKGGNVYGASKAFVSQFMRNLRTDLLGTRVRATNLEPGLCGGSEFSNVRLGDEAKAADVYRGTKPILPEDIAETVAWVLGLPEHVNINRIEMMPVCQAAGGLSVVRDVD

SEQ ID NO: 224 - protein, 251 aa, Gluconobacter oxydans
MPLTVLVTGATAGFGHAIATRLVREGHRVIATGRRKERLEALKDELGESVLPYTLDMTDREALRALPAALPEEWQDIDVLVNNAGLALGMEPAQDAEPDNWESMIATNVSGVVELTRALLPGMVQRNTGYIIALGSTAGTYPYTGGNVYGATKAFVHQFTQNLRTDLLGTRIRVTNIEPGLCGGSEFSNVRLGDDAKAAAVYKDTTPLTPEDIAGTVSWLIGLPWHVNVNYLEMMPVCQASGGLAVNRHVG

SEQ ID NO: 225 - protein, 249 aa, unidentified
  source 1..249: note = Oleiagrimonas soli
MTKTAMVTGATSGFGAAIAERLVKDGWRVVICGRREERLQTMVDRLGADKAHAAAFDIRDAEAMSEALDALPAAFADIDLLVNNAGLALGTEPAQFADLDEWRQMIDTNVTALVTLTHALLPALIERRGAIVNISSISGSYPYKGGNVYGGTKAFVTQFSLGLRTDLHGTGVRVTSVEPGMAETEFTLVRTGGDQAASDKLYTGAHPITAEDIAETVAWIAHLPPHLNVNRIEVMPTSQSAAGLQVHRD

SEQ ID NO: 226 - protein, 254 aa, Geobacter sp.
  source 1..254: note = Geobacter soli
MSRTVFITGASAGFGAACARLFADRGDRLVLAARREEPLLALRDELAPQAEVHTVTLDVRRRKAVQEAVEGLPDAFSRIDILVNNAGLALGLEPAHETNPDDWDAMVDTNIKGLIYCTRFLLPGMVARNRGHIVNIGSVAGSWPYPGGNVYGGTKAFVEQFSRNLRADLLGKRVRVTCLAPGMAETEFSMVRFKGDAERAAQVYAGTQALTAEDIAGIVAWVTELPPHVNVNALEVMSIFQSWGPLAVHRPGAS

SEQ ID NO: 227 - protein, 248 aa, Campylobacter concisus
MKKTAFVTGATSGFGEAIARRLSKEGYKIVALARREDRLKKLASELGDTHIIVADIRDKEAVFDAVDSLPENFKDIEVLVNNAGMALGLEKTIDAKVEDFEAMIDTNVKGLIYSTKAVLPLLYKQEKGYIFNIGSTAGSWPYPGSNVYGATKAFVKQFSLNLRNDLVGTNIRVTNIEPGLCKTEFSEVRFRGDKAKADSLYENTNFITSEDIATILVNCLNMPGSVNINRVEVMANTQTWAGLAIEKF

SEQ ID NO: 228 - protein, 456 aa, Pseudomonas sp.
  source 1..456: note = Pseudomonas nunensis
MELMIVQVTEQTQSLKKTDEKYLWHAMRGAAPSPTNLIITKAEGAWVTDIDGNRYLDGMSGLWCVNVGYGRKELARAAFEQLEEMPYFPLTQSHVPAIKLAEKLNEWLDDEYVIFFSNSGSEANETAFKIARQYHQQKGDHGRYKFISRYRAYHGNSMGALAATGQAQRKYKYEPLGQGFLHVAPPDTYRNPEDVHTLASAEEIDRVMTWELSQTVAGVIMEPIITGGGILMPPDGYMEKVKEICEKHGALLICDEVICGFGRTGKPFGFMNYGVKPDIITMAKGITSAYLPLSATAVRREVYEAFVGSDDYDRFRHVNTFGGNPAACALALKNLEIMENEKLIERSKELGERLLYELEDVKEHPNVGDVRGKGLLLGIELVEDKQTKEPASIEKMNKVINACKEKGLIIGKNGDTVAGYNNILQLAPPLSITEEDFTFIVKTMKECLAQHHHHHH

SEQ ID NO: 229 - protein, 422 aa, Pseudomonas sp.
  source 1..422: note = Pseudomonas nunensis
MSSETISQSINVVHPVTLSHGKNAEVWDTEGKRYIDFVGGIGVLNLGHCNPRIVEAIREQATRLTHYAFNAAPHTPYLELMDRLTAFIPVSYPVSGMLTNSGAEAAENALKIVRGATGRTAVIAFDGAFHGRTLATLNLNGKVAPYKQKVGVLPGPVYHLPFPSKDNGVTCAEALKAMDRLFSVEIDVNDVACFIVEPVQGEAGFLAMDVEFAQALRRFCDEKNILLIADEIQSGFGRTGERFAFSRLGIEPDLILLAKSIAGGVPLGAVVGRKSLLDTLPKGGLGGTYSGNPIACAAALATLDEMTDANLNAWGSQQEEAIVSRYEAWRVSKLSPYLGRLTGVGAMRGIELSNADGSPASAQLTQLLALARDAGLLLMPSGKSRNIIRLLAPLTTEATVLEEGLDIFEACLKKLTHHHHHH

SEQ ID NO: 230 - protein, 473 aa, Pichia sp.
  source 1..473: note = Pichia kudriavzevii
MSISEKYFPQEPQSPELKTAIPGPQSKAKLEELSAVYDTKAAYFVTDYYKSLGNYIVDADGNKLLDSYCQISSIALGYNNPALLKVAHSDEMAVALCNRPALACFPSTDYYEILKKGLLSVAPKGLDKVCTAHTGSDANEMAFKAAFLFQASKKRGDKPFTSEELESVMENKLPGTSDMVILSFEKGFHGRLFGSLSTTRSKAIHKLDIPAFEWPKAPFPQLKYPLDQFQAENKAEEERCLKALEEIIVNSPAKIAAAIIEPVQSEGGDNHASPEFFQGIREITKKHGVILIVDEVQTGGGASGKMWLHEHYGIVPDIMTFSKKMQNAGFFFSEAGLAGDQPFRQFNTWCGDPSKALIARTIIEEIKDKNLLTSVTETGDYLYSKLEAISAKYDKMINLRGKGRGFFIAFDAPTPELRNKFIAECKKLGLNIGGCGEQGVRLRPALVFEKKHADILASIIDQAFSKIHHHHHH

SEQ ID NO: 231 - protein, 439 aa, Burkholderia sp.
MSLNDDATFWRNARQHLVRYGGTFEPMIIERAKGSFVYDADGRAILDFTSGQMSAVLGHCHPEIVSVIGEYAGKLDHLFSGMLSRPVVDLATRLADITPDGLDRALLLSTGAESNEAAIRMAKLVTGKYEIVGFAQSWHGMTGAAASATYSAGRKGVGPAAVGSFAIPAPFTYRPRFERNGAYDYLAELDYAFDLIDRQSSGNLAAFIAEPILSSGGIIELPEGYMAALKRKCEARGMLLILDEAQTGVGRTGTMFACQRDGVTPDILTLSKTLGAGLPLAAIVTSAAIEERAHELGYLFYTTHVSDPLPAAVGLRVLDVVQRDGLVARANVMGDRLRRGLLDLMEQFDCIGDVRGRGLLLGVEIVKDRRTKEPADGLGAKITRECMNLGLSMNIVQLPGMGGVFRIAPPLTVSEDEIDLGLSLLGQAIERALHHHHHH

SEQ ID NO: 232 - protein, 446 aa, Mesorhizobium sp.
MENVKYQIPPAEVLDTLGKYILVDGFHTVIDLVKSHDNYIVDAVTGQEILDCYSYFASLPIGHNHPKLFYDDFLKALFRATIANPSNSDIYCPEYAWAVKTFAEFAKPAEFKYMFFVAGGAPAIGNALKVAFDWRTRKEMNANFLPPKAGYGVLHLKEAFHGRLGYTLSLTNTDPVKTEYFPKFDWPRITNPKIRFGKDGRISNATSVQRAEREAMIQIDRAMRRQEIAALILEPIQGEGGDNHFRPCFFKALRQKCDDNDIMLIFDEVQTGVGLTGRMWAYEHFGVTPDILVFGKKTQVCGIMATERVDEVPDNVFRKSGRINSTWGANLADFLRGARYLEIICEDNLVANAATVGKYFLDKLRAEVYDPKHAQNIRGRGLMIAFDFKDAATRNEFRRKAWDNGLATLSCGTYSVRFRPPLTFTKENVDEAIRIIKKSLHHHHHH

SEQ ID NO: 233 - protein, 468 aa, unidentified
  source 1..468: note = Candidatus Falkowbacteria bacterium RIFOXYA2_FULL_47_19
MEDKRKGERKRERKEEHGMGTGDKMNPQMQEIGMLDTIGPRSLEVIEEDCNVMSACETRPYPLVVDRAKGSVVTDLDGREYIDFVAGIAVLNAGHSNPAVQAAISAQLEKMTHCCSADFFAEPPLKLARKLRELSGYSKVFYCNSGTEAVEAAIKLATWKTGKQNLIGFYNSFHGRTLGSLSLTSSKARQKEHFPGIRTVHTHYAYCYRCPLNLEYPGCGVECAKQIENLIFRKELNPEDTAAVFVEPIQGEGGYIVPPPEFHKELRKICTDNDVLLAADEVQAGCFRTGPFLAMENFGVKPDISCLAKALGSGLPMGAMLADASLMDWPPGVHSNTFGGNLLASAGSLAALEFMEKEKTGAHARSLGSHIQQRLRELQDSYPCIGDVRGLGLMIGAEIVKPDKSIDPATRNRIVREGYKQGIMLLGCGESVIRFSPPLVMTEEEADKGLERFEEAVKRGVRHHHHHH

SEQ ID NO: 234 - protein, 432 aa, Klebsiella sp.
  source 1..432: note = Klebsiella michiganensis
MSKSENLYNAARELIPGGVNSPVRAFTGVGGTPLFIERADGAYLYDVDGKAYIDYVGSWGPMVLGHNHPAIRNAVIEAAERGLSFGAPTEMEVKMAALVTELVPTMDMVRMVNSGTEATMSAIRLARGFTGRDKIIKFEGCYHGHADCLLVKAGSGALTLGQPNSPGVPADFAKHTLTCTYNDLSSVRAAFEQYPQDIACIIVEPVAGNMNCIPPLPEFLPGLRALCDEFGALLIIDEVMTGFRVALAGAQDYYGVVPDLTCLGKIIGGGMPVGAFGGRREVMDALAPTGPVYQAGTLSGNPIAMAAGFACLTEVAQPGVHETLTELTNQLAQGLLDAARDAGIPLVVNNVGGMFGIFFTDAPTVTCYQDVVKCDVERFKRFFHLMLQEGVYLAPSAFEAGFMSIAHSEEDIDNTVAAARNAFAKLHHHHHH

SEQ ID NO: 235 - protein, 597 aa, synthetic construct
MNMKEKSFGPRQFKGNLQQHKQTILKTVLFLGLIWLFFCSYQSQGSSISKPSSKKFQKFFKKYFNELDKAIVHGDKLMFGTRANAEMENIYADVSETELKELEDFSPEIIKQLKVNHRKIVSFIPKEIHVEGYCGSGYVTIGGGNNDYKSMLLIKQLRETGAKNPIEVIIPPFYEPNLNLCDNIFPSYDATCVFMNGIFGKKNLQKLRYNSKLIGTLAIIASSFENALFISPDTIPLHNPDHLFEGKVISQFGLIVWPSVYRTSITPSYYEISDIQVENNPVRILNDRWVSPEDIHQHLQAKAQRDHENTQLSFHDLKGTSSDYAATSDLLLINKRKNLDVLLLMLYYTNDGPSRYFPLLNAVSSKGNGDYIVAAAHYFKKPYYQLFKTADKIGEEINSLTQVTSITGHYDPNEDYELLKGNLLSLFEQRRELKNDFKYDYFEKIRRPFNVFTSTPLFFHVEETLHPFKMKNSKVYKDNTGNQRRIFGNHIKFYGCDFELKLITSINELLCGAESTDFTFAGFENVDVLCSDYIQPRMEFLQGNSKQFWNKYVFEKRPPNSVPQTSVSTIEKAIKEGFDRDVEYQKDEPARPSILYL

SEQ ID NO: 236 - protein, 400 aa, synthetic construct
MLSIYKSIRLAKPDPILGLSAAFLRDPSPSKINLGVGAYRDNNGNPWTLPSVALAEEALARSHGNHEYVPILGSQKYIDSVQEFLFKEDKLGQLLREGKILTGQGLSGTGSLRVLGEFMHSHLKIKTIHVSTPTWPNHVNVFTNSSLKVAKYPYYDPQTKGLAFHEMLDSLSKVPYGDAILLHSCCHNPTGVDPTQDQWREILAIIKKRQLFPVIDMAYQGFAESLQKDMFVPQLTAKLGIPFILSQSFAKNMGLYGERIGSFSIILNDESEFEPVKSQISIVARAMYSSPPKHGAEIVSYILTNDKLLAQWETEVSIMRDRLANMRHSLYEELTRRTDNDWSHLIKQKGMFAFTGLNVNQVHKLREKNVYLTDNGRISIAGINNHNVNHLADAIAQVCK

SEQ ID NO: 237 - protein, 451 aa, synthetic construct
MELMIVQVTEQTQSLKKTDEKYLWAAPSPTNLIITKAEGAWVTDIDGNRYLDGMSGLWCVNVGYGRKELARAAFEQLEEMPYFPLTQSHVPAIKLAEKLNEWLDDEYVIFFSNSGSEANETAFKIARQYHQQKGDHGRYKFISRYRAYHGNSMGALAATGQAQRKYKYEPLGQGFLHVAPPDTYRNPEDVHTLASAEEIDRVMTWELSQTVAGVIMEPIITGGGILMPPDGYMEKVKEICEKHGALLICDEVICGFGRTGKPFGFMNYGVKPDIITMAKGITSAYLPLSATAVRREVYEAFVGSDDYDRFRHVNTFGGNPAACALALKNLEIMENEKLIERSKELGERLLYELEDVKEHPNVGDVRGKGLLLGIELVEDKQTKEPASIEKMNKVINACKEKGLIIGKNGDTVAGYNNILQLAPPLSITEEDFTFIVKTMKECLAQHHHHHH

SEQ ID NO: 238 - protein, 428 aa, synthetic construct
MENVKYQIPPAEVLDTLGKYILVDGFHTVIDLVKSHDNYIVDAVTGQEILDCYSYFASLPIGHNHPKLLKALMENVKYQIPPAEVLDTLGKYILVDGFHTVIDLVKSHDNYIVDAVTGQEILDCYSYFASLPIGHNHPKLLKALAFHGRLGYTLSLTNTDPVKTEYFPKFDWPRITNPKIRFGKDGRISNATSVQRAEREAMIQIDRQEIAALILEPIQGEGGDNHFRPCFFKALRQKCDDNDIMLIFDEVQTGVGLTGRMWAYEHFGVTPDILVFGKKTQVCGIMATERVDEVPDNVFRKSGRINSTWGANLADFLRGARYLEIICEDNLVANAATVGKYFLDKLRAEVYDPKHAQNIRGRGLMIAFDFKDAATRNEFRRKAWDNGLATLSCGTYSVRFRPPLTFTKENVDEAIRIIKKSLHHHHHH

SEQ ID NO: 239 - protein, 247 aa, synthetic construct
MIVLVTGATAGFGESITRRFVKNGHKVIATGRRQERLLELKEELGDNLFTLQLDVRNRAAIEEALASLPAEWQHIDILVNNAGLALGMEPAHKANVDDWENMIDTNNKGLVYMTRAVLPGMVERNRGHIINIGSTAGSWPYAGGNVYGATKAFVRQFSLNLRTDLHKTAVRVTDIEPGMVGGTEFSNVRFKGDDDKAGKTYENANALTPEDVTEAVWWVATLPKHVNINTLEMMPQTSEDKKTMPLS